SEQUENCE LISTING (21 sequences)

SEQ ID NO: 1 - protein, 5 aa, Artificial Sequence
  Artificially synthesized peptide sequence
  MOD_RES 5: Xaa = glycinamide
ALGPX

SEQ ID NO: 2 - protein, 146 aa, Phaseolus vulgaris
MGAFTEKQEALVNSSWEAFKGNIPQYSVVFYTSILEKAPAAKNLFSFLANGVDPTNPKLTAHAESLFGLVRDSAAQLRANGAVVADAALGSIHSQKALNDSQFLVVKEALLKTLKEAVGDKWTDELSTALELAYDEFAAGIKKAYA

SEQ ID NO: 3 - DNA, 756 bp, Phaseolus vulgaris
atgggcgccttcaccgagaagcaggaggccctggtgaacagcagctggccttcaagggcaacatcccccagtacagcgtggtgttctacaccagcaccgggaccacttgtcgtcgaccctccggaagttcccgttgtagggggttcgcaccacaagatgtggtcgtggagaaggcccccgccgccaagaacctgttcagcttcctggccaacgggaccccaccaaccccaagctgaccgcccacgccgagagcctgttaggacctcttccgggggcggcggttcacaagtcgaaggaccggttgccgcacctggggtggttggggttcgactggtgcggctctcggacaacggctgcgcgacagcgccgcccagctgcgcgccaacggcgccgtggtggccgcgccctgggcagcatccacagccagaaggccctgaacgacgccggaccacgcgctgtcgcggcgggtcgacggttgccgcggcaccaccggctgcggcgggacccgtcgtaggtgtcgtccgggacttgctgagccagttgtggtgaaggaggccctgctgaagaccctgaaggaggccgtgggcgacggaccgacgagctgagcaccgccctggagctggccttcggtcaaggaccaccacttcctccgggacgactggacttcctccggcacccgctgttcacctggctgctcgactcgtggcgctcgaccggatgctgctcaagcggcggccgtagttcttccggatgcgg

SEQ ID NO: 4 - DNA, 48 bp, Phaseolus vulgaris
atgggcgccttcaccgagaagcaggaggccctggtgaacagcagctgg

SEQ ID NO: 5 - DNA, 48 bp, Phaseolus vulgaris
tgggggatgttgcccttgaaggcctcccagctgctgttcaccagggcc

SEQ ID NO: 6 - DNA, 48 bp, Phaseolus vulgaris
ccttcaagggcaacatcccccagtacagcgtggtgttctacaccagca

SEQ ID NO: 7 - DNA, 48 bp, Phaseolus vulgaris
cttggcggcgggggccttctccaggatgctggtgtagaacaccacgct

SEQ ID NO: 8 - DNA, 48 bp, Phaseolus vulgaris
ggagaaggcccccgccgccaagaacctgttcagcttcctggccaacgg

SEQ ID NO: 9 - DNA, 48 bp, Phaseolus vulgaris
tcagcttggggttggtggggtccacgccgttggccaggaagctgaaca

SEQ ID NO: 10 - DNA, 48 bp, Phaseolus vulgaris
gaccccaccaaccccaagctgaccgcccacgccgagagcctgttcggc

SEQ ID NO: 11 - DNA, 48 bp, Phaseolus vulgaris
agctgggcggcgctgtcgcgcaccaggccgaacaggctctcggcgtgg

SEQ ID NO: 12 - DNA, 48 bp, Phaseolus vulgaris
tgcgcgacagcgccgcccagctgcgcgccaacggcgccgtggtggccg

SEQ ID NO: 13 - DNA, 48 bp, Phaseolus vulgaris
gctgtggatgctgcccagggcggcgtcggccaccacggcgccgttggc

SEQ ID NO: 14 - DNA, 48 bp, Phaseolus vulgaris
cgccctgggcagcatccacagccagaaggccctgaacgacagccagtt

SEQ ID NO: 15 - DNA, 48 bp, Phaseolus vulgaris
tcagcagggcctccttcaccaccaggaactggctgtcgttcagggcct

SEQ ID NO: 16 - DNA, 48 bp, Phaseolus vulgaris
gtggtgaaggaggccctgctgaagaccctgaaggaggccgtgggcgac

SEQ ID NO: 17 - DNA, 48 bp, Phaseolus vulgaris
gcggtgctcagctcgtcggtccacttgtcgcccacggcctccttcagg

SEQ ID NO: 18 - DNA, 48 bp, Phaseolus vulgaris
ggaccgacgagctgagcaccgccctggagctggcctacgacgagttcg

SEQ ID NO: 19 - DNA, 48 bp, Phaseolus vulgaris
ggcgtaggccttcttgatgccggcggcgaactcgtcgtaggccagctc

SEQ ID NO: 20 - DNA, 31 bp, Artificial Sequence
  Cloning oligonucleotide
aagaattctttctcgcacaagaaattattcg

SEQ ID NO: 21 - DNA, 26 bp, Artificial Sequence
  Cloning oligonucleotide
aagtcgacttattcgctgatacggcg

(19) United States
(12) Patent Application Publication
(10) Pub. No.: US 2008/0076824 A1
(43) Pub. Date: Mar. 27, 2008

(54) Compounds That Inhibit Replication Of Human Immunodeficiency Virus

(76) Inventors: Jan Maria Rene Balzarini, Heverlee (BE); Anders Vahlne, Bastad (SE); Marita Hogberg, Tullinge (SE); Weimin Tong, Uppsala (SE)

Correspondence Address: KNOBBE MARTENS OLSON & BEAR LLP, 2040 MAIN STREET, FOURTEENTH FLOOR, IRVINE, CA, 92614, US

(21) Appl. No.: 11/660,429

(22) PCT Filed: Aug. 16, 2005
(86) PCT No.: PCT/IB05/03755

§ 371 Date: Oct. 8, 2007

Related U.S. Application Data
(63) Continuation-in-part of application No. 10/920,831, filed on Aug. 18, 2004.

(51) Int. Cl. A61K 31/19; A61P 31/18; C12N 7/06

(52) U.S. Cl. 514/563; 435/238

(57) ABSTRACT

The present invention relates to the discovery of a novel class of compounds that inhibit the replication of human immunodeficiency virus (HIV) and approaches to identify these compounds. More specifically, it has been found that enzymatically prepared alpha-hydroxyglycinamide and synthetically prepared alpha-hydroxyglycinamide inhibit the replication of HIV in human serum. Embodiments include methods to identify modified glycinamide compounds that inhibit HUV, methods to isolate and synthesize modified glycinamide compounds, and therapeutic compositions comprising these compounds.

### FIELD OF THE INVENTION

Compounds that inhibit the replication of human immunodeficiency virus (HIV) have been discovered. Several methods to make and use these compounds are described. Embodiments include pharmaceuticals and dietary supplements that contain modified glycinamide compounds and compounds that contain modified glycinamide, methods to identify and synthesize these compounds and methods of using these compounds to inhibit HIV replication and/or treat or prevent HIV infection.

### BACKGROUND OF THE INVENTION

Human immunodeficiency virus (HIV) is the name given to a lentivirus that infects humans and that causes acquired immuno-deficiency syndrome (AIDS). HIV is a complex retrovirus containing at least nine genes. The viral structural genes, designated gag, pol, and env, respectively code for inter alia the viral core proteins, reverse transcriptase, and the viral glycoproteins of the viral envelope. The remaining HIV genes are accessory genes involved in viral replication. The gag and env genes encode polyproteins, i.e., the proteins synthesized from each of these genes are post-translationally cleaved into several smaller proteins.

Although the overall shape of HIV is spherical, the nucleocapsid is asymmetrical having a long dimension of about 100 nm, a wide free end about 40-60 nm, and a narrow end about 20 nm in width. The nucleocapsid within each mature virion is composed of two molecules of the viral single-stranded RNA genome encapsulated by proteins proteolytically processed from the Gag precursor polypeptide. Cleavage of the gag gene polyprotein Pr55^{gag} by a viral coded protease (PR) produces mature capsid proteins.

Since the discovery of HIV-1 as the etiologic agent of AIDS, significant progress has been made in understanding the mechanisms by which the virus causes disease. While many diagnostic tests have been developed, progress in HIV vaccine therapy has been slow largely due to the heterogeneous nature of the virus and the lack of suitable animal models. (See e.g., Martin, Nature, 345:572-573 (1990)).

A variety of pharmaceutical agents have been used in attempts to treat AIDS. HIV reverse transcriptase (RT) is one drug target because of its crucial role in viral replication, however, many, if not all, of the drugs that inhibit the enzyme are limited in their usefulness as therapeutic agents. These are nucleoside/nucleotide analogue RT inhibitors (NRTI:s) that will induce chain termination and agents that directly inhibit the enzyme, referred to as non-nucleoside analogue RT inhibitors (NNRTI:s). Nucleoside derivatives, such as azidothymidine (AZT, zidovudine®) and the other RT inhibitors cause serious side effects such that many patients cannot tolerate administration.

Another drug target is the HIV protease (PR) crucial to virus maturation. PR is an aspartic acid protease and can be inhibited by synthetic compounds. (See e.g., Richards, FEBS Lett., 253:214-216 (1989)). Protease inhibitors strongly inhibit the replication of HIV but prolonged therapy has been associated with metabolic diseases such as lipodystrophy, hyperlipidemia, and insulin resistance.

Additionally, HIV quickly develops resistance to NRTI:s, NNRT:s and protease inhibitors. Resistant virus can also spread between patients. Studies have shown, for example, that in the US one tenth to one fifth of the individuals recently infected by HIV already have virus that has developed resistance to one or more antiviral drug, probably because they were infected by a person that at the time of transmission carried a virus that had developed resistance.

Over the last decade several peptide amides that inhibit the replication of HIV have been discovered. (See, e.g., U.S. Pat. Nos. 5,627,035; 6,258,932; 6,455,670; and U.S. patent application Ser. Nos. 09/827,822; 09/938,806; 10/072,783; 10/217,933; and 10/235,158, all of which are herein expressly incorporated by reference in their entireties). These peptide amides appear to inhibit HIV replication in a manner that is different than reverse transcriptase inhibitors and protease inhibitors. The need for more agents that inhibit HIV replication is manifest.

### BRIEF SUMMARY OF THE INVENTION

Several compounds that inhibit replication of HIV have been discovered. Enzymatically prepared and synthetically prepared α-hydroxyglycinamide and compounds that contain this moeity have been found to inhibit replication of HIV. Accordingly, embodiments of the invention include compositions that consist, consist essentially of, or comprise modified glycinamide compounds. The term “modified glycinamide,” depending on the context, includes but is not limited to, glycinamide and glycinamide containing compounds, wherein the alpha carbon has been modified (e.g., oxidized). Modified glycinamides include, for example, alpha hydroxyglycinamide (also referred to as α-hydroxyglycinamide or AlphaHGA), or, amino acid conjugated or derivatized alphahydroxyglycinamide (e.g., Valine-alphahydroxyglycinamide), metabolites of glycinamide (also referred to as Metabolite X), the compounds of formulas A, B, C, D, E, F, G, H, I, J, K, L, M, N, O, P, Q, R, S, T, U, V, W, X, Y, Z, and AA) in either enantiomer (L or D) or both or either isomer (R or S) or both and mixtures of said enantiomers and/or isomers (e.g., 50% L or D or R or S).

The antiretroviral compounds described herein are provided as active ingredients of pharmaceuticals, dietary supplements, and medicaments (e.g., powders, liquids, intravenous or transdermal solutions, elixirs, tablets, pills, gelcaps, capsules, aerosols, inhalable preparations, sublingual preparations, gums, or patches) that inhibit the replication and/or propagation of HIV and/or improve the immune system in an individual (e.g., raise T cell count). Modified glycinamide compounds, such as α-hydroxyglycinamide (alpha-hydroxy-gly-NH₂, provided by formula C), α-peroxyglycinamide dimer (NH₂-gly-O—O-gly-NH₂, provided by formula E), diglycinamide ether (NH₂-gly-O-gly-NH₂, provided by formula F) and alpha-methoxyglycinamide (alpha-MeO-gly-NH₂, provided by formula G), the compounds of formulas K and M, and amino acid conjugated or derivatized alphahydroxyglycinamide (e.g., Valine-alphahydroxyglycinamide) or pharmaceutically acceptable salts thereof are preferred active ingredients. Particularly preferred active ingredients include compounds of formulas K and M and amino acid derivatized alphahydroxyglycinamide or amino acid-conjugated alphahydroxyglycinamide (e.g., Valine-alphahydroxyglycinamide, such as shown by formula Y). That is, particularly preferred active ingredients include Xⁿ-Z, Xⁿ¹-Z-Xⁿ², or Z-Xⁿ, wherein X is any amino acid, amino acid derivative, or modified amino acid, n, n1, and n2 are any integer; preferably, at least, equal to, or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 100, 125, 150, 175, 200, 250, 300, 350, 400, 450, 500, 550, 600, 700, 800, 900, 1000, 1500, 2000, 2500, 3000, 3500, 4000, 4500, 5000, 6000, 7000, 8000, 9000, or 10,000, and Z is alphahydroxyglycinamide.

The antiretroviral pharmaceuticals, dietary supplement, and medicaments of the invention can be provided in unit dosage form (e.g., tablets, capsules, gelcaps, liquid doses, injectable doses, transdermal or intranasal doses) and can contain, in addition to one or more of the antiretroviral compounds or prodrugs described herein, a pharmaceutically acceptable carrier, stabilizing agent (e.g., antioxidant), exipient, flavoring, cofactor, or catalyst. Containers comprising said compositions and formulations (e.g., sterile vials, septum sealed vials, bottles, jars, cans, syringes, atomizers, swabs) whether in bulk or in individual doses are also embodiments and, preferably, said compositions and formulations are prepared according to certified good manufacturing processes (GMP) (e.g., suitable for or accepted by a governmental regulatory body, such as the Federal Drug Administration (FDA)) and said containers can comprise a label or other indicia that reflects approval of said formulation from said governmental regulatory body. Dietary supplements containing said compounds with or without structure-function indicia, also made according to GMP, are embodiments.

Methods of selling or marketing a pharmaceutical or dietary supplement are also embodiments. By one approach, a market or plurality of consumers is identified as being in need of a composition that inhibits HIV replication or a composition that improves immune system function or a composition that promotes the general health and welfare of an individual. The identified market or plurality of consumers is then provided a product that is suitable for human consumption and said product comprises one or more of the compounds provided by formulas A, B, C, D, E, F, G, H, I, J, K, L, M, N, O, P, Q, R, S, T, U, V, W, X, Y, Z, and AA) in either enantiomer (L or D) or both or either isomer (R or S) or both and mixtures of said enantiomers and/or isomers (e.g., 50% L or D or R or S). In some embodiments, the product comprises preferred active ingredients include Xⁿ-Z, Xⁿ¹-Z-Xⁿ², or Z-Xⁿ, wherein X is any amino acid, amino acid derivative, or modified amino acid, n, n1, and n2 are any integer; preferably, at least, equal to, or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 100, 125, 150, 175, 200, 250, 300, 350, 400, 450, 500, 550, 600, 700, 800, 900, 1000, 1500, 2000, 2500, 3000, 3500, 4000, 4500, 5000, 6000, 7000, 8000, 9000, or 10,000, and Z is alphahydroxyglycinamide. Optionally, the method includes the step of measuring the number of products sold and/or a step of providing an advertising or promotional material (e.g., an electronic or written publication or website), wherein said advertising or promotional material discloses that said product inhibits HIV replication, improves the immune system of said consumer, or improves the general health and welfare of a consumer.

Some embodiments, as described above, include a precursor prodrug for one or more of the antiretroviral compounds described herein and one or more cofactors or catalysts that convert said prodrug into an antiretroviral active ingredient. Such precursors or prodrugs can include, for example, a glycinamide containing peptide or glycinamide itself. For example, some prodrugs include, but are not limited to: Gly-NH₂, Pro-Gly-NH₂, Gly-Pro-Gly-NH₂, Gly-Lys-Gly-NH₂, Arg-Gln-Gly-NH₂, Cys-Gln-Gly-NH₂, Lys-Gln-Gly-NH₂, Ala-Leu-Gly-NH₂, Gly-Val-Gly-NH₂, Val-Gly-Gly-NH₂, Ala-Ser-Gly-NH₂, Ser-Leu-Gly-NH₂, Arg-Gly-NH₂, Tyr-Arg-Gly-NH₂, Ala-Ile-Gly-NH₂, Gly-Phe-Gly-NH₂, Gly-Trp-Gly-NH₂, Phe-Leu-Gly-NH₂, Gly-Tyr-Gly-NH₂, Ala-Pro-Gly-NH₂, and α-t-butylglycine-Pro-Gly-NH₂, Ala-Leu-Gly-Pro-Gly-NH₂ (SEQ. ID. NO.: 1) or Xn-G-NH₂, wherein X can be any amino acid, preferably proline, and n can be any number of consecutive amino acids, between 1-100,000 consecutive amino acids, preferably 1-10, 2-20, 3-30, 4-40, 5-50, 6-60, 7-70, 8-80, 9-90, 10-100, 100-1000, 1000-10,000, or 10,000-100,000 or more consecutive amino acids or Xⁿ can represent any peptide of any length or protein containing glycinamide. That is, some peptide amides are metabolized into an antiretroviral compound in the body or by preparations containing certain enzymes and/or catalysts. For example, prodrugs that can be used in the embodiments described herein also include, but are not limited to: Ser-Ile-Leu-NH₂, Ile-Leu-Asp-NH₂, Gly-Pro-Lys-NH₂, Pro-Lys-Glu-NH₂, Lys-Glu-Pro-NH₂, Glu-Pro-Phe-NH₂, Arg-Asp-Tyr-NH₂, Asp-Tyr-Val-NH₂, Tyr-Lys-Thr-NH₂, Arg-Ala-Glu-NH₂, Ala-Glu-Gln-NH₂, Glu-Gln-Ala-NH₂, Val-Lys-Asn-NH₂, Thr-Glu-Thr-NH₂, Leu-Leu-Val-NH₂, Val-Gln-Asn-NH₂, Gln-Asn-Ala-NH₂, Asn-Ala-Asn-NH₂, Asn-Pro-Asp-NH₂, Pro-Asp-Cys-NH₂, Cys-Lys-Thr-NH₂, Thr-Ile-Leu-NH₂, Pro-Gly-Ala-NH₂, Thr-Leu-Glu-NH₂, Thr-Ala-Cys-NH₂, Ala-Cys-Gln-NH₂, Gln-Gly-Val-NH₂, Pro-Gly-His-NH₂, and Arg-Val-Leu-NH₂. (See also U.S. Pat. Nos. 5,627,035; 6,258,932; 6,455,670; 6,593,455; and U.S. patent application Ser. Nos. 09/827,822; 09/938,806; 10/072,783; 10/217,933; 10/406,012, 10/235,158 and 10/235,158, all of which are herein expressly incorporated by reference in their entireties).

Methods of making antiretroviral compositions comprising a prodrug, as described herein, and a cofactor catalyst are also embodiments. Some of the methods are performed by identifying a prodrug for incorporation into a therapeutic or dietary supplement that contains a cofactor catalyst that converts glycinamide to alphahydroxyglycinamide (e.g., an oxidoreductase), providing said prodrug, and formulating said antiretroviral composition by mixing said prodrug with said cofactor catalyst. Optionally, the method may require the step of identifying a cofactor catalyst (e.g., an oxidoreductase) for incorporation in an antiretroviral composition comprising a prodrug that comprises glycinamide. That is, in some methods, both the prodrug and the catalyst are identified for incorporation into an antiretroviral composition. The antiretroviral compositions made by these methods are also embodiments.

The precursors or prodrugs described herein can be provided separately or in conjunction with a cofactor (e.g., coadministration in a mixture or providing the prodrug before or after delivery of the cofactor, such as 1, 2, 3, 4, 5, 6, 7, 8, 9, 10, 11, or 12 hours before or after). Cofactors that can convert the prodrug to an active molecule that inhibits HIV replication include CD26 or a material containing CD26, which converts a peptide-GNH₂ to GNH₂, and a heat labile enzyme (e.g., an oxido-reduction catalyst) found in fetal calf serum, bovine serum, plasma, or milk, horse serum, plasma, or milk, cat or dog serum (in isolated, enriched, or raw form), extracts from root nodules of the Leguminosae family, desirably Phaseolus extracts (e.g., Phaseolus vulgaris) that include an oxido-reduction catalyst, such as leghemoglobin, which converts G-NH₂ to a modified glycinamide that exhibits the ability to inhibit HIV replication (e.g., α-hydroxyglycinamide).

As above, said prodrug/cofactor formulations can be prepared according to certified good manufacturing processes (GMP) (e.g., suitable for accepted by a governmental regulatory body, such as the Federal Drug Administration (FDA) or suitable for nutriceuticals) and said containers can comprise a label or other indicia that reflects approval of said formulation from said governmental regulatory body. Nutriceuticals or dietary supplements containing said formulations with or without structure-function indicia are also embodiments. For example, nutriceutical and dietary supplement formulations such as powders, liquids, intravenous or transdermal solutions, elixirs, tablets, pills, capsules, aerosols, inhalable solutions, sublingual preparations, gums, or patches that contain one of the aforementioned compounds (e.g., a prodrug or cofactor both) separately or in mixtures of cofactor and prodrug or cofactor containing compositions and prodrugs are embodiments and such preparations can be labeled for a use that improves the general health and welfare of subjects infected with HIV or subjects in need of a compound that boosts the immune system.

Methods of selling or marketing a pharmaceutical or dietary supplement that contains one or more prodrugs and one or more catalysts or cofactors are also embodiments. By one approach, a market or plurality of consumers is identified as being in need of a composition that inhibits HIV replication or a composition that improves immune system function or a composition that promotes the general health and welfare of an individual. The identified market or plurality of consumers is then provided a product that is suitable for human consumption and said product comprises one or more prodrugs and one or more cofactors or catalysts. Representative prodrugs include, for example: Gly-NH₂, Pro-Gly-NH₂, Gly-Pro-Gly-NH₂, Gly-Lys-Gly-NH₂, Arg-Gln-Gly-NH₂, Cys-Gln-Gly-NH₂, Lys-Gln-Gly-NH₂, Ala-Leu-Gly-NH₂, Gly-Val-Gly-NH₂, Val-Gly-Gly-NH₂, Ala-Ser-Gly-NH₂, Ser-Leu-Gly-NH₂, Arg-Gly-NH₂, Tyr-Arg-Gly-NH₂, Ala-Ile-Gly-NH₂, Gly-Phe-Gly-NH₂, Gly-Trp-Gly-NH₂, Phe-Leu-Gly-NH₂, Gly-Tyr-Gly-NH₂, Ala-Pro-Gly-NH₂, and α-t-butylglycine-Pro-Gly-NH₂, Ala-Leu-Gly-Pro-Gly-NH₂ (SEQ. ID. NO.: 1) or Xn-G-NH₂, wherein X can be any amino acid, preferably proline, and n can be any number of consecutive amino acids, between 1-100,000 consecutive amino acids, preferably 1-10, 2-20, 3-30, 4-40, 5-50, 6-60, 7-70, 8-80, 9-90, 10-100, 100-1000, 1000-10,000, or 10,000-100,000 or more consecutive amino acids or Xⁿ can represent any peptide of any length or protein containing glycinamide. Representative cofactors or catalysts include, for example: CD26 or a material containing CD26, which converts a peptide-GNH₂ to GNH₂, and a heat labile enzyme (e.g., an oxido-reduction catalyst) found in fetal calf serum, bovine serum, plasma, or milk, horse serum, plasma, or milk, cat or dog serum (in isolated, enriched, or raw form), extracts from root nodules of the Leguminosae family, desirably Phaseolus extracts (e.g., Phaseolus vulgaris) that include an oxido-reduction catalyst or oxidoreductase, such as leghemoglobin, which converts G-NH₂ to a modified glycinamide that exhibits the ability to inhibit HIV replication (e.g., α-hydroxyglycinamide). Optionally, the methods include the step of measuring the number of products sold and/or a step of providing an advertising or promotional material (e.g., an electronic or written publication or website), wherein said advertising or promotional material discloses that said product inhibits HIV replication, improves the immune system of said consumer, or improves the general health and welfare of a consumer.

Alpha-hydroxyglycinamide (α-hydroxyglycinamide) compounds or a pharmaceutically acceptable salt thereof (also referred to collectively as “alphaHGA”) is a preferred active ingredient for incorporation into pharmaceuticals, dietary supplements, and/or medicaments that can be used to inhibit the replication of HIV. Preferred active ingredients also include any number of amino acids, modified amino acids, or amino acid derivatives conjugated with alphahydroxyglycinaimde (e.g., Valine-alphahydroxyglycinaimide). Pharmaceuticals, dietary supplements, and medicaments that consist of, consist essentially of, or comprise L-alphaHGA (in R or S isomer) or D-alpha HGA (in R or S isomer) or both (with either R or S or both isomers) are embodiments. Various mixtures of enantiomers and/or isomers of alphahydroxyglycinamide or aminoacid conjugated alphahydroxyglycinamide are also embodiments. For example, a mixture of alphahydroxyglycinamide or amino acid conjugated alphahydroxyglycinamide can comprise at least, equal to, or more than 10%, 20%, 30%, 40%, 50%, 60%, 70%, 80%, 90%, or 100% L or D enantiomer and/or at least, equal to, or more than 10%, 20%, 30%, 40%, 50%, 60%, 70%, 80%, 90%, or 100% R or S isomer. Preferred mixtures include 50/50 racemic mixtures.

Accordingly, pharmaceuticals, dietary supplements, and medicaments that consist of, consist essentially of, or comprise any number of amino acids, modified amino acids, or amino acid derivatives conjugated with alphahydroxyglycinaimde (e.g., Val-alphahydroxyglycinaimide) in L conformation (in R or S isomer) or D conformation (in R or S isomer) or both (with either R or S or both isomers) or any combination of the afore-mentioned conformations or isomers with respect to the amino acid and the alphahydroxyglycinamide (e.g., Valine and alphahydroxyglycinamide) are embodiments. These compositions (e.g., ampules, powders, liquids, capsules, pills, dietary supplements, tablets, intravenous solutions, transdermal, intranasal solutions, and other pharmaceutically acceptable formulations) preferably contain, provide, or deliver an amount of enzymatically prepared (Metabolite X) or synthetically prepared (alphaHGA) alpha hydroxyglycinamide or analog, derivative thereof that inhibits the replication and/or propagation of HIV, ameliorates a condition associated with HIV infection, or otherwise improves the health or welfare of an individual infected with HIV or an individual in need of a boost in the immune system.

Embodiments include, for example, pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (A) in either enantiomer (L or D) or both or either isomer (R or S) or both: 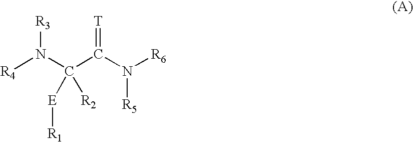
or a pharmaceutically acceptable salt, amide, ester, or prodrug thereof; wherein:

a) E is selected from the group consisting of oxygen, sulfur, and NR₇;

b) T is selected from the group consisting of oxygen, sulfur, and NR₈; and

c) R₁-R₆ and R₇-R₈ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl.

Desirable compositions include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (B) in either enantiomer (L or D) or both or either isomer (R or S) or both: 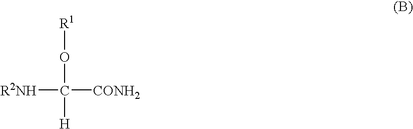
wherein, R¹ is a hydrogen atom, a lower alkyl group, a lower alkenyl group, a lower alkynyl group, a benzyl group, or a silyl group substituted with an alkyl group or an alkyl group and an aromatic group and R² is a hydrogen atom or an amino protecting group, or a salt thereof.

Preferred compositions include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (C) in either enantiomer (L or D) or both or either isomer (R or S) or both: 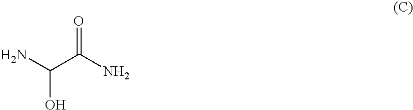
or a pharmaceutically acceptable salt, amide, ester, or prodrug thereof.

Particularly preferred compositions include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (D) in either enantiomer (L or D) or both or either isomer (R or S) or both:

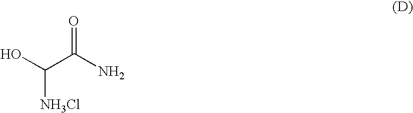

The compound of formula (C), α-hydroxyglycinamide, also referred to as Metabolite X or alphaHGA, has been produced by an enzymatic process and isolated using cation exchange HPLC and the compound of formula (D) has been made synthetically. In some contexts, both the compounds of formula (C) and (D) in either enantiomer (L or D) or both or either isomer (R or S) or both are referred to as “Metabolite X,” “alphaHGA,” or “modified glycinamide,” interchangeably.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (E) or (F) in either enantiomer (L or D) or both or either isomer (R or S) or both: 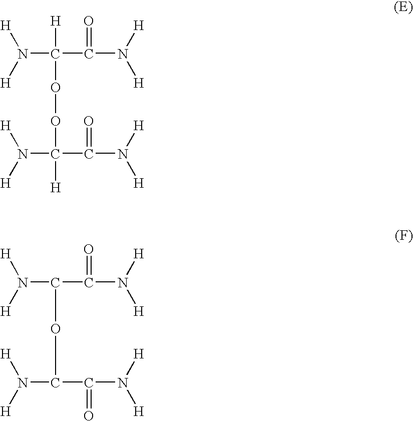
or a pharmaceutically acceptable salts, amides, esters, or prodrugs thereof.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (G) in either enantiomer (L or D) or both or either isomer (R or S) or both: 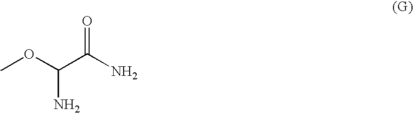
or a pharmaceutically acceptable salt, amide, ester, or prodrug thereof.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (H) in either enantiomer (L or D) or both or either isomer (R or S) or both or a pharmaceutically acceptable salt, amide, ester, or prodrug thereof: 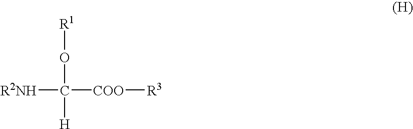
wherein, R¹ is a hydrogen atom, a lower alkyl group, a lower alkenyl group, a lower alkynyl group, a benzyl group, or a silyl group substituted with an alkyl group or an alkyl group and an aromatic group and R² is a hydrogen atom or an amino protecting group, or a salt thereof, and R³ is a hydrogen atom or a carboxyl protecting group that can be substituted with amino group by treatment with ammonia, such as lower alkyloxy groups, methoxy group (—OMe), ethoxy group (—OEt), benzyloxy group (—OBzl), or tert-butoxy group (-OtBu), or aryloxy group, such as p-nitrophenoxy group (—ONp), and the like.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (I) in either enantiomer (L or D) or both or either isomer (R or S) or both or a pharmaceutically acceptable salt, amide, ester, or prodrug thereof: 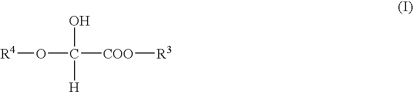
wherein, R³ is defined as described with reference to formula (H), and R⁴ is a lower alkyl group.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (J) in either enantiomer (L or D) or both or either isomer (R or S) or both or a pharmaceutically acceptable salt, amide, ester, or prodrug thereof:

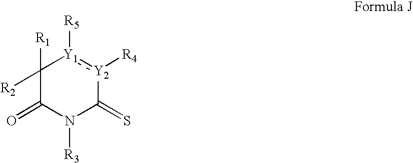

wherein,
a) R₁-R₅ are each independently selected from the group consisting of hydrogen; hydroxy; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl or may be absent, or R₁ and R₂ may together form an optionally substituted saturated or unsaturated six-membered ring;
b) Y₁ and Y₂ are each independently selected from the group consisting of carbon and nitrogen; and
c) the dashed bond indicates that the bond may be present or absent.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (K) in either enantiomer (L or D) or both or either isomer (R or S) or both or a pharmaceutically acceptable salt, amide, ester, or prodrug thereof.

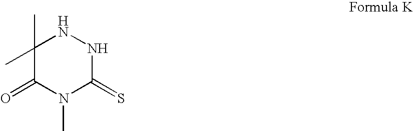

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (L) in either enantiomer (L or D) or both or either isomer (R or S) or both or a pharmaceutically acceptable salt, amide, ester, or prodrug thereof: 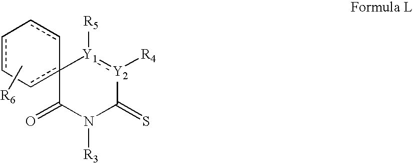
wherein,
a) R₃-R₆ are each independently selected from the group consisting of hydrogen; hydroxy; halogen; amine; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl or may be absent;
b) Y₁ and Y₂ are each independently selected from the group consisting of carbon and nitrogen;
c) the dashed bonds indicate that the bonds may be present or absent; and
d) the R₆ substituent may be present as one or more substituents at any of the 5 available carbon atoms on the six-membered carbon ring, including having multiple R₆ substituents independently selected.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (M) in either enantiomer (L or D) or both or either isomer (R or S) or both or a pharmaceutically acceptable salt, amide, ester, or prodrug thereof:

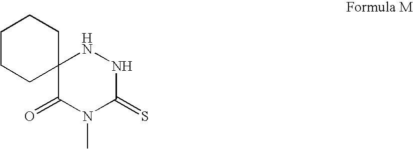

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (N) in either enantiomer (L or D) or both or either isomer (R or S) or both or a pharmaceutically acceptable salt, amide, ester, or prodrug thereof: 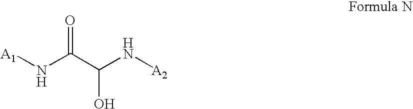
wherein, A₁ and A₂ are separately selected from the group consisting of a chain of one or more amino acids and hydrogen (e.g., 1, 2, 3, 4, 5, 6, 7, 8, 9, or 10 amino acids or hydrogen).

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (O) in either enantiomer (L or D) or both or either isomer (R or S) or both or a pharmaceutically acceptable salt, amide, ester, or prodrug thereof:

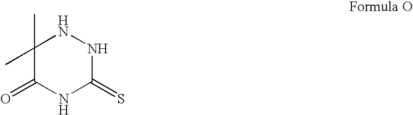

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (P) in either enantiomer (L or D) or both or either isomer (R or S) or both or a pharmaceutically acceptable salt, amide, ester, or prodrug thereof:

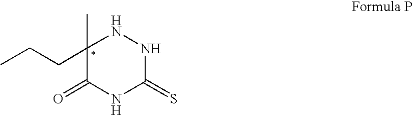

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (O) in either enantiomer (L or D) or both or either isomer (R or S) or both or a pharmaceutically acceptable salt, amide, ester, or prodrug thereof:

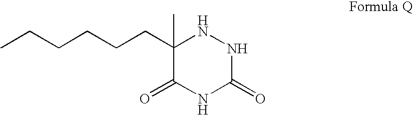

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (R) in either enantiomer (L or D) or both or either isomer (R or S) or both or a pharmaceutically acceptable salt, amide, ester, or prodrug thereof: 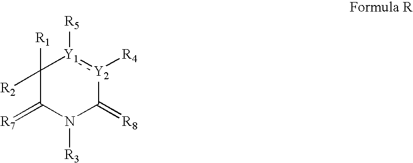
wherein,

a) R₁-R₅ are each independently selected from the group consisting of hydrogen; hydroxy; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl or may be absent, or R₁ and R₂ may together form an optionally substituted saturated or unsaturated six-membered ring;

b) R₇-R₈ are each independently selected from the group consisting of sulfur (S), oxygen (O), and imino (NH).

c) Y₁ and Y₂ are each independently selected from the group consisting of carbon and nitrogen; and

e) the dashed bond indicates that the bond may be present or absent.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (S) in either enantiomer (L or D) or both or either isomer (R or S) or both or a pharmaceutically acceptable salt, amide, ester, or prodrug thereof: 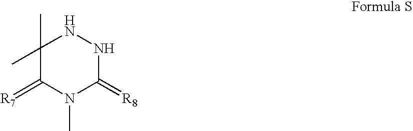
wherein, R₇-R₈ are each independently selected from the group consisting of sulfur (S), oxygen (O), and imino (NH).

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (T) in either enantiomer (L or D) or both or either isomer (R or S) or both or a pharmaceutically acceptable salt, amide, ester, or prodrug thereof: 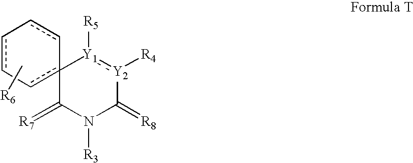
wherein,
a) R₃-R₆ are each independently selected from the group consisting of hydrogen; hydroxy; halogen; amine; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl or may be absent;
b) Y₁ and Y₂ are each independently selected from the group consisting of carbon and nitrogen;
c) the dashed bonds indicate that the bonds may be present or absent;
d) the R₆ substituent may be present as one or more substituents at any of the 5 available carbon atoms on the six-membered carbon ring, including having multiple R₆ substituents independently selected; and
f) R₇-R₈ are each independently selected from the group consisting of sulfur (S), oxygen (O), and imino (NH).

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (U) in either enantiomer (L or D) or both or either isomer (R or S) or both or a pharmaceutically acceptable salt, amide, ester, or prodrug thereof: 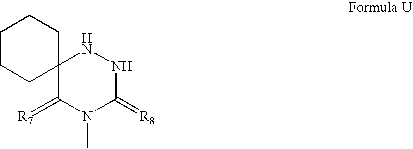
wherein, R₇-R₉ are each independently selected from the group consisting of sulfur (S), oxygen (O), and imino (NH).

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (V) in either enantiomer (L or D) or both or either isomer (R or S) or both or a pharmaceutically acceptable salt, amide, ester, or prodrug thereof: 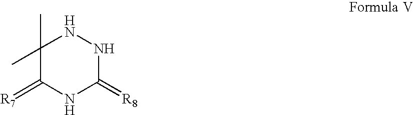
wherein, R₇-R₈ are each independently selected from the group consisting of sulfur (S), oxygen (O), and imino (NH).

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (W) in either enantiomer (L or D) or both or either isomer (R or S) or both or a pharmaceutically acceptable salt, amide, ester, or prodrug thereof: 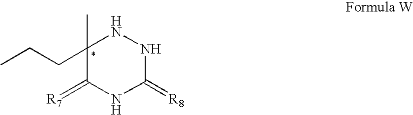
wherein, R₇-R₈ are each independently selected from the group consisting of sulfur (S), oxygen (O), and imino (NH).

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (X) in either enantiomer (L or D) or both or either isomer (R or S) or both or a pharmaceutically acceptable salt, amide, ester, or prodrug thereof: 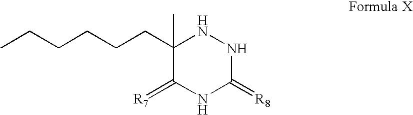
wherein, R₇-R₈ are each independently selected from the group consisting of sulfur (S), oxygen (O), and imino (NH).

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (A) in either enantiomer (L or D) or both or either isomer (R or S) or both or a pharmaceutically acceptable salt, amide, ester, or prodrug thereof: 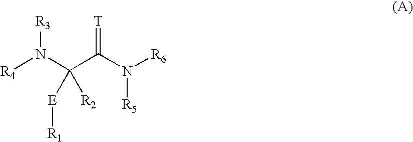
where E is selected from the group consisting of oxygen, sulfur, and NR₇; T is selected from the group consisting of oxygen, sulfur, and NR₈; R₁, R₂, R₅, R₆, R₇, and R₉ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl; and R₃ and R₄ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; optionally substituted perhaloalkyl; and an optionally substituted amino acid; wherein at least one of R₃ and R₄ is an optionally substituted amino acid.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (Y) in either enantiomer (L or D) or both or either isomer (R or S) or both or a pharmaceutically acceptable salt, amide, ester, or prodrug thereof:

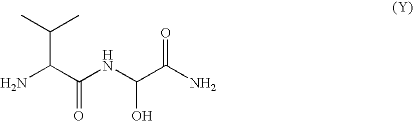

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (Z) in either enantiomer (L or D) or both or either isomer (R or S) or both or a pharmaceutically acceptable salt, amide, ester, or prodrug thereof: 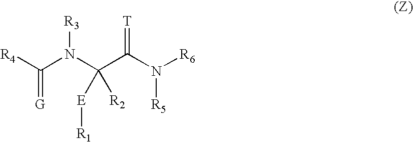
where E is selected from the group consisting of oxygen, sulfur, and NR₇; T is selected from the group consisting of oxygen, sulfur, and NR₉; G is selected from the group consisting of oxygen and sulfur; R₁, R₂, R₃, R₅, R₆, R₇, and R₈ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl; and R₄ is selected from the group consisting of optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl; wherein R₄ contains at least one amino functionality.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a modified glycinamide compound of formula (AA) in either enantiomer (L or D) or both or either isomer (R or S) or both or a pharmaceutically acceptable salt, amide, ester, or prodrug thereof: 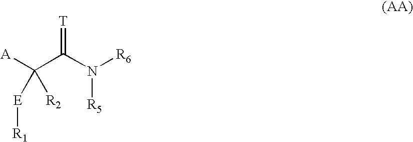
where E is selected from the group consisting of oxygen, sulfur, and NR₇; T is selected from the group consisting of oxygen, sulfur, and NR₈; R₁, R₂, R₅, and R₆ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl; and A is an optionally substituted amino acid.

Preferred compositions also include pharmaceuticals, dietary supplements, and medicaments (e.g., powders, liquids, intravenous or transdermal solutions, elixirs, tablets, pills, capsules, aerosols, inhalable solutions, sublingual preparations, gums, or patches) consisting, consisting essentially of, or comprising a product of a degradation, hydrolysis, or oxidation of a compound of formula (C) in either enantiomer (L or D) or both or either isomer (R or S) or both or a pharmaceutically acceptable salt, amide, ester, or prodrug thereof:

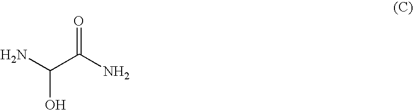

Alpha-methoxyglycinamide has been prepared synthetically and this compound was found to be more stable than alpha-hydroxyglycinamide. The compounds of formulas M, K, and Y have also been prepared synthetically and were found to inhibit the replication of HIV.

Some embodiments include mixtures of the compounds described herein. That is, in addition to the various mixtures of an antiretroviral compound described herein in L or D, or R, or S (e.g., a 50% racemic mixture) compositions comprising a plurality of the compounds described herein are also embodiments (e.g., compositions comprising alphahydroxyglycinamide and amino acid conjugated alphahydroxyglycinamide). Formulations comprising one or more of the compounds provided by formulas A, B, C, D, E, F, G, H, I, J, K, L, M, N, O, P, Q, R, S, T, U, V, W, X, Y, Z, and AA) in either enantiomer (L or D) or both or either isomer (R or S) or both and mixtures of said enantiomers and/or isomers (e.g., 50% L or D or R or S) are embodiments.

Embodiments also include several methods to identify, synthesize, or otherwise make or isolate more modified glycinamide compounds that inhibit the replication of HIV. By some approaches, a compound present in one or more of the formulas provided by A, B, C, D, E, F, G, H, I, J, K, L, M, N, O, P, Q, R, S, T, U, V, W, X, Y, Z, and AA) in either enantiomer (L or D) or both or either isomer (R or S) or both and mixtures of said enantiomers and/or isomers (e.g., 50% L or D or R or S) are incorporated into a pharmaceutical or dietary supplement that is formulated for human consumption. Optionally, the methods of making described herein can include the step of identifying the compound for incorporation into an antiretroviral pharmaceutical. Optionally, the methods of making can include the step of measuring the ability of the antiretroviral pharmaceutical or dietary supplement to inhibit the replication of HIV or promote the general health or welfare of a consumer (e.g., improve immune system function). Pharmaceuticals or dietary supplements made by the methods described herein are also embodiments.

Some embodiments concern methods to inhibit the replication and/or propagation of HIV, wherein a subject in need of an agent that inhibits the replication of HIV is provided an amount of enzymatically prepared (Metabolite X) or synthetically prepared alpha hydroxyglycinamide (alphaHGA) or one or more of the compounds of formulas A, B, C, D, E, F, G, H, I, J, K, L, M, N, O, P, Q, R, S, T, U, V, W, X, Y, Z, or AA in an amount that is sufficient to inhibit the propagation or replication of the virus. In some of these methods, the affect on HIV replication is measured (e.g., by observing or monitoring a reduction in viral load or another marker of HIV replication). In some embodiments, a subject is identified as a subject in need of an agent that inhibits replication of HIV or a boost in the immune system (e.g., diagnosis of HIV infection or compromised immune system) and the identified subject is then provided one or more of the afore-mentioned active ingredients.

Additional embodiments include approaches to treat and/or prevent HIV infection, wherein an afflicted patient or a person at risk for contracting HIV is provided an amount of modified glycinamide (e.g., alpha-hydroxyglycinamide, α-peroxyglycinamide dimer, diglycinamide ether, alpha-methoxyglycinamide, or one or more of the compounds of formulas A, B, C, D, E, F, G, H, I, J, K, L, M, N, O, P, Q, R, S, T, U, V, W, X, Y, Z, or AA) in an amount that is sufficient to inhibit the replication of HIV. As above, in some embodiments, the compound or a pharmaceutical containing the compound is provided to a subject in need of an agent that inhibits HIV replication and, in other embodiments, the affect on HIV replication is measured (e.g., by measuring a reduction in the viral load or marker thereof, such as p24 accumulation or reverse transcriptase activity). In some embodiments, a subject is identified as a subject in need of an agent that inhibits replication of HIV or a boost in the immune system (e.g., diagnosis of HIV infection or compromised immune system) and the identified subject is then provided one or more of the afore-mentioned active ingredients.

More embodiments include methods of improving the health and welfare of an individual afflicted with HIV. For example, approaches are described herein whereby a subject that is in need of an amelioration of a condition associated with HIV (e.g., a compromised immune system or a high HIV viral load) is provided an amount of modified glycinamide (e.g., alpha-hydroxyglycinamide, α-peroxyglycinamide dimer, diglycinamide ether, alpha-methoxyglycinamide, or one or more of the compounds of formulas A, B, C, D, E, F, G, H, I, J, K, L, M, N, O, P, Q, R, S, T, U, V, W, X, Y, Z, of AA) in an amount that is sufficient to ameliorate said condition or otherwise improve the health or welfare of the HIV infected individual. By virtue of the fact that the antiretroviral compounds described herein inhibit HIV replication, they also ameliorate conditions associated with HIV infection and improve the health and welfare of the HIV infected individual. Accordingly, the product containing the modified glycinamide (e.g., alpha-hydroxyglycinamide, α-peroxyglycinamide dimer, diglycinamide ether, alpha-methoxyglycinamide, or one or more of the compounds of formulas A, B, C, D, E, F, G, H, I, J, K, L, M, N, O, P, Q, R, S, T, U, V, W, X, Y, Z, of AA) or a prodrug thereof (e.g., any peptide-GNH₂, GPGNH₂ or GNH₂) with one or more cofactors that convert said prodrug to an antiretroviral active ingredient can be labeled and marketed as a dietary supplement. As above, in some embodiments, the active ingredient is provided to a subject in need of an agent that improves the health or welfare of an individual and, in other embodiments, the affect on health and welfare of the individual is measured (e.g., by measuring the amelioration of a condition associated with HIV infection, improvement in immune system function, or overall improvement of health and welfare of the individual). In some embodiments, a subject is identified as one in need of an agent that boosts the immune system or improves the general health and welfare (e.g., compromised immune system) and the identified subject is then provided one or more of the afore-mentioned active ingredients.

Still more embodiments, concern methods of improving the health and welfare of an individual at risk of becoming infected with HIV (e.g., intravenous drug users, prostitutes, and individuals that have unprotected sex). For example, approaches are described herein whereby a subject that is in need of maintenance of a healthy immune system or an immune system boost is provided an amount of modified glycinamide (e.g., alpha-hydroxyglycinamide, α-peroxyglycinamide dimer, diglycinamide ether, alpha-methoxyglycinamide, or one or more of the compounds of formulas A, B, C, D, E, F, G, H, I, J, K, L, M, N, O, P, Q, R, S, T, U, V, W, X, Y, Z, or AA) in an amount that is sufficient to maintain or boost a healthy immune system or otherwise improve the health or welfare of individual. By virtue of the fact that the antiretroviral compounds described herein inhibit viral replication, they also help to maintain and/or boost a subject's immune system that may be assaulted by the virus. Accordingly, the product containing the modified glycinamide (e.g., alpha-hydroxyglycinamide, α-peroxyglycinamide dimer, diglycinamide ether, alpha-methoxyglycinamide, or one or more of the compounds of formulas A, B, C, D, E, F, G, H, I, J, K, L, M, N, O, P, Q, R, S, T, U, V, W, X, Y, Z, or AA) or a prodrug thereof (e.g., any peptide-GNH₂, GPGNH₂ or GNH₂) with one or more cofactors that convert said prodrug to an antiretroviral active ingredient can be labeled and marketed as a dietary supplement.

By some approaches, for example, an HIV infected individual or a person in need of an improvement in health or an amelioration of a condition associated with HIV infection or an individual at risk of becoming infected with HIV is provided a pharmaceutical or dietary supplement in tablet, capsule, gelcap, liquid, or powder that comprises an effective amount of modified glycinamide (e.g., alpha-hydroxyglycinamide, α-peroxyglycinamide dimer, diglycinamide ether, alpha-methoxyglycinamide, or one or more of the compounds of formulas A, B, C, D, E, F, G, H, I, J, K, L, M, N, O, P, Q, R, S, T, U, V, W, X, Y, Z, or AA).

By another method, said individual is provided a pharmaceutical or dietary supplement that comprises an effective amount of a prodrug for a modified glycinamide (e.g., GPGNH₂, peptide-GNH₂, or GNH₂) and/or one or more cofactors that convert a prodrug into an antiretroviral active ingredient (e.g., one or more of the compounds of formulas A, B, C, D, E, F, G, H, I, J, K, L, M, N, O, P, Q, R, S, T, U, V, W, X, Y, Z, or AA). The cofactor can be CD26 or a CD26-containing material, which converts a peptide-GNH₂ to GNH₂ or a heat labile protein(s) found in fetal calf serum, bovine serum, plasma, or milk, horse serum, plasma, or milk, cat or dog serum in isolated, enriched, or raw form, a root extract obtained from a leguminous plant, preferably a Phaseolus species (e.g., Phaseolus vulgaris), or an oxidase enzyme (e.g., leghemoglobins, particularly leghemoglobins of the Phaseolus species), which converts the G-NH₂ to an antiretroviral compound, referred to as Metabolite X. Accordingly, a means for converting glycinamide to alphahydroxyglycinamide can be formulated with a prodrug that comprises a means for producing glycinamide so as to produce an antiretroviral composition. That is, some embodiments include an antiretroviral composition comprising glycinamide or a means for producing glycinamide and an oxidoreductase or a means for converting said glycinamide to alphahydroxyglycinamide.

Still further, aspects of the invention include antiretroviral compositions comprising glycinamide and a composition that inhibits HIV in the presence of glycinamide, such as one or more of the afore-mentioned cofactors, catalysts, oxidoreducatses, or a means for converting said glycinamide to alphahydroxyglycinamide. Accordingly, in some embodiments, the composition that inhibits HIV in the presence of glycinamide is one that inhibits replication of HIV by virtue of the ability of the composition to convert glycinamide to alphahydroxyglycinamide.

The cofactors and prodrugs can be present in the same composition or can be administered separately (e.g., the prodrug can be provided either before or after the cofactor is provided, such as 1, 2, 3, 4, 5, 6, 7, 8, 9, 10, 11, or 12 hours before or after). That is, aspects of the invention described herein include dietary supplements that can contain a glycinamide-containing prodrug (e.g., G-NH₂) or a compound that is metabolized by the body into glycinamide, and/or a cofactor that converts a glycinamide containing peptide (e.g., GPG-NH₂) into G-NH₂, such as CD26, and/or a cofactor that converts glycinamide into one or more of the compounds provided by formulas A, B, C, D, E, F, G, H, I, J, K, L, M, N, O, P, Q, R, S, T, U, V, W, X, Y, Z, or AA, such as a root nodule extract from a leguminous plant, a flavooxidase, a leghemoglobin, or a glycine oxidase, preferably a recombinant, synthetic, isolated, enriched, or purified, leghemoglobin obtained from a Phaseolus vulgaris.

### BRIEF DESCRIPTION OF THE DRAWINGS

FIG. 1 shows the structures of glycylprolylglycinamide (GPG-NH₂), sarcosylpyrolylglycinamide (SAR-PG-NH₂), cyclic pyrroglutaminylprolylglycinamide (PyrQPG-NH₂), glutaminylprolylglycinamide (QPG-NH₂), and glycinamide (G-NH₂).

FIG. 2 shows the CD26 activity in human T-lymphocytes (CEM, C8166, Molt4/C8, MT-4) and PBMC suspensions (panel A) or in several different serum (human (HS), murine (MS), bovine (BS) (panel B)) as a function of time. The substrate was glycylprolyl-p-nitroanilide (GP-pNA). Enzyme activity was measured by absorption at 400 nm.

FIG. 3 shows the purified CD26-mediated conversion of unlabeled GPG-NH₂ to GP-OH and G-NH₂. The detection was performed by mass spectrometry.

FIG. 4 shows the conversion of radiolabeled [¹⁴C]GPG-NH₂ to [¹⁴C]G-NH₂ by bovine serum (BS) at 5% in phosphate buffered saline (PBS), Human serum (HS) at 5% in PBS, and CEM cell suspensions (10⁶ cells).

FIG. 5 shows the inhibitory affect of the CD26-specific inhibitor IlePyr on the dipeptidylpeptidase activity of CD26 in 5% bovine serum (BS) in PBS and 10⁶ CEM cell suspensions in PBS using GP-pNA as the substrate.

FIG. 6 shows the effect of the CD26 inhibitor IlePyr on the anti-HIV-1 activity of GPG-NH₂ and G-NH₂ in CEM cell cultures.

FIG. 7 shows the results of an analysis of several lots of human sera (HS) and fetal bovine sera (FBS) for their ability to convert G-NH₂ to modified G-NH₂ (Metabolite X).

FIG. 8 shows the results of an analysis of several different animal sera for their ability to convert G-NH₂ to modified G-NH₂ (Metabolite X).

FIG. 9 also shows the results of an analysis of different animal sera for their ability to convert G-NH₂ to modified G-NH₂ (Metabolite X).

FIG. 10 shows the results of a competition assay, wherein the ability of different concentrations of glycine, L-serine-NH₂, L-alanine-NH₂, or GPG-NH₂ to inhibit the conversion of G-NH₂ to modified G-NH₂ (Metabolite X) were evaluated.

FIG. 11 shows the results of an experiment to evaluate the conversion of G-NH₂ to Metabolite X in bovine sera at different pH.

FIG. 12 shows the results of an analysis of different fractions of fetal bovine serum, obtained by size exclusion chromatography, to convert G-NH₂ to modified G-NH₂ (Metabolite X).

FIG. 13 shows the conversion of G-NH₂ to Metabolite X as determined by cation exchange chromatography and luminescence.

FIG. 14 illustrates the results of an experiment evaluating the percentage conversion of G-NH₂ to metabolite X by 10% pig serum; Glutathione, N-Acetyl Cysteine, and Dithiothreitol.

FIG. 15 shows the conversion of G-NH₂ to Metabolite X by Phaseoulus vulgaris root nodule extract.

FIG. 16 illustrates the results of a reverse transcriptase (RT) activity assay, wherein enzymatically prepared alpha-hydroxyglycinamide (Metabolite X or Met-X) inhibited the replication of HIV in cultures containing boiled fetal calf serum but G-NH₂ did not.

FIG. 17 shows the results of a reverse transcriptase (RT) assay, wherein enzymatically prepared alpha-hydroxyglycinamide (Metabolite X or Met-X) that had been dialysed five times inhibited the replication of HIV in cultures containing boiled fetal calf serum.

FIG. 18 shows the results of a reverse transcriptase (RT) assay, wherein the antiretroviral activity (IC₅₀) of various concentrations of enzymatically prepared alpha-hydroxyglycinamide (Metabolite X or Met-X) were analysed.

FIG. 19 shows the results of an HIV infectivity assay (in fetal calf serum), which monitored the accumulation of p24, wherein enzymatically prepared alpha-hydroxyglycinamide (Metabolite X or Met-X) inhibited HIV as effectively as GPG-NH₂.

FIG. 20 shows the results of an HIV infectivity assay (in fetal calf serum), which monitored the accumulation of p24, wherein synthetically prepared alpha-hydroxyglycinamide (AlphaHGA) inhibited HIV as effectively as GPG-NH₂.

FIGS. 21A and B shows the results of an HIV infectivity assay (in fetal calf serum (panel A) and human serum (panel B)), which monitored the accumulation of p24, wherein enzymatically prepared alpha-hydroxyglycinamide (Metabolite X or Met-X) and synthetically prepared alpha-hydroxyglycinamide (AlphaHGA) inhibited HIV as effectively as G-NH₂ in fetal calf serum (panel A) but only enzymatically prepared alpha-hydroxyglycinamide (Metabolite X or Met-X) and synthetically prepared alpha-hydroxyglycinamide (AlphaHGA) inhibited HIV replication in human serum (panel B).

FIG. 22 shows the results of a reverse transcriptase (RT) assay (in fetal calf serum), wherein the antiretroviral activity of G-NH₂, freshly diluted synthetically prepared alpha-hydroxyglycinamide (AlphaHGA), and synthetically prepared alpha-hydroxyglycinamide, which had been incubated at 37° C. for three days (AlphaHGA 37), was compared.

FIG. 23 shows the results of an assay that evaluated the mitogenic activity of α-HGA.

FIG. 24 shows the results of an assay that evaluated the mitogenic activity of formula K.

FIG. 25 shows the results of an assay that evaluated the mitogenic activity of formula M.

FIGS. 26A and 26B shows the results of a reverse transcriptase (RT) assay, wherein the antiretroviral activity (IC₅₀) of various concentrations of alpha-hydroxyglycinamide and a compound of formula Y were analysed.

### DETAILED DESCRIPTION OF THE INVENTION

It has been discovered that some peptide amides and glycinamide are prodrugs that are metabolized into compounds that inhibit the replication of HIV. These antiviral agents are highly selective inhibitors in cell culture (e.g., GPG-NH₂ and glycinamide or “G-NH₂” inhibit HIV replication in CEM cell cultures to an equal extent (50% effective concentration: ˜30 μM)). The focus of research in this area has been on the conversion of tripeptide amides to glycinamide (G-NH₂) since G-NH₂ also inhibits the replication of HIV. (See U.S. patent application Ser. No. 10/235,158, herein expressly incorporated by reference in its entirety). It is now known that the lymphocyte surface glycoprotein marker CD26 efficiently converts GPG-NH₂ to G-NH₂ releasing the dipeptide GP-OH and that this cleavage is required for GPG-NH₂ to exert its antiretroviral activity.

It has also been discovered that G-NH₂ is itself a prodrug that is metabolized to one or more compounds (e.g., cyclic, charged, or uncharged forms of glycinamide) that inhibit the replication of HIV. These metabolites that are derived from G-NH₂ are referred to as “modified glycinamide,” “glycinamide derivatives,” or “Metabolite X.” Mass spectrometry and nuclear magnetic resonance (NMR) spectrometry analysis of the modified glycinamide peak fraction isolated after chromatographic separation revealed that it contained α-hydroxyglycinamide (“AlphaHGA” or (C₂H₆N₂O₂) or (C₂H₇ClN₂O₂)). Both α-hydroxyglycinamide and α-methoxyglycinamide were prepared by organic synthesis, as well as, several of the compounds of formulas A, B, C, D, E, F, G, H, I, J, K, L, M, N, O, P, Q, R, S, T, U, V, W, X, Y, Z, or AA. It was found that enzymatically prepared alpha-hydroxyglycinamide (Metabolite X) and synthetically prepared alpha-hydroxyglycinamide (AlphaHGA) effectively inhibit HIV in human serum. The formulation of pharmaceuticals and medicaments containing these modified glycinamides is straightforward and the use of these compounds to inhibit replication of HIV in subjects in need thereof is provided herein.

Once it had been determined that alphaHGA effectively inhibited replication of HIV, several analogs and derivatives were evaluated for their ability to inhibit HIV replication. Approximately 250 compounds were obtained from commercial sources and/or were prepared using approaches such as that described in U.S. Pat. No. 6,365,752, herein expressly incorporated by reference in its entirety. From these experiments, several more compounds that inhibited replication of HIV were discovered. Preferred embodiments, for example, include compositions that comprise a compound of formula K (C₆H₁₁N₃OS) or of formula M (C₉H₁₅N₃OS), which were also found to efficiently inhibit HIV replication. Further analysis revealed that amino acid derivatized or conjugated alphaHGA (e.g., Val-alphahydroxyglycinamide, formula Y) was also stable and effective at inhibiting HIV replication.

During these studies it was further discovered that modified glycinamide could be produced by interacting a prodrug (e.g., GPGNH₂, peptide-GNH₂, or GNH₂) with one or more cofactors that converted said prodrug into an active form (e.g., one or more of the compounds of formulas A, B, C, D, E, F, G, H, I, J, K, L, M, N, O, P, Q, R, S, T, U, V, W, X, Y, Z, or AA). The cofactor can be a heat labile protein(s) found in fetal calf serum, bovine serum, plasma, or milk, horse serum, plasma, or milk, cat or dog serum in isolated, enriched, or raw form, a plant extract prepared from root nodules of Leguminosae, desirably extracts that contain flavooxidase enzymes, and preferably flavooxidase-containing extracts obtained from Phaseolus species (e.g., Phaseolus vulgaris), such as, leghemoglobins. Nutraceuticals or dietary supplements that contain one or more prodrugs and/or one or more cofactors are also embodiments and these compositions can be labeled with or without structure-function indicia.

Still further, the leghemoglobin gene of Phaseolus vulgaris was codon-optimized to improve the translation efficiency of the gene in humans. Although the codon-optimized leghemoglobin protein sequence is identical to the native Phaseolus vulgaris leghemoglobin protein provided in SEQ. ID NO. 2, Discontiguous MegaBlast revealed that the nucleic acid sequence shared homology to the native Phaseolus vulgaris DNA sequence over only two domains: a first domain at approximately nucleotide residues 1-100 (approx. 83% identity) and a second domain at approximately nucleotide residues 312-431 (approx. 75% identity). The codon-optimized leghemoglobin gene can be inserted into a vector, preferably an episomal expression vector that allows for high level of expression in a human subject with little or no interference with the genome, such as a replication deficient adenoviral vectors (AdV) (e.g., Clonetech and Qbiogene). Expression vectors containing the codon-optimized leghemoglobin gene can then be transferred to subjects and the ability to convert glycinamide to antiretroviral modified glycinamide (e.g., alpha hydroxyglycinamide) can be monitored (e.g., determining a reduction in viral load in an HIV infected subject). The section below describes the discovery that CD26 is a cofactor that converts GPG-NH₂ to G-NH₂, which is itself a prodrug for Metabolite X.

CD26 Mediates the Conversion of GPG-NH₂ to the Prodrug G-NH₂

The lymphocyte surface glycoprotein CD26 has been originally described as a T-cell activation/differentiation marker. (See Fox et al., J. Immunol., 132:1250-1256 (1984)). CD26 is abundantly expressed on the target cells of HIV (i.e., lymphocytic CEM, Molt, C8166 and MT-4, and peripheral blood mononuclear cells) and is also present in serum from bovine, murine and human origin. It is a membrane-associated peptidase identical to dipeptidyl-peptidase IV (DPP IV, EC3.4.14.5) and has a high (but not exclusive) selectivity for peptides that contain a proline or alanine as the penultimate amino acid at the N-terminus. (See Yaron and Naider, Biochem. Mol. Biol., 28:31-81 (1993); De Meester et al., Immunol. Today, 20:367-375 (1999) and Mentlein, Regul. Pept., 85:9-24 (1999)). It is not only expressed on a variety of leukocyte cell subsets, but also on several types of epithelial, endothelial and fibroblast cells. (Id.). A soluble form of CD26 also exists. It lacks the transmembrane regions and intracellular tail and is detected in plasma and cerebrospinal fluids at low amounts. (See Yaron and Naider, Biochem. Mol. Biol., 28:31-81 (1993); De Meester et al., Immunol. Today, 20:367-375 (1999)).

Several cytokines, hematopoietic growth factors, hormones and neuropeptides contain a X-Pro or X-Ala motif at their N-terminus. (See De Meester et al., Immunol. Today, 20:367-375 (1999)). The presence of a proline near the N-terminus serves as a structural protection against non-specific proteolytic degradation. (See Vanhoof et al., FASEB J, 9:736-744 (1995)). In particular, relatively small peptides may serve as natural substrates (e.g., the chemokines RANTES (68 amino acids) and SDF-110 (68 amino acids), and the glucagon/VIP (Vasoactive Intestinal Protein) family peptides such as GIP (42 amino acids) and GLP-2 (33 amino acids)). (See De Meester et al., Immunol. Today, 20:367-375 (1999)). In some cases, the peptides are very short (e.g., the neuropeptides endomorphin 2 (4 amino acids) and substrate P (11 amino acids)). Enterostatin, consisting of only 5 amino acids is also found to be a substrate for CD26.

Interestingly, in certain cases, CD26 was shown to alter the biological functions of natural peptides after it cleaved off a dipeptide from the N-terminal part of the molecule. (Oravecz et al., J. Exp. Med., 186:1865-1872 (1997); Proost et al., J. Biol. Chem., 273:7222-7227 (1998)). Indeed, truncated RANTES (3-68) was found to have a markedly increased anti-HIV-1 activity compared with intact RANTES (see Schols et al., Antiviral Res., 39:175-187 (1998)); whereas N-terminal processing SDF-lac by CD26 significantly diminished its anti-HIV-1 potency. (See Ohtsuki et al., FEBS Lett., 431:236-240 (1998); Proost et al., FEBS Lett., 432:73-76 (1998)). Also, it was recently shown that CD26 regulates SDF-1α-mediated chemotaxis of human cord blood CD34⁺ progenitor cells. (See Christopherson et al., J. Immunol., 169:7000-7008 (2002)).

The tripeptide glycylprolylglycinamide (GPG-NH₂) has been found to inhibit HIV replication at non-toxic concentrations. (See e.g., U.S. Pat. No. 5,627,035, herein expressly incorporated by reference in its entirety) but its association with CD26 was not known. Glycylprolylglycinamide blocks a wide variety of HIV-1 laboratory strains and clinical isolates within a range of 2-40 μM. Since there exist two GPG motifs in HIV p24 and one GPG motif in the V3 loop of the viral envelope protein gp120 initial research had been focussed on these viral proteins as potential targets for this novel tripeptide derivative. (See Su, Ph.D. thesis at the Karolinska Institute (ISBN 91-628-4326-5), Stockholm, Sweden (2000) and Su et al., AIDS Res. Human Retrovir., 16:37-48 (2000), herein expressly incorporated by reference in its entirety).

Although an increase in mobility of gp160/120 on SDS-PAGE was observed at high concentrations of GPG-NH₂, it was found that GPG-NH₂ did not affect an early event in the infection cycle of HIV. (See Su et al., J. Hum. Virol., 4:8-15 (2001)). In addition, binding of GPG-NH₂ with the p24 protein has been demonstrated and an increased number of misassembled core structures of virus particles was observed in GPG-NH₂-treated HIV-1-infected cells. (See Hoglund et al., Antimicrob. Agents Chemother., 46:3597-3605 (2002)). Also, viral capsid (p24) formation was found to be disturbed in the presence of the drug. (See Hoglund et al., Antimicrob. Agents Chemother., 46:3597-3605 (2002)). It became clear that GPG-NH₂ inhibited replication of HIV by a novel mechanism.

Given the presence of a proline residue in the middle (equivalent to the penultimate amino acid at the amino terminus) of the GPG-NH₂ peptide molecule, it was thought that GPG-NH₂ can be a substrate for CD26/dipeptidylpeptidase IV and that CD26 enzymatic activity can modulate the antiretroviral activity of the compound. Accordingly experiments were conducted to determine whether CD26/dipeptidylpeptidase IV could convert GPG-NH₂ to G-NH₂ and, indeed, it was discovered that CD26 selectively and efficiently cleaved GPG-NH₂ after the proline residue to release the dipeptide GP-OH and G-NH₂. Moreover, it was also demonstrated that this cleavage was required for GPG-NH₂ to exert its antiretroviral activity. EXAMPLE 1 describes these findings in greater detail.

Accordingly, it was discovered that the lymphocyte surface glycoprotein CD26, which is a membrane associated dipeptidyl peptidase, is the enzyme responsible for metabolizing glycinamide-containing peptide amides, such as peptide-G-NH₂, GPG-NH₂, QPG-NH₂, and sarcosylprolylglycinamide (SAR-PG-NH₂) to G-NH₂. More evidence that CD26 was responsible for metabolizing peptide amides into a form that inhibits the replication of HIV was obtained from experiments that employed the selective CD26 inhibitor L-isoleucinepyrrolidine (IlePyr), wherein a significant reduction in the anti-HIV activity of GPG-NH₂ and SAR-PG-NH₂ was observed. The IlePyr inhibitor had no affect on the ability of G-NH₂ to inhibit replication of HIV, however. Thus, X-Pro-glycinamide-containing peptide amides are antiretroviral prodrugs or precursors that are metabolized by the lymphocyte surface glycoprotein CD26 to G-NH₂, which is itself a prodrug, as described in the following sections.

Glycinamide Inhibits the Replication of HIV

Initially, it was determined that G-NH₂ efficiently inhibits the replication of HIV but compounds that are similar in structure do not. HIV-1 (III_{B})-infected CEM cell cultures were incubated with various concentrations of G-NH₂ or various concentrations of a compound that has a structure similar to G-NH₂ and the inhibition of HIV replication was evaluated using standard procedures. These experiments are described in the EXAMPLE 2.

It has also been discovered that G-NH₂ is itself a prodrug or a precursor that is metabolized by an enzyme or cofactor(s) present in the plasma and sera of some animals, extracts from leguminous plants (e.g., Phaseolus vulgaris) and flavooxidases, in particular leghemoglobin enzymes, to one or more compounds (e.g., cyclic, charged, or uncharged forms of glycinamide) that inhibit the replication of HIV. The section below describes these discoveries in greater detail.

Cofactor(s) Present in the Plasma and Sera of Some Animals and Extracts from Leguminous Plants Convert G-NH₂ to a Compound that Inhibits Replication of HIV

Evidence is provided herein that at least one cofactor present in the serum and plasma of some animals and extracts from leguminous plants, in particular of the Phaseolus species (e.g., extracts containing leghemoglobin) metabolize G-NH₂ to an active form (“modified glycinamide” or Metabolite X), which is transported into cells and inhibits the replication of HIV. Accordingly, G-NH₂ is itself a precursor prodrug for an antiretroviral compound and G-NH₂ can be formulated for administration with said cofactor or a material containing said cofactor.

Chromatographic methods were used to isolate and characterize the cofactor from bovine serum. Enriched preparations of the bovine cofactor were also prepared. The bovine cofactor can be purified, cloned, and sequenced using the approaches described herein and conventional techniques in molecular biology.

Extracts from Phaseolus vulgaris root nodules were also prepared and it was discovered that these extracts contained a cofactor that coverts GNH₂ to a modified GNH₂ that can inhibit the replication of HIV. It is contemplated that the leghemoglobin enzyme, a 16,900 Dalton protein that exhibits glycine oxidase activity at an alkaline pH, converts the glycinamide to a modified glycinamide that exhibits antiretroviral activity.

Leghaemoglobin from the root nodules of kidney bean (Phaseolus vulgaris) reacts in alkaline glycine solutions as a glycine oxidase in a reaction that may also be regarded as a coupled oxidation. (See LaRue et al., Anal Biochem. January 1; 92(1):11-5 (1979)). Leghaemoglobin is reduced to the ferrous form by glycinate, the oxygen complex is formed, and finally the haem is attacked to yield a green reaction product. Glycine is simultaneously oxidized to glyoxylate, and hydrogen peroxide is generated. The initial velocity of the formation of the green product is proportional to the concentrations of leghaemoglobin and glycine, and the optimum pH for the reaction is approximately pH 10.2. Isolated, enriched, purified, recombinant, or synthetic leghemoglobin can be used to enzymatically produce a modified glycinamide, Metabolite X, that exhibits an antiretroviral activity (e.g., an inhibition of the replication of HIV). In this manner, leghemoglobin is a cofactor that converts G-NH₂ into an antiretroviral active form (e.g., alphahydroxyglycinamide).

Accordingly, aspects of the invention concern providing to an HIV infected person or a person at risk of contacting HIV a pharmaceutical, dietary supplemental, or medicament (e.g., such as by capsule, tablet, powder, liquid, injection, dietary supplement, transdermal delivery, or gene transfer technique) that comprises glycinamide or a glycinamide-containing peptide or protein and/or an isolated, enriched, or purified leghemoglobin gene or protein (e.g., Kidney Bean (Phaseolus vulgaris), Soybean (Glycine max), Cowpea (Vigna unguiculata), or Winged Bean (Psophocarpus tetragonolobusor)) and/or a molecule that is at least, equal to, or greater than 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical in one or more domains or over the full-length of the gene or protein (e.g., a flavoxidase or glycine oxidase) and/or that can convert glycinamide to a modified glycinamide that has antiretroviral activity (e.g., a compound of the formula A, B, C, D, E, F, G, H, I, J, K, L, M, N, O, P, Q, R, S, T, U, V, W, X, Y, Z, or AA, such as alphahydroxyglycinamide). The glycinamide amino acid (G-NH₂), a peptide or protein containing said amino acid, or a peptide or protein that is metabolised into said amino acid can be coadministered with the leghemoglobin or leghemoglobin-related cofactor (e.g., Phaseolus vulgaris leghemoglobin gene or protein). Coadministration can be in the same composition, in the same mixture, or providing the prodrug 1, 2, 3, 4, 5, 6, 7, or 8 hours before or after the cofactor is provided. That is, the cofactors and prodrugs described herein can be provided in combination or separately, for example, in dietary supplements or pharmaceuticals.

Phaseolus vulgaris leghemoglobin has also been cloned and sequenced. See GenBank 004939 and P02234, which are hereby incorporated by reference in their entireties. The gene encoding the native protein can be inserted into a mammalian expression plasmid (e.g., an Adenovirus vector with a constutive or inducible promoter that drives expression of the leghemoglobin gene) and this vector can be used to provide leghemoglobin to subjects that do not naturally express this protein or proteins that can convert glycinamide to an antiretroviral active form (e.g., humans, primates, mice or rats). When the innoculated individuals are given glycinamide, a glycinamide-containing peptide, or a peptide that metabolizes to glycinamide, the cofactor produced from the expression vector metabolizes the prodrug to a form that inhibits the replication of HUV.

Although the native leghemoglobin gene can be expressed in a subject (e.g., a human) using the approach described above, so as to provide said subject with an enzyme that catalyzes the conversion of G-NH₂ to modified G-NH₂, a codon-optimized (e.g., suboptimal codons were replaced with codons that are preferentially translated in human) leghemoglobin gene was developed so as to improve the efficiency of translation in the human. Expression vectors that comprise a codon-optimized leghemoglobin gene can be developed and these constructs can be transferred to a subject in need of an enzyme that catalyzes the conversion of G-NH₂ to modified G-NH₂. Several commercial facilities perform codon optimization (e.g., Retrogen and Aptagen) and an approach is discussed infra.

Accordingly, some embodiments include pharmaceutical or dietary supplements that contain a compound that metabolizes to G-NH₂ (e.g., GPG-NH₂ or peptide-GNH₂) or G-NH₂ formulated separately or formulated in a mixture or administered in conjunction with (e.g., less than or equal to 1, 2, 3, 4, 5, 6, 7, 8, 9, 10, 11, or 12 hours before or after) a material (e.g., CD26 or a CD26-containing mixture) that converts the compound that metabolizes to GPG-NH₂ to G-NH₂ and/or a cofactor that converts the G-NH₂ to Metabolite X (e.g., pig serum, plasma, or milk, horse serum, plasma, or milk, bovine serum, plasma, or milk in purified, enriched, or isolated form, and/or an extract from a plant of Leguminosae, such as root nodule extract of Phaseolus vulgaris, and/or a flavooxidase, such as leghemoglobin or a leghemoglobin-containing preparation).

As discussed above, the active form of G-NH₂ (modified glycinamide or Metabolite X) is readily produced by incubation of G-NH₂ in certain serums, plasmas, and plant extracts and the modified glycinamide is easily isolated by the chromatographic methods described herein. Throughout this disclosure, glycinamide metabolites (the antiretrovirally active forms of glycinamide) are collectively referred to as “modified glycinamide,” “modified G-NH₂,” or “fast peak glycinamide.” Examples of modified G-NH₂ include, but are not limited to α-hydroxyglycinamide, α-peroxyglycinamide dimer (NH₂-gly-O—O-gly-NH₂), diglycinamide ether (NH₂-gly-O-gly-NH₂), α-methoxyglycinamide, α-ethoxyglycinamide, the compounds of formulas A, B, C, D, E, F, G, H, I, J, K, L, M, N, O, P, Q, R, S, T, U, V, W, X, Y, Z, or AA and salts and/or derivatives of these compounds. Mass spectrometry and nuclear magnetic resonance (NMR) spectrometry analysis of modified glycinamide prepared by incubation of G-NH₂ in bovine serum and isolation by column chromatography revealed that the modified glycinamide peak fraction contained α-hydroxyglycinamide. The compound α-peroxyglycinamide dimer (NH₂-gly-O—O-gly-NH₂) may be more stable than α-hydroxyglycinamide and both α-hydroxyglycinamide and α-methoxyglycinamide have been prepared by organic synthesis, as well as, several of the compounds of A, B, C, D, E, F, G, H, I, J, K, L, M, N, O, P, Q, R, S, T, U, V, W, X, Y, Z, or AA. Those of skill in the art can readily prepare other modified glycinamide compounds using the procedures described herein and other available synthetic approaches. (See e.g., JP 5097789A2 to Hayakawa et al., entitled “Alpha-hydroxyglycinamide Derivative and its Preparation,” filed Oct. 3, 1991, herein expressly incorporated by reference in its entirety). HIV infectivity studies conducted in the presence of synthetically or enzymatically produced AlphaHGA α-hydroxyglycinamide) revealed that the compound effectively inhibited HIV replication in human serum. Similarly, the compounds of formula K, M, and Y were also found to inhibit HIV replication.

Formulation of the modified G-NH₂ into pharmaceuticals, dietary supplements, and medicaments, whether the modified G-NH₂ is synthetically produced or produced enzymatically by incubation of G-NH₂ in an animal serum (e.g., bovine serum, porcine serum, or horse serum), plant extract (e.g., a root nodule extraction of a plant in Leguminosae, such as Phaseolus vulgaris), or isolated, enriched, or purified flavooxidase enzyme (e.g., leghemoglobin) are embodiments. Accordingly, antiretroviral pharmaceuticals, dietary supplements, and medicaments can be prepared by providing a modified glycinamide compound (e.g., a compound provided by formulas A, B, C, D, E, F, G, H, I, J, K, L, M, N, O, P, Q, R, S, T, U, V, W, X, Y, Z, or AA) or a pharmaceutically acceptable salt thereof in either enantiomer (L or D) or both or either isomer (R or S) or both. Preferred compounds for formulation into an antiretroviral pharmaceutical, dietary supplement, or medicament include, for example, α-hydroxyglycinamide (formula C), α-peroxyglycinamide dimer (formula E), diglycinamide ether (formula F), and alpha-methoxyglycinamide (formula G), and the compounds of formula K or formula M, or pharmaceutically acceptable salts thereof in either enantiomer (L or D) or both or either isomer (R or S) or both. The antiretroviral compounds described herein can be provided in unit dosage form (e.g., tablets, capsules, gelcaps, liquid doses, injectable doses, transdermal or intranasal doses) and can contain, in addition to the modified glycinamide compound, a pharmaceutically acceptable carrier or exipient. Containers comprising said compounds (e.g., sterile vials, septum sealed vials, bottles, jars, syringes, atomizers, swabs) whether in bulk or in individual doses are also embodiments and, preferably, said formulations are prepared according to certified good manufacturing processes (GMP) (e.g., suitable for accepted by a governmental regulatory body, such as the Federal Drug Administration (FDA)) and said containers comprise a label or other indicia that reflects approval of said formulation from said governmental regulatory body. Dietary supplements containing said compounds with or without structure-function indicia are also embodiments, however.

Some embodiments concern a dietary supplement that improves an immune system function or otherwise promotes a healthy immune system in a subject in need thereof. Preferred dietary supplements, for example, comprise, consist of, or consist essentially of a modified glycinamide compound (e.g., a compound provided by formulas A, B, C, D, E, F, G, H, I, J, K, L, M, N, O, P, Q, R, S, T, U, V, W, X, Y, Z, or AA or a pharmaceutically acceptable salt thereof in either enantiomer (L or D) or both or either isomer (R or S) or both). By one approach, said modified glycinamide compounds are generated enzymatically by mixing glycinamide with an oxido-reduction catalyst (e.g., an enzyme that oxidizes glycinamide so as to form α-hydroxy glycinamide, such as a leghemoglobin or an animal serum cofactor). The oxido-reduction catalyst that is mixed with the glycinamide can be purified, enriched, or isolated or can be present in a source material (e.g., animal sera, such as pig, bovine, and horse and root nodule extracts from legume plants, such as Phaseolus vulgaris). By another approach, the modified glycinamide compounds are made synthetically and are incorporated into a dietary supplement.

The dietary supplement aspects of the invention significantly improve the immune system function or otherwise promote a healthy immune system in a subject in need thereof in several ways. When HIV infects humans, the cells it infects most often are CD4+ cells. As the infection progresses, the number of CD4+ cells (T cell count) decreases, which is a sign that the immune system is being weakened. Thus, the CD4+ cell count is an important measure of the health of the immune system. The lower the count, the greater damage HIV has done. Anyone who has less than 200 CD4+ cells, or a CD4+ percentage less than 14%, is considered to have AIDS according to the US Centers for Disease Control.

T-cell tests are normally reported as the number of cells in a cubic millimeter of blood, or mm³. There is some disagreement about the normal range for T-cell counts, but the consensus is that normal CD4+ counts are between 500 and 1600, and CD8+ counts are between 375 and 1100. CD4+ counts drop dramatically in people with HIV, in some cases down to zero. Accordingly, some of the pharmaceuticals and dietary supplements described herein improve the immune system function by indirectly raising the T cell count in HIV infected subjects. That is, the pharmaceuticals and dietary supplements comprising modified glycinamide inhibit the replication of HIV and thereby promote the survival of greater numbers of T cells in infected individuals. Accordingly, some aspects of the invention concern pharmaceuticals and dietary supplements containing the active ingredients described herein that increase the T cell count in an HIV infected individual to at least, equal to, or greater than 10, 25, 50, 100, 200, 300, 400, 500, 600, 700, 800, 900, 1000, 1100, 1200, 1300, 1400, 1500 or more.

Stated another way, the ratio of CD4+ cells to CD8+ cells is often reported. This result is calculated by dividing the CD4+ value by the CD8+ value. In healthy individuals, this ratio is between 0.9 and 1.9, meaning that there are about 1 to 2 CD4+ cells for every CD8+ cell. In HIV infected individuals, this ratio drops dramatically, meaning that there are many times more CD8+ cells than CD4+ cells. Accordingly, some aspects of the invention concern pharmaceuticals and dietary supplements containing the active ingredients disclosed herein that increase the ratio of CD4+ cells to CD8+ cells in an HIV infected individual to at least, equal to, or greater than 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or more.

Because the T-cell counts can be variable, it is also preferred to monitor T-cell percentages. These percentages refer to total lymphocytes. For example, a CD4+%=34%, means that 34% of the lymphocytes are CD4+ cells. The normal range is between 20% and 40% and a CD4+ percentage below 14% indicates serious immune damage. It is a sign of AIDS in people with HIV infection. Accordingly, some aspects of the invention concern pharmaceuticals and dietary supplements containing the active ingredients described herein that increase the T cell percentage in an HIV infected individual by at least, equal to, or greater than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% or more.

Although there are several different families of T-cells, as HIV reduces the number of T-cells, some of these families can be totally wiped out, which can result in the appearance of opportunistic infections. Many physicians begin to provide drugs to combat these opportunistic pathogens when the T cell count reaches certain hallmark levels. Because the appearance of opportunistic infections is directly related to the prevalence of HIV infection, which reduces the numbers of T cells in the body, the embodiments of the invention described herein also ameliorate a condition associated with HIV infection (e.g., an opportunistic infection associated with the reduction of T cells in the body mediated by HIV infection, such as pneumocystis carinii pneumonia (PCP), toxoplasmosis, cryptococcosis, and mycobacterium avium complex (MAC).

The pharmaceuticals and dietary supplements described herein may consist of, consist essentially of, or comprise, an enriched, isolated, purified, or synthesized modified glycinamide compound (e.g., one or more of the compounds of formulas A, B, C, D, E, F, G, H, I, J, K, L, M, N, O, P, Q, R, S, T, U, V, W, X, Y, Z, or AA). As used herein, “enriched” means that the concentration of the material is up to or at least 2 times, 5 times, 10 times, 30 times, 40 times, 50 times, 60, times, 70 times, 80 times 90 times, 100 times, 200 times, 300 times, 400 times, 500 times or 1000 times its natural concentration (for example), advantageously 0.01%, by weight, preferably at least about 0.1% by weight. Enriched preparations from about 0.5%, 1%, 5%, 10%, and 20% by weight are also contemplated. The term “isolated” requires that the material be removed from its original environment (e.g., the natural environment if it is naturally occurring). The term “purified” does not require absolute purity; rather, it is intended as a relative definition. Isolated proteins can be conventionally purified by chromatography and/or gel electrophoresis. Purification of starting material or natural material to at least one order of magnitude, preferably two or three orders, and more preferably four or five orders of magnitude is expressly contemplated.

EXAMPLE 3 describes an approach that was used to purify commercially obtained glycinamide. Aspects of this approach were used to purify metabolites of glycinamide produced after mixing in various animal sera or root nodule extract of Phaseolus vulgaris, as described infra.

As discussed in the sections that follow, it was discovered that modified G-NH₂ (fractions 2-3) can be made from unmodified G-NH₂ (fractions 15-17) by incubating unmodified G-NH₂ in various serums or plasma or root nodule extracts of leguminous plants (e.g., Phaseolus vulgaris). Modified G-NH₂ that is made in this manner (enzymatically prepared) can then be isolated using one of the approaches above. Using conventional techniques in structure analysis, it was determined that the modified G-NH₂ isolated by the chromatographic procedure above comprised α-hydroxyglycinamide.

Initially, it was observed that if cell culture medium containing fetal bovine serum was heated for 30 minutes at 95° C., the ability of G-NH₂ to inhibit the replication of HIV was lost. In some experiments, human T-lymphocytic CEM cells (approx. 4.5×10⁵ cells/ml) were suspended in fresh medium and were infected with HIV-1 (III_{B}) at approx. 100CCID₅₀ per ml of cell suspension. Subsequently, the infected cells were provided various concentrations of G-NH₂ that had been dissolved in serum (10% fetal bovine serum in PBS) containing RPMI-1640 medium or G-NH₂ that had been dissolved in heat inactivated serum (10% fetal bovine serum in PBS that had been heated to 95° C. for 30 minutes) containing RPMI-1640 medium. The cell resuspensions were then incubated at 37° C. and, after 4 to 5 days, HIV replication was evaluated. It was discovered that the G-NH₂ that had been incubated in heat inactivated serum containing medium had lost its ability to inhibit the replication of HIV. These results provided strong evidence that a heat labile protein present in bovine sera metabolized G-NH₂ to a modified G-NH₂ form that inhibited replication of HIV.

Following the discovery that a heat labile cofactor(s), present in fetal calf serum, could convert G-NH₂ to a antiretrovirally-active form of glycinamide, experiments were conducted to determine if this cofactor(s) was present in human serum and sera from other animals. EXAMPLE 4 describes these experiments in greater detail.

Several experiments were also performed to better characterize the cofactor(s) found in certain animal sera. In one set of experiments, amino acid competition studies were employed to determine if the cofactor(s) present in pig serum was specific for G-NH₂. In these experiments, approximately 10% pig serum in PBS was incubated for 6 hours at 37° C. in the presence of 18 μM G-NH₂ and a competitor (10 μM, 40 μM, 100 μM, 400 μM, 1000 μM, 4,000 μM, or 10,000 μM glycine, 10,000 μM L-serine-NH₂, 10,000 μM L-alanine-NH₂, 1000 μM, 4,000 μM, or 110,000 μM GPG-NH₂). A control without competitor was also evaluated. Subsequently, the conversion of G-NH₂ to modified G-NH₂ was analyzed by cation exchange HPLC, as before. The results shown in FIG. 10 provide evidence that the cofactor(s) present in pig serum was specific for G-NH₂. The data also show that GPG-NH₂ and L-Serine-NH₂ can provide a substrate for the cofactor to generate modified glycinamide. It is likely that pig serum contains a CD26 or a CD26-like molecule that generates G-NH₂ from GPG-NH₂ and a serine hydroxymethyltransferase or related molecule, which generates G-NH₂ from L-Serine-NH₂. (See Scarsdale et al., J. Mol. Biol. 296: 155-168 (2000)). This observation also supports the finding that amidated amino acids or peptide amides can be prodrugs for glycinamide.

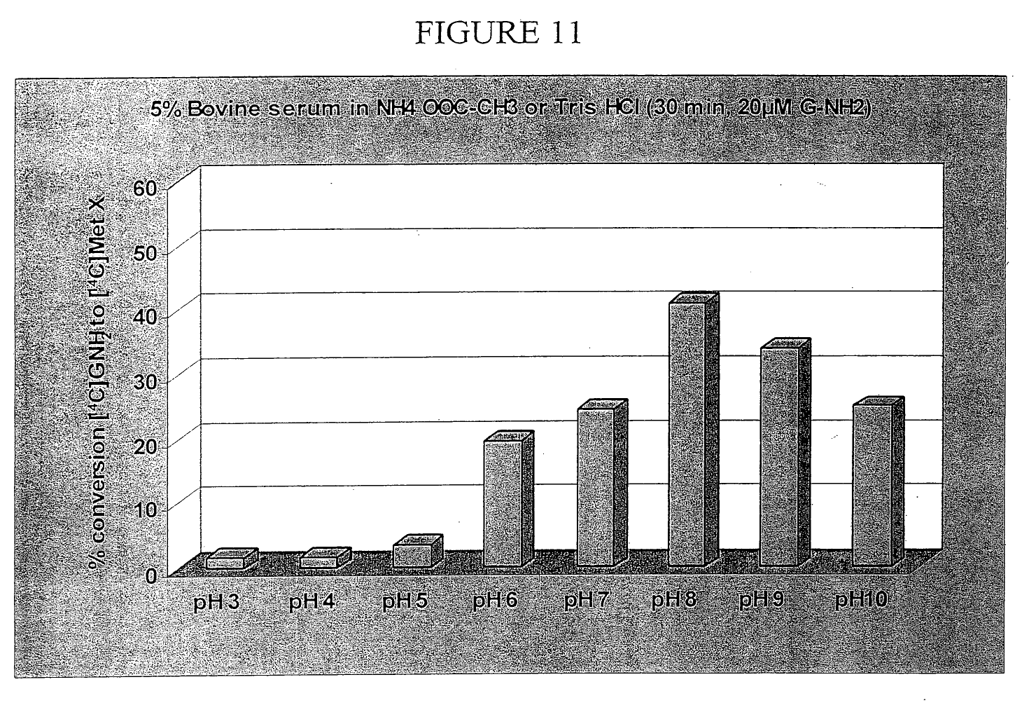

Experiments were also performed to characterize the pH parameters for the cofactor found in bovine serum. In these experiments, approximately 20 μM radiolabeled (¹⁴C) G-NH₂ was mixed with 5% bovine serum in NH₄OOCCH₃ or Tris HCl at various pH for 30 minutes. Following the reaction, samples were separated and analyzed by cation exchange HPLC, as before. As shown in FIG. 11, appreciable enzymatic activity began at pH 5 and significant enzymatic activity was seen at pH 6, pH 7, pH 10, and probably greater, however, better catalysis was observed at pH 8 and pH 9, with the best being at pH 8.

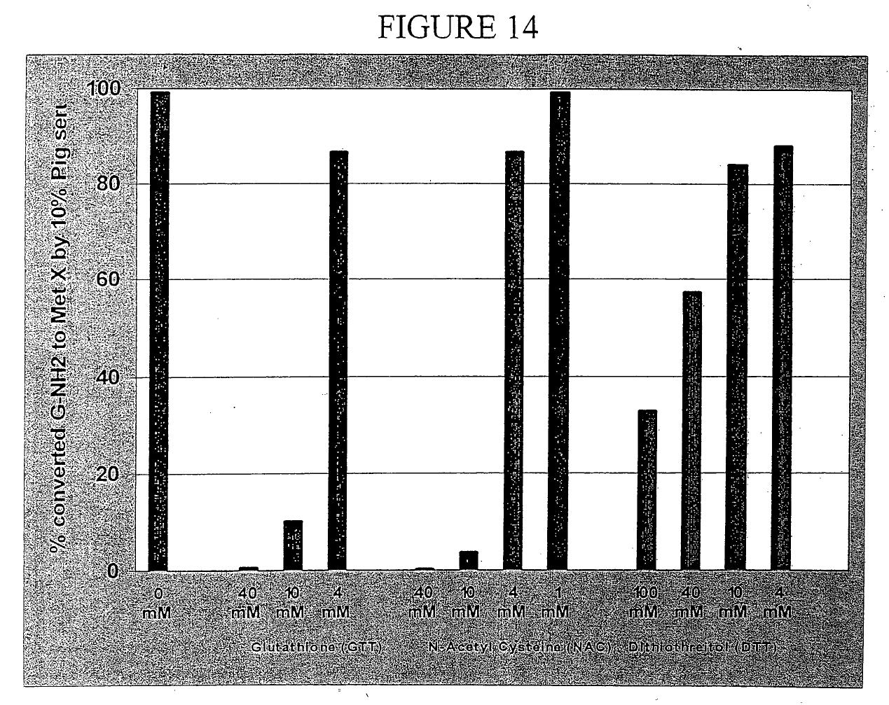

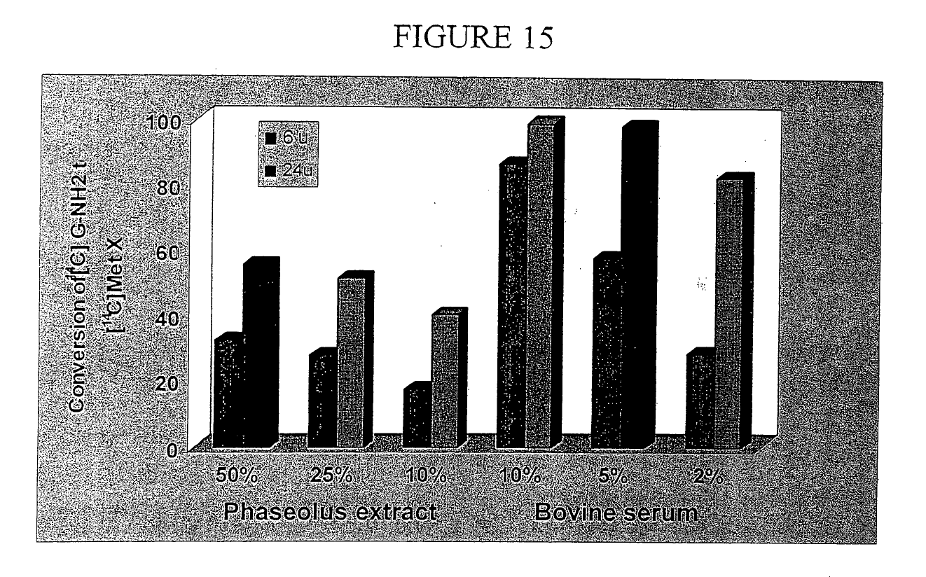

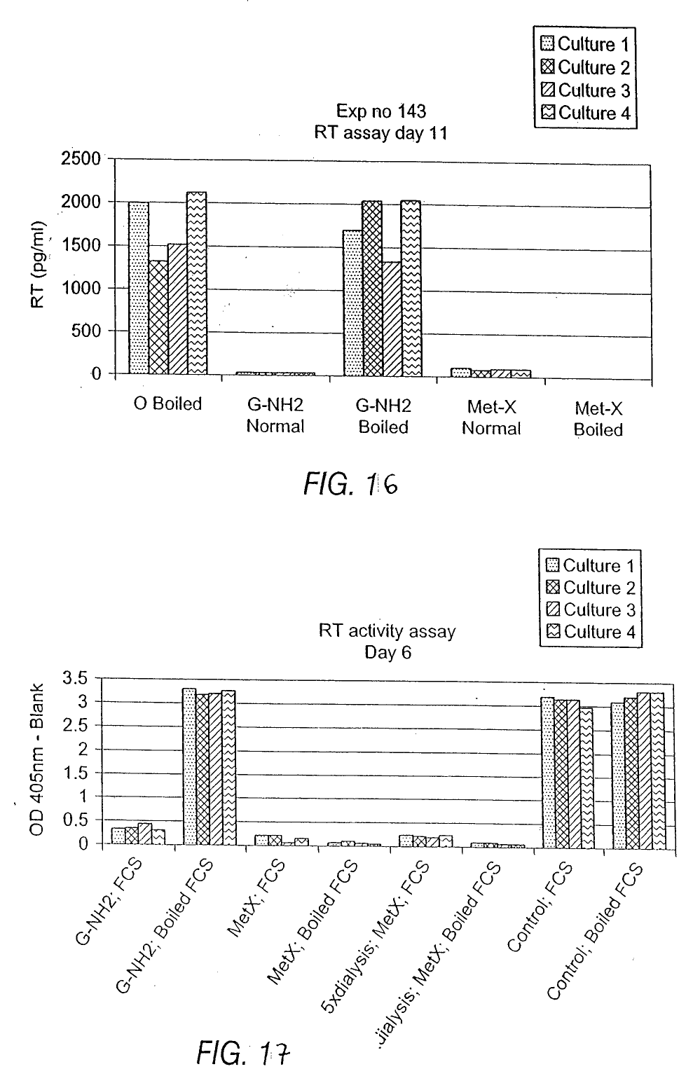

In another set of experiments, the conversion of GNH₂ to modified glycinamide (Metabolite X) in pig serum (10%) was monitored over time in the presence and absence of differing concentrations (e.g., 40 mM, 10 mM, 4 mM) of reducing agents (glutathione (GTT), N-acetyl cysteine (NAC), and dithiothreitol (DTT)). As shown in FIG. 14, when the concentration of the reducing agent rises (GTT, NAC or DTT), the inhibition of the cofactor that converts G-NH₂ to Metabolite X is greater, which provides strong evidence that the serum cofactor is an oxidase. See Han et al., Bull. Korean. Chem. Soc., 17, 659-661 (1996). In the absence of GTT, NAC and DTT, almost 100% coversion to Metabolite X was seen.

In still another set of experiments, a first step isolation of the cofactor present in pig plasma was performed and the enzymatic activity of the isolated product was compared to the enzymatic activity of the crude product. An aliquot of pig plasma was placed in dialysis tubing (MW cut off 10,000) and the serum was subjected to dialysis. Subsequently, the pig plasma dialysate was evaluated for the ability to convert G-NH₂ to modified G-NH₂.

Various concentrations of G-NH₂ were mixed with either 90% pig plasma or 90% dialyzed pig plasma in PBS and the reactions were conducted at 37° C. for 24 hours. Subsequently, aliquots of the mixtures were separated by cation exchange HPLC, as described previously, and the conversion of G-NH₂ to modified G-NH₂ was evaluated. TABLE 7 shows the results of these experiments. The data show that the conversion of G-NH₂ to modified G-NH₂ was almost identical in both the pig plasma and dialyzed pig plasma samples. Saturation of the enzyme activity of cofactor(s) in pig plasma (90% in PBS) occurred between 1,000 μM and 10,000 μM G-NH₂. Significantly, the isolated porcine serum cofactor maintained its ability to convert G-NH₂ to Metabolite X. These results also provided more evidence that the cofactor(s) that metabolizes G-NH₂ to modified G-NH₂ is a protein found in plasma or serum of some animals.

TABLE 7
Conversion of G-NH₂ to modified G-NH₂ by dialyzed pig plasma
(24 hr)

| | |
|---|---|
| | conversion to modified |
| | G-NH₂ (24 hr) |
| Concentration G-NH₂ | (percent conversion) |

| (μM) | Pig plasma^{a} | Dialysed Pig plasma |
|---|---|---|
| 18 | 99.7 | 99.8 |
| 100 | 99.7 | 99.8 |
| 1,000 | 98.7 | 99.8 |
| 10,000 | ˜24.5 | 24.7 |

^{a}Plasma: 90% in PBS.

In still another set of experiments, the saturation point of the isolated cofactor found in pig plasma was more closely scrutinized. Dialyzed pig plasma (90% in PBS) was mixed with concentrations of G-NH₂ between 2,000 M and 10,000 μM. Subsequently, the mixtures were incubated at 37° C. for 6 hours and aliquots were separated by cation exchange HPLC, as before. The results shown in TABLE 8 confirmed that the saturation point of the cofactor(s) in pig plasma was near 2,000 μM G-NH₂.

TABLE 8
Conversion of G-NH₂ to modified G-NH₂ by dialyzed pig plasma^{a}
(6 hr)

| Concentration G-NH₂ | | |
|---|---|---|
| (μM) | Percent conversion | μM formation |
| 2,000 | 82.6 | 1,652 |
| 4,000 | 42.1 | 1,684 |
| 6,000 | 24.9 | 1,494 |
| 8,000 | 21.0 | 1,680 |
| 10,000 | 17.0 | 1,700 |

^{a}Plasma: 90% in PBS.

Once it had been confirmed that certain sera contained the cofactor(s) that could convert G-NH₂ to modified G-NH₂, experiments were conducted to purify the bovine cofactor. EXAMPLE 5 describes these experiments in greater detail.

The data above provides evidence that the serum cofactor that converts glycinamide to modified glycinamide is a heat labile enzyme present in some animals. The enzyme efficiently converts glycinamide at a wide range of pH (e.g., pH 5 to greater than pH 10) but the best activity occurred at pH 8-9. The enzyme was found to be sensitive to reducing agents, indicating that oxygen was required for the reaction. The enzyme was isolated and purified and the product obtained was found to efficiently convert G-NH₂ to a modified G-NH₂ that exhibits an antiretroviral activity (e.g., an inhibition of HIV replication). Based on this information, it was contemplated that the serum cofactor was an oxidase, oxidoreductase, or oxido-reduction catalyst.

Glycine oxidase catalyzes the oxidative deamination of various primary and secondary amino acids (e.g., sarcosine, N-ethylglycine, and glycine) and d-amino acids (e.g., d-alanine, d-proline, d-valine, etc.) to form the corresponding α-keto acids and hydrogen peroxide. (See Molla et al. Eur. J. Biochem. 270: 1474-1482 (2003)). Glycine oxidase seems to partially share substrate specificity with various flavooxidases, such as d-amino acid oxidase (DAAO, EC 1.4.3.3) and sarcosine oxidase (SOX, EC 1.5.3.1), and also appears to be stereospecific in the oxidation of the d-isomer of the amino acids. Id. D-Amino acid oxidase also catalyzes the oxidative deamination of neutral and (less efficiently) basic d-amino acids to give the corresponding α-keto acids, ammonia, and hydrogen peroxide Id. Accordingly, it is contemplated that the animal serum cofactor is an enzyme that may catalyze the following reaction:
glycinamide+H₂O+O₂=α-hydroxy glycinamide+H₂O₂

To confirm that the serum cofactor was indeed a oxido-reduction catalyst, a hydrogen peroxide detection system was employed. That is, Horseradish peroxidase (HRP) was used in a chemiluminescence detection system that employed luminol as secondary substrate. Overtime, an oxido-reduction catalyst decomposes luminol and detectable photons are emitted. The amount of light emitted can be recorded with xray film or a fluorescence detector and the amount of detected signal can be quantified, which is then proportional to the amount of enzyme present.

As described in EXAMPLE 6, it was confirmed that the serum cofactor was indeed an oxido-reduction catalyst by using this chemiluminescent technique. EXAMPLE 7 also provides an approach to prepare and isolate an oxido-reduction catalyst fusion protein.

Accordingly, aspects of the invention concern an isolated oxido-reduction catalyst, (e.g., a dialyzed animal sera or plasma, such as pig plasma) that converts glycinamide to a modified glycinamide (e.g., α-hydroxy glycinamide) at a rate that is at least, equal to, or greater than 80% of a concentration of glyciamide of 10 μM-100 μM, 100-500 μM, 500-1000 μM, 1000-1500 μM 1500-2000 μM in 24 hours. That is, some embodiments concern an isolated oxido-reduction enzyme or use thereof as described herein that converts glycinamide to a modified glycinamide, such as α-hydroxy glycinamide, at a rate that is at least, equal to, or greater than 80% of a concentration of glycinamide of 10 μM, 25 μM, 50 μM, 100 μM, 200 μM, 300 μM, 400 μM, 500 μM, 600 μM, 700 μM, 800 μM, 900 μM, 1000 μM, 1200 μM, 1500 μM, 1800 μM, or 2000 μM or more in 24 hours.

More embodiments concern a purified oxido-reduction catalyst (e.g., an (NH₄)₂SO₄ precipitated bovine serum or a fraction of eluant of bovine serum or (NH₄)₂SO₄ precipitated bovine serum that was separated and fractioned on a chromatographic column (e.g., size exclusion or ion exchange) that converts glycinamide to a modified glycinamide (e.g., α-hydroxy glycinamide) at a rate that is at least, equal to, or greater than 0.1 units/μg/hour, wherein 1 unit is the amount of enzyme that converts 1.7 nmol of glycinamide in one hour. That is, some embodiments concern a purified and/or concentrated or enriched oxido-reduction enzyme or use thereof as described herein that converts glycinamide to a modified glycinamide, such as α-hydroxy glycinamide, at a rate that is at least, equal to, or greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, 5.0, 6.0, 7.0, 8.0, 9.0, 10.0, 20.0, 30.0 μg/hour. That is, some embodiments include a pharmaceutical or dietary supplement comprising a means for converting glycinamide to alphahydroxyglycinamide and examples of said means include, but are not limited to, oxidoreductases, oxidases, and oxido-reduction catalysts, as described herein.

Now that the oxido-reduction catalyst that converts glycinamide to a modified glycinamide (e.g., α-hydroxy glycinamide) has been purified, it can be sequenced and/or the gene that encodes this enzyme can be cloned using techniques that are routine in the field of molecular biology. Furthermore, aspects of the invention concern a G-NH₂-converting enzyme (i.e., an enzyme that converts G-NH₂ to α-hydroxyglycinamide) fusion protein. These fusion proteins, among other functions, facilitate isolation of the enzyme and such approaches can be used to rapidly generate copious amounts of the enzyme.

In light of the findings above, experiments were conducted to determine whether other oxido-reduction catalysts could be identified as having the ability to convert glycinamide to a modified glycinamide. Accordingly, in one experiment, purified glycine oxidase from Bacillus subtilis was reacted with radiolabled (¹⁴C) glycinamide and the presence or absence of radiolabled modified glycinamide was monitored using the column chromatography approach detailed in EXAMPLES 3 and 4. The results showed that purified glycine oxidase from Bacillus subtilis was unable to convert G-NH₂ to Metabolite X.

In another set of experiments, an extract of root nodules from Phaseolus vulgaris (kidney bean), which contains leghemoglobin, was tested for the ability to convert glycinamide to modified glycinamide. Leghemoglobin behaves as a glycine oxidase in alkaline conditions. This reaction has been characterized as a coupled oxidation. (See LaRue et al., Anal Biochem. January 1; 92(1):111-5 (1979)). Glycine oxidase catalyzes the oxidative deamination of various primary and secondary amino acids (e.g. sarcosine, N-ethylglycine, and glycine) and D-amino acids (e.g. D-alanine, D-proline, D-valine, etc.) to form the corresponding α-keto acids and hydrogen peroxide. Glycine oxidase may also share substrate specificity with various flavooxidases, such as D-amino acid oxidase and sarcosine oxidase. Id.

It was contemplated that various leghemoglobins and other members of the flavooxidase gene family may react with glycinamide to produce modified glycinamide. Accordingly, experiments were conducted to evaluate the ability of a root nodule extract obtained from Phaseolus vulgaris to convert glycinamide to a modified glycinamide. EXAMPLE 8 describes these experiments in greater detail.

Accordingly, some embodiments concern an isolated or purified plant oxido-reduction catalyst (e.g., a Phaseolus vulgaris root nodule extract) that converts glycinamide to a modified glycinamide (e.g., α-hydroxy glycinamide). That is, some embodiments concern an isolated plant oxido-reduction enzyme or use thereof, as described herein, that converts glycinamide to a modified glycinamide, such as α-hydroxy glycinamide, at a rate that is at least, equal to, or greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, 5.0, 6.0, 7.0, 8.0, 9.0, 10.0, 20.0, 30.0 μg glycinamide/hour.

Several oxido-reduction catalysts (e.g., oxidases and leghemoglobins) from animals and plants have been cloned and sequenced. These genes can be inserted into an expression vector, the constructs can be transferred to bacteria, and the expressed proteins can be isolated and analyzed for the ability to convert glycinamide to modified glycinamide, as described above.

By one approach, a high throughput analysis is conducted whereby various isolated or purified recombinant oxido-reduction catalysts from different organisms (e.g., leghemoglobins from Soybean (Glycine max), Cowpea (Vigna unguiculata), and Winged Bean (Psophocarpus tetragonolobus)), for example proteins expressed and isolated from bacteria) are reacted with AcriGlow 301 (Capricorn Products of Scarborough Me.) and glycinamide. AcriGlow 301 allows for the direct quantitation of hydrogen peroxide without the need of additional enzymes. If the candidate oxido-reduction catalyst exhibits the ability to convert glycinamide to a modified glycinamide, hydrogen peroxide will be generated and the chemiluminescent substrate will generate a detectable signal. Detection can be accomplished with a tube luminometer (Digene of Beltsville, Md.). Alternatively, a chemiluminescence detection system employing luminol, as described supra, can be used or the accumulation of radiolabled modified glycinamide can be monitored in reactions containing the candidate oxido-reduction catalyst and (¹⁴C) glycinamide. Accordingly, methods of identifying an enzyme (e.g., an oxido-reduction catalyst) that converts glycinamide to modified glycinamide are embodiments.

Similarly, techniques in rational drug design and molecular biology can be employed to identify more oxido-reduction catalysts that convert glycinamide to modified glycinamide or to generate synthetic or mutant oxido-reduction catalysts that convert glycinamide to modified glycinamide more efficiently than wild-type enzymes. Rational drug design involving polypeptides requires identifying and defining a first peptide (e.g., leghemoglobin) with which the designed drug (e.g., glycinamide) is to interact, and using the first target peptide to define the requirements for a second peptide (e.g., a modified oxido-reduction catalyst). With such requirements defined, one can find or prepare an appropriate peptide or non-peptide that meets all or substantially all of the defined requirements. Thus, one goal of rational drug design is to produce structural or functional analogs of biologically active polypeptides of interest (e.g., oxido-reduction catalysts) or of small molecules with which they interact (e.g., prodrugs) in order to fashion compounds that are, for example, more or less potent forms. (See, e.g., Hodgson, Bio. Technology 9:19-21 (1991)). An example of rational drug design is shown in Erickson et al., Science 249:527-533 (1990).

Combinatorial chemistry is the science of synthesizing and testing compounds for bioactivity en masse, instead of one by one, the aim being to discover drugs and materials more quickly and inexpensively than was formerly possible. Rational drug design and combinatorial chemistry have become more intimately related in recent years due to the development of approaches in computer-aided protein modeling and drug discovery. (See e.g., U.S. Pat. Nos. 4,908,773; 5,884,230; 5,873,052; 5,331,573; and 5,888,738, herein expressly incorporated by reference in there entireties).

The use of molecular modeling as a tool for rational drug design and combinatorial chemistry has dramatically increased due to the advent of computer graphics. Not only is it possible to view molecules on computer screens in three dimensions but it is also possible to examine the interactions of macromolecules such as enzymes and receptors and rationally design derivative molecules to test. (See Boorman, Chem. Eng. News 70:18-26 (1992). A vast amount of user-friendly software and hardware is now available and virtually all pharmaceutical companies have computer modeling groups devoted to rational drug design. Molecular Simulations Inc., for example, sells several sophisticated programs that allow a user to start from an amino acid sequence, build a two or three-dimensional model of the protein or polypeptide, compare it to other two and three-dimensional models, and analyze the interactions of compounds, drugs, and peptides with a three dimensional model in real time.

Accordingly, in some embodiments of the invention, software is used to compare regions of oxido-reduction catalysts (e.g., leghemoglobin or the bovine serum cofactor) and glycinamide, so that therapeutic interactions can be predicted and designed. (See Schneider, Genetic Engineering News December: page 20 (1998), Tempczyk et al., Molecular Simulations Inc. Solutions April (1997) and Butenhof, Molecular Simulations Inc. Case Notes (August 1998) for a discussion of molecular modeling).

For example, the protein sequence of a oxido-reduction catalyst or a domain of these molecules (or nucleic acid sequence encoding these polypeptides or both), can be entered onto a computer readable medium for recording and manipulation. It will be appreciated by those skilled in the art that a computer readable medium having these sequences can interface with software that converts or manipulates the sequences to obtain structural and functional information, such as protein models. That is, the functionality of a software program that converts or manipulates these sequences includes the ability to compare these sequences to other sequences or structures of molecules that are present on publicly and commercially available databases so as to conduct rational drug design.

The oxido-reduction catalyst or nucleic acid sequence encoding the catalyst or binding partner (e.g., glycinamide) or both can be stored, recorded, and manipulated on any medium that can be read and accessed by a computer. As used herein, the words “recorded” and “stored” refer to a process for storing information on computer readable medium. A skilled artisan can readily adopt any of the presently known methods for recording information on a computer readable medium to generate manufactures comprising the nucleotide or polypeptide sequence information of this embodiment. A variety of data storage structures are available to a skilled artisan for creating a computer readable medium having recorded thereon a nucleotide or polypeptide sequence. The choice of the data storage structure will generally be based on the component chosen to access the stored information. Computer readable media include magnetically readable media, optically readable media, or electronically readable media. For example, the computer readable media can be a hard disc, a floppy disc, a magnetic tape, zip disk, CD-ROM, DVD-ROM, RAM, or ROM as well as other types of other media known to those skilled in the art. The computer readable media on which the sequence information is stored can be in a personal computer, a network, a server or other computer systems known to those skilled in the art.

Embodiments of the invention utilize computer-based systems that contain the sequence information described herein and convert this information into other types of usable information (e.g., protein models for rational drug design). The term “a computer-based system” refers to the hardware, software, and any database used to analyze an oxido-reduction catalyst or a binding partner or both, or fragments of these biomolecules so as to construct models or to conduct rational drug design. The computer-based system preferably includes the storage media described above, and a processor for accessing and manipulating the sequence data. The hardware of the computer-based systems of this embodiment comprise a central processing unit (CPU) and a database. A skilled artisan can readily appreciate that any one of the currently available computer-based systems are suitable.

In one particular embodiment, the computer system includes a processor connected to a bus that is connected to a main memory (preferably implemented as RAM) and a variety of secondary storage devices, such as a hard drive and removable medium storage device. The removable medium storage device can represent, for example, a floppy disk drive, a DVD drive, an optical disk drive, a compact disk drive, a magnetic tape drive, etc. A removable storage medium, such as a floppy disk, a compact disk, a magnetic tape, etc. containing control logic and/or data recorded therein can be inserted into the removable storage device. The computer system includes appropriate software for reading the control logic and/or the data from the removable medium storage device once inserted in the removable medium storage device. The oxido-reduction catalyst nucleic acid or polypeptide sequence or both can be stored in a well known manner in the main memory, any of the secondary storage devices, and/or a removable storage medium. Software for accessing and processing these sequences (such as search tools, compare tools, and modeling tools etc.) reside in main memory during execution.

As used herein, “a database” refers to memory that can store an oxido-reduction catalyst nucleotide or polypeptide sequence information, protein model information, information on other peptides, chemicals, peptidomimetics, and other agents that interact with oxido-reduction catalyst proteins, and values or results from functional assays. Additionally, a “database” refers to a memory access component that can access manufactures having recorded thereon oxido-reduction catalyst or binding partner nucleotide or polypeptide sequence information, protein model information, information on other peptides, chemicals, peptidomimetics, and other agents that interact with oxido-reduction catalysts, and values or results from functional assays. In other embodiments, a database stores an “oxido-reduction catalyst functional profile” comprising the values and results (e.g., glycinamide conversion data, such as chemiluminescent values or conversion units) from one or more “oxido-reduction catalyst functional assays”, as described herein or known in the art, and relationships between these values or results. The sequence data and values or results from oxido-reduction catalyst functional assays can be stored and manipulated in a variety of data processor programs in a variety of formats. For example, the sequence data can be stored as text in a word processing file, such as Microsoft WORD or WORDPERFECT, an ASCII file, a html file, or a pdf file in a variety of database programs familiar to those of skill in the art, such as DB2, SYBASE, or ORACLE.

A “search program” refers to one or more programs that are implemented on the computer-based system to compare an oxido-reduction catalyst or binding partner nucleotide or polypeptide sequence with other nucleotide or polypeptide sequences and agents including but not limited to peptides, peptidomimetics, and chemicals stored within a database. A search program also refers to one or more programs that compare one or more protein models to several protein models that exist in a database and one or more protein models to several peptides, peptidomimetics, and chemicals that exist in a database. A search program is used, for example, to compare one oxido-reduction catalyst functional profile to one or more oxido-reduction catalyst functional profiles that are present in a database. Still further, a search program can be used to compare values or results from oxido-reduction catalyst functional assays and agents that modulate oxido-reduction catalyst-mediated signal transduction.

A “retrieval program” refers to one or more programs that can be implemented on the computer-based system to identify a homologous nucleic acid sequence, a homologous protein sequence, or a homologous protein model. A retrieval program can also used to identify compounds (e.g., glycinamide) peptides, peptidomimetics, and chemicals that interact with an oxido-reduction catalyst protein sequence, or an oxido-reduction catalyst protein model stored in a database.

As a starting point to rational drug design, a two or three dimensional model of a polypeptide of interest is created (e.g., leghemoglobin or the bovine serum cofactor). In the past, the three-dimensional structure of proteins has been determined in a number of ways. Perhaps the best known way of determining protein structure involves the use of x-ray crystallography. A general review of this technique can be found in Van Holde, K. E. Physical Biochemistry, Prentice-Hall, N.J. pp. 221-239 (1971). Using this technique, it is possible to elucidate three-dimensional structure with good precision. Additionally, protein structure can be determined through the use of techniques of neutron diffraction, or by nuclear magnetic resonance (NMR). (See, e.g., Moore, W. J., Physical Chemistry, 4^{th} Edition, Prentice-Hall, N.J. (1972)).

Alternatively, protein models of a polypeptide of interest can be constructed using computer-based protein modeling techniques. By one approach, the protein folding problem is solved by finding target sequences that are most compatible with profiles representing the structural environments of the residues in known three-dimensional protein structures. (See, e.g., U.S. Pat. No. 5,436,850). In another technique, the known three-dimensional structures of proteins in a given family are superimposed to define the structurally conserved regions in that family. This protein modeling technique also uses the known three-dimensional structure of a homologous protein to approximate the structure of a polypeptide of interest. (See e.g., U.S. Pat. Nos. 5,557,535; 5,884,230; and 5,873,052). Conventional homology modeling techniques have been used routinely to build models of proteases and antibodies. (Sowdhamini et al., Protein Engineering 10:207, 215 (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods and “fuzzy” approaches now enables the identification of likely folding patterns and functional protein domains in a number of situations where the structural relatedness between target and template(s) is not detectable at the sequence level. By one method, fold recognition is performed using Multiple Sequence Threading (MST) and structural equivalences are deduced from the threading output using the distance geometry program DRAGON that constructs a low resolution model. A full-atom representation is then constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalences obtained from the MST output are converted into interresidue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and organised to energy minimization using the molecular modeling package QUANTA. (See e.g., Aszódi et al., Proteins:Structure, Function, and Genetics, Supplement 1:38-42 (1997)).

In a preferred approach, the commercially available “Insight II 98” program (Molecular Simulations Inc.) and accompanying modules are used to create a two and/or three dimensional model of a polypeptide of interest (e.g., leghemoglobin) from an amino acid sequence (e.g., SEQ. ID. No. 2) with and without bound glycinamide. Insight II is a three-dimensional graphics program that can interface with several modules that perform numerous structural analysis and enable real-time rational drug design and combinatorial chemistry. Modules such as Builder, Biopolymer, Consensus, and Converter, for example, allow one to rapidly create a two dimensional or three dimensional model of a polypeptide, carbohydrate, nucleic acid, chemical or combinations of the foregoing from their sequence or structure. The modeling tools associated with Insight II support many different data file formats including Brookhaven and Cambridge databases; AMPAC/MOPAC and QCPE programs; Molecular Design Limited Molfile and SD files, Sybel Mol2 files, VRML, and Pict files.

Additionally, the techniques described above can be supplemented with techniques in molecular biology to synthesize more robust enzymes. For example, a polypeptide of interest can be analyzed by an alanine scan (Wells, Methods in Enzymol. 202:390-411 (1991)) or other types of site-directed mutagenesis analysis to identify residues critical for catalysis. In alanine scan, each amino acid residue of the polypeptide of interest is sequentially replaced by alanine in a step-wise fashion (i.e., only one alanine point mutation is incorporated per molecule starting at position #1 and proceeding through the entire molecule), and the effect of the mutation on the peptide's activity in a functional assay is determined. Each of the amino acid residues of the peptide is analyzed in this manner and the regions important for catalysis of glycinamide are identified. These functionally important regions can be recorded on a computer readable medium, stored in a database in a computer system, and a search program can be employed to generate a protein model of the functionally important regions.

Once a model of the polypeptide of interest is created, it can be compared to other models so as to identify new members of the oxido-reduction catalyst family or can be used to synthesize variant enzymes, which can then be tested for the ability to convert glycinamide to modified glycinamide. By repeating the process of developing a model, performing mutatgenisis to the molecule and analyzing the effect on the catalysis of glycinamide, more robust enzymes can be created.

By starting with the amino acid sequence or protein model of a oxido-reduction catalyst, for example, molecules having two-dimensional and/or three-dimensional homology can be rapidly identified. In one approach, a percent sequence identity can be determined by standard methods that are commonly used to compare the similarity and position of the amino acid of two polypeptides. Using a computer program such as BLAST or FASTA, two polypeptides can be aligned for optimal matching of their respective amino acids (either along the full length of one or both sequences, or along a predetermined portion of one or both sequences). Such programs provide “default” opening penalty and a “default” gap penalty, and a scoring matrix such as PAM 250 (a standard scoring matrix; see Dayhoff et al., in: Atlas of Protein Sequence and Structure, Vol. 5, Supp. 3 (1978)) can be used in conjunction with the computer program. The percent identity can then be calculated as:

$$\frac{total number of identical matches}{[ \begin{matrix}length of the longer sequence \\ within the matched span + \\ number of gaps introduced into the longer \\ sequence in order to align the two sequences\end{matrix} ]} \times 100$$

Accordingly, the protein sequence corresponding to an oxido-reduction catalyst or a fragment or derivative thereof can be compared to known sequences on a protein basis. Protein sequences corresponding to a oxido-reduction catalyst or a fragment or derivative thereof are compared, for example, to known amino acid sequences found in Swissprot release 35, PIR release 53 and Genpept release 108 public databases using BLASTP with the parameter W=8 and allowing a maximum of 10 matches. In addition, the protein sequences are compared to publicly known amino acid sequences of Swissprot using BLASTX with the parameter E=0.001. The molecules identified as members of the family of oxido-reduction catalysts that convert glycinamide to a modified glycinamide desirably have at least 35% homology and preferably have 40%, 45%, 50% or 55% or greater homology to a oxido-reduction catalyst. Preferred oxido-reduction catalysts that react with glycinamide to form modified glycinamide have the following degrees of homology to leghemoglobin (e.g., Phaseolus vulgaris) greater than or equal to: 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The oxido-reduction catalyst family members having greater than or equal to 35% homology are identified and are subsequently examined using an oxido-reduction catalyst functional assay.

In another embodiment, computer modeling and the sequence-to-structure-to-function paradigm is exploited to identify more enzymes that catalyse the conversion of glycinamide to modified glycinamide. By this approach, first the structure of an oxido-reduction catalyst (e.g., leghemoglobin) having a known response in a characterization assay is determined from its sequence using a threading algorithm, which aligns the sequence to the best matching structure in a structural database. Next, the protein's active site (i.e., the site important for a desired response in the characterization assay) is identified and a “fuzzy functional form” (FFF)—a three-dimensional descriptor of the active site of a protein—is created. (See e.g., Fetrow et al., J. Mol. Biol. 282:703-711 (1998) and Fetrow and Skolnick, J. Mol. Biol. 281: 949-968 (1998).

The FFFs are built by iteratively superimposing the protein geometries from a series of functionally related proteins with known structures. The FFFs are not overly specific, however, and the degree to which the descriptors can be relaxed is explored. In essence, conserved and functionally important residues for a desired response are identified and a set of geometric and conformational constraints for a specific function are defined in the form of a computer algorithm. The program then searches experimentally determined protein structures from a protein structural database for sets of residues that satisfy the specified constraints. In this manner, homologous three-dimensional structures can be compared and degrees (e.g., percentages of three-dimensional homology) can be ascertained. The ability to search three-dimensional structure databases for structural similarity to a protein of interest can also be accomplished by employing the Insight II using modules such as Biopolymer, Binding Site Analysis, and Profiles-3D.

By using this computational protocol, genome sequence data bases can be rapidly screened for specific protein active sites and for identification of the residues at those active sites that resemble a desired molecule. The Molecular Modelling Database (MMDB), and the Protein Data Bank can use short stretches of sequence information to identify sequence patterns that are specific for a given function; thus they avoid the problems arising from the necessity of matching entire sequences.

If an incomplete or insufficiently accurate structure is determined, the methods of computer based numerical modeling can be used to complete the structure or improve its accuracy. Any recognized modeling method can be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Many more computer programs and databases can be used with embodiments of the invention to identify and/or develop new enzymes that convert glycinamide to modified glycinamide. The following list is intended not to limit the invention but to provide guidance to programs and databases that are useful with the approaches discussed above. The programs and databases that can be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403 (1990), herein incorporated by reference), FASTA (Pearson and Lipman, Proc. Natl. Acad. Sci. USA, 85: 2444 (1988), herein incorporated by reference), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), Modeller 4 (SalI and Blundell J. Mol. Biol. 234:217-241 (1997)), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), Biopendiurn (Inpharmatica), SBdBase (Structural Bioinformatics), the EMBL/Swissprotein database, the MDL Available Chemicals Directory database, the MDL Drug Data Report data base, the Comprehensive Medicinal Chemistry database, Derwents's World Drug Index database, and the BioByteMasterFile database. Many other programs and data bases would be apparent to one of skill in the art given the present disclosure.

The identified wild-type oxido-reduction catalysts and the developed or synthesized oxido-reduction catalysts that convert glycinamide to modified glycinamide can be isolated, enriched, or purified from a source or the genes encoding these enzymes can be cloned and inserted into various expression vectors designed, for example, expression in a host animal or for large-scale in vitro protein production in bacteria or plants. A variety of host-expression vector systems can be utilized to express the oxido-reduction catalysts described herein. Where the oxido-reduction catalyst is a soluble protein it can be recovered from the culture, i.e., from the host cell in cases where the peptide or polypeptide is not secreted, and from the culture media in cases where the peptide or polypeptide is secreted by the cells. However, the expression systems also encompass engineered host cells that express the oxido-reduction catalyst in situ, i.e., anchored in the cell membrane. Purification or enrichment of the oxido-reduction catalyst from such expression systems can be accomplished using appropriate detergents and lipid micelles and methods well known to those skilled in the art. However, such engineered host cells themselves can be used in situations where it is important not only to retain the structural and functional characteristics of the oxido-reduction catalyst, but to assess biological activity.

The expression systems that can be used for purposes of the invention include, but are not limited to, microorganisms such as bacteria (e.g., E. coli or B. subtilis) transformed with recombinant bacteriophage DNA, plasmid DNA or cosmid DNA expression vectors containing oxido-reduction catalyst nucleotide sequences; yeast (e.g., Saccharomyces, Pichia) transformed with recombinant yeast expression vectors containing the oxido-reduction catalyst nucleotide sequences; insect cell systems infected with recombinant virus expression vectors (e.g., baculovirus) containing the oxido-reduction catalyst sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing oxido-reduction catalyst nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5 K promoter).

In bacterial systems, a number of expression vectors can be advantageously selected depending upon the use intended for the oxido-reduction catalyst gene product being expressed. For example, when a large quantity of such a protein is to be produced, for the generation of pharmaceutical compositions or dietary supplements comprising an oxido-reduction catalyst or for raising antibodies to the oxido-reduction catalyst protein, for example, vectors that direct the expression of high levels of fusion protein products that are readily purified can be desirable. Such vectors include, but are not limited, to the E. coli expression vector pUR278 (Ruther et al., EMBO J., 2:1791 (1983), in which the oxido-reduction catalyst coding sequence can be ligated individually into the vector in frame with the lacZ coding region so that a fusion protein is produced; pIN vectors (Inouye & Inouye, Nucleic Acids Res., 13:3101-3109 (1985); Van Heeke & Schuster, J. Biol. Chem., 264:5503-5509 (1989)); and the like. pGEX vectors can also be used to express foreign polypeptides as fusion proteins with glutathione S-transferase (GST). In general, such fusion proteins are soluble and can be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The oxido-reduction catalyst gene coding sequence can be cloned individually into non-essential regions (for example the polyhedrin gene) of the virus and placed under control of an AcNPV promoter (for example the polyhedrin promoter). Successful insertion of oxido-reduction catalyst gene coding sequence will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted gene is expressed. (See e.g., Smith et al., J. Virol. 46: 584 (1983); and Smith, U.S. Pat. No. 4,215,051).

In mammalian host cells, a number of viral-based expression systems can be utilized. In cases where an adenovirus is used as an expression vector, the oxido-reduction catalyst nucleotide sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the oxido-reduction catalyst gene product in infected hosts. (See e.g., Logan & Shenk, Proc. Natl. Acad. Sci. USA 81:3655-3659 (1984)). Specific initiation signals can also be required for efficient translation of inserted oxido-reduction catalyst nucleotide sequences. These signals include the ATG initiation codon and adjacent sequences. In cases where an entire oxido-reduction catalyst gene or cDNA, including its own initiation codon and adjacent sequences, is inserted into the appropriate expression vector, no additional translational control signals are needed.

However, in cases where only a portion of the oxido-reduction catalyst coding sequence is inserted, exogenous translational control signals, including, perhaps, the ATG initiation codon, should be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See Bittner et al., Methods in Enzymol., 153:516-544 (1987)).

In addition, a host cell strain can be chosen which modulates the expression of the inserted sequences, or modifies and processes the gene product in the specific fashion desired. Such modifications (e.g., glycosylation) and processing (e.g., cleavage) of protein products are important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, 3T3, and WI38.

For long-term, high-yield production of recombinant proteins, stable expression is preferred. For example, cell lines that stably express the oxido-reduction catalyst sequences described above can be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells are allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn are cloned and expanded into cell lines. This method is advantageously used to engineer cell lines which express the oxido-reduction catalyst gene product. Such engineered cell lines are particularly useful in screening and evaluation of compounds that affect the endogenous activity of the oxido-reduction catalyst gene product.

A number of selection systems can be used, including but not limited to the herpes simplex virus thymidine kinase (Wigler, et al., Cell 11:223 (1977), hypoxanthine-guanine phosphoribosyltransferase (Szybalska & Szybalski, Proc. Natl. Acad. Sci. USA 48:2026 (1962), and adenine phosphoribosyltransferase (Lowy, et al., Cell 22:817 (1980) genes can be employed in tk.⁻, hgprt⁻ or aprt⁻ cells, respectively. Also, antimetabolite resistance can be used as the basis of selection for the following genes: dhfr, which confers resistance to methotrexate (Wigler, et al., Proc. Natl. Acad. Sci. USA 77:3567 (1980); O'Hare, et al., Proc. Natl. Acad. Sci. USA 78:1527 (1981); gpt, which confers resistance to mycophenolic acid (Mulligan & Berg, Proc. Natl. Acad. Sci. USA 78:2072 (1981); neo, which confers resistance to the aminoglycoside G-418 (Colberre-Garapin, et al., J. Mol. Biol. 150:1 (1981); and hygro, which confers resistance to hygromycin (Santerre, et al., Gene 30:147 (1984)).

Alternatively, any fusion protein can be readily purified by utilizing an antibody specific for the fusion protein being expressed. For example, a system described by Janknecht et al. allows for the ready purification of non-denatured fusion proteins expressed in human cell lines. (Janknecht, et al., Proc. Natl. Acad. Sci. USA 88: 8972-8976 (1991)). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The oxido-reduction catalyst can also be expressed in plants, insects, and animals so as to create a transgenic organism. Plants and insects of almost any species can be made to express an oxido-reduction catalyst, fragments of oxido-reduction catalyst, or oxido-reduction catalyst-like hybrids. Desirable transgenic plant systems having an introduced oxido-reduction catalyst, fragments of oxido-reduction catalyst, or oxido-reduction catalyst-like molecule include Arabadopsis, Maize, Chlamydomonas, Leguminosae, particularly Phaseolus, preferably, Phaseolus vulgaris. Desirable insect systems an oxido-reduction catalyst, fragments of oxido-reduction catalyst, or oxido-reduction catalyst-like hybrid include, for example, D. melanogaster and C. elegans. Animals of any species, including, but not limited to, amphibians, reptiles, birds, mice, rats, rabbits, guinea pigs, pigs, micro-pigs, goats, dogs, cats, and non-human primates, e.g., baboons, monkeys, and chimpanzees can be used to generate oxido-reduction catalyst transgenic animals. Transgenic organisms of the invention desirably exhibit germline transfer of wild-type or mutant oxido-reduction catalysts, fragments of oxido-reduction catalyst, or oxido-reduction catalyst-like hybrids. Other transgenic organisms of the invention are engineered to express human or humanized oxido-reduction catalysts, fragments of oxido-reduction catalysts, or oxido-reduction catalyst-like hybrid molecules. Still other transgenic organisms of the invention exhibit complete knockouts or point mutations of one or more existing oxido-reduction catalyst genes.

Any technique known in the art is preferably used to introduce the oxido-reduction catalyst transgene into animals to produce the founder lines of transgenic animals or to knock out or replace existing oxido-reduction catalyst genes. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al:, Proc. Natl. Acad. Sci., USA 82:6148-6152 (1985); gene targeting in embryonic stem cells (Thompson et al., Cell 56:313-321 (1989); electroporation of embryos (Lo, Mol. Cell. Biol. 3:1803-1814 (1983); and sperm-mediated gene transfer (Lavitrano et al., Cell 57:717-723 (1989); etc. For a review of such techniques, see Gordon, Transgenic Animals, Intl. Rev. Cytol. 115:171-229 (1989), which is incorporated by reference herein in its entirety.

The invention provides for transgenic animals that carry a oxido-reduction catalyst transgene in all their cells, as well as animals that carry the transgene in some, but not all their cells, i.e., mosaic animals. The transgene can be integrated as a single transgene or in concatamers, e.g., head-to-head tandems or head-to-tail tandems. The transgene can also be selectively introduced into and activated in a particular cell type by following, for example, the teaching of Lasko et al. (Lasko, M. et al., Proc. Natl. Acad. Sci. USA 89: 6232-6236 (1992)). The regulatory sequences required for such a cell-type specific activation will depend upon the particular cell type of interest, and will be apparent to those of skill in the art.

When it is desired that the oxido-reduction catalyst gene transgene be integrated into the chromosomal site of the endogenous oxido-reduction catalyst gene, gene targeting is preferred. Briefly, when such a technique is to be utilized, vectors containing some nucleotide sequences homologous to the endogenous oxido-reduction catalyst gene are designed for the purpose of integrating, via homologous recombination with chromosomal sequences, into and disrupting the function of the nucleotide sequence of the endogenous oxido-reduction catalyst gene. The transgene can also be selectively introduced into a particular cell type, thus inactivating the endogenous oxido-reduction catalyst gene in only that cell type, by following, for example, the teaching of Gu et al. (Gu, et al., Science 265: 103-106 (1994)). The regulatory sequences required for such a cell-type specific inactivation will depend upon the particular cell type of interest, and will be apparent to those of skill in the art.

Once transgenic animals have been generated, the expression of the recombinant oxido-reduction catalyst gene can be assayed utilizing standard techniques. Initial screening can be accomplished by Southern blot analysis or PCR techniques to analyze animal tissues to assay whether integration of the transgene has taken place. The level of mRNA expression of the transgene in the tissues of the transgenic animals can also be assessed using techniques which include, but are not limited to, Northern blot analysis of tissue samples obtained from the animal, in situ hybridization analysis, and RT-PCR. Samples of oxido-reduction catalyst gene-expressing tissue can also be evaluated immunocytochemically using antibodies specific for the oxido-reduction catalyst transgene product.

In addition to the naturally occurring or wild-type oxido-reduction catalysts or peptide-based hybrids (e.g., peptidomimetics), embodiments of the invention include derivative or modified molecules (e.g., mutant oxido-reduction catalysts) that convert glycinamide to modified glycinamide more robustly. For example, a derivative oxido-reduction catalyst can include a polypeptide that has been engineered to have one or more cystine residues incorporated into the protein so as to promote the formation of a more stable derivative through disulfide bond formation. (See e.g., U.S. Pat. No. 4,908,773). In the past, investigators have employed computers and computer graphics programs to aid in assessing the appropriateness of potential cystine linkage sites. (Perry, L. J., & Wetzel, R., Science, 226:555-557 (1984); Pabo, C. O., et al., Biochemistry, 25:5987-5991 (1986); Bott, R., et al., European Patent Application Ser. No. 130,756; Perry, L. J., & Wetzel, R., Biochemistry, 25:733-739 (1986); Wetzel, R. B., European Patent Application Ser. No. 155,832). The introduction of a cystine residue in a polypeptide can be accomplished using conventional molecular biology techniques.

Preferably, the oxido-reduction catalysts used to convert glycinamide to a modified glycinamide that inhibits replication of HIV, or improves immune system function, or ameliorates a condition associated with HIV infection (e.g, reduced T cell count) convert at least, equal to, or greater than 80% of a concentration of glycinamide of 10 μM, 25 μM, 50 μM, 100 μM, 200 μM, 300 μM, 400 μM, 500 μM, 600 μM, 700 μM, 800 μM, 900 μM, 1000 μM, 1200 μM, 1500 μM, 1800 μM, or 2000 μM or more in 24 hours or that converts glycinamide to a modified glycinamide, such as α-hydroxy glycinamide, at a rate that is at least, equal to, or greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, 5.0, 6.0, 7.0, 8.0, 9.0, 10.0, 20.0, 30.0 μg/hour

Additional oxido-reduction catalysts and hybrid molecules include peptidomimetics that resemble a polypeptide of interest. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Synthetic compounds that mimic the conformation and desirable features of a particular peptide, e.g., an oligopeptide, once such peptide has been found, but that avoids the undesirable features, e.g., flexibility (loss of conformation) and bond breakdown are known as a “peptidomimetics”. (See, e.g., Spatola, A. F. Chemistry and Biochemistry of Amino Acids. Peptides, and Proteins (Weistein, B, Ed.), Vol. 7, pp. 267-357, Marcel Dekker, New York (1983), which describes the use of the methylenethio bioisostere [CH₂ S] as an amide replacement in enkephalin analogues; and Szelke et al., In peptides: Structure and Function, Proceedings of the Eighth American Peptide Symposium, (Hruby and Rich, Eds.); pp. 579-582, Pierce Chemical Co., Rockford, Ill. (1983), which describes renin inhibitors having both the methyleneamino [CH₂ NH] and hydroxyethylene [CHOHCH₂] bioisosteres at the Leu-Val amide bond in the 6-13 octapeptide derived from angiotensinogen).

In general, the design and synthesis of a peptidomimetic involves starting with the amino acid sequence of the peptide and conformational data (e.g., geometry data, such as bond lengths and angles) of a desired peptide (e.g., the most probable simulated peptide). That data is then used to determine the geometries that should be designed into the peptidomimetic. Numerous methods and techniques are known in the art for performing this step, any of which could be used. (See, e.g., Farmer, P. S., Drug Design, (Ariens, E. J. ed.), Vol. 10, pp. 119-143 (Academic Press, New York, London, Toronto, Sydney and San Francisco) (1980); Farmer, et al., in TIPS, 9/82, pp. 362-365; Verber et al., in TINS, 9/85, pp. 392-396; Kaltenbronn et al., in J. Med. Chem. 33: 838-845 (1990); and Spatola, A. F., in Chemistry and Biochemistry of Amino Acids, Peptides, and Proteins, Vol. 7, pp. 267-357, Chapter 5, “Peptide Backbone Modifications: A Structure-Activity Analysis of Peptides Containing Amide Bond Surrogates. Conformational Constraints, and Relations” (B. Weisten, ed.; Marcell Dekker: New York, pub.) (1983); Kemp, D. S., “Peptidomimetics and the Template Approach to Nucleation of β-sheets and α-helices in Peptides,” Tibech, Vol. 8, pp. 249-255 (1990). Additional teachings can be found in U.S. Pat. Nos. 5,288,707; 5,552,534; 5,811,515; 5,817,626; 5,817,879; 5,821,231; and 5,874,529.

In some embodiments, a gene encoding a oxido-reduction catalyst (e.g., a leghemoglobin or serum cofactor that converts glycinamide to modified glycinamide) is transferred to a subject in need of an ability to convert glycinamide to a modified glycinamide (e.g., α-hydroxyglycinamide) or in need of a greater ability to produce modified glycinamide. Although any native oxido-reduction catalyst can be transferred using the approaches described herein, it is preferred that the gene is codon-optimized for the particular host so as to improve the translation effeiciency therein. That is, for example, if the gene encoding the oxido-reduction catalyst is to be expressed in bacteria (e.g., for large scale production of modified glycinamide) the gene to be transferred is codon-optimized for expression in bacteria. Similarly, if the gene encoding the oxido-reduction catalyst is to be expressed in humans, (e.g., for gene therapy) the gene to be transferred is codon-optimized for expression in humans. EXAMPLE 9 describes an approach that was used to develop a human codon-optimized leghemoglobin gene.

Compounds that Inhibit Replication of HIV

Several HIV infectivity studies were conducted to evaluate the ability of the cofactors described herein to convert glycinamide into a modified glycinamide that has an antiretroviral activity and to evaluate the ability of the various modified glycinamide compounds described herein (e.g., compounds of formulas A, B, C, D, E, F, G, H, I, J, K, L, M, N, O, P, Q, R, S, T, U, V, W, X, Y, Z, or AA) to inhibit the replication of HIV. Although many types of HIV infectivity assays can be conducted, by one approach, human T-lymphocytic CEM cells (approx. 4.5×10⁵ cells/ml) are suspended in fresh medium and are infected with HIV-1 (III_{B}) at approx. 100CCID₅₀ per ml of cell suspension. Then, 100 μl of the infected cell suspension is transferred to individual wells of a microtiter plate (100 μl/well) and is mixed with 100 μl of freshly diluted modified G-NH₂ (fraction 2-3), G-NH₂ (fraction 15-17), α-hydroxyglycinamide and/or another compound of formulas A, B, C, D, E, F, G, H, I, J, K, L, M, N, O, P, Q, R, S, T, U, V, W, X, Y, Z, or AA (e.g., 2000, 400, 80, 16, 3.2, and 0.62 μM). Subsequently, the mixtures are incubated at 37° C. After 4 to 5 days, giant cell formation is recorded microscopically in the CEM cultures and the 50% effective concentration (EC₅₀) is determined.

The results from this type of HIV infectivity study will show that modified G-NH₂ (fraction 2-3), α-hydroxyglycinamide, and/or a compound of formula A, B, C, D, E, F, G, H, I, J, K, L, M, N, O, P, Q, R, S, T, U, V, W, X, Y, Z, or AA has a comparable or lower EC₅₀ than G-NH₂ (fraction 15-17). For example, modified G-NH₂, α-hydroxyglycinamide and/or a compound of formula A, B, C, D, E, F, G, H, I, J, K, L, M, N, O, P, Q, R, S, T, U, V, W, X, Y, Z, or AA will have an EC₅₀ of approximately 25 μM or less, whereas, G-NH₂ will have an EC₅₀ of approximately 30 μM.

By another approach, the ability of modified G-NH₂ to inhibit the replication of HIV in heat inactivated serum (30 minutes at 95° C.) or human serum-containing medium is compared. Human T-lymphocytes (e.g., approx. 4.5×10⁵ cells/ml of CEM cells) are suspended in fresh medium containing fetal bovine serum and are infected with HIV-1 (III_{B}) at approx. 100CCID₅₀ per ml of cell suspension. Then, the infected cells are washed in PBS and resuspended in medium containing 10% fetal bovine serum that was heated for 30 minutes at 95° C. or human serum. Next, 100 μl of the infected cell suspension is transferred to individual wells of a microtiter plate (100 μl/well) and is mixed with 100 μl of freshly diluted purified, modified G-NH₂ (fraction 2-3), α-hydroxyglycinamide and/or a compound of formulas A, B, C, D, E, F, G, H, I, J, K, L, M, N, O, P, Q, R, S, T, U, V, W, X, Y, Z, or AA (e.g., 2000, 400, 80, 16, 3.2, and 0.62 μM). Subsequently, the mixtures are incubated at 37° C. After 4 to 5 days of incubation, giant cell formation is recorded microscopically in the cultures. The 50% effective concentration (EC₅₀) is then determined. The results from this set of experiments will show that the purified, modified G-NH₂ (fraction 2-3), α-hydroxyglycinamide and/or a compound of formulas A, B, C, D, E, F, G, H, I, J, K, L, M, N, O, P, Q, R, S, T, U, V, W, X, Y, Z, or AA efficiently inhibits replication of HIV in the boiled fetal bovine serum or human serum samples, whereas purified G-NH₂ (fraction 15-17) does not.

By still another approach, the ability of a cofactor (e.g. recombinant, isolated, purified or enriched animal serum or plasma cofactor plant cofactor, such as leghemoglobin) to convert glycinamide to a modified glycinamide that inhibits replication of HIV is evaluated. Human T-lymphocytes (e.g., approx. 4.5×10⁵ cells/ml of CEM cells) are suspended in fresh medium containing fetal bovine serum and are infected with HIV-1 (III_{B}) at approx. 100CCID₅₀ per ml of cell suspension. Then, the infected cells are washed in PBS and resuspended in medium containing 10% fetal bovine serum that was heated for 30 minutes at 95° C. or human serum. Next, 100 μl of the infected cell suspension is transferred to individual wells of a microtiter plate (100 μl/well) and is mixed with 100 μl of freshly diluted purified, glycinamide (e.g., 2000, 400, 80, 16, 3.2, and 0.62 μM). Additionally, various quantities (5, 10, 25, 50, 100, 1000 μg) of recombinant, isolated, purified or enriched animal serum or plasma cofactor plant cofactor are added to reaction tubes. Subsequently, the mixtures are incubated at 37° C. After 4 to 5 days of incubation, giant cell formation is recorded microscopically in the cultures. The 50% effective concentration (EC₅₀) is then determined. The results from this set of experiments will show that the glycinamide is efficiently converted to modified glycinamide and that the modified glycinamide created by the reaction has an EC50 that is comparable to α-hydroxyglycinamide and/or a compound of formulas A, B, C, D, E, F, G, H, I, J, K, L, M, N, O, P, Q, R, S, T, U, V, W, X, Y, Z, or AA. EXAMPLE 10 describes experiments that demonstrated that enzymatically prepared α-hydroxyglycinamide (Metabolite X) effectively inhibits the replication of HIV.

In more experiments, the 50% inhibitory concentration (IC₅₀) of Metabolite X was analysed in cell cultures containing fetal calf serum. EXAMPLE 11 describes these experiments in greater detail.

In more experiments, enzymatically prepared modified glycinamide (Metabolite X) was incubated with HIV infected H9 cells (SF2 virus) and the morphology of the treated virus was sent to be analysed by electron microscopy. As a positive control, GPG-NH₂ was used. (See U.S. Pat. No. 6,258,932, herein expressly incorporated by reference in its entirety, for an approach to perform these type of electron microscopy experiments). EXAMPLE 12 describes these experiments in greater detail.

In still more experiments, the antiretroviral activity of G-NH₂, GPG-NH₂, enzymatically prepared modified glycinamide (Metabolite X), and synthetically prepared modified glycinamide (AlphaHGA) were compared. EXAMPLE 13 describes these experiments in greater detail.

In still more experiments, the antiretroviral activity of enzymatically prepared and synthetically prepared alpha hydroxyglycinamide, in the presence of human or fetal calf serum, were compared. EXAMPLE 14 describes these experiments in greater detail.

In another series of experiments, the stability of synthetically prepared alpha-hydroxy glycinamide (AlphaHGA) to prolonged heating at 37° C. was analysed. Diluted samples of synthesized AlphaHGA (C₂H₇ClN₂O₂), were incubated at 37° C. for periods of time and then the antiretroviral activity of the incubated compound was compared to that of freshly diluted AlphaHGA. These experiments are described in greater detail in EXAMPLE 15.

Once it had been determined that alphaHGA effectively inhibited replication of HIV, several analogs and derivatives were evaluated for their ability to inhibit HIV replication. Approximately 250 compounds were obtained from commercial sources and/or were prepared using approaches such as that described in U.S. Pat. No. 6,365,752, herein expressly incorporated by reference in its entirety. These compounds were tested in HIV infectivity assays similar to those described in the Examples. In one set of experiments, the anti-HIV activity of the compounds of formulas K (C₆H₁₁N₃OS) and M (C₉H₁₅N₃OS) were compared to AlphaHGA, G-NH₂, GPG-NH₂, Oxamide (H₂NCOCONH₂), and Glycolamide (HO—CH₂—CO—NH₂). EXAMPLE 16 describes these studies in greater detail.

Once the antiretroviral properties of the compounds of formulas K and M had been determined, toxicity assays were performed to assess and compare the toxicity of alpha-HGA and compounds of formulas K and M. EXAMPLE 17 describes mitogenic assays that were conducted on the compounds of formulas K and M and α-hydroxy glycinamide.

In another series of experiments, it was discovered that amino acid conjugated or amino acid derivatized alphahydroxyglycinamide (e.g., Valine-alphahydroxyglycinamide) effectively inhibits replication of HIV. An amino acid conjugated alphahydroxyglycinamide (Valine-alphahydroxyglycinamide) was analyzed in an HIV infectivity assay, as described herein, and the ability of this class of compounds to inhibit replication of HIV was confirmed. EXAMPLE 18 describes these experiments in greater detail.

The section that follows describes the preparation of pharmaceuticals and dietary supplements that contain modified glycinamide and the use of these compositions to treat, prevent, and/or inhibit replication of HIV or to improve the function of the immune system or to otherwise promote the general health or welfare of an individual that consumes said products.

Antiretroviral Compounds

As discussed above, the compounds described herein inhibit the replication of HIV and therefore promote the maintenance of a healthy immune system in individuals that are at risk of becoming infected with HIV or improve the function of the immune system in individuals that are infected with HIV by improving or promoting the survival of T cells. Accordingly, the modified glycinamide compounds described herein can be provided as both a dietary supplement that promotes maintenance or improvement of the immune system of an individual or as a pharmaceutical that treats or prevents replication of HIV. Some pharmaceuticals or dietary supplements consist of, consist essentially of, or comprise a compound of formula A: 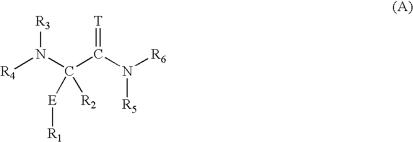
or a pharmaceutically acceptable salt, amide, ester, or prodrug thereof; wherein:

a) E is selected from the group consisting of oxygen, sulfur, and NR₇;

b) T is selected from the group consisting of oxygen, sulfur, and NR₈; and

c) R₁-R₆ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl.

Accordingly, the term “modified G-NH₂ or modified glycinamide compound” includes derivatives and metabolites of glycinamide, such as those of formula A, B, C, D, E, F, G, H, I, J, K, L, M, N, O, P, Q, R, S, T, U, V, W, X, Y, Z, or AA, as described herein, whether enriched or isolated from a cell or synthetically prepared (e.g., α-hydroxyglycinamide, α-peroxyglycinamide dimer (NH₂-gly-O—O-gly-NH₂), α-methoxyglycinamide, α-ethoxyglycinamide, and/or derivatives thereof).

Some of these compounds have been extracted from the HPLC column after glycinamide was incubated in serum, as described above, and identified by mass spectrometry and nuclear magnetic resonance (NMR) spectrometry. These compounds and derivatives or related compounds can be synthesized from available starting materials, as described below.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility or stability in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated by reference herein in its entirety, including any drawings.

The term “aromatic” refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” refers to an aromatic group which contains at least one heterocyclic ring.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds of the invention may be designated as “C₁₋₆ alkyl” or similar designations. By way of example only, “C₁₋₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, t-butyl, pentyl (straight chain or branched), and hexyl (straight chain or branched).

The alkyl group may be substituted or unsubstituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substitutent may be substituted with one of the above substituents.

The substituent “R” appearing by itself and without a number designation refers to a substituent selected from the group consisting of alkyl, cycloalkyl, aryl, heteroaryl (bonded through a ring carbon) and heteroalicyclic (bonded through a ring carbon).

An “O-carboxy” group refers to a RC(═O)O— group, where R is as defined herein.

A “C-carboxy” group refers to a —C(═O)OR groups where R is as defined herein.

An “acetyl” group refers to a —C(═O)CH₃, group.

A “trihalomethanesulfonyl” group refers to a X₃CS(═O)₂— group where X is a halogen.

A “cyano” group refers to a —CN group.

An “isocyanato” group refers to a —NCO group.

A “thiocyanato” group refers to a —CNS group.

An “isothiocyanato” group refers to a —NCS group.

A “sulfinyl” group refers to a —S(═O)—R group, with R as defined herein.

A “S-sulfonamido” group refers to a —S(═O)₂NR, group, with R as defined herein.

A “N-sulfonamido” group refers to a RS(═O)₂NH— group with R as defined herein.

A “trihalomethanesulfonamido” group refers to a X₃CS(═O)₂NR— group with X and R as defined herein.

An “O-carbamyl” group refers to a —OC(═O)—NR, group with R as defined herein.

An “N-carbamyl” group refers to a ROC(═O)NH— group, with R as defined herein.

An “O-thiocarbamyl” group refers to a —OC(═S)—NR, group with R as defined herein.

An “N-thiocarbamyl” group refers to an ROC(═S)NH— group, with R as defined herein.

A “C-amido” group refers to a —C(═O)—NR₂ group with R as defined herein.

An “N-amido” group refers to a RC(═O)NH— group, with R as defined herein.

The term “perhaloalkyl” refers to an alkyl group where all of the hydrogen atoms are replaced by halogen atoms.

In the present context the term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Some examples of “aryl” rings include optionally substituted phenyl, naphthalenyl, phenanthrenyl, anthracenyl, tetralinyl, fluorenyl, indenyl, and indanyl. The term “aryl” relates to aromatic, preferably benzenoid groups, connected via one of the ring-forming carbon atoms, and optionally carrying one or more substituents selected from heterocyclyl, heteroaryl, halo, hydroxy, amino, cyano, nitro, alkylamido, acyl, C₁₋₆ alkoxy, C₁₋₆ alkyl, C₁₋₆ hydroxyalkyl, C₁₋₆ aminoalkyl, C₁₋₆ alkylamino, alkylsulfenyl, alkylsulfinyl, alkylsulfonyl, sulfamoyl, or trifluoromethyl. The aryl group may be substituted at the para and/or meta positions. Representative examples of aryl groups include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl 3-cyanophenyl, 4-cyanophenyl, dimethylphenyl, naphthyl, hydroxynaphthyl, hydroxymethylphenyl, trifluoromethylphenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

In the present context, the term “heteroaryl” is intended to mean a heterocyclic aromatic group where one or more carbon atoms in an aromatic ring have been replaced with one or more heteroatoms selected from the group comprising nitrogen, sulfur, phosphorous, and oxygen.

Furthermore, in the present context, the term “heteroaryl” comprises fused ring systems wherein at least one aryl ring and at least one heteroaryl ring, at least two heteroaryl rings, at least one heteroaryl ring and at least one heterocyclyl ring, or at least one heteroaryl ring and at least one C₃₋₈-cycloalkyl ring share at least one chemical bond.

The term “heteroaryl” is understood to relate to aromatic, C₃₋₈ cyclic groups further containing one oxygen or sulfur atom or up to four nitrogen atoms, or a combination of one oxygen or sulfur atom with up to two nitrogen atoms, and their substituted as well as benzo- and pyrido-fused derivatives, preferably connected via one of the ring-forming carbon atoms. Heteroaryl groups may carry one or more substituents, selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. In some embodiments, heteroaryl groups may be five- and six-membered aromatic heterocyclic systems carrying 0, 1, or 2 substituents, which may be the same as or different from one another, selected from the list above. Representative examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine and pyrazine, which are all preferred, as well as furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, amino-C₁₋₆-alkyl.

In the present context, the term “alkyl” and “C₁₋₆-alkyl” are intended to mean a linear or branched saturated hydrocarbon chain wherein the longest chain has from one to six carbon atoms, such as methyl, ethyl, n-propyl, isopropyl, n-butyl, isobutyl, sec-butyl, tert-butyl, pentyl, isopentyl, neopentyl, and hexyl. An alkyl chain may be optionally substituted.

The term “heterocyclyl” is intended to mean three-, four-, five-, six-, seven-, and eight-membered rings wherein carbon atoms together with from 1 to 3 heteroatoms constitute said ring. A heterocyclyl may optionally contain one or more unsaturated bonds situated in such a way, however, that an aromatic π-electron system does not arise. The heteroatoms are independently selected from oxygen, sulfur, and nitrogen.

A heterocyclyl may further contain one or more carbonyl or thiocarbonyl functionalities, so as to make the definition include oxo-systems and thio-systems such as lactams, lactones, cyclic imides, cyclic thioimides, cyclic carbamates, and the like.

Heterocyclyl rings may optionally also be fused to aryl rings, such that the definition includes bicyclic structures. Preferred such fused heterocyclyl groups share one bond with an optionally substituted benzene ring. Examples of benzo-fused heterocyclyl groups include, but are not limited to, benzimidazolidinone, tetrahydroquinoline, and methylenedioxybenzene ring structures.

Some examples of “heterocyclyls” include, but are not limited to, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Binding to the heterocycle may be at the position of a heteroatom or via a carbon atom of the heterocycle, or, for benzo-fused derivatives, via a carbon of the benzenoid ring.

The term “(heterocyclyl)C₁₋₆-alkyl” is understood as heterocyclyl groups connected, as substituents, via an alkyl, each as defined herein. The heterocyclyl groups of (heterocyclyl)C₁₋₆-alkyl groups may be substituted or unsubstituted. The term “(heterocyclyl)C₁₋₆-alkyl” is intended to mean an alkyl chain substituted at least once with a heterocyclyl group, typically at the terminal position of the alkyl chain.

In the present context, the term “C₂₋₈-alkenyl” is intended to mean a linear or branched hydrocarbon group having from two to eight carbon atoms and containing one or more double bonds. Some examples of C₂₋₈-alkenyl groups include allyl, homo-allyl, vinyl, crotyl, butenyl, pentenyl, hexenyl, heptenyl and octenyl. Some examples of C₂₋₈-alkenyl groups with more than one double bond include butadienyl, pentadienyl, hexadienyl, heptadienyl, heptatrienyl and octatrienyl groups as well as branched forms of these. The position of the unsaturation (the double bond) may be at any position along the carbon chain.

In the present context the term “C₂₋₈-alkynyl” is intended to mean a linear or branched hydrocarbon group containing from two to eight carbon atoms and containing one or more triple bonds. Some examples of C₂₋₈-alkynyl groups include ethynyl, propynyl, butynyl, pentynyl, hexynyl, heptynyl and octynyl groups as well as branched forms of these. The position of unsaturation (the triple bond) may be at any position along the carbon chain. More than one bond may be unsaturated such that the “C₂₋₈-alkynyl” is a di-yne or enedi-yne as is known to the person skilled in the art.

In the present context, the term “C₃₋₈-cycloalkyl” is intended to cover three-, four-, five-, six-, seven-, and eight-membered rings comprising carbon atoms only. A C₃₋₈-cycloalkyl may optionally contain one or more unsaturated bonds situated in such a way, however, that an aromatic π-electron system does not arise.

Some examples of preferred “C₃₋₈-cycloalkyl” are the carbocycles cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

The terms “(aryl)C₁₋₆-alkyl” is intended to mean an aryl group connected, as a substituent, via a C₁₋₆-alkyl, each as defined herein. The aryl groups of (aryl)C₁₋₆-alkyl may be substituted or unsubstituted. Examples include benzyl, substituted benzyl, 2-phenylethyl, 3-phenylpropyl, and naphthylalkyl.

The terms “(cycloalkyl)C₁₋₆-alkyl” is intended to mean a cycloalkyl groups connected, as substituents, via an alkyl, each as defined herein.

When used herein, the term “O—C₁₋₆-alkyl” is intended to mean C₁₋₆-alkyloxy, or alkoxy, such as methoxy, ethoxy, n-propoxy, isopropoxy, n-butoxy, isobutoxy, sec-butoxy, tert-butoxy, pentyloxy, isopentyloxy, neopentyloxy and hexyloxy The term “halogen” includes fluorine, chlorine, bromine and iodine.

In the present context, i.e. in connection with the terms “C₁₋₆-alkyl”, “aryl”, “heteroaryl”, “heterocyclyl”, “C₃₋₈-cycloalkyl”, “heterocyclyl(C₁₋₆-alkyl)”, “(cycloalkyl)alkyl”, “O—C₁₋₆-alkyl”, “C₂₋₈-alkenyl”, and “C₂₋₈-alkynyl”, the term “optionally substituted” is intended to mean that the group in question may be substituted one or several times, such as 1 to 5 times, or 1 to 3 times, or 1 to 2 times, with one or more groups selected from C₁₋₆-alkyl, C₁₋₆-alkoxy, oxo (which may be represented in the tautomeric enol form), carboxyl, amino, hydroxy (which when present in an enol system may be represented in the tautomeric keto form), nitro, alkylsulfonyl, alkylsulfenyl, alkylsulfinyl, C₁₋₆-alkoxycarbonyl, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, C₁₋₆-alkylhydroxyimino, cyano, guanidino, carbamido, C₁₋₆-alkanoyloxy, C₁₋₆-alkylsulphonyloxy, dihalogen-C₁₋₆-alkyl, trihalogen-C₁₋₆-alkyl, heterocyclyl, heteroaryl, and halo. In general, the above substituents may be susceptible to further optional substitution.

Unless otherwise indicated, when a substituent is deemed to be “optionally substituted,” it is meant that the substitutent is a group that may be substituted with one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

In certain embodiments, in the compound of formula A, E is oxygen. In some embodiments, T is also oxygen.

In some embodiments, the term “heterocyclyl” refers to a substituent selected from the group consisting of tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

In certain embodiments, the term “heteroaryl” refers to a substituent selected from the group consisting of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine, pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

In some embodiments, the term “aryl” refers to a substituent selected from the group consisting of phenyl, naphthalenyl, phenanthrenyl, anthracenyl, tetralinyl, fluorenyl, indenyl, and indanyl.

In other embodiments, the term “cycloalkyl” refers to a substituent selected from the group consisting of cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

Some embodiments of the compounds of formula A include those in which R₁ is selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments, however, R₁ is hydrogen.

In some embodiments, R₂ is selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments, however, R₂ is hydrogen.

In some embodiments, R₃-R₆ are each independently selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments, however, R₃-R₆ are hydrogen.

In further embodiments, R₇ and R₈ are each independently selected from hydrogen and C₁₋₆ alkyl. In some of these embodiments, R₇ and R₈ are hydrogen.

Preferred pharmaceuticals or dietary supplements consist of, consist essentially of, or comprise a compound of formula C, 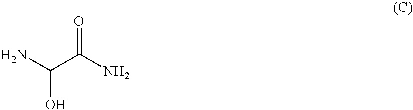
or a pharmaceutically acceptable salt, amide, ester, or prodrug thereof. This compound was isolated using cation exchange HPLC after incubating unmodified G-NH₂ in cofactor-containing serum, as described herein (See EXAMPLE 10). The compound of formula C was identified as modified G-NH₂ (Metabolite X) after the chromatography isolate described above using its NMR spectra.

The analysis was based on a doubly labeled i.e., ¹³C/¹⁵N, sample. The ¹H NMR spectrum consisted of two broad NH-amide signals located at 7.65 and 7.15 ppm and a CH-proton doublet (J=163 Hz) centered at 5.21 ppm. The intensity ratios of all three signals were close to 1:1:1. In the spectrum taken without presaturation of water solvent signal, it was possible to observe extra NH₃⁺ group signal at ˜7.4 ppm. This indicated that one proton in glycine methylene group was replaced by electronegative substituent causing significant downfield shift in ¹H NMR spectrum, as compared to the original glycine amide.

The ¹³C NMR spectrum showed two signals of equal intensity: a doublet for ¹³C═O (J=62 Hz) at 177.6 ppm and eight lines for the aliphatic carbon signal at 89.0 ppm with three different coupling constants (J=7.1; 62 and 163 Hz). J=163 Hz is the one bond ¹³C-¹H coupling, J=62 Hz is the one bond ¹³C-¹³C coupling, while the third coupling 7.1 Hz was in agreement with a one bond ¹⁵N-¹³C coupling. All possible two bond couplings were close to zero as expected from theoretical considerations. Both ¹H-¹³C and ¹³C-¹³C couplings were relatively large, in agreement with the introduction of a strongly electronegative substituent at the glycine aliphatic carbon. The same conclusion came from analysis of the ¹³C chemical shift of that aliphatic carbon, using the existing additive schemes for chemical shift prediction.

¹⁵N-¹H HSQC spectrum consisted of a strong signal from the ¹⁵N labeled amine located ˜20 ppm and a weak signal from unlabelled amide nitrogen at ˜105 ppm. These are expected typical values for NH₃⁺ and CONH₂ nitrogen resonances. The total measurement time for the doubly labeled sample was ˜10 hours.

Thus, the best agreement between the ¹H and ¹³C spectra was obtained for the structure of the compound of formula C. Accordingly, preferred embodiments include pharmaceuticals and medicaments that consist of, consist essentially of (e.g., an enriched or isolated preparation containing the compound of formula C in either enantiomer (D or L) and/or isomer (R or S)), or comprise the compound of formula C and derivatives thereof, in particular, derivatives wherein the hydroxyl group is replaced by a methoxy, ethoxy or alkoxy.

Additional preferred embodiments include pharmaceutical and dietary supplements that consist of, consist essentially of, or comprise α-peroxyglycinamide dimer (NH₂-gly-O—O-gly-NH₂), having the structure set forth in formula E or diglycinamide ether (NH₂-gly-O-gly-NH₂) having the structure set forth in formula F:

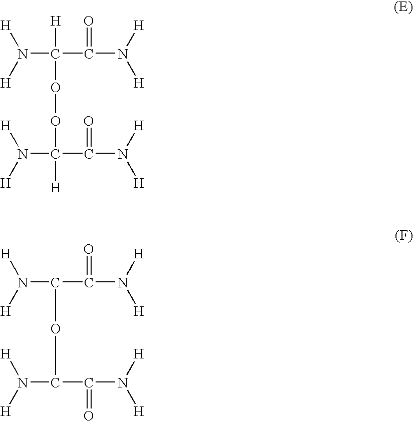

Preferred compositions also include pharmaceuticals and dietary supplements that consist of, consist essentially of, or comprise alpha-methoxyglycinamide (alpha-MeO-gly-NH₂) having the structure set forth in formula (G):

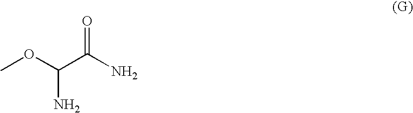

More embodiments include pharmaceuticals or dietary supplements that consist of, consist essentially of, or comprise derivatives of G-NH₂ having the formula J: 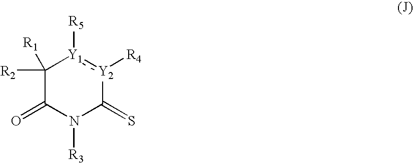
or a pharmaceutically acceptable salt, amide, ester, or prodrug thereof; wherein:

a) R₁-R₅ are each independently selected from the group consisting of hydrogen; hydroxy; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl or may be absent, or R₁ and R₂ may together form an optionally substituted saturated or unsaturated six-membered ring;

b) Y₁ and Y₂ are each independently selected from the group consisting of carbon and nitrogen; and

c) the dashed bond indicates that the bond may be present or absent.

In some embodiments of the compound of formula J, the term “heterocyclyl” refers to a substituent selected from the group consisting of tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

In certain embodiments, the term “heteroaryl” refers to a substituent selected from the group consisting of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine, pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

In some embodiments, the term “aryl” refers to a substituent selected from the group consisting of phenyl, naphthalenyl, phenanthrenyl, anthracenyl, tetralinyl, fluorenyl, indenyl, and indanyl.

In other embodiments, the term “cycloalkyl” refers to a substituent selected from the group consisting of cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

Some embodiments of the compounds of formula J include those in which R₁₋₅ are each independently selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments of the compound of formula J, R₁-R₅ are each independently selected from the group consisting of hydrogen, hydroxy, methyl, —CH₂OH, —CH₂NH₂, —CH₂CN, and —CH₂X, wherein X is a halogen. In some embodiments of the compound of formula J, Y₁ and Y₂ are nitrogen and there is a double bond between Y₁ and Y₂. In these embodiments, R₄ and R₅ are absent. In other embodiments, Y₁ and Y₂ are carbon and R₄ and R₅ are hydrogen such that the Y₁ and Y₂ carbons each have two hydrogen substituents.

In further embodiments of the compound of formula J, R₁ and R₂ each preferably can independently be selected from the group consisting of hydroxy, methyl, CH2OH, CH₂NH₂, CH₂C≡N, and CH₂Cl; R₃ preferably is selected from hydrogen, hydroxy, methyl, CH2OH, and CH₂NH₂; and R₄ and R₅ each preferably are selected from hydrogen, hydroxy, methyl, CH2OH, CH₂NH₂ and CH₂X, where X is CH₂ or N.

In a particular embodiment of the compound of formula J, Y₁ and Y₂ are both N, the dashed bond is absent, and R₄ and R₅ are selected from the group consisting of hydrogen, hydroxy, methyl, CH₂OH, CH₂NH₂ and CH₂X, where X is CH₂ or N; preferably Y₁ and Y₂ are both N, the dashed bond is absent, and R₄ and R₅ are both hydrogen.

One embodiment of the compound of formula J is the compound of formula K:

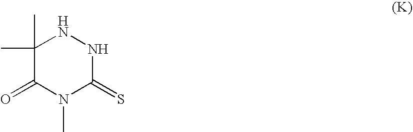

Another embodiment of the compound of formula J is the compound of formula 0:

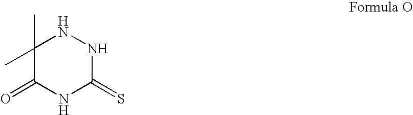

A further embodiment of the compound of formula J is the compound of formula P:

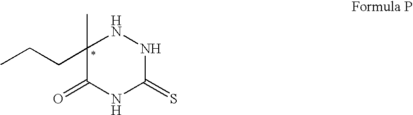

Still another embodiment of the compound of formula J is the compound of formula Q:

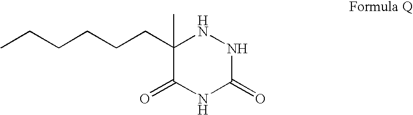

Compounds of the formulas K, O, P, and Q can also be the active ingredients in a pharmaceutical or dietary supplement. Still more embodiments concern pharmaceuticals or dietary supplements that consist of, consist essentially of, or comprise derivatives of G-NH₂ having the formula L: 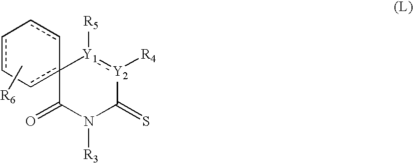
or a pharmaceutically acceptable salt, amide, ester, or prodrug thereof; wherein:
a) R₃-R₆ are each independently selected from the group consisting of hydrogen; hydroxy; halogen; amine; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl or may be absent;
b) Y₁ and Y₂ are each independently selected from the group consisting of carbon and nitrogen;
c) the dashed bonds indicate that the bonds may be present or absent; and
d) the R₆ substituent may be present as one or more substituents at any of the 5 available carbon atoms on the six-membered carbon ring, including having multiple R₆ substituents independently selected.

In some embodiments of the compound of formula L, the term “heterocycle” refers to a substituent selected from the group consisting of tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

In certain embodiments, the term “heteroaryl” refers to a substituent selected from the group consisting of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine, pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

In some embodiments, the term “aryl” refers to a substituent selected from the group consisting of phenyl, naphthalenyl, phenanthrenyl, anthracenyl, tetralinyl, fluorenyl, indenyl, and indanyl.

In other embodiments, the term “cycloalkyl” refers to a substituent selected from the group consisting of cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

Some embodiments of the compounds of formula L include those in which R₃₋₅ are each independently selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments of the compound of formula L, R₃-R₅ are each independently selected from the group consisting of hydrogen, hydroxy, methyl, —CH₂OH, —CH₂NH₂, —CH₂CN, and —CH₂X, wherein X is a halogen. In some embodiments of the compound of formula L, Y₁ and Y₂ are nitrogen and there is a double bond between Y₁ and Y₂. In these embodiments, R₄ and R₅ are absent. In other embodiments, Y₁ and Y₂ are carbon and R₄ and R₅ are hydrogen such that the Y₁ and Y₂ carbons each have two hydrogen substituents. In some embodiments of the compound of formula L, all of the dashed bonds on the six-membered carbon ring are present such that the six-membered ring is a phenyl ring. In some embodiments, each R₆ is independently selected from the group consisting of hydrogen, hydroxy, —NH₂, methyl, methoxy, and halogen.

In further embodiments of the compound of formula L, R₃ preferably is selected from hydrogen, hydroxy, methyl, CH₂OH, and CH₂NH₂; R₄ and R₅ each preferably are selected from hydrogen, hydroxy, methyl, CH₂OH, CH₂NH₂ and CH₂X, where X is CH₂ or N; and R₆ preferably is selected from hydrogen, halogen, hydroxyl, methyl, NH₂, and OCH₃. Also, the cyclohexyl ring can be modified to be a phenyl ring.

One embodiment of the compound of formula L is the compound of formula M:

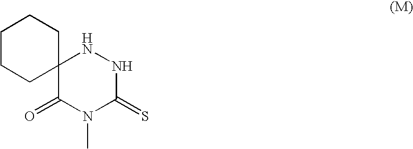

Accordingly, embodiments include pharmaceuticals and dietary supplements that consist of, consist essentially of, or comprise a compound of formula L or M. Other derivatives of G-NH₂ include modifications of AlphaHGA wherein a chain of one or more amino acids are N-terminally linked to either side of AlphaHGA. In some embodiments, these derivatives have the formula of compound N: 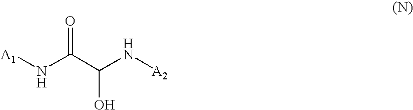
where A₁ and A₂ are separately selected from the group consisting of a chain of one or more amino acids and hydrogen. In some embodiments, A₁ and A₂ are separately selected from the group consisting of a chain of 1 to 5 amino acids and hydrogen. In some embodiments, A₁ and A₂ are separately selected from the group consisting of a chain of 1 to 4 amino acids and hydrogen. In some embodiments, A₁ and A₂ are separately selected from the group consisting of a chain of 1 to 3 amino acids and hydrogen. In some embodiments, A₁ and A₂ are separately selected from the group consisting of a chain of 1 to 2 amino acids and hydrogen. In some embodiments, A₁ and A₂ are separately selected from the group consisting of an amino acid and hydrogen. Accordingly, embodiments include pharmaceuticals and dietary supplements that consist of, consist essentially of, or comprise a compound of formula N.

As used herein a “chain of amino acids” is understood to mean a chain of two or more amino acids linked via peptide bonds. Where A₁ and/or A₂ in the compound of formula N are one or more amino acids, they are attached to the rest of the compound of formula N via a peptide bond.

As used herein “amino acids” are understood to mean any naturally occurring or synthetically produced amino acid and radicals thereof. “Amino acids” include forms that contain amido functionalities involved in peptide linkage. “Amino acids” also include forms containing carboxylic and amino functionalities but no amido functionalities. Accordingly, “amino acids” as used herein include molecular fragments that contain at least one amido functionality and one carboxylic functionality; at least one amido functionality and one amino functionality; and at least one carboxylic functionality and one amino functionality. In general, unless otherwise specified, amino acid groups may be attached to other molecular fragments by either peptide or non-peptide linkage. The amino acids used herein may also be protected with appropriate protecting groups such as boc of fmoc. Non-limiting of examples of amino acids for use herein include both natural and non-natural amino acids and include, but are not limited to, the D or L isomers of Alanine, Arginine, Asparagine, Aspartic acid, Cysteine, Glutamine, Glutamic acid, Glycine, Histidine, Isoleucine, Leucine, Lysine, Methionine, Phenylalanine, Proline, Serine, Threonine, Tryptophan, Tyrosine, Valine, Carnitine, Citrulline, Cystine, gamma-Aminobutyric acid, Glutathione, Hydroxyproline, Ornithine, Taurine, the amino acids depicted in TABLE 21, and derivatives thereof. It is to be understood that where the amino acids depicted in TABLE 21 contain a boc or finoc functionality, boc groups may be replaced by fmoc groups and vice versa. In addition, the boc or fmoc groups may also be removed in their entirety, such as to leave a free amine group.

TABLE 21
Exemplary Non-natural Amino Acids

| | |
|---|---|
| 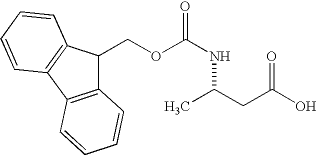 | 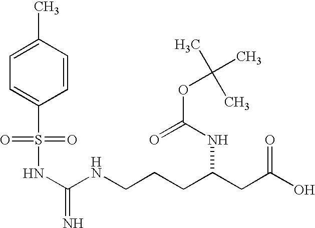 |
| (S)-3-(Fmoc-amino)butyric acid | Boc-β-Homoaig(Tos)-OH |
| 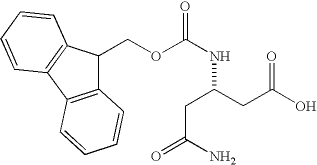 | 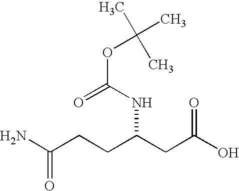 |
| Fmoc-L-β-Homoasn-OH | Boc-β-Homogln-OH |
| 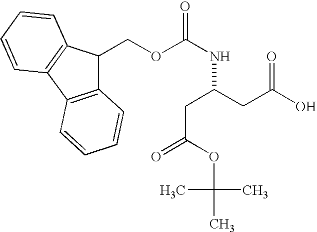 | 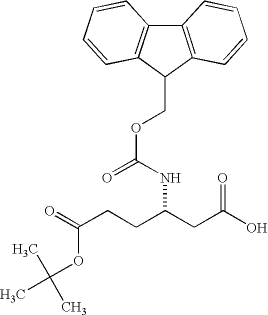 |
| Fmoc-β-Glu(OtBu)-OH | Fmoc-β-Homoglu(OtBu)-OH |
| 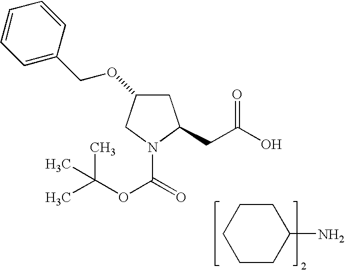 | 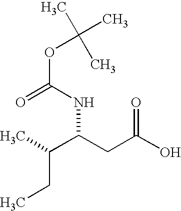 |
| Boc-β-Homohyp(Bzl)-OH DCHA | Boc-β-Homoile-OH |
| 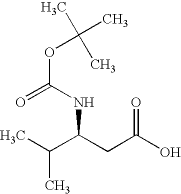 | 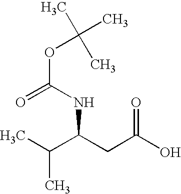 |
| Boc-D-β-Leu-OH | Boc-D-β-Leu-OH |
| 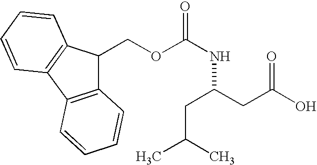 | 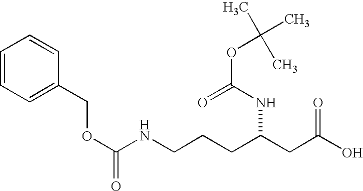 |
| Fmoc-β-Homoleu-OH | Boc-L-β-Homoorn(Z)-OH |
| 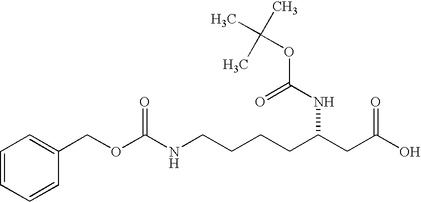 | 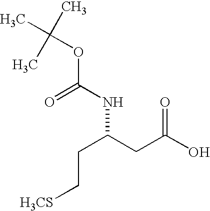 |
| Boc-β-Homolys(Z)-OH | Boc-β-Homomet-OH |
| 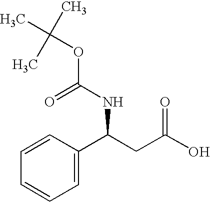 | 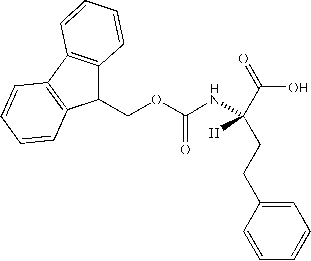 |
| Boc-β-Phe-OH | Fmoc-Homophe-OH |
| 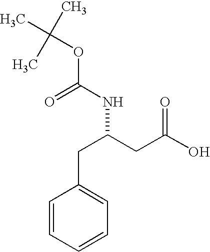 | 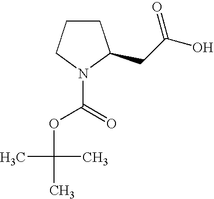 |
| Boc- Homophe-OH | Boc-β-Homopro-OH |
| 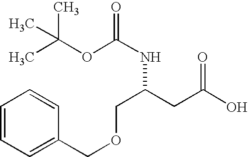 | 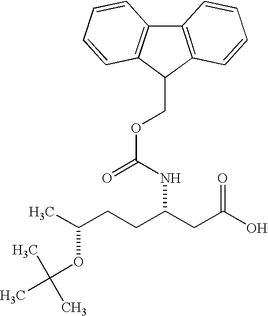 |
| Boc-β-Homoser(Bzl)-OH | Fmoc-β-Homothr(tBu)-OH |
| 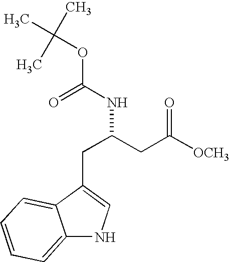 | 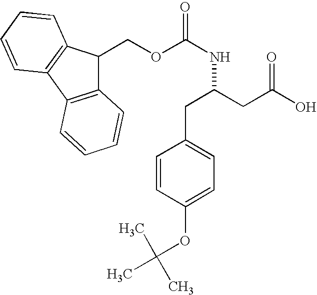 |
| Boc-β-Homotrp-OMe | Fmoc-β-Homotyr(tBu)-OH |
| 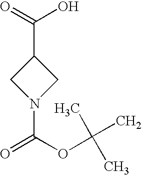 | 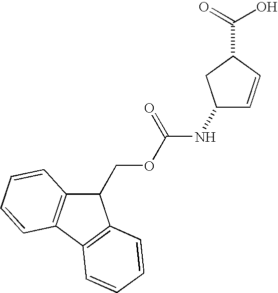 |
| 1-Boc-azetidine-3-carboxylic acid | (1S,4R)-(−)-4-(Fmoc-amino)-2-cyclopentene- |
| | 1-carboxylic acid |
| 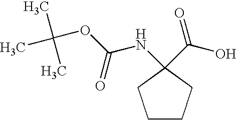 | 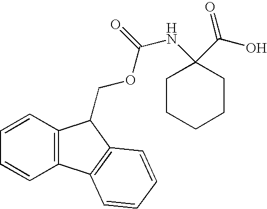 |
| 1-(Boc-amino)cyclopentanecarboxylic acid | 1-(Fmoc-amino)cyclohexanecarboxylic acid |
| 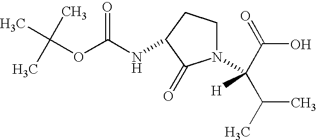 | 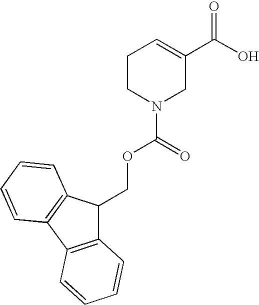 |
| N-Boc-Freidinger's lactam | 1-Fmoc-1,2,5,6-tetrahydropyridine-3- |
| | carboxylic acid |
| 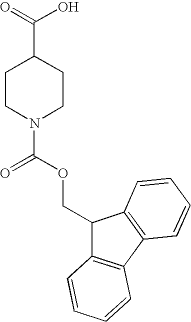 | 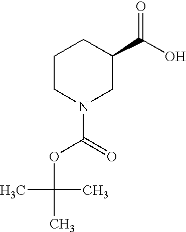 |
| 1-Fmoc-piperidine-4-carboxylic acid | (S)-N-Boc-piperidine-3-carboxylic acid |
| 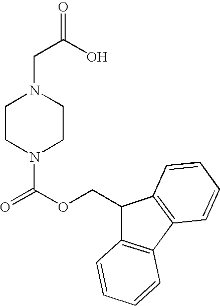 | 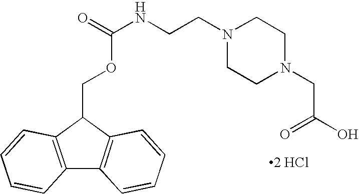 |
| 4-Fmoc-1-piperidineacetic acid | 4-[2-(Fmoc-amino)ethyl]piperazin-1-ylacetic |
| | acid |
| 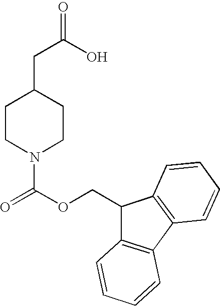 | 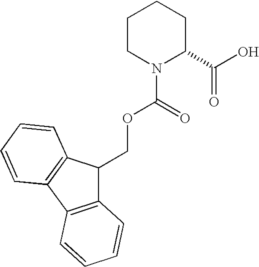 |
| 1-Fmoc-4-piperidineacetic acid | (R)-N-Fmoc-piperidine-2-carboxylic acid |
| 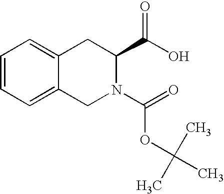 | 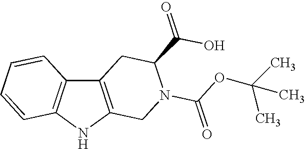 |
| (S)-N-Boc-1,2,3,4-tetrahydroisoquinoline-3 - | (S)-2-Boc-1,2,3,4-tetrahydronorharman-3- |
| carboxylic acid | carboxylic acid |
| 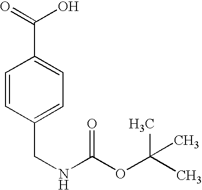 | 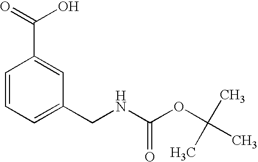 |
| 4-(Boc-aminomethyl)benzoic acid | 3-(Boc-aminomethyl)benzoic acid |
| 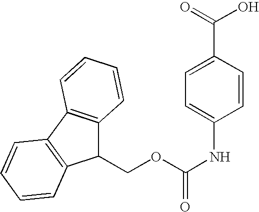 | 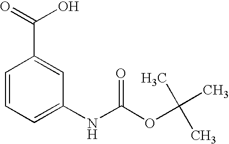 |
| 4-(Fmoc-amino)benzoic acid | 3-(Boc-amino)benzoic acid |
| 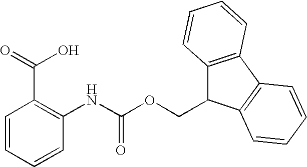 | 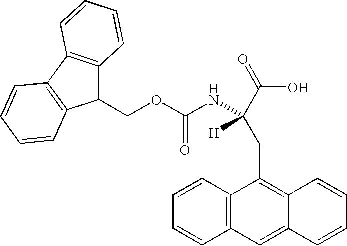 |
| 2-(Fmoc-amino)benzoic acid | Fmoc-3-(9-anthryl)-Ala-OH |
| 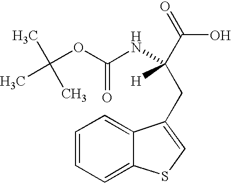 | 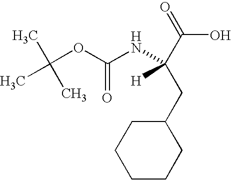 |
| Boc-3-(3-benzothienyl)-Ala-OH | Boc-3-cyclohex-yl-L-alanine methyl ester |
| 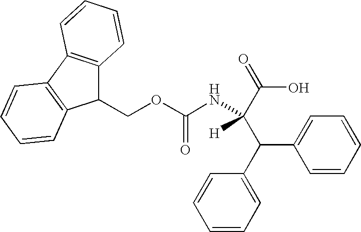 | 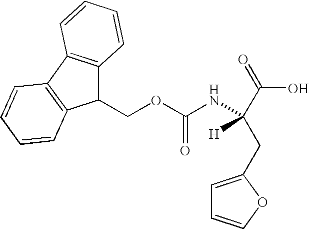 |
| Fmoc-3,3-diphenyl-Ala-OH | Fmoc-3-(2-furyl)-Ala-OH |
| 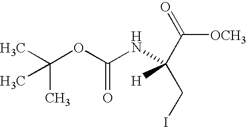 | 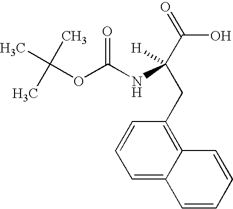 |
| Boc-3-iodo-Ala-OMe | Boc-3-(1-naphthyl)-D-alanine |
| 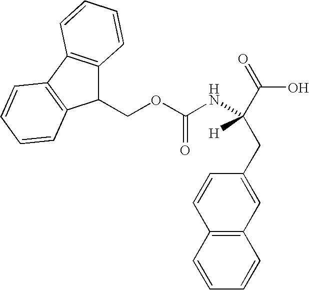 | 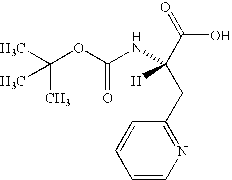 |
| Fmoc-3-(2-naphthyl)-L-alanine | Boc-3-(2-pyridyl)-Ala-OH |
| 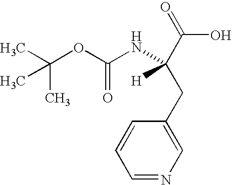 | 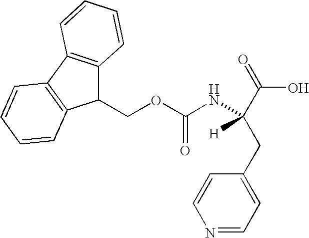 |
| Boc-3-(3-pyridyl)-Ala-OH | Fmoc-3-(4-pyridyl)-Ala-OH |
| 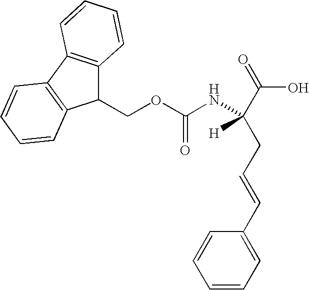 | 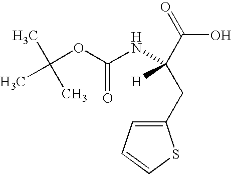 |
| Fmoc-3-styryl-Ala-OH | Boc-3-(2-thienyl)-Ala-OH |
| 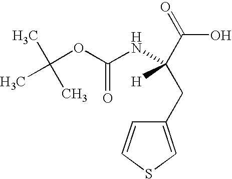 | 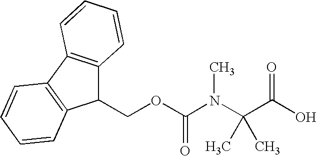 |
| Boc-3-(3-thienyl)-Ala-OH DCHA | Fmoc-N-Me-Aib-OH |
| 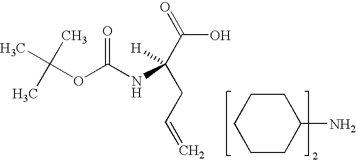 | 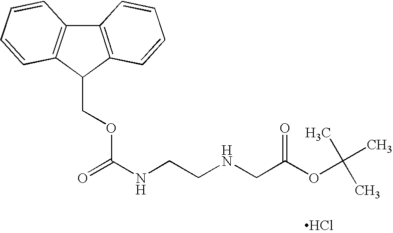 |
| Boc-D-allyl-Gly-OH DCHA | N-[2-(Fmoc-amino)-ethyl]-Gly-O-tBu HCl |
| 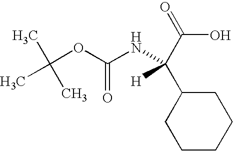 | 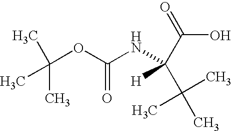 |
| Boc-L-2-cyclohexylglycine | (S)-N-Boc-2-amino-3,3-dimethylbutyric acid |
| 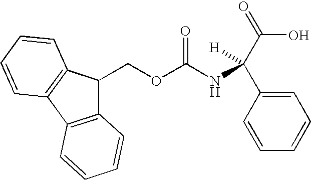 | 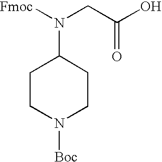 |
| Fmoc-L-2-phenylglycine | Fmoc-N-(Boc-4-piperidyl)glycine |
| 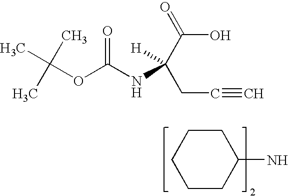 | 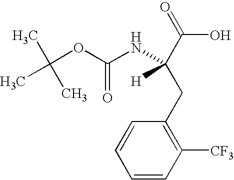 |
| Boc-D-propargyl-Gly-OH DCHA | Boc-2-(trifluoromethyl)-L-phenylalanine |
| 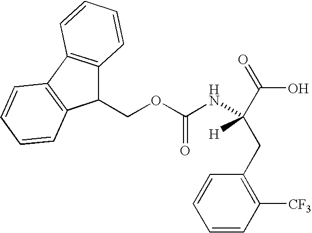 | 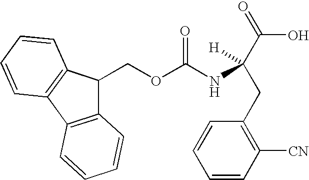 |
| Fmoc-2-chloro-L-phenylalanine | Fmoc-2-cyano-D-phenylalanine |
| 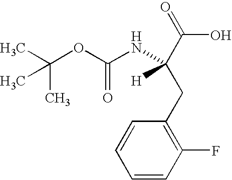 | 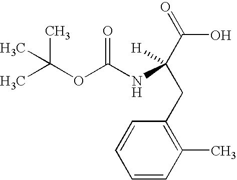 |
| Boc-2-fluoro-L-phenylalanine | Boc-2-methyl-D-phenylalauine |
| 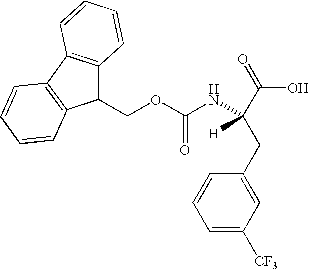 | 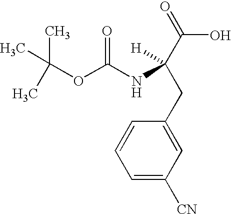 |
| Fmoc-3-(trifluoromethyl)-L-phenylalanine | Boc-3-cyano-D-phenylalanine |
| 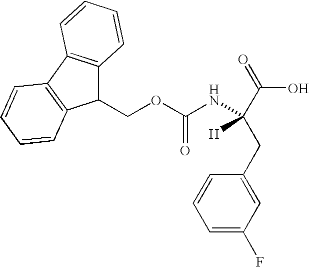 | 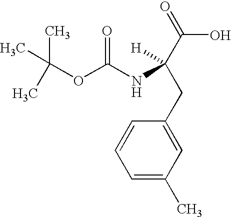 |
| Fmoc-3-fluoro-L-phenylalanine | Boc-3-methyl-D-phenylalanine |
| 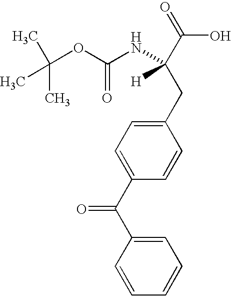 | 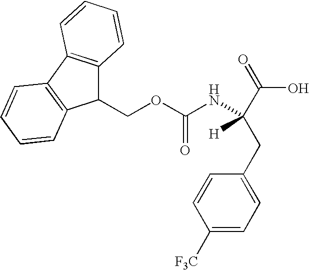 |
| Boc-4-benzoyl-L-phenylalanine | Fmoc-4-(trifluoromethyl)-L-phenylalanine |
| 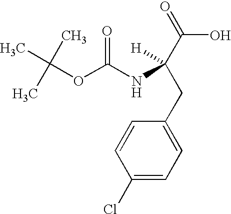 | 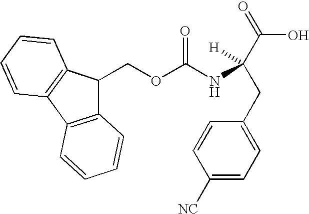 |
| Boc-4-chloro-D-phenylalanine | Fmoc-4-cyano-D-phenylalanine |
| 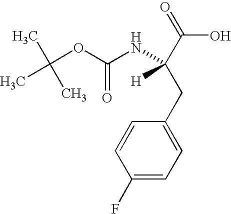 | 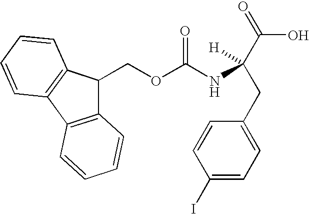 |
| Boc-4-fluoro-L-phenylalanine | Fmoc-4-iodo-D-phenylalanine |
| 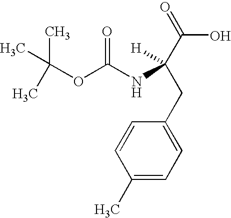 | 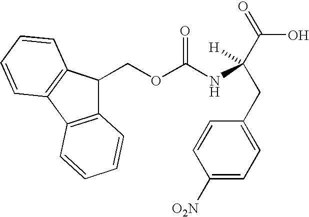 |
| Boc-4-methyl-D-phenylalanine | Fmoc-4-nitro-D-phenylalanine |
| 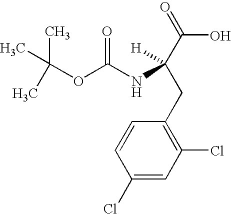 | 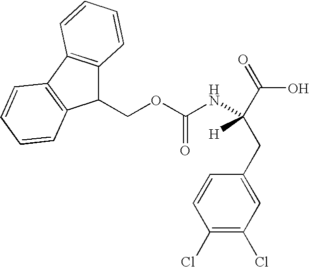 |
| Boc-2,4-dichloro-D-phenylalanine | Fmoc-3,4-dichloro-L-phenylalanine |
| 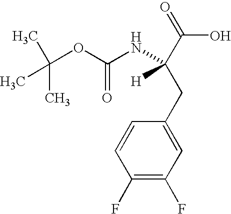 | 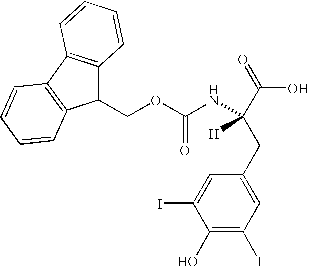 |
| Boc-3,4-difluoro-L-phenylalanine | Fmoc-3,5-diiodo-L-tyrosine |
| 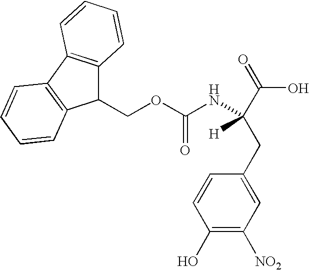 | 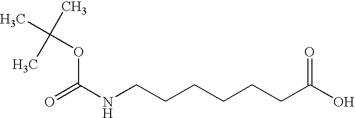 |
| Fmoc-3-nitro-L-tyrosine | 7-(Boc-amino)enanthic acid |
| 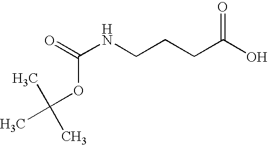 | 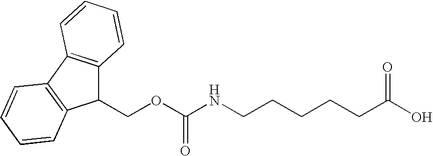 |
| 4-(Boc-amino)butyric acid | 6-(Fmoc-amino)caproic acid |
| 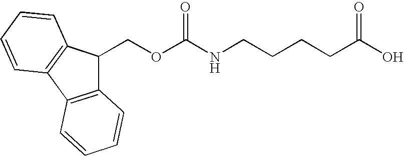 | 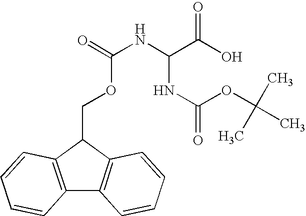 |
| 5-(Fmoc-amino)pentanoic acid | N-Boc-N′-Fmoc-diaminoacetic acid |
| 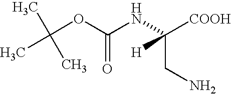 | 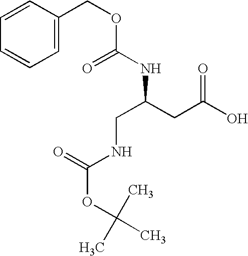 |
| N-a-Boc-L-2,3-diaminopropionic acid | 4-(Boc-amino)-3-(Z-amino)butyric acid |
| 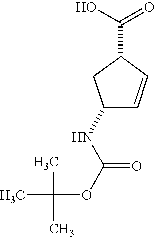 | 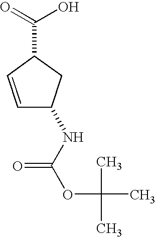 |
| (1S,4R)-(−)-4-(Boc-amino)-2-cyclopentene-1- | (1S,3R)-(+)-3-(Boc- |
| carboxylic acid | amino)cyclopentanecarboxylic acid |
| 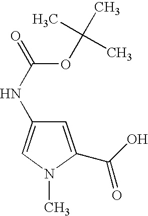 | 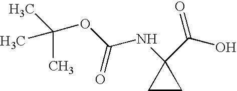 |
| 4-(Boc-amino)-1-methylpyrrole-2-carboxylic | 1-(BOC-amino)cyclopropanecarboxylic acid |
| acid | |
| 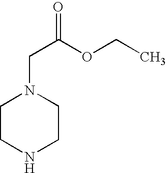 | 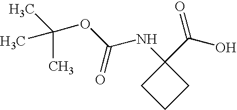 |
| | 1-(Boc-amino)cyclobutanecarboxylic acid |
| 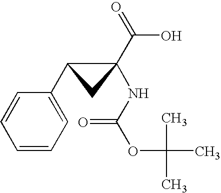 | 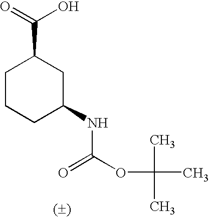 |
| (1R,2R)-cis-1-(BOC-amino)-2- | cis-3-(Boc-amino)cyclohexanecarboxylic acid |
| phenylcyclopropanecarboxylic acid | |
| 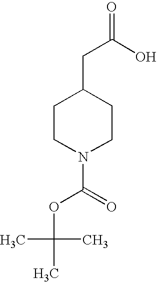 | 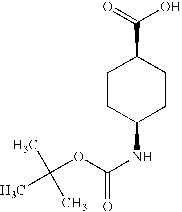 |
| 1-Boc-4-piperidylacetic acid | cis-4-(Boc-amino)cyclohexanecarboxylic acid |
| 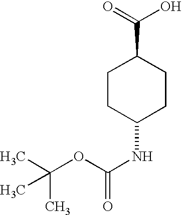 | 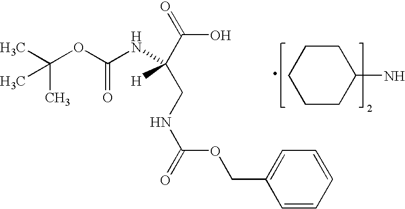 |
| trans-4-(Boc-amino)cyclohexanecarboxylic acid | Nα-Boc-Nβ-Z-L-2,3-diaminopropionic acid |
| 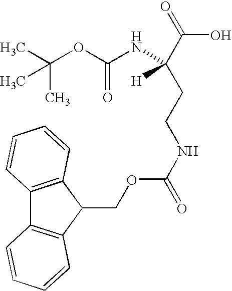 | 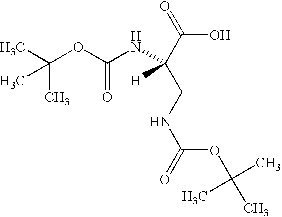 |
| Nγ-Fmoc-Nα-Boc-L-2,4-diaminobutyric acid | Nα,Nβ-Di-Boc-L-2,3-diaminopropionic acid |
| | Dicyclohexylamine salt |
| 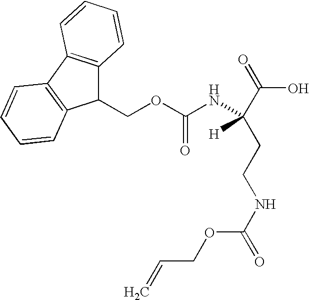 | 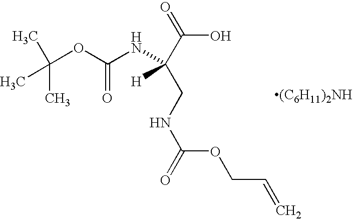 |
| Nγ-Alloc-Nα-Fmoc-L-2,4-diaminobutyric acid | Nβ-Alloc-Nα-Boc-L-2,3-diaminopropionic |
| | acid Dicyclohexylamine salt |
| 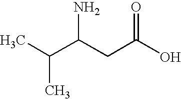 | 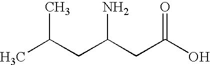 |
| (±)-3-Amino-4-methylpentanoic acid | (±)-3-Amino-5-methylcaproic acid |
| 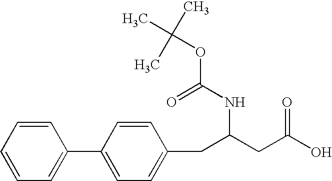 | 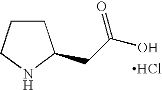 |
| (±)-3-(Boc-amino)-4-(4-biphenylyl)butyric acid | (S)-2-(2-Pyrrolidinyl)acetic acid hydrochloride |
| 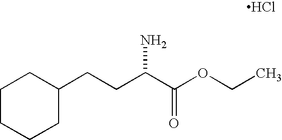 | 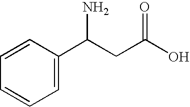 |
| (+)-Ethyl (S)-2-amino-4-cyclohexylbutyrate | (±)-3-Amino-3-phenylpropionic acid |
| hydrochloride | |
| 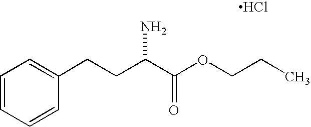 | 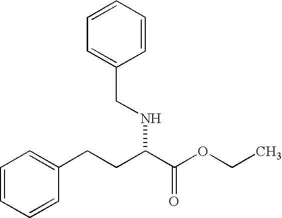 |
| L-Homophe-OEt HCl | N-Benzyl-L-Homophe-O] |
| 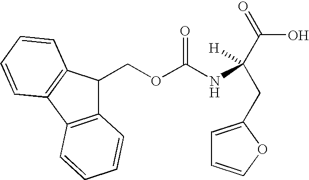 | 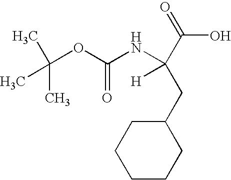 |
| Fmoc-3-(2-furyl)-D-Ala-OH | Boc-3-cyclohexyl-DL-alanine |
| 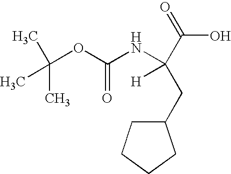 | 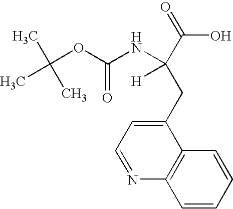 |
| 3-Cyclopentyl-DL-Ala-OH | Boc-3-(4-quinolyl)-DL-Ala-OH |
| 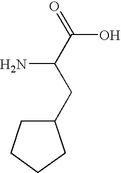 | 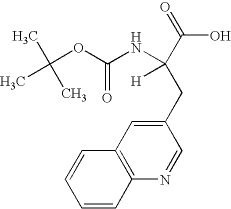 |
| 3-Cyclopentyl-DL-Ala-OH | Boc-3-(3-quinolyl)-DL-Ala-OH |
| 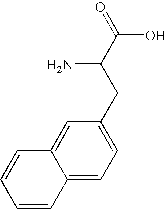 | 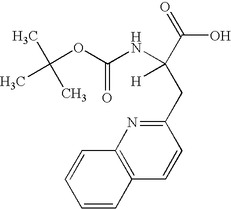 |
| 3-(2-quinolyl)-DL-Ala-OH | Boc-3-(2-quinolyl)-DL-Ala-OH |
| 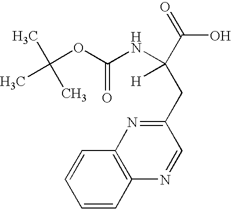 | 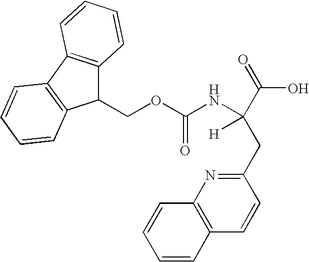 |
| Boc-3-(2-quinoxalyl)-DL-Ala-OH | Fmoc-3-(2-quinolyl)-DL-Ala-OH |
| 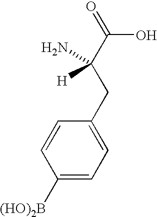 | 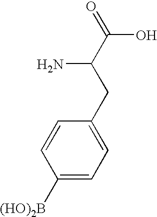 |
| 4-Borono-L-phenylalanine | 4-Borono-DL-phenylalanine |
| 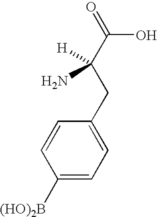 | 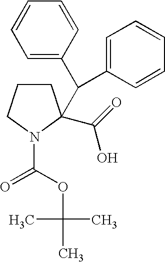 |
| 4-Borono-D-phenylalanine | Boc-α-(diphenylmethyl)-DL-Pro-OH |
| 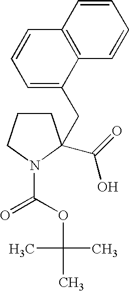 | 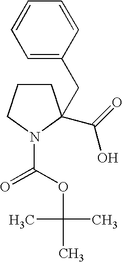 |
| Boc-α-(1-naphthylmethyl)-DL-Pro-OH | Boc-α-benzyl-DL-Pro-OH |
| 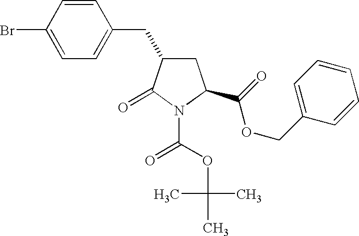 | 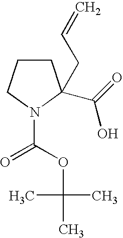 |
| (4R)-Boc-4-(4-bromobenzyl)-L-pyroglutamic | Boc-α-allyl-DL-Pro-OH |
| acid benzyl ester | |
| 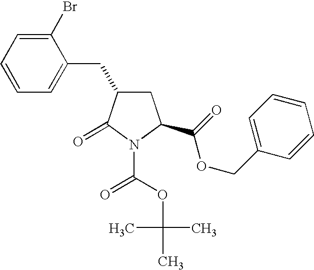 | 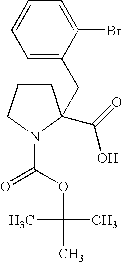 |
| (4R)-Boc-4-(2-bromobenzyl)-L-pyroglutamic | Boc-α-(2-bromobenzyl)-DL-Pro-OH |
| acid benzyl ester | |
| 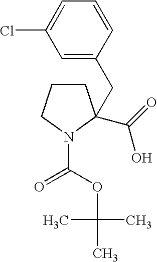 | 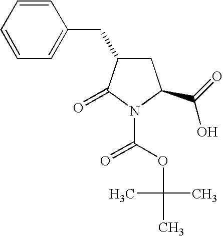 |
| Boc-á-(3-ch1orobenzy1)-DL-Pro-OH | (4R)-Boc-4-benzyl-L-pyroglutamic acid |
| 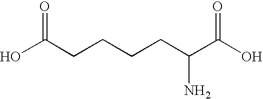 | 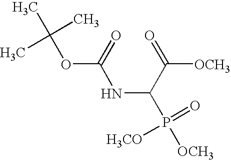 |
| (±)-2-Aminoheptanedioic acid | (±)-Boc-2-phosphonoglycine trimethyl ester |
| 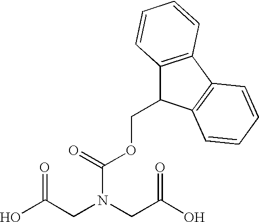 | 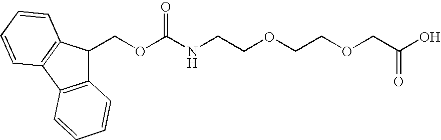 |
| N-Fmoc-iminodiacetic acid | {2-[2-(Fmoc-amino)ethoxy]ethoxy}acetic acid |

Some embodiments include pharmaceuticals or dietary supplements that consist of, consist essentially of, or comprise amino acid derivatives of AlphaHGA having the formula A presented previously: 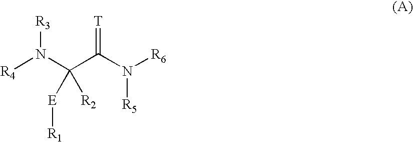
where E is selected from the group consisting of oxygen, sulfur, and NR₇; T is selected from the group consisting of oxygen, sulfur, and NR₈; R₁, R₂, R₅, R₆, R₇, and R₈ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl; and R₃ and R₄ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; optionally substituted perhaloalkyl; and an optionally substituted amino acid; wherein at least one of R₃ and R₄ is an optionally substituted amino acid.

When R₃ or R₄ is an amino acid, the amino acid may be linked to the rest of the compound of formula A via a peptide bond such that the amino acid is bonded to the nitrogen atom through its carbonyl group. Alternatively, the amino acid may be linked via another linker such that the amino acid's carboxylic functionality does not participate in bonding of the amino acid to the rest of the compound of formula A.

In some embodiments, E and/or T are oxygen. In some embodiments, R₁, R₂, R₅, and/or R₆ are selected from the group consisting of hydrogen; C₁₋₆ alkyl; C₂₋₆ alkenyl; C₂₋₆ alkynyl; and C₃₋₈ cycloalkyl. In some embodiments, R₁, R₂, R₅, and/or R₆ are hydrogen. In some embodiments, R₃ is hydrogen and R₄ is an amino acid. In a particular embodiment, E and T are oxygen and R₁, R₂, R₅, and R₆ are hydrogen. In some embodiments, the compound of formula A has the structure of formula Y:

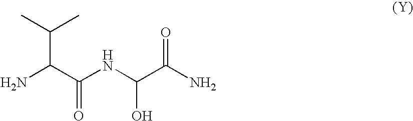

Some embodiments include pharmaceuticals or dietary supplements that consist of, consist essentially of, or comprise amino acid derivatives of AlphaHGA having the formula Z: 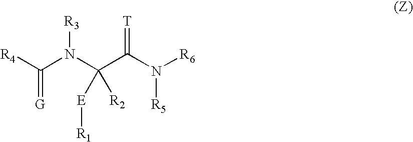
where E is selected from the group consisting of oxygen, sulfur, and NR₇; T is selected from the group consisting of oxygen, sulfur, and NR₈; G is selected from the group consisting of oxygen and sulfur; R₁, R₂, R₃, R₅, &, R₇, and R₈ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl; and R₄ is selected from the group consisting of optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl; wherein R₄ contains at least one amino functionality.

In some embodiments, E, G, and/or T are oxygen. In some embodiments, R₁, R₂, R₃, R₅, and/or R₆ are selected from the group consisting of hydrogen; C₁₋₆ alkyl; C₂₋₆ alkenyl; C₂₋₆ alkynyl; and C₃₋₈ cycloalkyl. In some embodiments, R₁, R₂, R₃, R₅, and/or R₆ are hydrogen. In a particular embodiment, E and T are oxygen and R₁, R₂, R₅, and R₆ are hydrogen. In some embodiments, the compound of formula A or Z has the structure of formula Y:

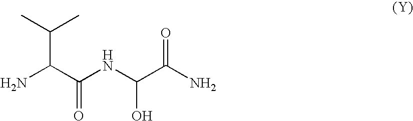

Some embodiments include pharmaceuticals or dietary supplements that consist of, consist essentially of, or comprise amino acid derivatives of AlphaHGA having the formula AA: 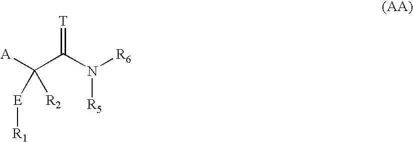
where E is selected from the group consisting of oxygen, sulfur, and NR₇; T is selected from the group consisting of oxygen, sulfur, and NR₈; R₁, R₂, R₅, and R₆ are each independently selected from the group consisting of hydrogen; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl; and A is an optionally substituted amino acid.

The optionally substituted amino acid A may be linked to the rest of the compound of formula AA through either a C-terminal or N-terminal amido group. Alternatively, the amino acid A may be linked via another linker such that the amino acid contains a carboxylic and amino functionality that does not participate in bonding of the amino acid to the rest of the compound of formula AA.

In some embodiments, E and/or T are oxygen. In some embodiments, R₁, R₂, R₅, and/or R₆ are selected from the group consisting of hydrogen; C₁₋₆ alkyl; C₂₋₆ alkenyl; C₂₋₆ alkynyl; and C₃₋₈ cycloalkyl. In some embodiments, R₁, R₂, R₅, and/or R₆ are hydrogen. In a particular embodiment, E and T are oxygen and R₁, R₂, R₅, and R₆ are hydrogen. In some embodiments, the compound of formula AA has the structure of formula Y:

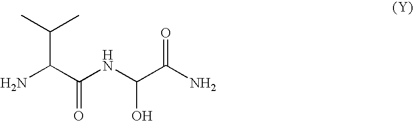

Still more embodiments include pharmaceuticals or dietary supplements that consist of, consist essentially of, or comprise derivatives of G-NH₂ having the formula R: 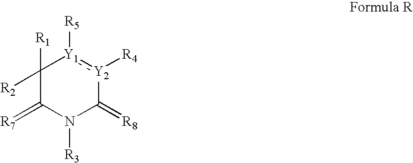
or a pharmaceutically acceptable salt, amide, ester, or prodrug thereof; wherein:

a) R₁-R₅ are each independently selected from the group consisting of hydrogen; hydroxy; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl or may be absent, or R₁ and R₂ may together form an optionally substituted saturated or unsaturated six-membered ring;

b) R₇-R₈ are each independently selected from the group consisting of sulfur (S), oxygen (O), and imino (NH).

c) Y₁ and Y₂ are each independently selected from the group consisting of carbon and nitrogen; and

g) the dashed bond indicates that the bond may be present or absent.

In some embodiments of the compound of formula R, the term “heterocyclyl” refers to a substituent selected from the group consisting of tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

In certain embodiments, the term “heteroaryl” refers to a substituent selected from the group consisting of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine, pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

In some embodiments, the term “aryl” refers to a substituent selected from the group consisting of phenyl, naphthalenyl, phenanthrenyl, anthracenyl, tetralinyl, fluorenyl, indenyl, and indanyl.

In other embodiments, the term “cycloalkyl” refers to a substituent selected from the group consisting of cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

Some embodiments of the compounds of formula R include those in which R₁₋₅ are each independently selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments of the compound of formula R, R₁-R₅ are each independently selected from the group consisting of hydrogen, hydroxy, methyl, —CH₂OH, —CH₂NH₂, —CH₂CN, and —CH₂X, wherein X is a halogen. In some embodiments of the compound of formula J, Y₁ and Y₂ are nitrogen and there is a double bond between Y₁ and Y₂. In these embodiments, R₄ and R₅ are absent. In other embodiments, Y₁ and Y₂ are carbon and R₄ and R₅ are hydrogen such that the Y₁ and Y₂ carbons each have two hydrogen substituents.

In further embodiments of the compound of formula R, R₁ and R₂ each preferably can independently be selected from the group consisting of hydroxy, methyl, CH₂OH, CH₂NH₂, CH₂C≡N, and CH₂Cl; R₃ preferably is selected from hydrogen, hydroxy, methyl, CH2OH, and CH₂NH₂; and R₄ and R₅ each preferably are selected from hydrogen, hydroxy, methyl, CH2OH, CH₂NH₂ and CH₂X, where X is CH₂ or N.

In a particular embodiment of the compound of formula R, Y₁ and Y₂ are both N, the dashed bond is absent, and R₄ and R₅ are selected from the group consisting of hydrogen, hydroxy, methyl, CH₂OH, CH₂NH₂ and CH₂X, where X is CH₂ or N; preferably Y₁ and Y₂ are both N, the dashed bond is absent, and R₄ and R₅ are both hydrogen.

One embodiment of the compound of formula R is the compound of formula S: 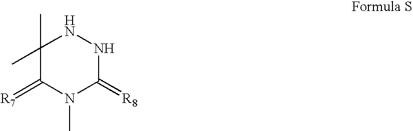
wherein, R₇-R₈ are each independently selected from the group consisting of sulfur (S), oxygen (O), and imino (NH).

Another embodiment of the compound of formula R is the compound of formula V: 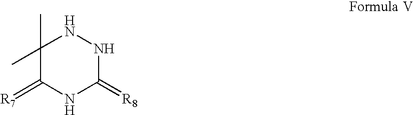
wherein, R₇-R₈ are each independently selected from the group consisting of sulfur (S), oxygen (O), and imino (NH).

A further embodiment of the compound of formula R is the compound of formula W: 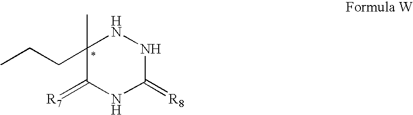
wherein, R₇-R₈ are each independently selected from the group consisting of sulfur (S), oxygen (O), and imino (NH).

Still another embodiment of the compound of formula R is the compound of formula X: 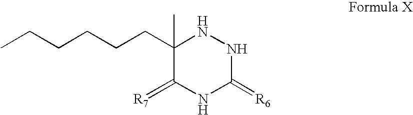
wherein, R₇—R are each independently selected from the group consisting of sulfur (S), oxygen (O), and imino (NH).

Compounds of the formulas S, V, W, and X can also be the active ingredients in a pharmaceutical or dietary supplement. Still more embodiments concern pharmaceuticals or dietary supplements that consist of, consist essentially of, or comprise derivatives of G-NH₂ having the formula T: 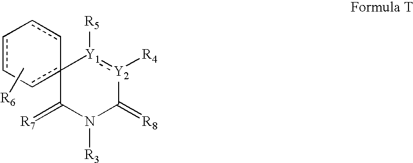
or a pharmaceutically acceptable salt, amide, ester, or prodrug thereof; wherein:
a) R₃-R₆ are each independently selected from the group consisting of hydrogen; hydroxy; halogen; amine; optionally substituted alkyl; optionally substituted alkenyl; optionally substituted alkynyl; optionally substituted cycloalkyl; optionally substituted heterocyclyl; optionally substituted cycloalkylalkyl; optionally substituted heterocyclylalkyl; optionally substituted aryl; optionally substituted heteroaryl; optionally substituted alkylcarbonyl; optionally substituted alkoxyalkyl; and optionally substituted perhaloalkyl or may be absent;
b) Y₁ and Y₂ are each independently selected from the group consisting of carbon and nitrogen;
c) the dashed bonds indicate that the bonds may be present or absent;
d) the R₆ substituent may be present as one or more substituents at any of the 5 available carbon atoms on the six-membered carbon ring, including having multiple R₆ substituents independently selected; and
e) R₇-R₈ are each independently selected from the group consisting of sulfur (S), oxygen (O), and imino (NH).

In some embodiments of the compound of formula T, the term “heterocycle” refers to a substituent selected from the group consisting of tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 0,1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

In certain embodiments, the term “heteroaryl” refers to a substituent selected from the group consisting of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine, pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

In some embodiments, the term “aryl” refers to a substituent selected from the group consisting of phenyl, naphthalenyl, phenanthrenyl, anthracenyl, tetralinyl, fluorenyl, indenyl, and indanyl.

In other embodiments, the term “cycloalkyl” refers to a substituent selected from the group consisting of cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

Some embodiments of the compounds of formula T include those in which R₃₋₄ are each independently selected from the group consisting of hydrogen; C₁₋₆ alkyl; C₂₋₆ alkenyl; C₂₋₆ alkynyl; C₃₋₈ cycloalkyl; C₃₋₈ heterocyclyl; cycloalkyl(C₁₋₆)alkyl; heterocyclyl(C₁₋₆)alkyl; aryl; heteroaryl; (C₁₋₆)alkylcarbonyl; (C₁₋₆)alkoxy(C₁₋₆)alkyl; and perhalo(C₁₋₆)alkyl. In some of these embodiments, the alkyl group of the various substituents listed above is selected from the group consisting of methyl, ethyl, propyl, n-butyl, sec-butyl, and tert-butyl.

In certain embodiments of the compound of formula T, R₃-R₅ are each independently selected from the group consisting of hydrogen, hydroxy, methyl, —CH₂OH, —CH₂NH₂, —CH₂CN, and —CH₂X, wherein X is a halogen. In some embodiments of the compound of formula L, Y₁ and Y₂ are nitrogen and there is a double bond between Y₁ and Y₂. In these embodiments, R₄ and R₅ are absent. In other embodiments, Y₁ and Y₂ are carbon and R₄ and R₅ are hydrogen such that the Y₁ and Y₂ carbons each have two hydrogen substituents. In some embodiments of the compound of formula T, all of the dashed bonds on the six-membered carbon ring are present such that the six-membered ring is a phenyl ring. In some embodiments, each R₆ is independently selected from the group consisting of hydrogen, hydroxy, —NH₂, methyl, methoxy, and halogen.

In further embodiments of the compound of formula T, R₃ preferably is selected from hydrogen, hydroxy, methyl, CH₂OH, and CH₂NH₂; R₄ and R₅ each preferably are selected from hydrogen, hydroxy, methyl, CH₂OH, CH₂NH₂ and CH₂X, where X is CH₂ or N; and R₆ preferably is selected from hydrogen, halogen, hydroxyl, methyl, NH₂, and OCH₃. Also, the cyclohexyl ring can be modified to be a phenyl ring.

One embodiment of the compound of formula T is the compound of formula U: 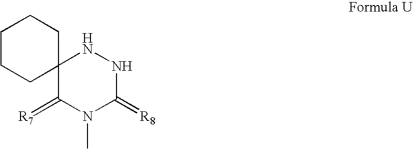
wherein, R₇-R₈ are each independently selected from the group consisting of sulfur (S), oxygen (O), and imino (NH).

Some embodiments include pharmaceuticals or dietary supplements that consist of, consist essentially of, or comprise a product of the degradation, hydrolysis, or oxidation of AlphaHGA. Non-limiting examples of such products include the structures:

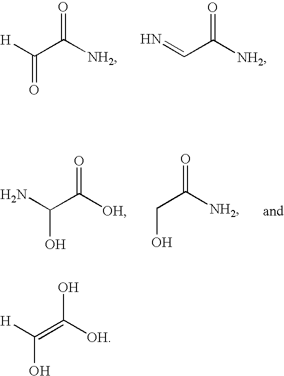

Methods of Making Antiretroviral Compounds

Several approaches to make the antiretroviral compounds described herein are described in the EXAMPLES 19-31. Some of the antiretroviral compounds described herein are also commercially available (e.g., compounds of formulas K and M). Various approaches to synthesize some of the antiretroviral compounds described herein are known in the art. (See e.g., JP 5097789A2 to Hayakawa et al., entitled “Alpha-hydroxyglycinamide Derivative and its Preparation,” filed Oct. 3, 1991, herein expressly incorporated by reference in its entirety). By one approach, an α-hydroxyglycinamide derivative represented by the following formula (B) is prepared: 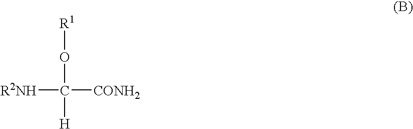
(wherein R¹ is a hydrogen atom, a lower alkyl group, a lower alkenyl group, a lower alkynyl group, a benzyl group, or a silyl group substituted with an alkyl group or an alkyl group and an aromatic group; R² is a hydrogen atom or an amino protecting group) and a salt thereof.

By another approach, an α-hydroxyglycinamide derivative or salt thereof represented by the following formula (H): 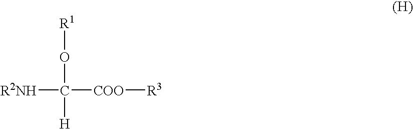
(wherein R¹ and R² are defined in formula (B); R³ is a hydrogen atom or a carboxyl protecting group) is treated with ammonia in a solvent, the amino protecting group is removed if desired, and the compound obtained is further converted into a salt thereof if desired.

In accordance with some of the preferred embodiments described herein, the lower alkyl group represented by reference symbol R¹ is an alkyl group containing no more than 6, preferably no more than 4 carbon atoms. Examples of such groups include methyl group, ethyl group, n-propyl group, isopropyl group, n-butyl group, isobutyl group, tert-butyl group, pentyl group that may be branched, and hexyl group that may be branched.

The lower alkenyl group represented by reference symbol R¹ is an alkenyl group containing no more than 6, preferably no more than 4 carbon atoms. Examples of such groups include ethenyl group, allyl group, and butenyl group having a double bond in any position. The lower alkynyl group represented by reference symbol R¹ is an alkynyl group containing no more than 6, preferably no more than 4 carbon atoms. Examples of such groups include ethynyl group and the like.

The silyl group substituted with a lower alkyl group, which is represented by reference symbol R₁, is a silyl group substituted with 1 to 3 lower alkyl groups. The lower alkyl substituents used in this case are any of the lower alkyl groups described hereinabove with reference to R₁ or combinations thereof. The silyl group substituted with a lower alkyl group is preferably a tert-butyldimethylsilyl group. The silyl group substituted with an alkyl and an aromatic group is a silyl group substituted with the above-described alkyl group and phenyl group, for example, tert-butyldiphenylsilyl group.

Protecting groups that have been used in the field of amino acid or peptide chemistry can be used as the amino protecting group represented by R₂. Examples of such groups include oxycarbonyl-type protecting groups, for example, benzyloxycarbonyl (Cbz-), p-methoxybenzyloxycarbonyl [Z(OMe)-], tert-butoxycarbonyl (Boc-), or 2-biphenylisopropoxycarbonyl (Bpoc-), and the like; acyl protecting groups, for example, HCO—, phthalate group (Pht-), or o-nitrophenylthio group (Nps-), and the like; and alkyl protecting groups, for example, triphenylmethyl group (Trt-), and the like.

Salts of the α-hydroxyglycinamide derivative in accordance with some of the embodiments described herein are acid-added salts, for example, inorganic salts such as hydrohalides, e.g., hydrofluorides, hydrochlorides, hydrobromides, nitrates, sulfates, or phosphates, organic acid salts such as fumarates, acetates, and the like.

The compounds represented by formula (C) can be prepared by treating an α-hydroxyglycine derivative represented by the following formula (H): 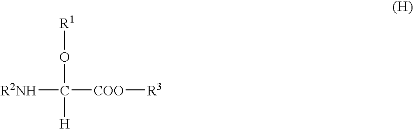
(wherein R¹ and R² are defined in formula (B); R³ is a hydrogen atom or a carboxyl protecting group) with ammonia in a solvent and optionally removing the amino protecting group.

The carbonyl protecting group R³ is an ordinary carboxy protecting group that can be substituted with amino group by treatment with ammonia. Examples of such groups include lower alkyloxy groups, for example, methoxy group (—OMe), ethoxy group (—OEt), benzyloxy group (—OBzl), or tert-butoxy group (-OtBu), or aryloxy group, such as p-nitrophenoxy group (—ONp), and the like.

Ordinary organic solvents such as lower alcohols, for example methanol, ethanol, propanol, ethers such as methyl ethyl ether, diethyl ether, isopropyl ether, and the like can be used as the solvents for the reaction. The reaction can be conducted by dissolving the compound represented by formula (H) in the above-mentioned solvent and blowing ammonia under reduced, normal, or increased pressure at a temperature, for example, from −78° C. to 40° C., preferably from 0° C. to 25° C., e.g. at room temperature.

This reaction makes it possible to obtain the compound (B), in which R² is an amino protecting group. In order to remove the amino protecting group R² from this compound and to obtain the compound (B), in which R² is hydrogen, usual deprotecting treatment may be conducted according to the type of the amino protecting group R². For example, when the protecting group R² is benzyloxycarbonyl, P-methoxybenzyloxycarbonyl, and the like, deprotecting can be carried out by conducting treatment with hydrogen gas in the presence of a hydrogenation catalyst, for example, palladium/carbon or the like. Furthermore, when the protecting group R² is tert-butoxycarbonyl, deprotecting can be conducted with hydrochloric acid-dioxane. A salt of the compound (B) can be produced, for example, by conducting the above-described deprotecting treatment in the presence of an acid such as hydrochloric acid.

A compound according to formula (H), in which R¹ is not a hydrogen atom, can be produced, for example, by the following two methods. With the first method, it can be produced by introducing R¹ other than hydrogen into the compound among the compounds represented by formula (H), in which R¹ is hydrogen. The introduction of the group R¹ other than hydrogen can be conducted with the respective functional derivative of the group, for example, a halogen derivative. For example, for introducing a lower alkyl substituted silyl group, a halide of silyl group can be used, for example, tert-butyldimethylsilyl chloride can be used for introducing a tert-butoxydimethylsilyl group. This reaction can be conducted at a temperature of from 0° C. to 30° C. in a solvent such as dimethylformamide.

Furthermore, in order to introduce a lower alkenyl or lower alkynyl group, a halogen derivative of alkene or alkyl respectively can be used. For example, an allyl group can be introduced by using an allyl halide such as allyl iodide in the presence of a catalyst such as silver oxide. This reaction can be conducted at a temperature from −10 to 50° C., preferably from 0° C. to 25° C., in a solvent such as dimethylformamide.

With the other method for producing the compound of formula (H) in which R¹ is not hydrogen, the compound represented by formula (H) in which both R¹ and R² are hydrogen atoms is treated with thionyl chloride by using a lower alcohol, for example methanol or ethanol as a solvent. In this case, a compound represented by formula (H) in which R¹ and R² are the same lower alkyl group corresponding to the lower alcohol solvent can be obtained. The reaction can be conducted at a temperature from −10° C. to 40° C., preferably from 0° C. to 25° C.

The compound represented by formula (H) in which R¹ is hydrogen can be produced, for example, by the following two methods. With the first method, it can be obtained by reacting glyceraldehydes CHO—COOH with an amine R²NH₂ protected with amino protecting group R². This reaction can be conducted at a temperature of 20° C. to 75° C. in a solvent such as acetone, ether, and the like, for example, by a method described in U.S. Pat. No. 3,668,121 issued to Philip X. Masciantonio et al., and by Stanlen D. Young et al., J. Am. Chem. Soc. 111, 1933 (1989), both of which are expressly incorporated by reference in their entireties. In this case, a compound represented by formula (H) in which both the R¹ and the R³ are hydrogen atoms can be obtained.

With the other method for the preparation of the compound represented by formula (H) in which R¹ is hydrogen, a compound represented by the following formula (I): 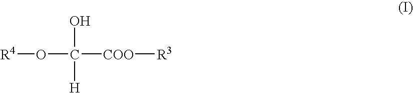
(wherein R³ is defined as described with reference to formula (H), and R⁴ is a lower alkyl group) is reacted with an amine R²NH₂ protected with amino protecting group R². This reaction can be conducted in a solvent such as tetrahydrofuran at a temperature of 20° C. to 80° C., for example, at the reflux temperature of the solvent used. The lower alkyl group R⁴ is defined as the lower alkyl group R¹.

Amino acid derivatives of AlphaHGA may be synthesized by reacting the amino-protected amide form of the amino acid with methyl α-hydroxymethoxyacetate followed by reaction with ammonia and deprotection such as with HCl. This method is illustrated for α-amino acids by the following scheme: 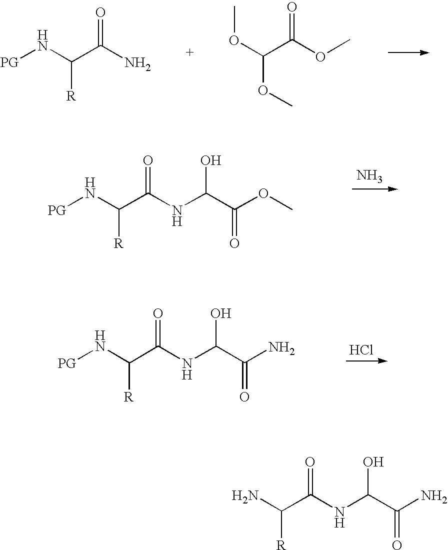
where PG represents the protecting group and R represents any side chain. If an amino acid chain is desired, additional amino acids can be added using any suitable peptide chemistry. The protecting group can be any suitable amino protecting group. Non-limiting examples include boc and fmoc.

Methods of Making and Using Pharmaceuticals and Dietary Supplements

Methods of making and using pharmaceuticals, medicaments, and dietary supplements comprising one or more of the active ingredients disclosed herein are also embodiments. The modified glycinamide compounds described herein are suitable for use as a biotechnological tool to study the interaction of a compound with HIV and also as a pharmaceutical or medicament for the treatment of subjects already infected with HIV, or as a preventive preparation to avoid HIV infection or as a dietary supplement for improving the function of the immune system or to promote a healthy immune system in subjects at risk of becoming infected with HIV or individuals that are already infected with the virus. The cofactor(s) obtainable by the methods described herein (either alone or in conjunction or combination with G-NH₂ or a G-NH₂ containing peptide, such as GPG-NH₂) are also suitable for use as biotechnological tools and as medicaments for the treatment and prevention of HIV replication.

By one approach, for example, a prodrug therapy is contemplated, wherein G-NH₂ or a G-NH₂ containing peptide, such as GPG-NH₂, is provided to a subject in need and the cofactor is provided by co-administration. Alternatively, the G-NH₂ or G-NH₂ containing peptide, such as GPG-NH₂ and the cofactor can be combined in a pharmaceutical (e.g., a pharmaceutical or dietary supplement composition comprising G-NH₂ or a G-NH₂ containing peptide, such as GPG-NH₂, and the cofactor). In this vein, cofactor and/or G-NH₂ and/or GPG-NH₂ and/or other glycinamide containing peptides can be administered as prodrugs when, for example, time release or long term treatments are desired.

Although anyone could be treated with compositions described herein, for example, as part of a daily dietary supplement program so as to promote immune system fitness and/or overall general health, the most suitable subjects are people at risk for HIV infection or people already infected with the virus. Such subjects include, but are not limited to, the elderly, the chronically ill, homosexuals, prostitutes, intravenous drug users, hemophiliacs, children, and those in the medical profession who have contact with patients or biological samples.

One aspect of the present invention relates to any one of the compounds or compositions described herein for use as a medicament. A further aspect of the invention relates to the use of any one of the compounds of the invention for the preparation of medicament for the treatment or prevention of AIDS or for the general promotion of the immune system, in people at risk of HIV infection, or people infected with HIV. A further aspect of the invention relates to the use of any one of the compounds of the invention for the preparation of dietary supplement or medicament for the general promotion of the immune system in athletes, the elderly, the chronically ill, homosexuals, prostitutes, intravenous drug users, hemophiliacs, children, and those in the medical profession who have contact with patients or biological samples.

The modified G-NH₂-containing compounds obtainable by the methods described herein (e.g., synthetic approaches or enzymatic approaches) can be processed in accordance with conventional methods of galenic pharmacy to produce medicinal agents and dietary supplements for administration to patients, e.g., mammals including humans. The modified G-NH₂ containing compounds can be incorporated into a pharmaceutical product or a dietary supplement with and without modification. Further, the manufacture of pharmaceuticals or therapeutic agents or dietary supplements that deliver modified G-NH₂-containing compounds by several routes is included within the scope of the present invention.

Some of the methods of making the antiretroviral compositions described herein, optionally, include the step of identification of a compound selected from the group consisting of compounds of formulas A, B, C, D, E, F, G, H, I, J, K, L, M, N, O, P, Q, R, S, T, U, V, W, X, Y, Z, or AA for incorporation in an antiretroviral pharmaceutical. Optionally, some of the methods of making the antiretroviral compositions described herein include the step of measuring the inhibition of HIV (e.g., the impact on viral lode is measured or the accumulation of a marker for HIV is measured, such as p24 presence or absence and/or rec\verse transcriptase activity). Accordingly, the compositions made the methods described herein are also embodiments.

The active ingredients described herein can be employed in admixture with conventional excipients, i.e., pharmaceutically acceptable organic or inorganic carrier substances suitable for parenteral, enteral (e.g., oral) or topical application that do not deleteriously react with the compounds. Suitable pharmaceutically acceptable carriers include, but are not limited to, water, salt solutions, alcohols, gum arabic, vegetable oils, benzyl alcohols, polyethylene glycols, gelatine, carbohydrates such as lactose, amylose or starch, magnesium stearate, talc, sialicic acid, viscous paraffin, perfume oil, fatty acid monoglycerides and diglycerides, pentaerythritol fatty acid esters, hydroxy methylcellulose, polyvinyl pyrrolidone, etc. Antioxidants (e.g., tocopherols, tocotrienols, and metabolites thereof, lycopene, ascorbic acid, whey protein, glutathione, and manitol) can be formulated with one or more of the compounds described herein. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the modified G-NH₂.

In some embodiments, compositions comprising modified G-NH₂-containing compounds are formulated with or administered in conjunction with other agents that inhibit viral infections, such as HIV infection, so as to achieve a better viral response. At present four different classes of drugs are in clinical use in the antiviral treatment of HIV-1 infection in humans. These are (i) nucleoside analogue reverse transcriptase inhibitors (NRTIs), such as zidovudine, lamivudine, stavudine, didanosine, abacavir, and zalcitabine; (ii) nucleotide analogue reverse transcriptase inhibitors, such as tenofovir; (iii) non-nucleoside reverse transcriptase inhibitors (NNRTIs), such as efavirenz, nevirapine, and delavirdine; (iv) protease inhibitors, such as indinavir, nelfinavir, ritonavir, saquinavir and amprenavir; and (v) entry (fusion) inhibitors, such as enfuvirtide. By simultaneously using two, three, or four different classes of drugs in conjunction with administration of the modified G-NH₂, HIV is less likely to develop resistance, since it is less probable that multiple mutations that overcome the different classes of drugs and the modified G-NH₂ will appear in the same virus particle.

It is thus preferred that pharmaceuticals and medicaments comprising modified G-NH₂-containing compounds are formulated with or given in combination with nucleoside analogue reverse transcriptase inhibitors, nucleotide analogue reverse transcriptase inhibitors, non-nucleoside reverse transcriptase inhibitors, and protease inhibitors at doses and by methods known to those of skill in the art. Medicaments and pharmaceuticals comprising the modified G-NH₂ and nucleoside analogue reverse transcriptase inhibitors, nucleotide analogue reverse transcriptase inhibitors, non-nucleoside reverse transcriptase inhibitors, and protease inhibitors can be formulated to contain other ingredients to aid in delivery, retention, or stability of the modified G-NH₂.

The effective dose and method of administration of a particular formulation can vary based on the individual needs of the subject. Therapeutic efficacy and toxicity of such compounds can be determined by standard pharmaceutical procedures in cell cultures or experimental animals, e.g., ED₅₀ and LD₅₀ (the dose lethal to 50% of the population). The dose ratio of toxic to therapeutic effects is the therapeutic index, and it can be expressed as the ratio, LD₅₀/ED₅₀. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the subject, and the route of administration.

The exact dosage is chosen by the individual or physician in view of the desired purpose. Dosage and administration can be adjusted to provide sufficient levels of the active moiety or to maintain the desired effect. Additional factors that may be taken into account include the severity of the disease state, age, weight and gender of the patient; diet, time and frequency of administration, drug combination(s), reaction sensitivities, and tolerance/response to therapy. Dietary supplements comprising one or more of the compounds described herein, for example, can be taken daily whereas long acting pharmaceutical compositions can be administered every 2, 3 to 4 days, every week, or once every two weeks. Depending on half-life and clearance rate of the particular formulation, the pharmaceutical and dietary supplements described herein are consumed, taken, or provided once, twice, three, four, five, six, seven, eight, nine, ten or more times per day.

Normal dosage amounts may vary from approximately 1 to 100,000 micrograms, up to a total dose of about 20 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 5000 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5, 6 g, 7 g, 8 g, 9 g, 10 g, 11 g, 12 g, 13 g, 14 g, 15 g, 16 g, 17 g, 18 g, 19 g, and 20 g. Preferred dosing regimens include 450 mg formulations three-times daily, 800 mg formulations three-times daily, and 1200 mg formulations twice daily. Additionally, the concentrations of the modified G-NH₂ can be quite high in embodiments that administer the agents in a topical form. Molar concentrations of may be used with some embodiments. Desirable concentrations for topical administration and/or for coating medical equipment range from 100:M to 800 mM. Preferable concentrations for these embodiments range from 500:M to 500 mM. For example, preferred concentrations for use in topical applications and/or for coating medical equipment include 500 μM, 550 μM, 600 μM, 650 μM, 700 μM, 750 μM, 800 μM, 850 μM, 900 μM, 1 mM, 5 mM, 10 mM, 15 mM, 20 mM, 25 mM, 30 mM, 35 mM, 40 mM, 45 mM, 50 mM, 60 mM, 70 mM, 80 mM, 90 mM, 100 mM, 120 mM, 130 mM, 140 mM, 150 mM, 160 mM, 170 mM, 180 mM, 190 mM, 200 mM, 300 mM, 325 mM, 350 mM, 375 mM, 400 mM, 425 mM, 450 mM, 475 mM, and 500 mM. Guidance as to particular dosages and methods of delivery is provided in the literature and below. (See e.g., U.S. Pat. Nos. 4,657,760; 5,206,344; or 5,225,212, herein expressly incorporated by reference in their entireties).

More specifically, the dosage of the modified G-NH₂ is one that provides sufficient modified G-NH₂ to attain a desirable effect including inhibition of proper viral release and/or inhibition of HIV replication or an improvement in immune system function. Accordingly, the dose of modified G-NH₂ preferably produces a tissue or blood concentration or both from approximately 0.1 nM to 500 mM. Desirable doses produce a tissue or blood concentration or both of about 0.1 nM to 800 μM. Preferable doses produce a tissue or blood concentration of greater than about 10 nM to about 300 μM. Preferable doses are, for example, the amount of modified G-NH₂ required to achieve a tissue or blood concentration or both of 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 200 nM, 300 nM, 400 nM, 500 nM, 600 nM, 700 nM, 800 nM, 900 nM, 1 μM, 10 μM, 15 μM, 20 μM, 25 μM, 30 μM, 50 μM, 100 μM, 200 μM, and 300 μM. Particularly preferred dosing regimens allow for 50 μM, 100 μM, or 200 μM stable concentrations in the blood and such levels can be obtained by providing a subject in need 12, 24, or 48 mg compound/kg bodyweight. That is, preferred dosing provides an amount of active ingredient that delivers between 50-200 μM of active ingredient to the blood, which can be obtained with a regimen that provides 12-48 mg of compound/kg bodyweight. However, doses greater than 12-48 mg of compound/kg bodyweight are also contemplated (e.g., 50 mg/kg, 100 mg/kg, 150 mg/kg, 200 mg/kg, 250 mg/kg, 300 mg/kg, 350 mg/kg, 400 mg/kg, 450 mg/kg, 500 mg/kg). Although doses that produce a tissue concentration of greater than 800 μM are not preferred, they can be used with some embodiments. A constant infusion of the modified G-NH₂ can also be provided so as to maintain a stable concentration in the tissues as measured by blood levels.

Routes of administration of the modified G-NH₂ include, but are not limited to, topical, transdermal, parenteral, gastrointestinal, transbronchial, and transalveolar. Topical administration is accomplished via a topically applied cream, gel, rinse, etc. containing modified G-NH₂. Transdermal administration is accomplished by application of a cream, rinse, gel, etc. capable of allowing the modified G-NH₂ to penetrate the skin and enter the blood stream. Parenteral routes of administration include, but are not limited to, electrical or direct injection such as direct injection into a central venous line, intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally.

Compositions of modified G-NH₂ containing compounds suitable for topical application include, but are not limited to, physiologically acceptable implants, ointments, creams, rinses, and gels. Any liquid, gel, or solid pharmaceutically acceptable base in which the compounds are at least minimally soluble is suitable for topical use in the present invention. Compositions for topical application are particularly useful during sexual intercourse to prevent transmission of HIV. Suitable compositions for such use include, but are not limited to, vaginal or anal suppositories, creams, jellies, lubricants, oils, and douches.

Compositions of the modified G-NH₂ suitable for transdermal administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference and are well known in the art. Examples of suitable transdermal devices are described, for instance, in U.S. Pat. No. 4,818,540, issued Apr. 4, 1989 to Chinen, et al., hereby incorporated by reference in its entirety.

Compositions of the modified G-NH₂ suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, or subcutaneous injection of the modified G-NH₂.

Compositions of the modified G-NH₂ suitable for transbronchial and transalveolar administration include, but are not limited to, various types of aerosols for inhalation. For instance, pentamidine is administered intranasally via aerosol to AIDS patients to prevent pneumonia caused by pneumocystis carinii. Devices suitable for transbronchial and transalveolar administration of the modified G-NH₂, including but not limited to atomizers and vaporizers, are also included within the scope of the present invention. Many forms of currently available atomizers and vaporizers can be readily adapted to deliver modified G-NH₂-Compositions of the modified G-NH₂ suitable for gastrointestinal administration include, but not limited to, pharmaceutically acceptable or dietary supplement suitable powders, pills, sachets, or liquids for ingestion and suppositories for rectal administration. Due to the most common routes of HIV infection and the ease of use, gastrointestinal administration, particularly oral, is preferred. Pharmaceuticals and dietary supplements for gastorintestinal administration, for example, are formulated in capsule, pill, or tablet form, wherein the active ingredient, modified glycinamide (e.g., α-hydroxyglycinamide, α-peroxyglycinamide dimer, diglycinamide ether, or α-methoxyglycinamide or one or more of the compounds of formulas A, B, C, D, E, F, G, H, I, J, K, L, M, N, O, P, Q, R, S, T, U, V, W, X, Y, Z, or AA), is in an amount effective to inhibit HIV replication.

The modified G-NH₂ is also suitable for use in situations where prevention of HIV infection is important or where an individual desires to maintain a healthy immune system. For instances, medical personnel are constantly exposed to patients who may be HIV positive and whose secretions and body fluids contain the HIV virus. Further, the modified G-NH₂ can be formulated into antiviral compositions for use during sexual intercourse so as to prevent transmission of HIV or to otherwise promote maintenance of a healthy immune system. Such compositions are known in the art and also described in the international application published under the PCT publication number WO90/04390 on May 3, 1990 to Modak et al., which is incorporated herein by reference in its entirety.

Embodiments of the invention also include a coating for medical equipment such as gloves, sheets, and work surfaces that protects against viral transmission. Alternatively, the modified G-NH₂ can be impregnated into a polymeric medical device. Particularly preferred are coatings for medical gloves and condoms. Coatings suitable for use in medical devices can be provided by a powder containing the peptides or by polymeric coating into which the peptide agents are suspended. Suitable polymeric materials for coatings or devices are those that are physiologically acceptable and through which a therapeutically effective amount of the modified G-NH₂ can diffuse. Suitable polymers include, but are not limited to, polyurethane, polymethacrylate, polyamide, polyester, polyethylene, polypropylene, polystyrene, polytetrafluoroethylene, polyvinyl-chloride, cellulose acetate, silicone elastomers, collagen, silk, etc. Such coatings are described, for instance, in U.S. Pat. No. 4,612,337, issued Sep. 16, 1986 to Fox et al., which is incorporated herein by reference in its entirety. Accordingly, methods of making a pharmaceutical, medicament, or dietary supplement that inhibits HIV replication or promotes maintainence of a health immune system, are practiced by providing modified G-NH₂, which can be prepared enzymatically or synthetically, and formulating said compound for delivery to a subject, including a human, as described above (e.g., preparing the compound according to GMP practices and formulating said compound into a tablet, capsule, powder etc.).

Methods of identification of compounds that inhibit HIV replication and/or otherwise improve or maintain the immune system of a subject are also provided. By one approach, for example, a compound for incorporation into a pharmaceutical or dietary supplement is identified by incubating G-NH₂ with serum, plasma, or a plant extract for a time sufficient to metabolize modified G-NH₂ and isolating the modified G-NH₂ by cation exchange HPLC. Preferably, human sera, pig sera, bovine sera, cat sera, dog sera, horse sera, monkey sera, pig plasma or a root nodule extract from a plant of Leguminosae, preferably Phaseolus, is used. By this approach, modified G-NH₂ rapidly elutes from the column, whereas unreacted G-NH₂ is retained on the column for a considerably longer period of time. The isolation of modified G-NH₂ can be further confirmed by conducting HIV infectivity studies in the presence of the isolated compound, as described above. Similarly, synthetic compounds that are related to α-hydroxyglycinamide, such as the compounds of formulas A, B, C, D, E, F, G, H, I, J, K, L, M, N, O, P, Q, R, S, T, U, V, W, X, Y, Z, or AA and derivatives of these compounds can be screened using the HIV infectivity studies presented herein. Depending on the purity of the modified G-NH₂ isolated or the structure of the synthetic modified glycinamide, the ED₅₀ of the compound is between less than 1 μM and less than 30 μM. That is, the ED₅₀ of pure modified G-NH₂ is less than 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10M, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM. Thus, in some embodiments, the modified G-NH₂ identified by the methods above is incorporated in a pharmaceutical. Furthermore, the methods above can be supplemented by providing an antiviral compound selected from the group consisting of nucleoside analogue reverse transcriptase inhibitors, nucleotide analogue reverse transcriptase inhibitors, non-nucleoside reverse transcriptase inhibitors, and protease inhibitors into the pharmaceutical. Additionally, the methods above can be supplemented by incorporating a carrier into the pharmaceutical or dietary supplement.

Although the modified G-NH₂ can be used as a research tool to analyze the inhibition of HIV, desirably modified G-NH₂ is used to inhibit HIV replication and infection in a subject. By one method, for example, a subject at risk of becoming infected by HIV or who is already infected with HIV is identified and said subject is provided a pharmaceutical or medicament containing modified G-NH₂, which can be in a unit dosage form. By an additional method, a subject is provided modified G-NH₂ and the effect on HIV replication or infection, is determined (e.g., by analyzing the amount of p24 or reverse transcriptase activity in a biological sample). By still another method, a subject that desires to improve the function of their immune system or to promote the maintenance of their immune system is provided a dietary supplement that comprises, consists of, or consists essentially of a modified glycinamide, such as one or more of the compounds of formulas A, B, C, D, E, F, G, H, I, J, K, L, M, N, O, P, Q, R, S, T, U, V, W, X, Y, Z, or AA or a modified glycinamide obtained by or obtainable by mixing glycinamide with an animal serum or plasma (e.g., bovine, pig, or horse), an extract from root nodules of a plant (e.g., Phaseolus vulgaris) or a recombinant oxido-reduction protein expressed in a host (e.g., bacteria, insect cells, or animal cells) and isolated or purified therefrom.

Not wishing to be bound by any theory or mechanism and offered only as an example of one possible mode of action, it is contemplated that modified glycinamide inhibits replication of HIV by disrupting capsid assembly, a mechanism that is different than conventional nucleoside analogues, protease inhibitors, and entry (fusion) inhibitors. (See U.S. Pat. Nos. 6,258,932; 6,455,670; 6,537,967; all of which are hereby expressly incorporated by reference in their entireties). Accordingly, preferred subjects to receive pharmaceuticals and dietary supplements containing modified glycinamide are HIV infected individuals that have developed resistance to conventional therapies.

By one approach, nine HIV infected patients are provided differing amounts of a modified glycinamide (e.g., alpha-hydroxyglycinamide, Metabolite X, such as a modified glycinamide obtained by an enzymatic approach described herein, alpha-peroxyglycinamide dimer, diglycinamide ether or alpha-methoxyglycinamide or one or more of the compounds of formulas A, B, C, D, E, F, G, H, I, J, K, L, M, N, O, P, Q, R, S, T, U, V, W, X, Y, Z, or AA) and the inhibition of HIV replication is analyzed. Group I, which contains three individuals, is provided a dietary supplement or pharmaceutical with 1.0 g of modified glycinamide by capsule form three times a day; whereas Group II, which contains three individuals, is provided a dietary supplement or pharmaceutical with 1.5 g of modified glycinamide by capsule form three times a day; and Group III, which contains three individuals is provided a dietary supplement or pharmaceutical with 2.0 g of modified glycinamide by capsule form throughout the day. The reduction in viral load is monitored daily by conventional techniques that detect the amount of HIV RNA (e.g., Roche AMPLICOR MONITOR™). A reduction in viral load will be observed, as indicated by a reduction in the amount of HIV RNA detected. The improvement or maintenance of an aspect of the immune system (e.g., T cell count) is also monitored and it will be seen that subjects receiving the dietary supplement or pharmaceutical will experience an increase in CD4+ T cells over time, an improvement in Tcell percentage, or an improvement in resistance to an opportunistic infection.

The methods above can be supplemented with administration of an antiviral treatment selected from the group consisting of nucleoside analogue reverse transcriptase inhibitors, nucleotide analogue reverse transcriptase inhibitors, non-nucleoside reverse transcriptase inhibitors, protease inhibitors, and entry (fusion) inhibitors. Further, the modified G-NH₂ used in these methods can be joined to a support or can be administered in a pharmaceutical comprising a pharmaceutically acceptable carrier.

EXAMPLE 32 describes a microdosing study that was performed to determine the safety and bioavailabiity of alphahydroxyglycinamide and EXAMPLE 33 describes a protocol that was submitted for a clinical trial designed to evaluate the safety and efficacy of alphahydroxyglycinamide in HIV infected patients.

### EXAMPLES

### Example 1

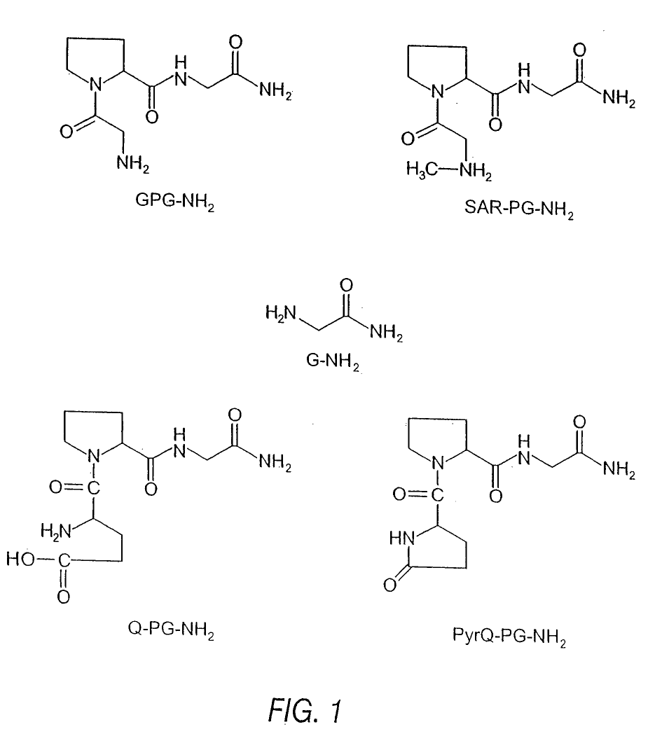

In initial experiments, several HIV-1 and HIV-2 strains were evaluated for their sensitivity to the inhibitory activity of GPG-NH₂, G-NH₂ and related compounds. (See TABLE 1 and FIG. 1). Glycylprolylglycinamide (GPG-NH₂), glutaminylprolylglycinamide (Q-PG-NH₂), sarcosinylprolylglycinamide (Sar-PG-NH₂) and glycinamide (G-NH-₂) were provided by TRIPEP AB (Huddinge, Sweden); whereas, Pyrroglutaminylprolylglycinamine (PyrQ-PG-NH₂) was synthesized at the Rega Institute. Human T-lymphocytic CEM cells were obtained from the American Type culture Collection (Rockville, Md.) and cultured in RPMI-1640 medium (Gibco, Paisley, Scotland supplemented with 10% fetal bovine serum (FBS) (BioWittaker Europe, Verviers, Belgium), 2 mM L-glutamine (Gibco) and 0.075 M NaHCO₃ (Gibco). HIV-1 (III_{B}) was obtained from Dr. R. C. Gallo and Dr. M. Popovic (at that time at the National Cancer Institute, NIH, Bethesda, Md.). HIV-1 (NL4.3) was from the National Institute of Allergy and Infectious Disease AIDS Reagent Program (Bethesda, Md.). The HIV-2 isolates ROD and EHO were provided by Dr. L. Montagnier (Pasteur Institute, Paris, France).

Human T-lymphocytic CEM cells (4.5×10⁵ cells per ml) were suspended in fresh cell culture medium and infected with HIV-1 (III_{B} and NL4.3) or HIV-2 (ROD or EHO) at 100 CCID₅₀ (1 CCID₅₀ being the virus dose infective for 50% of the cell cultures) per ml of cell suspension. Then, 100 μl of the infected cell suspension were transferred to microplate wells, mixed with 100 μl of appropriate (freshly prepared) dilutions of the test compounds (i.e., at final concentrations of 2000, 400, 80, 16, 3.2 and 0.62 μM), and were further incubated at 37° C. After 4 to 5 days, giant cell formation was recorded microscopically in the CEM cell cultures. The 50% effective concentration (EC₅₀) corresponded to the compound concentrations required to prevent syncytium formation in the virus-infected CEM cell cultures by 50%. (See Table 1).

TABLE 1
Inhibitory activity of tripeptide derivatives against several virus
strains in CEM cell cultures

| | EC₅₀^{a} (μM) | | | | |
|---|---|---|---|---|---|
| | HIV-1 | | HIV-2 | | |
| Compound | III_{B} | NL3.4 | ROD | EHO | CEM |
| GPG-NH₂ | 35 ± 8.7 | 50 ± 0.0 | 30 ± 10 | 42 ± 14 | >2000 |
| G-NH₂ | 32 ± 7.6 | 45 ± 7.1 | 35 ± 8.7 | 37 ± 5.8 | >2000 |
| PyrQ-PG-NH₂ | >2000 | >2000 | >2000 | >2000 | >2000 |
| SAR-PG-NH₂ | 31 ± 4.9 | 49 | 35 ± 9.8 | 56 | >1500 |
| Q-PG-NH₂ | 86 | 265 | 89 | 82 | >1500 |

^{a}50% Effective concentration, or compound concentration required to inhibit HIV-reduced syncytia formation in T-lymphocytic CEM cell cultures

Interestingly, both GPG-NH₂ and G-NH₂ were equally effective in suppressing virus replication on a molar basis, regardless the nature of the virus used in the antiviral assays. Their EC₅₀ (50% effective concentration) ranked between 30 and 50 μM in CEM cell cultures. Both compounds did not show cytotoxicity at concentrations as high as 1500 to 2000 μM. Sar-PG-NH₂ and Q-PG-NH₂ were also inhibitory to HIV replication, although to a lower extent than GPG-NH₂. A novel tripeptide (PyrQ-PG-NH₂) derivative was synthesized containing G-NH₂ at its carboxy terminal end but a cyclic pyrroglutamine at its amino terminal end. In contrast with GPG-NH₂ and the other tripeptide amide derivatives, PyrQ-PG-NH₂ was found to be ineffective at inhibiting HIV replication in cell culture

Next, it was confirmed that CD26 dipeptidylpeptidase activity could be detected in purified CD26 and bovine, murine and human serum and with human lymphocytic or peripheral blood mononuclear cell suspensions. CD26 enzyme activity was recorded by conversion of the synthetic substrate glycylprolyl p-nitroanilide (GP-pNA) to glycylproline (GP-OH) and p-nitroaniline (pNA), a yellow dye, whose formation could be monitored by an increase of the absorption at 400 nm. Approximately, two hundred microliters of purified CD26 (1 milliUnit/ml) in phosphate buffered saline (PBS), or human, murine or bovine serum (5% in PBS) or 10⁶ human lymphocytic CEM, C8166, Molt4/C8, MT-4 or peripheral blood mononuclear cell suspensions in PBS were added to 200 μl-microtiter plate wells after which the substrate for measuring the CD26 enzymatic activity (glycylprolyl-para-nitroanilide) (GP-pNA) at 3 mM final concentration was added. Glycylprolyl-p-nitroanilide (GP-pNA) and glycylphenylalaninyl-p-nitroanilide (GF-pNA) were obtained from Sigma Chemicals (St. Louis, Mo.). The release of p-nitro-aniline (pNA) was monitored at 37° C. in function of time by measuring the amount of (yellow-colored) para-nitroaniline (pNA) released from GlyPro-pNA. The pNA release was recorded by the increase of absorption [optical density (OD) at 400 nm] in a Spectramax microplate spectrometer (Molecular Devices, Sunnyvale, Calif.). Under the experimental conditions, the reaction proceeded linearly for at least 60 min. The OD₄₀₀ values of blank reaction mixtures (lacking the CD26 enzyme, serum or cells) were subtracted from the obtained OD₄₀₀ values to represent the real increase of OD₄₀₀ value as a measurement of the enzyme activity.

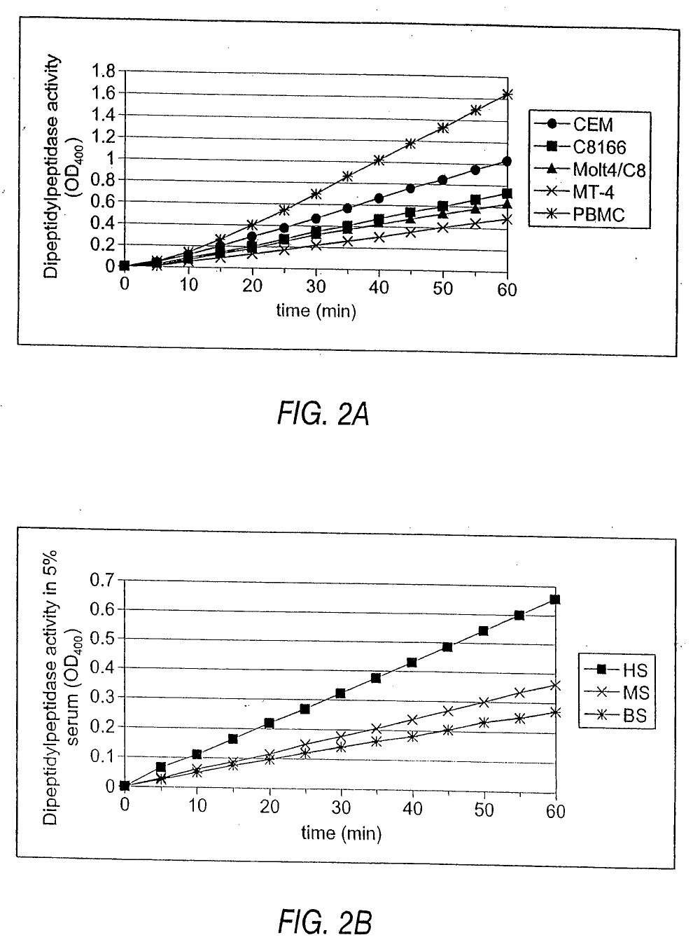

It was found that GP-pNA was only converted by CD26 and not by the action of other dipeptidyl/peptidases since the addition of a specific inhibitor of CD26 to the cell suspensions virtually completely blocked the release of p-nitroaniline from the synthetic substrate GP-pNA (infra). All lymphocytic cell suspensions (CEM, C8166, MT-4, Molt4/C8) and also PBMC at which GP-pNA had been administered efficiently converted GP-pNA to p-nitroaniline in a time-dependent fashion. (See FIG. 2A). The CD26 activity was highest in CEM cell suspensions and lowest in the MT-4 cell suspensions. Also, fetal bovine and murine serum and in particular human serum efficiently released p-nitroaniline from GP-pNA (See FIG. 2B). Thus, both human T-lymphocytic cell suspensions and serum display a prominent CD26/dipeptidylpeptidase enzyme activity. Once it was determined that CD26 activity could be efficiently monitored, experiments were conducted to determine if CD26 could convert GPG-NH₂ to G-NH₂.

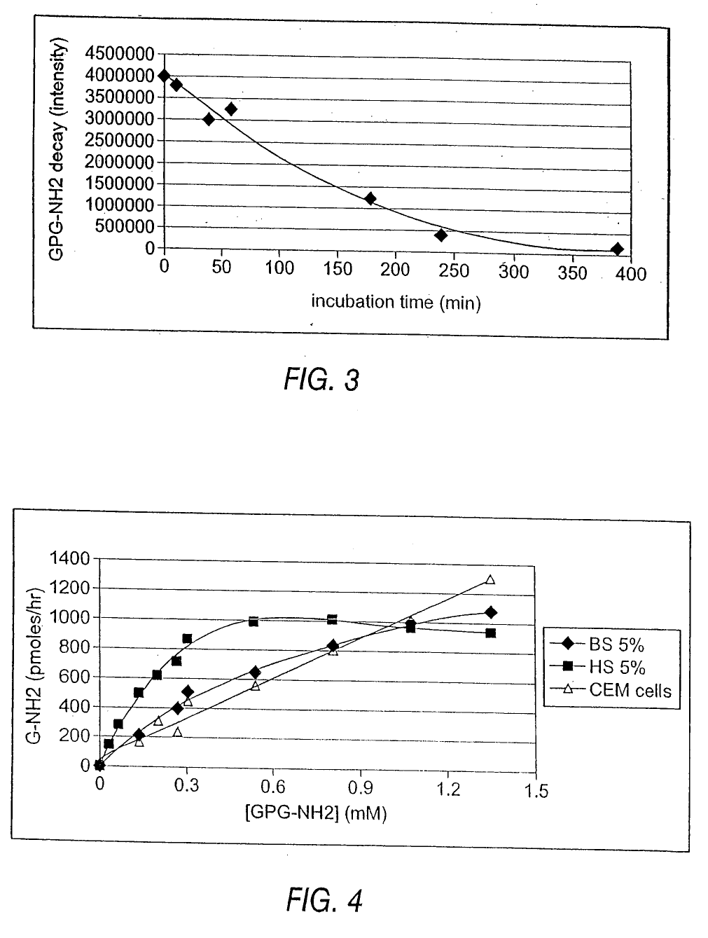

In some experiments, approximately, 100 μM GPG-NH₂ was exposed to 25 units/l of purified CD26 and the mixture was incubated for up to 400 minutes at room temperature. The lymphocyte surface glycoprotein CD26/dipeptidylpeptidase IV was purified as described before. (See De Meester, J. Immunol. Methods, 189:99-105 (1996), herein expressly incorporated by reference in its entirety). At different time points, an aliquot of the reaction mixture was withdrawn and analyzed on an electrospray ion trap mass spectrometer (Esquire, Bruker, Bremen, Germany). The appearance of the dipeptide GP-OH upon release from the amino terminal end of the GPG-NH₂ molecule, as well as, the disappearance of intact GPG-NH₂ from the reaction mixture was determined and monitored by electrospray ion trap mass spectometric analysis at different time points (See FIG. 3). Under these experimental conditions, CD26 released GP-OH in a time-dependent manner from GPG-NH₂, and virtually completely converted GPG-NH₂ to GP-OH and G-NH₂ within 4 to 6 hrs of the reaction. In contrast, CD26 was unable to release G-NH₂ from PyrroQ-PG-NH₂.

Next, the conversion of radiolabeled [¹⁴C]GPG-NH₂ to [¹⁴C]G-NH₂ by purified CD26, fetal bovine serum (FBS), human serum (HS) and CEM cell suspensions was analyzed. Radiolabeled [¹⁴C]GPG-NH₂ (radiospecificity: 58 mCi/mmol), in which the radiolabeled carbon is located in the main chain carbon of the glycine at the carboxylic acid end of the tripeptide, and [¹⁴C]G-NH₂ (radiospecificity: 56 mCi/mmol) in which carbon-2 was radiolabeled were synthesized by Amersham Pharmacia Biotech (Buckinghamshire, England). A variety of these [¹⁴C]GPG-NH₂ concentrations were exposed to purified CD26, FBS, HS and CEM cell suspensions and the conversion to G-NH₂ was analyzed.

In one set of experiments, for example, five-ml CEM cell cultures (5×10⁵ cells/ml) were exposed to 20 μM [¹⁴C]GPG-NH₂ for 24 hrs. Then, the cells were centrifuged for 10 min at 1,200 rpm, washed, and the cell pellet was treated with 60% ice-cold methanol for 10 min. The methanol cell extract was centrifuged for 10 min at 15,000 rpm, after which the supernatant was injected on a cation exchange Partisphere-SCX column (Whattman) to separate GPG-NH₂ from G-NH₂. The following gradient was used: 0-15 min: isocratic buffer A (7 mM sodium phosphate, pH 3.5); 15-40 min linear gradient from buffer A to buffer B (250 mM sodium phosphate, pH 3.5); 40-45 min linear gradient from buffer B to buffer A; 45-55 min: isocratic buffer A. The retention time of [¹⁴C]GPG-NH₂ and [¹⁴C]G-NH₂ under these elution conditions were 26-28 min and 14-16 min, respectively.

In another set of experiments, after one hour of exposure, disappearance of intact [¹⁴C]GPG-NH₂ was determined by HPLC analysis, as described above, using a cation-exchange Partisphere SCX column and a sodium phosphate buffer gradient at pH 3.5. GPG-NH₂ was well-separated from G-NH₂ (retention times: 25-27 min and 15-17 min, respectively). The Kₘ value of CD26-catalyzed conversion of GPG-NH₂ to G-NH₂ was calculated to be 0.183 mM. The estimated Kₘ values of GPG-NH₂ for dipeptidylpeptidase activity associated with HS and FBS were 0.45 and 1.4 mM, respectively, as derived from the GPG-NH₂ disappearance curves depicted in FIG. 4. The GPG-NH₂ conversion by the CEM cell suspensions proceeded linearly up to 1.5 mM. Only at higher GPG-NH₂ concentrations (e.g., 3 and 5.4 mM), did the conversion curve for the CEM cell suspensions start to level-off slightly.

Next, the inhibitory effect of L-isoleucinepyrrolidine (IlePyr) on CD26 was analyzed. Isoleucinepyrrolidine (IlePyr) has recently been reported to be a relatively potent and selective inhibitor of purified CD26-associated dipeptidylpeptidase activity. (See De Meester, J. Immunol. Methods, 189:99-105 (1996)). All enzyme activity assays were performed in 96-well microtiter plates (Falcon, Becton Dickinson, Franklin Lakes, N.J.). To each well were added 5 μl purified CD26 in PBS (final concentration of 0.2 milliUnits/200 μl-well), 10 μl fetal bovine serum (BS) (final concentration: 5% in PBS; preheated at 56° C. for 30 min), or one million CEM cells in PBS, 5 μl of an appropriate concentration of the IlePyr inhibitor solution in PBS (500 and 200 μM) and PBS to reach a total volume of 150 μl. The reaction was started by the addition of 50 μl substrate GP-pNA at 4 mg/ml (final concentration in the 200 μl reaction mixture: 1 mg/ml or 3 mM) and carried out at 37° C. The 50% inhibitory concentration of IlePyr against dipeptidylpeptidase activity associated with CD26, BS and CEM cell suspensions was defined as the compound concentration required to inhibit the enzyme-catalyzed hydrolysis of GP-pNA to pNA and GP-OH by 50%.

In initial experiments, CD26 inhibition in CEM cell suspensions (in fetal bovine serum) subjected to IlePyr using GP-pNA as the substrate was analyzed. Purified CD26 was included as a positive control. (See FIG. 5). The inhibitor IlePyr dose-dependently prevented release of p-nitroaniline from GP-NA exposed to CEM cell suspensions as well as to fetal bovine serum at a 50% inhibitory concentration (IC₅₀) of 110 and 99 μM, respectively. Purified CD26 was inhibited at an IC₅₀ value of 22 μM. Thus, the 50% inhibitory concentration (IC₅₀) value of the inhibitor IlePyr exposed to serum and CEM cell suspensions was ˜5-fold higher than the inhibitor concentrations required to inhibit purified CD26 by 50%.

Then, experiments were conducted to determine if the antiretroviral activity observed with GPG-NH₂ was associated with the CD26-catalyzed release of G-NH₂ from the tripeptide derivative. HIV-1-infected CEM cell cultures were exposed to different concentrations of GPG-NH₂ in the presence of non-toxic concentrations of IlePyr (500 μM and 200 μM). Similar combinations of G-NH₂ with IlePyr were included in this study. In these experiments, the CD26-specific inhibitor L-isoleucinepyrrolidine (IlePyr), was added to each cell culture microplate prior to the addition of the test compounds and the virus-infected cells.

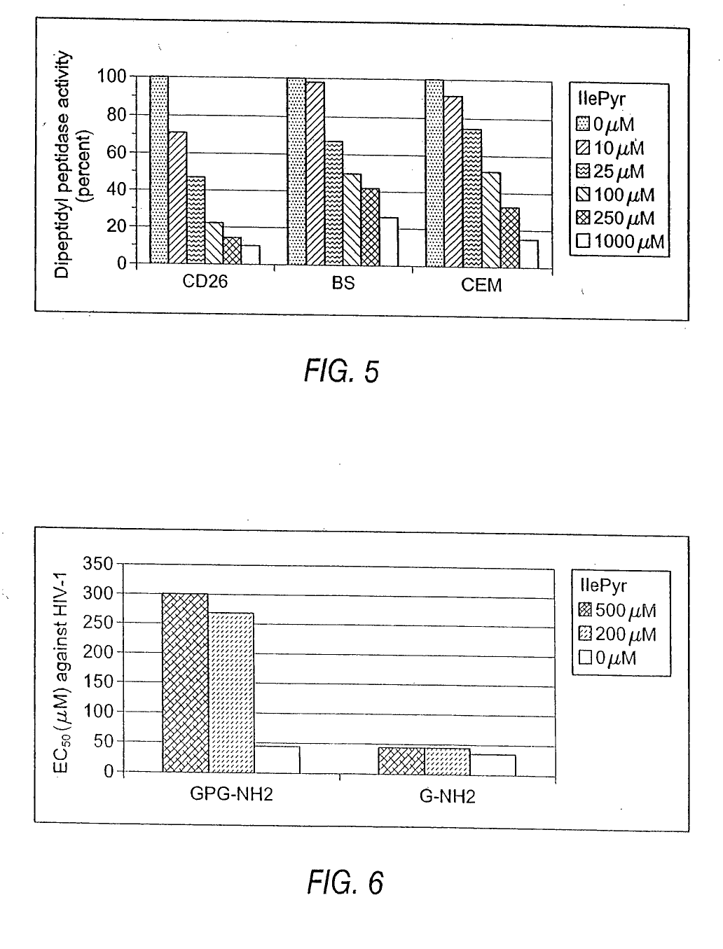

In contrast with G-NH₂, which fully preserved its anti-HIV activity in CEM cell cultures in the presence of 200 and 500 μM of IlePyr (EC₅₀-35-43 μM), GPG-NH₂ markedly lost its inhibitory activity against virus-induced cytopathicity in the presence of the specific CD26 inhibitor. (See FIG. 6). The highest inhibitor concentration (500 μM) was slightly more efficient in reversing the anti-HIV-1 activity of the tripeptide GPG-NH₂ than the lower (200 μM) inhibitor concentration. A similar result was observed for Sar-GP-NH₂, another tripeptide amide derivative that is also endowed with antiretroviral activity in cell culture.

The results presented this example, demonstrate that GPG-NH₂ requires hydrolysis to release glycinamide before it is able to exert its anti-HIV activity in cell culture. The data also provide evidence that the release of G-NH₂ from GPG-NH₂ is induced by the enzymatic activity of the lymphocyte surface glycoprotein activation/differentiation marker CD26. The formation of G-NH₂ from GPG-NH₂ was conducted with purified CD26, human T-lymphocyte cell suspensions and human and bovine serum. Moreover, the pronounced antiviral activity of Q-PG-NH₂, the complete lack of antiviral activity of PyrQ-PG-NH₂ (that is resistant to enzymatic attack by CD26) and the loss of antiviral efficacy of GPG-NH₂ and Sar-GP-NH₂ in the presence of a specific inhibitor of CD26 provide strong evidence that GPG-NH₂ acts as an efficient prodrug of G-NH₂ and that CD26-catalyzes the conversion of GPG-NH₂ to G-NH₂.

### Example 2

Human T-lymphocytic CEM cells (approx. 4.5×10⁵ cells/ml) were suspended in fresh medium and were infected with HIV-1 (III_{B}) at approx. 100CCID₅₀ per ml of cell suspension (1CCID₅₀ being the virus dose infective for 50% of the cell cultures). Then, 100 μl of the infected cell suspension was transferred to individual wells of a microtiter plate (100 μl/well) and was mixed with 100 μl of freshly diluted test compound (2000, 400, 80, 16, 3.2, or 0.62 μM). Subsequently, the mixtures were incubated at 37° C. After 4 to 5 days of incubation, giant cell formation was recorded microscopically in the CEM cultures. The 50% effective concentration (EC₅₀) corresponded to the concentrations of the compounds required to prevent syncytium formation in the virus-infected CEM cell cultures by 50%.

The results of these experiments are shown in TABLE 2. Glycinamide was found to be the only compound that appreciably inhibited HIV replication in cell culture. The EC₅₀ for G-NH₂ was approximately 21.3 μM, whereas the other compounds tested showed no inhibition of HIV. These results confirmed that G-NH₂ has a particular structure that inhibits HIV replication.

TABLE 2
Inhibitory activity of compounds against HIV-1(III_{B}) in
CEM cell cultures

| | EC₅₀(μM)^{a} |
|---|---|
| Glycinamide | 21.3 ± 16.3 |
| Glycin-thioamide | >500 |
| Cyclic glycin-thioamide | >500 |
| L-Alaninamide | >500 |
| L-Leucinamide | >500 |
| L-Isoleucinamide | >500 |
| L-Valinamide | >500 |
| L-Lysinamide | >500 |
| L-Asparaginamide | >500 |
| L-Val β-naphthylamide | >100 |
| Ala-Pro-Gly-Trp-amide | >500 |
| DL-Leucinamide | >500 |
| DL-Tryptophanamide | >500 |
| L-Tyrosinamide | >500 |
| D-Asparagine | >500 |
| L-Phenylalaninamide | >500 |
| L-Methioninamide | >500 |
| L-Threoninamide | >500 |
| L-Argininamide | >500 |
| L-Tryptophanamide | >200 |
| L-Prolinamide | >1000 |
| L-Asparaginamide | >1000 |
| DL-Phenylalaninamide | >1000 |
| D-Leucine | >1000 |
| Sarcosinamide | >1000 |
| L-Serinamide | >1000 |
| L-Alanine | >500 |
| L-Leucine | >500 |
| L-Proline | >500 |
| Glycine | >500 |
| 1,3-diaminoaceton | >1000 |
| Ethylene diamine | >1000 |
| 1,4-diamino-2-butanone | — |
| 1,3-diamino-2-hydroxypropane | >1000 |
| DL-2,3-diaminopropionic acid | >1000 |
| Glycine methylamide | >500 |

^{a}50% effective concentration

Subsequent analysis revealed that G-NH₂ was a specific inhibitor of HIV. The cytotoxicity and antiviral activity of various concentrations of G-NH₂ and GPG-NH₂ were evaluated in cell cultures that were infected with various types of viruses. Conventional host cell culture, viral infection, and infectivity analysis for each different type of cell and virus were followed. Compounds that were known to inhibit replication of the particular types of viruses analyzed were used as controls.

TABLES 3-5 show the results of these experiments. The data show that G-NH₂ and GPG-NH₂ were ineffective at inhibiting the replication of Herpes simplex virus-1 (KOS), Herpes simplex virus-2 (G), Herpes simplex virus-1 TK⁻ KOS ACV^{r}, Vaccinia virus, Vesicular stomatis virus, Coxsackie virus B4, Respiratory syncytial virus, Parainfluenza-3 virus, Reovirus-1, Sindbis virus, and Punta Toro virus. These results confirmed that G-NH₂ and GPG-NH₂ are selective inhibitors of HIV.

TABLE 3
Cytotoxicity and antiviral activity of compounds in HEL cell cultures

| | | Minimum inhibitory concentration^{b} | | | | |
|---|---|---|---|---|---|---|
| | | | | | | Herpes |
| | Minimum | Herpes | Herpes | | | simplex |
| | Cytotoxic | simplex | simplex | | Vesicular | virus-1 |
| | Concentration^{a} | virus-1 | virus-2 | Vaccinia | stomatitis | TK⁻ KOS |
| Compound | (μg/ml) | (KOS) | (G) | virus | virus | ACV^{r} |
| G-NH₂ (μM) | >2000 | >2000 | >2000 | >2000 | >2000 | >2000 |
| GPG-NH₂ | >400 | >400 | >400 | >400 | >400 | >400 |
| (μM) | | | | | | |
| BVDU | >400 | 0.0256 | >400 | 0.64 | 400 | 400 |
| (μg/ml) | | | | | | |
| Ribavirin | >400 | 48 | >400 | 240 | >400 | 80 |
| (μg/ml) | | | | | | |
| ACG (μg/ml) | >400 | 0.0768 | 0.0768 | >400 | >400 | 9.6 |
| DHPG | >100 | 0.0038 | 0.0192 | 60 | >400 | 0.48 |
| (μg/ml) | | | | | | |

^{a}Required to cause a microscopically detectable alteration of normal cell morphology.
^{b}Required to reduce virus-induced cytopathogenicity by 50%.

TABLE 4
Cytotoxicity and antiviral activity of compounds in HeLa cell cultures

| | Minimum | Minimum inhibitory concentration^{b} | | |
|---|---|---|---|---|
| | cytotoxic | Vesicular | | Respiratory |
| | concentration^{a} | stomatitis | Coxsackie | syncytial |
| Compound | (μg/ml) | virus | virus B4 | virus |
| G-NH₂ (μM) | >2000 | >2000 | >2000 | >2000 |
| GPG-NH₂ | >400 | >400 | >400 | >400 |
| (μM) | | | | |
| Brivudin | ≧400 | >400 | >400 | >400 |
| (μg/ml) | | | | |
| (S)-DHPA | >400 | 240 | >400 | >400 |
| (μg/ml) | | | | |
| Ribavirin | >400 | 9.6 | 48 | 16 |
| (μg/ml) | | | | |

^{a}Required to cause a microscopically detectable alteration of normal cell morphology.
^{b}Required to reduce virus-induced cytopathogenicity by 50%.

TABLE 5
Cytotoxicity and antiviral activity of compounds in Vero cell cultures

| | Minimum | Minimum inhibitory concentration^{b} | | | | |
|---|---|---|---|---|---|---|
| | cytotoxic | | | | | Punta |
| | concentration^{a} | Parainfluenza-3 | | Sindbis | Coxsackie | Toro |
| Compound | (μg/ml) | virus | Reovirus-1 | virus | virus B4 | virus |
| G-NH₂ | >2000 | >2000 | >2000 | >2000 | >2000 | >2000 |
| (μM) | | | | | | |
| GPG-NH₂ | >400 | >400 | >400 | >400 | >400 | >400 |
| (μM) | | | | | | |
| BVDU | >400 | >400 | >400 | >400 | >400 | >400 |
| (μg/ml) | | | | | | |
| (S)-DHPA | >400 | 240 | 80 | >400 | >400 | >400 |
| (μg/ml) | | | | | | |
| Ribavirin | >400 | 48 | 16 | >400 | >400 | 48 |
| (μg/ml) | | | | | | |

^{a}Required to cause a microscopically detectable alteration of normal cell morphology.
^{b}Required to reduce a virus-induced cytopathogenicity by 50%

### Example 3

It was observed that when unpurified preparations of [¹⁴C]G-NH₂ were separated by cation exchange high performance liquid chromatography (HPLC) two populations of G-NH₂ were resolved. (See TABLE 6). Crude preparations of radiolabeled G-NH₂ and radiolabeled GPG-NH₂ were separated by HPLC using a cation exchange column (e.g., Partisphere SCX-Whattman). The following gradient was used: 0-15 minutes (isocratic Buffer A composed of 5 mM ammonium phosphate, pH 3.5); 15-40 minutes linear gradient from Buffer A to Buffer B (composed of 250 mM ammonium phosphate, pH 3.5); 40-45 minutes Buffer B; 45-55 minutes linear gradient to Buffer A; and 55-60 minutes isocratic Buffer A to equilibrate the column for the next run.

By this separation approach, the majority of crude [¹⁴C]GPG-NH₂ typically eluted in 26-28 minutes (fractions 26-28), however, trace amounts of radiolabeled compounds eluted in 20-22 minutes (fractions 20-22), 15-17 minutes (fractions 15-17), and 2-3 minutes (fractions 2-3). Approximately 89% of the crude [¹⁴C]G-NH₂ typically eluted in 15-17 minutes (fractions 15-17) but approximately 11% of the crude [¹⁴C]G-NH₂ eluted in 2-3 minutes (fractions 2-3). Trace amounts of crude [¹⁴C]G-NH₂ were also detected in fractions 20-22 and fractions 5-6.

Slight alterations in the buffers and the gradient led to slight shifts in the time of elution of the compounds but, in all preparations, two main populations of glycinamide were detected, a first population that quickly eluted from the column (referred to as the fast peak, fraction 2-3 or fraction 3-4, or impurity in radiolabeled G-NH₂, or modified G-NH₂) and a second population that strongly bound to the column (referred to as the slow peak, fraction 13-14 or fraction 15-17 or G-NH₂). For example, another protocol to isolate modified G-NH₂ used also Buffer A (5 mM ammoniumphosphate pH 3.5) and Buffer B (250 mM ammoniumphosphate pH 3.5). The gradient used with these buffers was as follows: 10 minutes Buffer A; linear gradient to Buffer B for 6 minutes; 2 minutes at Buffer B; then linear gradient to Buffer A for 6 minutes; and equilibration in Buffer A for 6 minutes. By this approach, as well, the G-NH₂, and impurity in radiolabeled G-NH₂ eluted at 10-11 minutes and 2-3 minutes, respectively.

TABLE 6
Purity of [¹⁴C]radiolabeled stock of GPG-NH₂ and G-NH₂

| | Fraction Number on HPLC (cation exchange) | | | | | |
|---|---|---|---|---|---|---|
| Drug | 2-3 | 5-6 | 15-17 | 20-22 | 26-28 | Total |
| G-NH₂ | 53,000 (11%) | 1,700 | 435,000 (89%) | 1,300 (<0.5%) | — | 490,000 (100%) |
| | | (<0.5%) | | | | |
| GPG-NH₂ | 5,100 (1.5%) | — | 700 (<0.5%) | 10,600 (3%) | 339,000 | 355,400 (100%) |
| | | | | | (95%) | |

In this example, an approach to purify commercially obtained G-NH₂ is provided. A modification of this approach has been used to purify modified glycinamide, as described infra. It should be understood that many different cation exchange columns are available for these procedures and many different buffers and gradients can be used. Given the disclosure herein, one of skill in the art can rapidly adapt a particular type of cation exchange column, FPLC or HPLC, buffer, or gradient to isolate modified G-NH₂ (Metabolite X). That is, modifications of the procedures described above are within the skill in the art and are equivalent to the methods described herein.

### Example 4

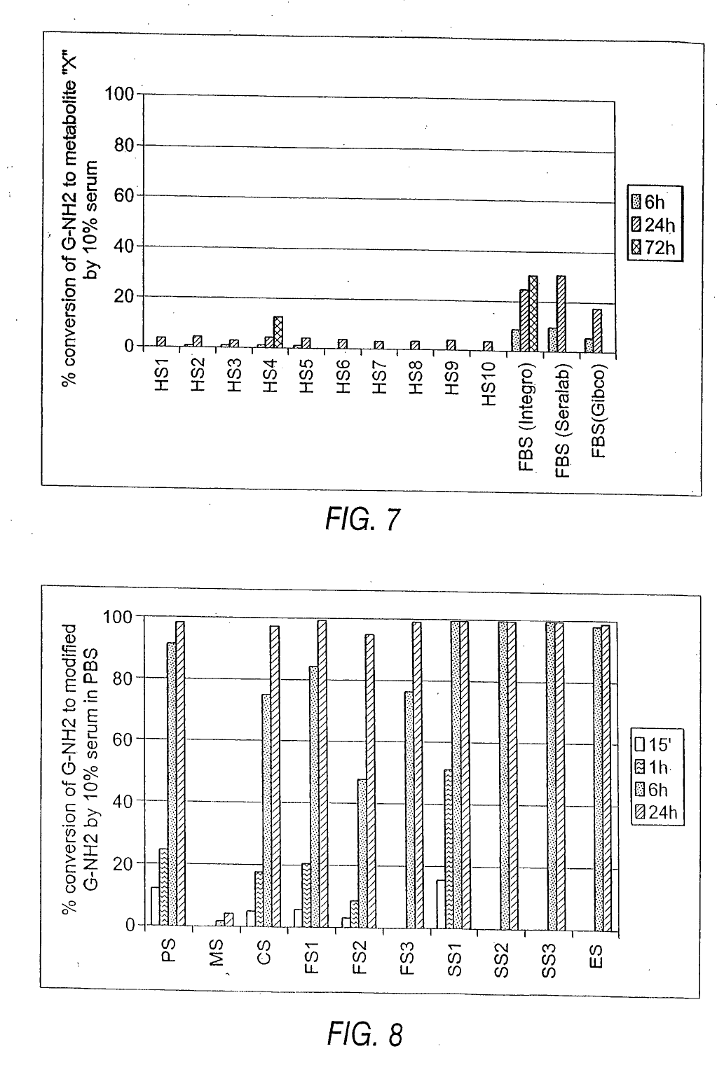

Several lots of human sera and fetal bovine sera were analyzed for their ability to convert G-NH₂ to modified G-NH₂. Radiolabeled cation exchange HPLC purified G-NH₂ (see EXAMPLE 3) was incubated with the various sera at a 10% final concentration in PBS at 37° C. for 15 minutes and 1, 6, 24, or 72 hours. Subsequently, the amount of radiolabeled modified G-NH₂ was evaluated using the cation exchange HPLC approach described above. The results are shown in FIG. 7. Each of the 10 different human serum samples showed less than 10% conversion of G-NH₂ to modified G-NH₂ after 24 hours of incubation. All of the fetal bovine sera tested showed significant conversion of G-NH₂ to modified G-NH₂ after 6 hours (6-10%) and 24 hours (18-32%) of incubation. The results confirmed that fetal bovine sera contained the cofactor(s) that significantly metabolizes G-NH₂ to modified G-NH₂ but human serum virtually does not.

Next, an evaluation of sera obtained from other animals was analyzed for their ability to convert G-NH₂ to modified G-NH₂. Serum obtained from pigs (PS), mice (MS), dogs (CS), cats (FS), horse (ES), and monkey (SS) was incubated with HPLC purified G-NH₂ and at 15 minutes, 1 hour, 6 hours, and/or 24 hours an aliquot of the mixture was removed and analyzed by cation exchange HPLC, as described above. Approximately a 10% dilution of serum in PBS was used. As shown in FIG. 8, the sera obtained from pigs, dogs, cats, horse, and monkeys rapidly converted G-NH₂ to modified G-NH₂, whereas, the mouse serum poorly metabolized G-NH₂. The data showed that although several animals were able to metabolize G-NH₂ to modified G-NH₂, the ability of the cofactor(s) to metabolize G-NH₂ was not evolutionarily conserved in humans and mice.

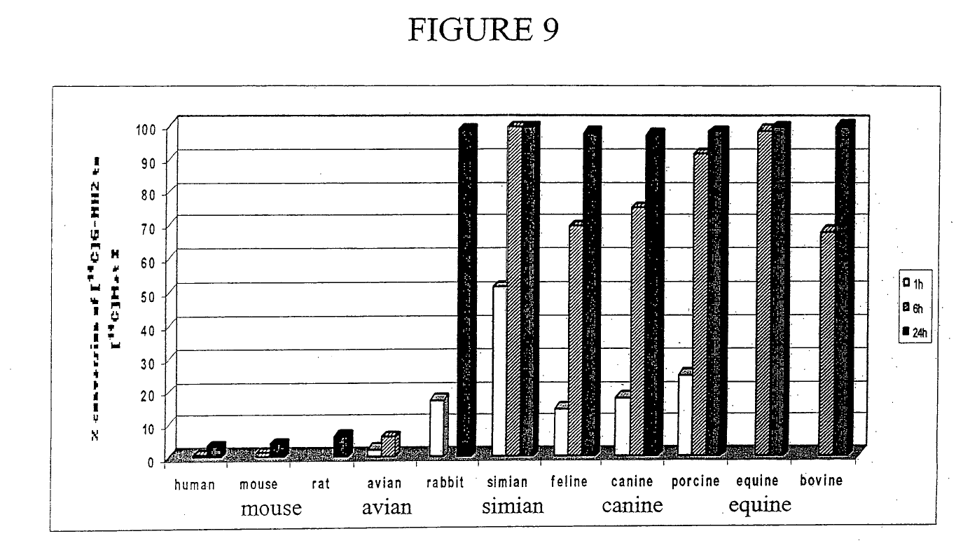

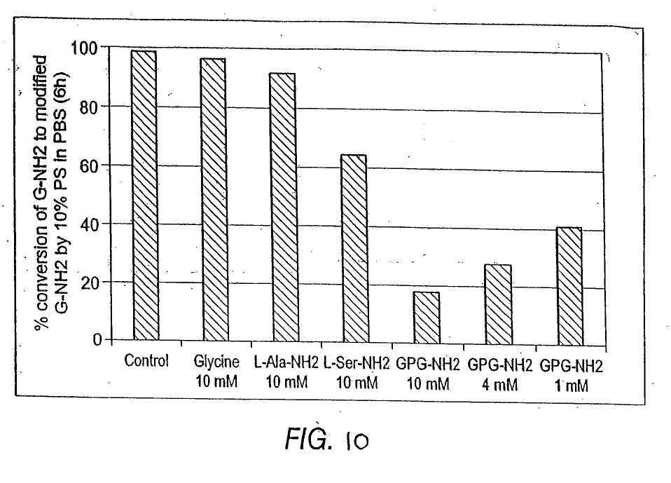

This is further illustrated in FIG. 9, which shows the results of another assay to evaluate the ability of different sera to convert radiolabeled G-NH₂ to Metabolite X. The conversion of ¹⁴C glycinamide to ¹⁴C metabolite X was measured as follows. Approximately, 0.1 μCi [¹⁴C]G-NH₂ (1.8 mmole) is added to a reaction mixture containing 10% serum in phosphate-buffered saline (PBS) (pH 7.5) (total volume: 100 μl). The reaction was carried out for different time periods. Sera, used in the study were from human, murine (mouse and rat), avian (chicken), rabbit, simian, feline, canine, porcine, equine or bovine (cow) origin. At the completion of the time period for each reaction, approximately 200 μl cold methanol was added to stop the reaction and the samples were placed on ice for 10 minutes. Subsequently, the samples were subjected to centrifugation (5 min, 15000 rpm). The supernatants were then injected on a Cation Exchange Column (Whatman) (Partisphere-SCX) and G-NH₂ and Metabolite X were separated on a gradient of ammonium phosphate buffer 5 mM pH 3.5 (Buffer A) and ammonium phosphate buffer 0.300 M pH 3.5 (Buffer B) [10 min A+6 min to 83% B+17% H₂O) (C)+2 min C-+6 min to A)]. Under these conditions, G-NH₂ eluted at approximately 8-9 minutes and Metabolite X eluted at approximately 2 minutes.

The data showed that G-NH₂ was readily converted to Metabolite X in rabbit, simian, feline, canine, porcine, equine and bovine sera. Human, mouse, rat, and avian sera, however, did not effectively catalyze G-NH₂ to modified glycinamide.

### Example 5

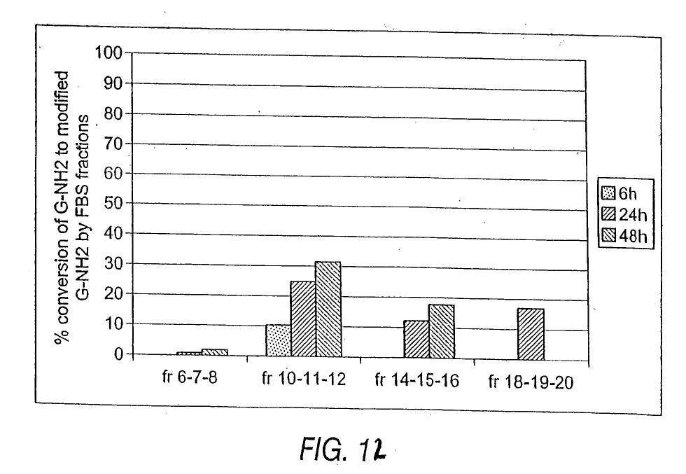

In a first set of experiments designed to purify the bovine serum cofactor(s) that converts G-NH₂ to modified G-NH₂, size exclusion chromatography (Superdex 200) was employed to separate the fetal bovine serum components. The separation was for 60 minutes in milli Q water and 30 fractions (0.5 ml/min) were collected. The presence of cofactor(s) in the various fractions was ascertained by incubating an aliquot of the isolated fraction with HPLC purified G-NH₂ followed by an analysis of the presence or absence of modified G-NH₂, as determined by cation exchange HPLC. As shown in FIG. 12, the majority of the cofactor eluted from the size exclusion column in fractions 10-12. Fractions 10-12 were found to efficiently convert G-NH₂ to modified G-NH₂, as determined by monitoring the accumulation of modified G-NH₂ by HPLC cation exchange chromatography, as described previously. Fractions 10-12 were also found to restore the anti-HIV activity of G-NH₂ in heated serum. The activity detected in later fractions may be a result of partially degraded co-factor cofactor that non-specifically interacted with the resin employed. This data confirmed that the cofactor that converts G-NH₂ to modified G-NH₂ had been purified. The cofactor can now be sequenced and cloned using conventional techniques.

### Example 6

To purify the cofactor from bovine serum, approximately 100 ml of bovine serum was precipitated with 16.4 gram (NH₄)₂SO₄ (30% saturation). After centrifugation (30 min; 20,000 rpm; 4° C.), the supernatant was further exposed to an additional 8.6 gram (NH₄)₂SO₄ (45% saturation) and centrifuged again. To this supernatant an additional 9.0 gram (NH₄)₂SO₄ (saturation: 60%) was added. The 30→45% precipitate was then solubilized in 25 ml potassium phosphate (50 mM) buffer pH 8.0 and dialysed overnight at 4° C. against 1 liter of the same buffer. From this dialysed enzyme fraction, 1 ml was separated on a DEAE Sepharose CL-6B column and chromatographed using a linear gradient from 50 mM buffer pH 8.0 (Buffer A) to 50 mM buffer pH 8.0+KCl 0.5 M (Buffer B). One-ml fractions were collected, and the enzyme activity was determined by measuring the luminescence that appeared in the reaction mixtures after 6 hrs. Each reaction mixture contained 0.5 ml enzyme fraction, 50 μl luminol (50 mM), 100 μl horseradish peroxidase (0.1 mg/ml) and potassium phosphate buffer 50 mM pH 8.0 to a total volume of 10 ml. The G-NH₂ conversion to Metabolite X can also be monitored by separation through cation exchange chromatography and by luminescence formation pointing to the release of H₂O₂ during the reaction process.

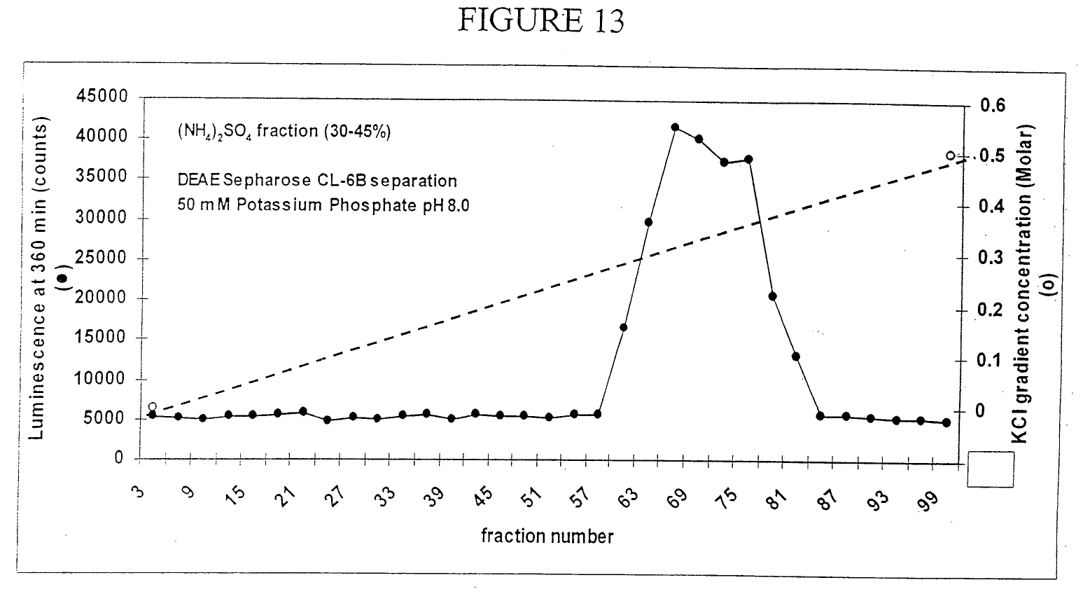

As shown in FIG. 13, the bovine serum cofactor began eluting from the column at approximately 30% Buffer B (approx. 0.15M KCl, fraction 57) and the modified glycinamide completely eluted from the column at 40% Buffer B (approx. 0.2M KCl, fraction 87). The broad peak may be the result of different conformations of the enzyme (e.g., oxygen or ligand bound or unbound enzyme).

The purified bovine sera cofactor was then analyzed for the ability to convert G-NH₂ to Metabolite X. Radiolabeled glycinamide (¹⁴C) was reacted with either crude bovine serum, the (NH₄)₂SO₄ fraction, or the purified bovine sera cofactor and the radiolabeled products of the reaction (modified glycinamide) were detected by column chromatography, as described above. See Examples 3 and 4. The amount of conversion of glycinamide to modified glycinamide per microgram of protein was calculated and this value for each step of the purification is provided in TABLE 9.

TABLE 9
Enzyme Purification from Bovine Serum

| | [¹⁴C] conversion units/ | |
|---|---|---|
| | μg protein | fold purification |
| Bovine Serum | 0.027 | 1 |
| (NH₄)₂SO₄ Precipitate | 0.38 | 14 |
| (30 → 45%) | | |
| DEAE-Seph. CL-6B | 1.96 | 73 |
| Sephadex G-50 | . . . | . . . |

As the data show, a 73-fold purification of the purified bovine sera cofactor was obtained. Accordingly, it was determined that the bovine sera cofactor is a heat labile oxido-reduction catalyst with 1.96 conversion units/μg protein, wherein one conversion unit completely converts 1.7 nmole glycinamide to modified glycinamide in 60 minutes. Thus, these experiments demonstrated that a purified bovine serum cofactor is a oxido-reduction catalyst with an activity that can be characterized as possessing the ability to efficiently convert glycinamide (e.g., 1.96 conversion units/μg protein in one hour) to a modified glycinamide that inhibits HIV replication.

### Example 7

This example describes the preparation of a G-NH₂-converting enzyme fused to a glutathione-S-transferase (GST) protein. This fusion protein can be expressed in bacteria and the enzyme can be rapidly purified from a Glutathione Sepharose 4B column. Accordingly, once the gene encoding the G-NH₂-converting enzyme is isolated from a genomic or cDNA library, it will be subcloned into the pGEM-T vector (Promega, Madison, Wis.) using a forward primer (AAG AAT TCT TTC TCG CAC AAG AAA TTA TTC G (SEQ. ID. NO. 20) and a reverse primer (AAG TCG ACT TAT TCG CTG ATA CGG CG (SEQ. ID. NO. 21) (Gibco, Paisley, U.K.), which introduce an EcoRI and SalI site, respectively, and the constructs will be transferred to E. Coli K12. The gene will then again be subcloned between the EcoRI and SalI sites of the pGEX-5X-1 vector (Amersham Pharmacia Biotech, Uppsala, Sweden). The resulting plasmid vector (pGEX-5X-1-TP) will be checked by automated fluorescence sequencing (ALFexpress, Amersham Pharmacia Biotech) and transfected into E. Coli BL21 (DE3)pLysS. Bacteria will be grown overnight at 37° C. in 2YT medium containing ampicillin (100 μg/ml) and chloramphenicol (40 μg/ml), and then diluted 1:10 in fresh medium. After further growth of the baceria at 27° C. (for 1 hr), isopropyl-β-D-thiogalactopyranoside (IPTG, Sigma) will be added to a final concentration of 0.1 mM to induce the production of the GST-TP fusion protein. After 15 hrs of further growth at 27° C., cells will be pelleted (6,000×g for 10 min at 4° C.) and resuspended in lysis buffer [50 mM Tris, pH 7.5, 1 mM DTT, 5 mM EDTA, 10% glycerol, 1% Triton X-100, 0.1 mM phenylmethylsulfonylfluoride (PMSF) and 0.15 mg/ml lysosyrne]. Bacterial suspensions will be homogenized and lysed by means of a “French Pressure cell press”, and ultracentrifuged (20,000×g for 15 min at 4° C.). GST-fusion protein will be purified from the supernatant using Glutathione Sepharose 4B (Amersham Pharmacia Biotech) as described by the Supplier. Briefly, a 50% slurry of Glutathione Sepharose will be added to the bacterial supernatant (1.5 ml/10.51 of broth), incubated for 30 min at 4° C., and then washed 3 times with 10 bed volumes (7.5 ml) of lysis buffer without lysosyme and PMSF. Bound proteins will be eluted in 50 mM Tris (pH 8.0) containing 0.1% Triton X-100 and 10 mM glutathione. Protein content of the purified fractions will be assessed using Bradford reagent (Sigma Chemical Co.). By using the approach described above, one will obtain purified, bacterially expressed, recombinant G-NH₂-converting enzyme, which can be used to convert glycinamide to α-hydroxyglycinamide.

### Example 8

An extract from Phaseolus vulgaris root nodule was prepared as follows: approximately 20 g of root nodules of 3-4 week old kidney beans (Phaseolus vulgaris) were removed, washed with potassium phosphate buffer 50 mM pH 8.0 and homogenized with 30 ml buffer. Then, the homogenate was sonicated (3×20 sec) on ice, and centrifuged for 15 min, 4° C. at 3,000 rpm. The supernatant was then centrifuged at 15,000 rpm during 20 min at 4° C., aliquoted and stored at −80° C. before use. To the reaction mixture (50 mM potassium phosphate buffer pH 8.0) was added: 0.1 μCi [¹⁴C]G-NH₂ and 50 μl extract supernatant (100%, 50% and 20%) and 50 μl buffer. The reaction proceeded overnight (˜18 hrs) at 37° C. Then, methanol was added (200 μl) to stop the reaction and the reaction mixture was subjected to cation exchange column chromatography, as described above. A reaction mixture containing bovine serum was also evaluated as a control.

The results showed that the root nodule extract, which contains leghemoglobin (MW16,900), efficiently converts glycinamide to modified glycinamide at a level that was ATGGGCGCCTTCACCGAGAAGCAGGAGGCCCTGGTGAACAGCAGCTGGCCTTCAAGGGCAACAT CCCCCAGTACAGCGTGGTGTTCTACACCAGCACCGGGACCACTTGTCGTCGACCCTCCGGAAGTT CCCGTTGTAGGGGGTTCGCACCACAAGATGTGGTCGTGGAGAAGGCCCCCGCCGCCAAGAACCT GTTCAGCTTCCTGGCCAACGGGACCCCACCAACCCCAAGCTGACCGCCCACGCCGAGAGCCTGTT AGGACCTCTTCCGGGGGCGGCGGTTCACAAGTCGAAGGACCGGTTGCCGCACCTGGGGTGGTTG GGGTTCGACTGGTGCGGCTCTCGGACAACGGCTGCGCGACAGCGCCGCCCAGCTGCGCGCCAAC GGCGCCGTGGTGGCCGCGCCCTGGGCAGCATCCACAGCCAGAAGGCCCTGAACGACGCCGGACC ACGCGCTGTCGCGGCGGGTCGACGGTTGCCGCGGCACCACCGGCTGCGGCGGGACCCGTCGTAG GTGTCGTCCGGGACTTGCTGAGCCAGTTGTGGTGAAGGAGGCCCTGCTGAAGACCCTGAAGGAG GCCGTGGGCGACGGACCGACGAGCTGAGCACCGCCCTGGAGCTGGCCTTCGGTCAAGGACCACC ACTTCCTCCGGGACGACTGGACTTCCTCCGGCACCCGCTGTTCACCTGGCTGCTCGACTCGTGGC GCTCGACCGGATGCTGCTCAAGCGGCGGCCGTAGTTCTTCCGGATGCGG (SEQ.ID. NO. 3).

The DNABuilder software assists in the design of codon-optimized DNA and generates oligonucleotides that can be assembled to generate the full-length gene. That is, DNA Builder takes either a DNA or protein sequence and redesigns it to contain the optimal codons for translation in humans and allows for the creation or deletion restriction sites to facilitate cloning. By this approach, overlapping oligonucleotides are created from the designed DNA sequence. The program automatically checks the set of oligonucleotides for any undesired homology (e.g., hairpins, etc). Assembly problems predicted by the program are remedied automatically by reiteratively substituting alternate codons until an acceptable design is found. The final output is a list of oligonucleotides sequences that can be submitted to a DNA synthesizer. See TABLE 10.

TABLE 10

| | | |
|---|---|---|
| | | Seq. ID. |
| Name | Oligonucleotide sequence | No. |

| | | |
|---|---|---|
| Leghemoglobin | ATGGGCGCCTTCACCGAGAAGCAGGAGGCCCTGGTGAACAGCAG | 4 |
| O04939-1: | CTGG | |
| Leghemoglobin | TGGGGGATGTTGCCCTTGAAGGCCTCCCAGCTGCTGTTCACCAG | 5 |
| O04939-2 | GGCC | |
| Leghemoglobin | CCTTCAAGGGCAACATCCCCCAGTACAGCGTGGTGTTCTACACC | 6 |
| O04939-3 | AGCA | |
| Leghemoglobin | CTTGGCGGGGGGGGCCTTCTCCAGGATGCTGGTGTAGAACACCA | 7 |
| O04939-4 | CGCT | |
| Leghemoglobin | GGAGAAGGCCCCGCCGCCAAGAACCTGTTCAGCTTCCTGGCCA | 8 |
| O04939-5 | ACGG | |
| Leghemoglobin | TCAGCTTGGGGTTGGTGGGGTCCACGCCGTTGGCCAGGAAGCTG | 9 |
| O04939-6 | AACA | |
| Leghemoglobin | GACCCCACCAACCCCAAGCTGACCGCCCACGCCGAGAGCCTGTT | 10 |
| O04939-7 | CGGC | |
| Leghemoglobin | AGCTGGGCGGCGCTGTCGCGCACCAGGCCGAACAGGCTCTCGGC | 11 |
| O04939-8 | GTGG | |
| Leghemoglobin | TGCGCGACAGCGCCGCCCAGCTGCGCGCCAACGGCGCCGTGGTG | 12 |
| O04939-9 | GCCG | |
| Leghemoglobin | GCTGTGGATGCTGCCCAGGGCGGCGTCGGCCACCACGGCGCCGT | 13 |
| O04939-10 | TGGC | |
| Leghemoglobin | CGCCCTGGGCAGCATCCACAGCCAGAAGGCCCTGAACGACAGCC | 14 |
| O04939-11 | AGTT | |
| Leghemoglobin | TCAGCAGGGCCTCCTTCACCACCAGGAACTGGCTGTGGTTCAGG | 15 |
| O04939-12 | GCCT | |
| Leghemoglobin | GTGGTGAAGGAGGCCCTGCTGAAGACCCTGAAGGAGGCCGTGG | 16 |
| O04939-13 | GCGAC | |
| Leghemoglobin | GGGGTGCTCAGCTCGTCGGTCCACTTGTCGCCCACGGCCTCCTTC | 17 |
| O04939-14 | AGG | |
| Leghemoglobin | GGACCGACGAGCTGAGCACCGCCCTGGAGCTGGCCTACGACGAG | 18 |
| O04939-15 | TTCG | |
| Leghemoglobin | GGCGTAGGCCTTCTTGATGCCGGCGGCGAACTCGTCGTAGGCCA | 19 |
| O04939-16 | GCTC | |

These overlapping oligonucleotides are then synthesized and used to assemble the full-length codon optimized gene.

Although the codon-optimized leghemoglobin protein sequence was found to be identical to the native Phaseolus vulgaris leghemoglobin protein provided in SEQ. ID NO. 2, Discontiguous MegaBlast revealed that the nucleic acid sequence shared homology to the native Phaseolus vulgaris DNA sequence over only two domains: a first domain at approximately nucleotide residues 1-100 (approx. 83% identity) and a second domain at approximately nucleotide residues 312-431 (approx. 75% identity).

The codon-optimized leghemoglobin gene can be inserted into virtually any vector, however, it is preferably inserted into an episomal expression vector so as to allow for high level of expression in a human subject with little or no interference with the genome. (See Van Craenenbroeck, et al., European Journal of Biochemistry, 267(18)5665 (2000)), herein expressly incorporated by reference in its entirety. Recombinant, replication deficient adenoviral vectors (AdV) and Adenovirus expression systems that reproducibly allow for high level protein expression in human cells are commercially available from companies such as Clonetech and Qbiogene. Expression vectors containing the codon-optimized leghemoglobin gene can then be transferred to subjects and the ability to convert glycinamide to antiretroviral modified glycinamide (e.g., alpha hydroxyglycinamide) can be evaluated using one of the protocols described herein.

For example, mice, which cannot convert glycinamide to an antiretroviral compound (see FIG. 8), are provided an expression vector that comprises a codon-optimized leghemoglobin gene. Positive transformants can be identified by PCR, transcription of the construct can be verified by RT PCR, and protein expression can be analyzed by monitoring the conversion of glycinamide to modified glycinamide. By one approach, mice are provided various amounts of ¹⁴C glycinamide and the conversion to modified glycinamide (e.g., α-hydroxyglycinamide) is determined by removing blood from the animals, isolating the serum and monitoring the presence or absence of radiolabeled modified glycinamide by thin-layer chromatography or the chromatographic approach described in EXAMPLES 3 and 4. Additionally, serum from the mice can be obtained and the conversion of glycinamide to modified glycinamide in the presence of the mouse serum can be determined in vitro, as described in EXAMPLE 4.

HIV infectivity studies in the presence of glycinamide that had been incubated with mouse serum obtained from a mouse that was transiently infected with a protein expression vector comprising a codon-optimized leghemoglobin gene can be compared to an HIV infectivity study in the presence of glycinamide that had been incubated with mouse serum obtained from a mouse that had not been introduced to the expression plasmid and it will be determined that the mice that received the codon-optimized leghemoglobin gene gain the ability to metabolize glycinamide to a modified glycinamide that has an antiretroviral activity. An experimental protocol similar to that employed in EXAMPLES 10 and 11 can be employed.

Similar experiments can be performed in primates that are infected with SIV and it will be determined that SIV-infected primates that receive the protein expression vectors that comprise the codon-optimized leghemoglobin gene will experience a reduction in viral load or another marker (e.g., accumulation of p24 or reverse transcriptase activity) that indicates an inhibition of viral replication.

### Example 10

Modified glycinamide was enzymatically produced, isolated, and analysed for its ability to inhibit the replication of HIV. Dialysis tubing (3500 kD molecular weight cut-off) was shaken in distilled water with PEST buffer (RPMI with streptomycin and penicillin) for 30 min at room temperature followed by shaking in 2% sodium bicarbonate and 1 mM EDTA for 30 min at 60° C. The tubing was rinsed two times in distilled water with PEST. After that, the tubing was boiled in distilled water with PEST for 5 min. After boiling, the tubing was transferred to a beaker filled with PBS+PEST, and stored at +4° C. until used.

The tubing was used 20 days after boiling. On a sterile bench, the dialysis tubing was washed with sterile and deionised water. Approximately, 10 ml of porcine serum (Promeda corp.) was added to the tubing. The tubing was put in a glass beaker filled with 200 ml PBS-A/PEST (1 ml PEST+1L PBS-A). The beaker was taken out of the sterile bench and placed on an orbital shaker. After 1 h, the PBS-A/PEST was replaced with 200 ml fresh PBS-A=“pre-wash”. The tubing was pre-washed five times with five portions of PBS-A for 1 h as described above. After the pre-wash, the dialysis tubing containing serum, was transferred to a sterile glass bottle filled with 100 ml of sterile filtrated 1 mM glycinamide (Bachem) and a magnetic stirring bar. The bottle containing the glycinamide and serum was incubated on a magnetic stirring plate at 37° C. After approximately 48 h, the dialysis was stopped, the dialysis solution was divided into three portions (10 ml+38 ml+50 ml) and was transferred to labelled glass bottles, which were sealed and frozen at −85° C. A portion of the frozen dialysis solution was then freeze dried.

The freeze-drying system (Vacuum oil (Heto 88900100), Milli-Q water, water purification equipment, Freeze-dryer, and −85° C. freezer) were prepared. Frozen dialysis solution (the 38 ml portion from 1-1) was transferred from the −85° C. freezer to the freeze-drying chamber. The lid was placed over the chamber and the vacuum was turned on. The freeze-drying process was stopped after approximately 72 h. The vacuum was turned off and the glass bottle was removed from the freeze-drying chamber.

Next, freeze-dried product was purified by HPLC. Approximately, 2L of 0.1M KH₂PO₄ (Merck no. 14873-250/Lot: A397373251) was prepared by weighing 27.22 g KH₂PO₄ and dissolving it in 2 L water (pH4.06). The column (Hypersil SCX ion-exchange column 5 um/250×10 mm (ThermoQuest 3-34087/Batch: 5/100/5580) and HPLC-system including software D-7000 HSM) was equilibrated with mobile phase (90% 0.1M KH₂PO₄/10% acetonitrile (Scharlau AC0329/Batch:57048)) for 60 min at 5 ml/min. The UV-detector wavelength was set for 206 nm. The dried dialysis “sample” was dissolved in 2 ml water (19 mM glycine-amide was present at the start of dialysis) and was injected and analysed (RUN-1) with a 10 min isocratic run of mobile phase (see above) at 5 ml/min. The injection volume for RUN 1 was approximately 100 μl.

After calibration, 200 μl of sample was injected nine more times (RUN-2→10) and fractions eluting at 2.5-3.1 min were collected for each run using a TIME-mode collection set for 0.1 min/fraction. Between RUN-8 and 9, 1L 0.1M KH₂PO₄ was prepared by weighing 13.61 g KH₂PO₄ and dissolving it in 1 L water. The corresponding fractions collected in RUN-2→10, were pooled and were injected over the column (RUN-11→16). In RUN-11→16 each injection contained approximately 100 μl. The fractions were collected between 2.6-2.8 min and were pooled. Approximately, 1.25 mg of modified glycinamide (Metabolite X) was obtained, as determined from the amount of original glycinamide and the area of the collected peaks. The pooled 2.6-2.8 min fractions in 7.5 ml of mobile phase (90% 0.1M KH₂PO₄/10% acetonitrile) were transferred to a labelled glass bottle that was sealed and frozen at −85° C. Additionally, 7.5 ml mobile phase was frozen at −85° C. as a salt control. HPLC-analysis revealed that all detectable glycinamide (retention time ˜5.9 min) had been converted to modified glycinamide (˜2.7 min). After analysis/purification, the column was washed with 40% acetonitrile/water for 31 min at 5 ml/min and the enzymatically prepared modified glycinamide (“Metabolite X”) was freeze-dried using the approach described above.

An HIV infectivity assay was then performed with the enzymatically prepared modified glycinamide (MetX). The lyophilised MetX (1.25 mg) was dissolved in 7.5 ml sterile distilled water (2.24 mM MetX). Approximately, 3.7 ml of 2.24 mM MetX was mixed with 4.8 ml each of normal and boiled RPMI++ (RPMI-medium with 10% FCS and 0.1% PEST). That is, two lots of 8.5 ml of 1 mM MetX were prepared. Then, approximately 3 ml 1 mM MetX was mixed with 3 ml each of normal and boiled RPMI++ (i.e., 2×6 ml of 500 μM MetX). Approximately, 1 ml 500 μM MetX was then mixed with 4 ml each of normal and boiled RPMI++ yielding 2×5 ml of 100 μM MetX. The lyophilised salt control was dissolved and diluted exactly the same as MetX, above. A 1 mM stock solution of unmodified glycinamide was also used to prepare 100 μM glycinamide in normal and boiled RPMI++ (controls) as described for MetX, as well.

H9 cells were counted in three A-squares of a Burke chamber (a mean of 1.2×10⁶ cells/ml, which is 4×10⁶ cells in 3.3 ml). Approximately, 4×10⁶ cells (3.3 ml) were added to two 50 ml tubes. Next, approximately 14.7 ml of normal RPMI++was added to the first tube and approximately 14.7 ml boiled RPMI++was added to the second tube (i.e., 18 ml H9 cells+ normal/boiled RPMI++). Then approximately 2 ml of virus stock (SF2+H9, day 9: 22/3-02 2) was added to each 50 ml tube containing the cells and medium, about 20 ml/tube, and the solutions were mixed. The two virus/cell mixtures were split into two new 50 ml tubes (i.e., four tubes with 10 ml of cell/virus (two tubes with normal RPMI++ and two with boiled RPMI++)). The cell/virus tubes were incubated at 37° C. for 90 min with mixing after 50 min. The infection was stopped by collecting the cells (5 min at 1200 rpm). The cells were then resuspended and transferred to 12 10 ml tubes (0.5×10⁶ cells/tube). That is, six tubes of cells suspended in normal RPMI++ and six tubes of cells suspended in boiled RPMI++. The cells were washed with RPMI (without additives) and collected (5 min at 1500 rpm). The supernatants were discarded and the cells were resuspended in 4.5 ml each of:

Normal RPMI++

Boiled RPMI++

100 μM glycine-amide in normal RPMI++

100 μM glycine-amide in boiled RPMI++

500 μM MetX in normal RPMI++

500 μM MetX in boiled RPMI++

100 μM MetX in normal RPMI++

100 μM MetX in boiled RPMI++

500 μM salt in normal RPMI++

500 μM salt in boiled RPMI++

100 μM salt in normal RPMI++

100 μM salt in boiled RPMI++

Approximately, 0.9 ml/well of each cell suspension (four replicates of each) was added to a 48-well plate as follows:

PLATE-1:

4 wells with 100 μM glycine-amide in normal RPMI++

4 wells with 100 μM glycine-amide in boiled RPMI++

4 wells with 500 μM MetX in normal RPMI++

4 wells with 500 μM MetX in boiled RPMI++

4 wells with 100 μM MetX in normal RPMI++

4 wells with 100 μM MetX in boiled RPMI++

PLATE-2:

4 wells untreated normal RPMI++

4 wells untreated boiled RPMI++

4 wells “100 μM” salt in normal RPMI++

4 wells “100 μM” salt in boiled RPMI++

4 wells “500 μM” salt in normal RPMI++

4 wells “500 μM” salt in boiled RPMI++

The remaining wells were filled with sterile distilled water. The cell culture plates were incubated at 37° C. and 5% CO₂. After four days the medium was changed, after eight days the medium was changed and the cells were collected. After 11 days, the infection was stopped, the cells were viewed in a 10× magnification microscope and 650 μl of each cell supernatant was collected and frozen at −80° C. for further analysis. After five more days, the supernatants were thawed and used in a conventional reverse transcriptase (RT) activity assay (e.g., Roche AMPLICOR MONITOR™) or a p24 quantification assay (e.g., Abbott Laboratories, Chicago). (See U.S. Pat. No. 6,258,932 and U.S. patent application Ser. No. 10/235,158, both of which are hereby expressly incorporated by reference in its entireties). The results are shown in FIG. 16 and TABLE 11.

TABLE 11

| Sample | Visible syncytia |
|---|---|
| 100 μM MetX in normal RPMI++ | negative |
| 100 μM MetX in boiled RPMI++ | negative |
| 500 μM MetX in normal RPMI++ | negative |
| 500 μM MetX in boiled RPMI++ | negative |
| 100 μM glycinamide in normal RPMI++ control | negative |
| 100 μM glycinamide in boiled RPMI++ control | positive |
| Untreated normal RPMI control | positive |
| Untreated boiled RPMI control | positive |
| 100 μM salt control in normal RPMI++ | positive |
| 100 μM salt control in boiled RPMI++ | positive |
| 500 μM salt control in normal RPMI++ | negative |
| 500 μM″ salt in boiled RPMI++ | negative |

By visual inspection, modified glycinamide (Metabolite X) effectively inhibited replication and/or propagation of HIV in the boiled fetal calf serum but glycinamide did not (TABLE 11). The reverse transcriptase (RT) activity data (FIG. 17) confirmed that modified glycinamide (Met-X or Metabolite X) effectively inhibited replication HIV in the boiled fetal calf serum sample even though G-NH₂ was unable to inhibit replication of HIV under these conditions. That is, the antiviral activity of modified glycinamide (MetX) does not require a cofactor(s) that is present in fetal calf serum but glycinamide does. This data also indicates that the heating of the fetal calf serum denaturated the enzyme (cofactor(s)) that converts glycinamide to modified glycinamide.

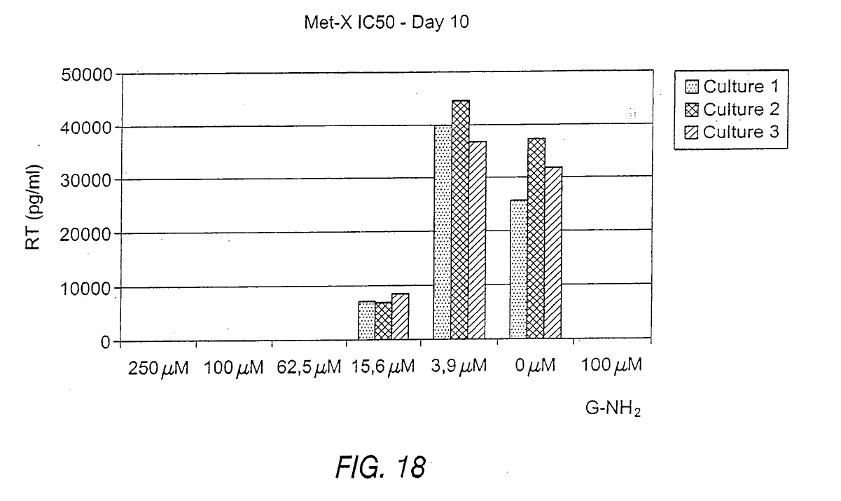

In another set of related experiments, the antiretroviral activity of Metabolite X that had been dialysed five times was compared to Metabolite X prepared by the approach above. In brief, HIV infectivity assays were performed with G-NH₂ in fetal calf serum, as above, with the five-times dialysed Metabolite X and the Metabolite X prepared by the approach above. The results of these experiments are shown in FIG. 18. A significant change in the activity of the five-time dialysed alpha-hydroxyglycinamide (Metabolite X), as compared to the standard preparation of the enzymatically produced alpha-hydroxyglycinamide (Metabolite X) was not observed. Accordingly, isolated bovine serum cofactor possessed similar antiretroviral activity as crude bovine serum cofactor.

The modified glycinamide obtained according to the enzymatic approach described above has been analysed by mass spectroscopy and NMR and the structure analysis revealed alpha-hydroxy glycinamide (“AlphaHGA”). Thus, the experiments in this example have shown that modified glycinamide (alpha-hydroxy glycinamide or Metabolite X) effectively inhibits the replication of HIV in the absence of the cofactor(s) present in fetal calf serum that is required for the antiretroviral activity of G-NH₂. Alpha hydroxy glycinamide (“AlphaHGA”) has also been prepared synthetically and was found to inhibit HIV replication in the absence of the cofactor(s), as described infra.

### Example 11

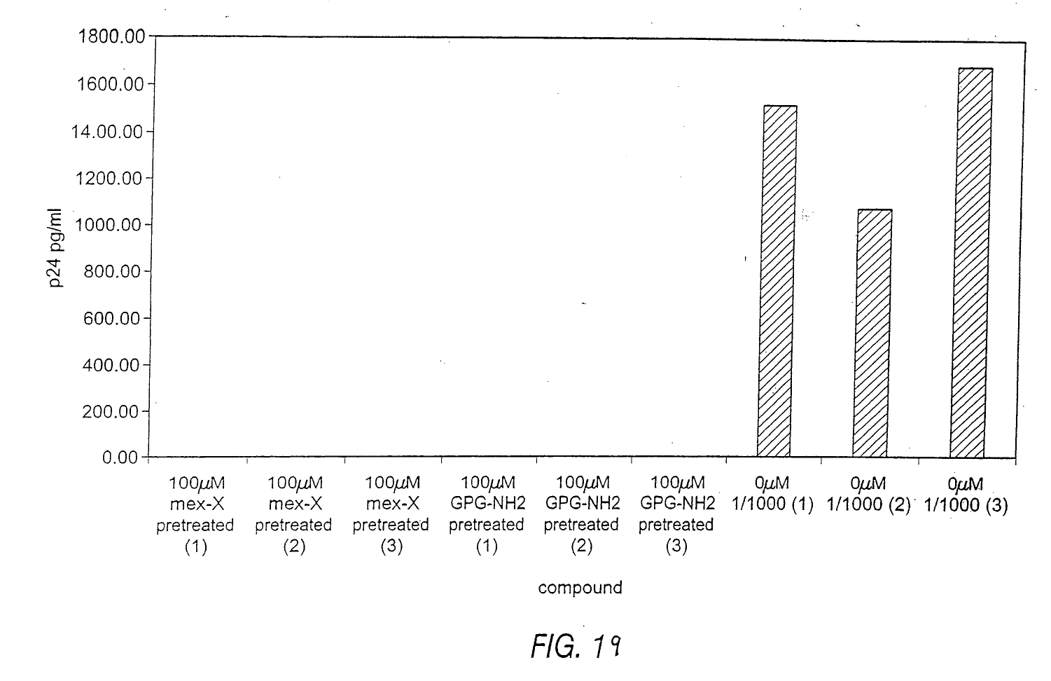

Approximately, 0.1×10⁶ H9 cells were infected with 50 TCID₅₀ HIV (SF2 virus) and the infected cells were mixed with enzymatically prepared Metabolite X (see EXAMPLE 10) at various concentrations. Fetal bovine serum was included in the assay. The cells were cultured for 10 days (fresh medium was added to the cultures day 7), after which the supernatants were collected and analyzed by a conventional reverse transcriptase (RT) quantification assay. The data is shown in FIG. 19. The results show that effective inhibition of HIV replication occurs at low concentrations of Metabolite X (e.g., between 3.9 μM-15.6 μM) and that when concentrations reach 15.6 μM or higher, the inhibition of HIV replication is virtually complete. EXAMPLE 12

By one approach, modified glycinamide (Metabolite X) was enzymatically prepared by the dialysis of purified G-NH₂ against pig serum (see EXAMPLE 10); the modified glycinamide was then used to treat HIV (SF2 virus) infected H9 cells, and the infected cells were sent for analysis by electron microscopy. In brief, dialysis tubing (3500 MW cut-off-Spectrum) was loaded with pig serum (Biomedia) and the pig serum was pre-dialyzed against RPMI 1640 buffer four times for one hour each to remove molecules that were less than 3500 daltons. The pre-washed serum was then dialysed against 1 mM purified G-NH₂ in RPMI 1640 at 37° C. for 48 hours. The dialysed buffer containing the modified G-NH₂ (Metabolite X) was then sterile filtered, aliquoted, and frozen, as described in EXAMPLE 10.

Next, a 100 μm Metabolite X or 100 μM GPG-NH₂ concentration was established in four bottles containing (each) approximately 0.5×10⁶H9 cells in 10 ml of RPMI (containing fetal calf serum). The cells in the samples were counted and then centrifuged. The cells were then resuspended in 10 ml of RPMI 1640 (containing fetal calf serum) and either 100 μm Metabolite X or 100 μM GPG-NH₂. Uninfected control and untreated control samples were also included in the experiment. The samples were then incubated overnight at 37° C. at 5% CO₂.

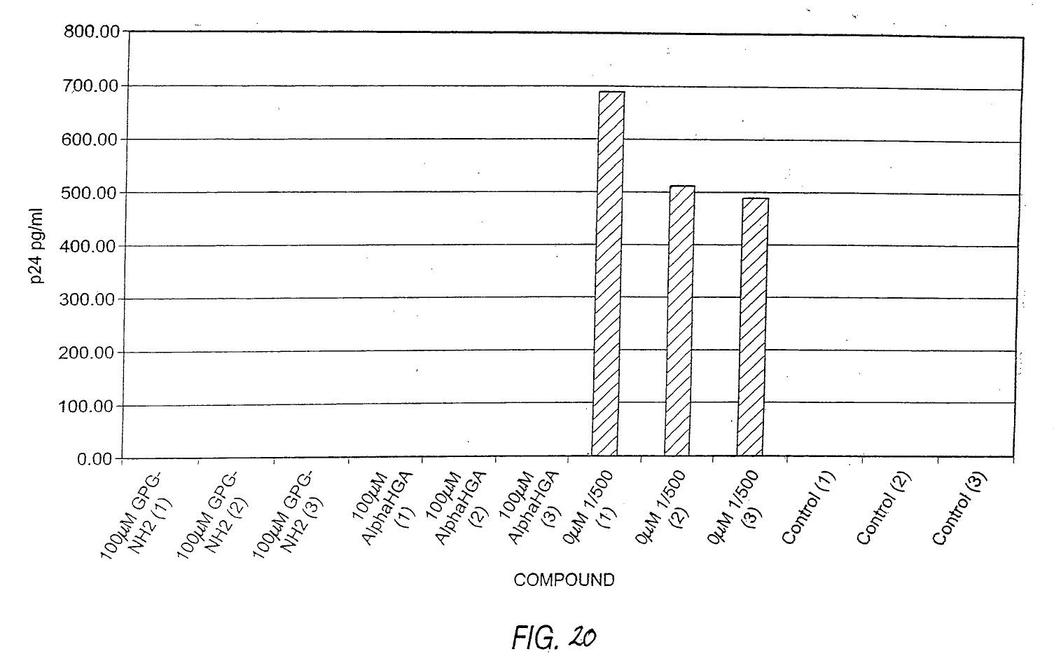

Then, the amount of p24 in the samples was analysed using a conventional p24 detection assay (see U.S. Pat. No. 6,258,932). As shown in FIG. 20, 100 μM modified glycinamide (Metabolite X) or 100 μM GPG-NH₂ effectively inhibited HIV replication in the presence of fetal calf serum; whereas, the untreated control samples showed appreciable HIV replication. These results were confirmed by a conventional reverse transcriptase (RT) activity assay, which showed appreciable amounts of reverse transcriptase activity in the untreated control samples but no reverse transcriptase activity in the samples treated with 100 μM modified glycinamide or 100 μM GPG-NH₂. Having verified that the samples treated with 100 μM modified glycinamide or 100 μM GPG-NH₂ contained virus that had been inhibited, the samples were sent to be analysed by electron microscopy.

By one approach, H9 cells that were infected by SF2 virus can be fixed in 2.5% glutaraldehyde by conventional means. The fixed cells are then postfixed in 1% OsO₄ and are dehydrated, embedded with epoxy resins, and the blocks are allowed to polymerize. Epon sections of virus infected cells are made approximately 60-80 nm thin in order to accommodate the width of the nucleocapsid. The sections are mounted to grids stained with 1.0% uranyl acetate and were analyzed in a Zeiss CEM 902 microscope at an accelerating voltage of 80 kV. The microscope is equipped with a spectrometer to improve image quality and a liquid nitrogen cooling trap is used to reduce beam damage. The grids having sections of control GPG-NH₂ incubated cells and metabolite X incubated cells are examined in several blind studies.

The electron microscopy of untreated HW particles will show the characteristic conical-shaped nucleocapsid and enclosed uniformly stained RNA that stretched the length of the nucleocapsid; whereas, the cells having HIV-1 particles that are treated with GPG-NH₂ or Metabolite X will show HIV-1 particles having conical-shaped capsid structures that appear to be relatively intact but the RNA was amassed in a ball-like configuration either outside the capsid or at the top (wide-end) of the capsid. Some capsids from the GPG-NH₂ or Metabolite X treated samples may be observed to have misshapen structures with little or no morphology resembling a normal nucleocapsid and the RNA may be either outside the structure or inside the structure at one end.

### Example 13

HIV infectivity assays were performed in the presence of fetal calf serum, as described in the preceding examples (see EXAMPLES 10-12), however, various concentrations of G-NH₂, GPG-NH₂, and enzymatically prepared modified glycinamide (Metabolite X), and 100 μM synthetically produced modified glycinamide (AlphaHGA) were used. (See TABLE 12). Three replicate samples (“replicates”) of uninfected samples and untreated samples were also included in the experiment as controls. The inhibition of HIV replication was monitored by quantifying the levels of p24 using a conventional detection kit.

TABLE 12

| Peptide | Conc. | | Samples |
|---|---|---|---|
| GPG-NH₂ | 100 | μM | 3 replicates at each |
| | 50 | μM | concentration |
| | 25 | μM | |
| | 12.5 | μM | |
| | 6.25 | μM | |
| | 3.1 | μM | |
| | 1.6 | μM | |
| | 0.8 | μM | |
| G-NH₂ | 100 | μM | 3 replicates at each |
| | 50 | μM | concentration |
| | 25 | μM | |
| | 12.5 | μM | |
| | 6.25 | μM | |
| | 3.1 | μM | |
| | 1.6 | μM | |
| | 0.8 | μM | |
| Met-X (enzymatically prepared by | 100 | μM | 3 replicates at each |
| dialysis) | 50 | μM | concentration |
| | 25 | μM | |
| | 12.5 | μM | |
| | 6.25 | μM | |
| | 3.1 | μM | |
| | 1.6 | μM | |
| | 0.8 | μM | |
| AlphaHGA (synthetically | 100 | μM | 3 replicates |
| produced by Chemilia) | | | |

FIG. 20 shows some of the results of these experiments. As shown, on day 11 of the experiment, the synthetically produced alpha-hydroxy glycinamide (AlphaHGA) inhibited HIV replication as effectively as GPG-NH₂ in fetal calf serum-containing media. Similar results were also observed at day 7. This data demonstrate that synthetically produced alpha-hydroxy glycinamide (AlphaHGA) effectively inhibits HIV replication.

### Example 14

HIV infectivity assays were performed in the presence of human serum or fetal calf serum, as described in the preceding examples (see EXAMPLES 10-12), however, various concentrations of G-NH₂, enzymatically prepared modified glycinamide (Metabolite X), and 100 μM synthetically produced modified glycinamide (AlphaHGA) were used. (See TABLES 13 and 14). Three replicates of unninfected samples and untreated samples were also included in the experiment as controls.

TABLE 13
Human serum

| Peptide | Conc. | Samples |
|---|---|---|
| G-NH₂ | 100 μM | 3 replicates at each |
| | 50 μM | concentration |
| Met-X (enzymatically prepared by | 100 μM | 3 replicates at each |
| dialysis) | 50 μM | concentration |
| Alpha HGA (synthetically | 50 μM | 3 replicates |
| prepared by Chemilia) | | |
| Uninfected control | 0 μM | 3 replicates |
| Infected control | 0 μM | 3 replicates |

TABLE 14
Fetal calf serum

| Peptide | Conc. | Samples |
|---|---|---|
| G-NH₂ | 100 μM | 3 replicates at each |
| | 50 μM | concentration |
| Met-X (enzymatically prepared by | 100 μM | 3 replicates at each |
| dialysis) | 50 μM | concentration |
| Alpha HGA (synthetically | 50 μM | 3 replicates |
| prepared by Chemilia) | | |
| Uninfected control | 0 μM | 3 replicates |
| Infected control | 0 μM | 3 replicates |

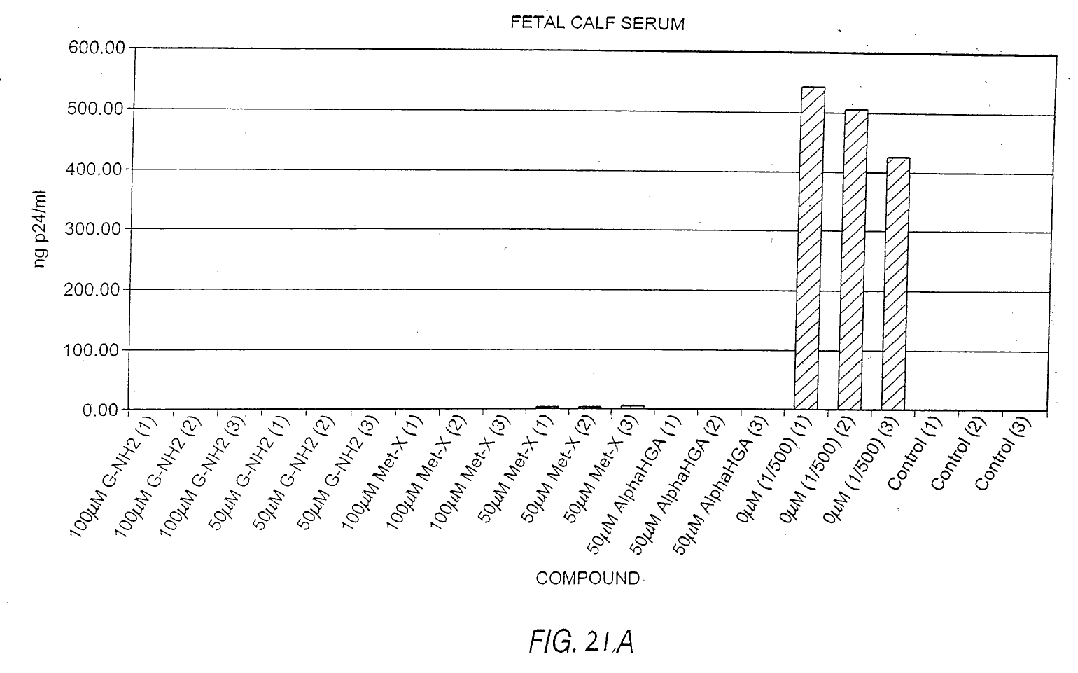

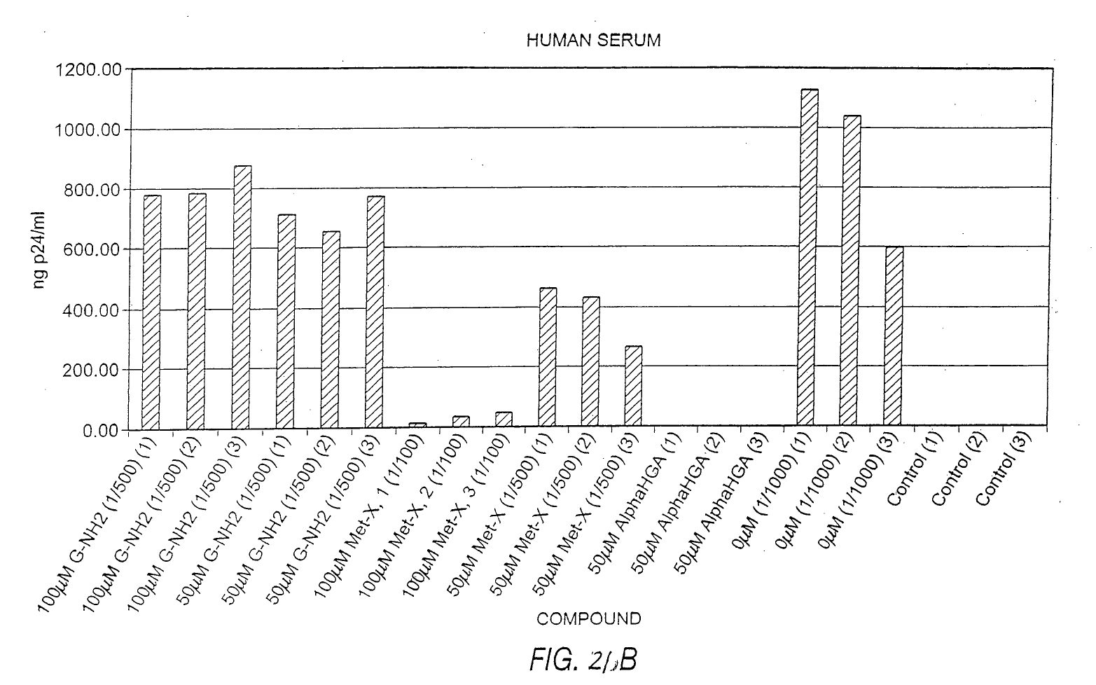

The results of these experiments are provided in TABLES 15 and 16 and in FIGS. 21A and 21B. The data show that on day 12, the enzymatically prepared modified glycinamide (Metabolite X), and the synthetically produced alpha-hydroxyglycinamide (AlphaHGA) inhibited HIV replication as effectively as G-NH₂ in fetal calf serum-containing media; however, only the enzymatically prepared modified glycinamide (Metabolite X), and synthetically produced alpha-hydroxy glycinamide (AlphaHGA) were able to inhibit HIV replication in human serum. That is, G-NH₂ was unable to inhibit HIV replication in human serum but both enzymatically prepared modified glycinamide (Metabolite X), and synthetically produced alpha hydroxy glycinamide (AlphaHGA) were effective inhibitors of HIV replication in human serum. Similar results were observed at day 7. These data provide strong evidence that both enzymatically prepared modified glycinamide (Metabolite X), and synthetically produced alpha hydroxy glycinamide (AlphaHGA) are potent inhibitors of HIV replication in infected humans.

TABLE 15
Fetal Calf serum

| | | | mean | mean OD - | conc p24 |
|---|---|---|---|---|---|
| | OD1 | OD2 | OD | blank | (ng/ml) |
| 100 μM G-NH2 (1) | 0.078 | 0.075 | 0.077 | 0.035 | 0.09 |
| 100 μM G-NH2 (2) | 0.071 | 0.069 | 0.070 | 0.028 | 0.08 |
| 100 μM G-NH2 (3) | 0.077 | 0.071 | 0.074 | 0.032 | 0.09 |
| 50 μM G-NH2 (1) | 0.319 | 0.335 | 0.327 | 0.285 | 0.49 |
| 50 μM G-NH2 (2) | 0.182 | 0.183 | 0.183 | 0.141 | 0.26 |
| 50 μM G-NH2 (3) | 0.105 | 0.103 | 0.104 | 0.062 | 0.14 |
| 100 μM Met-X (1) | 0.193 | 0.343 | 0.268 | 0.226 | 0.40 |
| 100 μM Met-X (2) | 0.081 | 0.107 | 0.094 | 0.052 | 0.12 |
| 100 μM Met-X (3) | 0.144 | 0.152 | 0.148 | 0.106 | 0.21 |
| 50 μM Met-X (1) | 1.105 | 1.089 | 1.097 | 1.055 | 1.71 |
| 50 μM Met-X (2) | 1.895 | 1.887 | 1.891 | 1.849 | 2.98 |
| 50 μM Met-X (3) | 2.351 | 2.230 | 2.291 | 2.249 | 3.61 |
| 50 μM AlphaHGA (1) | 0.183 | 0.185 | 0.184 | 0.142 | 0.26 |
| 50 μM AlphaHGA (2) | 0.232 | 0.216 | 0.224 | 0.182 | 0.33 |
| 50 μM AlphaHGA (3) | 0.147 | 0.139 | 0.143 | 0.101 | 0.20 |
| 0 μM (1/500) (1) | 0.691 | 0.717 | 0.704 | 0.662 | 544.90 |
| 0 μM (1/500) (2) | 0.673 | 0.637 | 0.655 | 0.613 | 505.98 |
| 0 μM (1/500) (3) | 0.544 | 0.568 | 0.556 | 0.514 | 427.33 |
| Control (1) | 0.042 | 0.039 | 0.041 | −0.001 | 0.04 |
| Control (2) | 0.042 | 0.037 | 0.040 | −0.002 | 0.03 |
| Control (3) | 0.046 | 0.045 | 0.046 | 0.004 | 0.04 |

TABLE 16
Human serum

| | | | | mean | |
|---|---|---|---|---|---|
| | | | mean | OD - | conc p24 |
| | OD1 | OD2 | OD | blank | (ng/ml) |
| 100 μM G-NH2 (1/500) (1) | 1.194 | 1.196 | 1.195 | 1.111 | 780.21 |
| 100 μM G-NH2 (1/500) (2) | 1.184 | 1.221 | 1.203 | 1.119 | 785.24 |
| 100 μM G-NH2 (1/500) (3) | 1.315 | 1.362 | 1.339 | 1.255 | 876.34 |
| 50 μM G-NH2 (1/500) (1) | 1.079 | 1.114 | 1.097 | 1.013 | 714.23 |
| 50 μM G-NH2 (1/500) (2) | 0.996 | 1.015 | 1.006 | 0.922 | 653.27 |
| 50 μM G-NH2 (1/500) (3) | 1.176 | 1.194 | 1.185 | 1.101 | 773.51 |
| 100 μM Met-X (1/100) (1) | 0.117 | 0.114 | 0.116 | 0.032 | 11.41 |
| 100 μM Met-X (1/100) (2) | 0.269 | 0.281 | 0.275 | 0.191 | 32.78 |
| 100 μM Met-X (1/100) (3) | 0.377 | 0.378 | 0.378 | 0.294 | 46.52 |
| 50 μM Met-X (1/500) (1) | 0.698 | 0.728 | 0.713 | 0.629 | 457.33 |
| 50 μM Met-X (1/500) (2) | 0.676 | 0.662 | 0.669 | 0.585 | 427.85 |
| 50 μM Met-X (1/500) (3) | 0.418 | 0.422 | 0.420 | 0.336 | 261.05 |
| 50 μM AlphaHGA (1) | 1.546 | 1.546 | 1.546 | 1.462 | 2.03 |
| 50 μM AlphaHGA (2) | 1.183 | 1.219 | 1.201 | 1.117 | 1.57 |
| 50 μM AlphaHGA (3) | 0.665 | 0.679 | 0.672 | 0.588 | 0.86 |
| 0 μM (1/1000) (1) | 0.887 | 0.857 | 0.872 | 0.788 | 1127.68 |
| 0 μM (1/1000) (2) | 0.827 | 0.791 | 0.809 | 0.725 | 1043.27 |
| 0 μM (1/1000) (3) | 0.472 | 0.472 | 0.472 | 0.388 | 591.77 |
| Control (1) | 0.095 | 0.089 | 0.092 | 0.008 | 0.08 |
| Control (2) | 0.091 | 0.089 | 0.090 | 0.006 | 0.08 |
| Control (3) | 0.081 | 0.089 | 0.085 | 0.001 | 0.07 |

### Example 15

HIV infectivity assays were performed in the presence of fetal calf serum, as described in the preceding examples (see EXAMPLES 10-12), however, various concentrations of G-NH₂, synthetically produced modified glycinamide (AlphaHGA), and synthetically produced modified glycinamide that had been incubated at 37° C. for three days were used (AlphaHGA 37). (See TABLE 17). Three replicates of unninfected samples and untreated samples were also included in the experiment as controls.

TABLE 17

| Peptide | Conc. | | Samples |
|---|---|---|---|
| αHGA | 32 | μM | 3 replicates at each |
| | 16 | μM | concentration |
| | 8 | μM | |
| | 4 | μM | |
| | 2 | μM | |
| | 1 | μM | |
| | 0.5 | μM | |
| αHGA 37 | 32 | μM | 3 replicates at each |
| (incubated at 37° C. | 16 | μM | concentration |
| for three days) | 8 | μM | |
| | 4 | μM | |
| | 2 | μM | |
| | 1 | μM | |
| | 0.5 | μM | |
| G-NH₂ | 32 | μM | 3 replicates at each |
| | 16 | μM | concentration |
| | 8 | μM | |
| | 4 | μM | |
| | 2 | μM | |
| | 1 | μM | |
| | 0.5 | μM | |

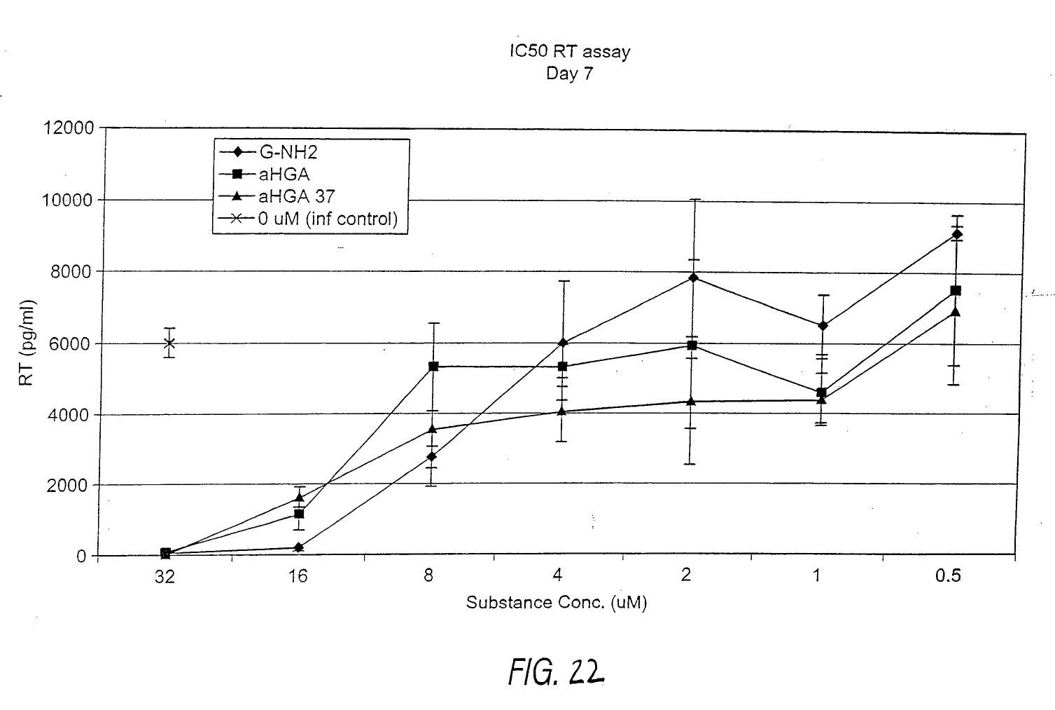

The results of these experiments are shown in FIG. 22 and TABLE 18. FIG. 22 shows a plot of the RT activity detected at day 7. Similar results were obtained when the RT activity was analysed at day 11. The data show that synthetically prepared AlphaHGA is stable to incubation at 37° C. for at least three days. Very little difference in the antiretroviral activity of freshly diluted AlphaHGA and the incubated compound was observed. Further, these data show that appreciable inhibition of HIV replication occurs with synthetic AlphaHGA (whether heat-treated or not) at concentrations above 8 μM, better antiretroviral activity was observed at concentrations above 16 μM, and very efficient inhibition of HIV replication was seen at concentrations above 30 μM. Interestingly, the Metabolite X formed from the conversion of G-NH₂ by the fetal calf serum in the assay (see the data on the G-NH₂ sample) was more active than the synthetically purified AlphaHGA, which provides evidence that one enantiomer and/or isomer of AlphaHGA has more antiretroviral activity than the other.

TABLE 18

| | Conc. | | | OD₄₀₅₋₆₂₀ - | | | | |
|---|---|---|---|---|---|---|---|---|
| Compound | (μM) | | OD₄₀₅₋₆₂₀ | Blank | RT (pg/ml) | StAv | Conc. | mean |
| Control | 0 | | 0.631 | 0.605 | 6318 | 420 | 0 | 6029 |
| | 0 | | 0.622 | 0.596 | 6221 | | | |
| | 0 | | 0.56 | 0.534 | 5547 | | | |
| G-NH2 | 32 | | * | * | * | 2 | 32 | 21 |
| | 32 | | 0.155 | 0.129 | 23 | | | |
| | 32 | | 0.141 | 0.115 | 20 | | | |
| | 16 | | 0.563 | 0.537 | 112 | 40 | 16 | 158 |
| | 16 | | 0.861 | 0.835 | 176 | | | |
| | 16 | | 0.902 | 0.876 | 185 | | | |
| | 8 | | 0.274 | 0.248 | 2438 | 315 | 8 | 2750 |
| | 8 | | 0.302 | 0.276 | 2742 | | | |
| | 8 | | 0.332 | 0.306 | 3068 | | | |
| | 4 | | 0.781 | 0.755 | 7949 | 1682 | 4 | 6029 |
| | 4 | | 0.493 | 0.467 | 4818 | | | |
| | 4 | | 0.539 | 0.513 | 5318 | | | |
| | 2 | | 0.868 | 0.842 | 8895 | 2252 | 2 | 7789 |
| | 2 | | 0.903 | 0.877 | 9275 | | | |
| | 2 | | 0.528 | 0.502 | 5199 | | | |
| | 1 | | 0.563 | 0.537 | 5579 | 838 | 1 | 6514 |
| | 1 | | 0.672 | 0.646 | 6764 | | | |
| | 1 | | 0.712 | 0.686 | 7199 | | | |
| | 0.5 | | 0.871 | 0.845 | 8927 | 205 | 0.5 | 9152 |
| | 0.5 | | 0.896 | 0.87 | 9199 | | | |
| | 0.5 | | 0.908 | 0.882 | 9329 | | | |
| αHGA | 32 | μM | 0.269 | 0.243 | 48 | 25 | 32 | 72 |
| | 32 | μM | 0.373 | 0.347 | 70 | | | |
| | 32 | μM | 0.497 | 0.471 | 97 | | | |
| | 16 | μM | 0.189 | 0.163 | 1514 | 431 | 16 | 1134 |
| | 16 | μM | 0.111 | 0.085 | 666 | | | |
| | 16 | μM | 0.162 | 0.136 | 1221 | | | |
| | 8 | μM | 0.665 | 0.639 | 6688 | 1256 | 8 | 5300 |
| | 8 | μM | 0.507 | 0.481 | 4971 | | | |
| | 8 | μM | 0.44 | 0.414 | 4242 | | | |
| | 4 | μM | 0.541 | 0.515 | 5340 | 615 | 4 | 5315 |
| | 4 | μM | 0.481 | 0.455 | 4688 | | | |
| | 4 | μM | 0.594 | 0.568 | 5916 | | | |
| | 2 | μM | 0.786 | 0.76 | 8003 | 2397 | 2 | 5934 |
| | 2 | μM | 0.647 | 0.621 | 6492 | | | |
| | 2 | μM | 0.354 | 0.328 | 3308 | | | |
| | 1 | μM | 0.564 | 0.538 | 5590 | 945 | 1 | 4594 |
| | 1 | μM | 0.462 | 0.436 | 4482 | | | |
| | 1 | μM | 0.391 | 0.365 | 3710 | | | |
| | 0.5 | μM | 0.692 | 0.666 | 6982 | 2153 | 0.5 | 7539 |
| | 0.5 | μM | 0.962 | 0.936 | 9916 | | | |
| | 0.5 | μM | 0.576 | 0.55 | 5721 | | | |
| αHGA 37 | 32 | μM | 0.198 | 0.172 | 32 | 11 | 32 | 43 |
| | 32 | μM | 0.243 | 0.217 | 42 | | | |
| | 32 | μM | 0.296 | 0.27 | 54 | | | |
| | 16 | μM | 0.171 | 0.145 | 1318 | 282 | 16 | 1641 |
| | 16 | μM | 0.219 | 0.193 | 1840 | | | |
| | 16 | μM | 0.212 | 0.186 | 1764 | | | |
| | 8 | μM | 0.549 | 0.523 | 5427 | 1654 | 8 | 3558 |
| | 8 | μM | 0.322 | 0.296 | 2960 | | | |
| | 8 | μM | 0.26 | 0.234 | 2286 | | | |
| | 4 | μM | 0.33 | 0.304 | 3047 | 909 | 4 | 4050 |
| | 4 | μM | 0.444 | 0.418 | 4286 | | | |
| | 4 | μM | 0.493 | 0.467 | 4818 | | | |
| | 2 | μM | 0.64 | 0.614 | 6416 | 1847 | 2 | 4329 |
| | 2 | μM | 0.317 | 0.291 | 2905 | | | |
| | 2 | μM | 0.387 | 0.361 | 3666 | | | |
| | 1 | μM | 0.512 | 0.486 | 5025 | 713 | 1 | 4420 |
| | 1 | μM | 0.473 | 0.447 | 4601 | | | |
| | 1 | μM | 0.384 | 0.358 | 3634 | | | |
| | 0.5 | μM | 0.891 | 0.865 | 9145 | 2147 | 0.5 | 6978 |
| | 0.5 | μM | 0.496 | 0.47 | 4851 | | | |
| | 0.5 | μM | 0.688 | 0.662 | 6938 | | | |

### Example 16

The compounds of formulas K and M are provided below.

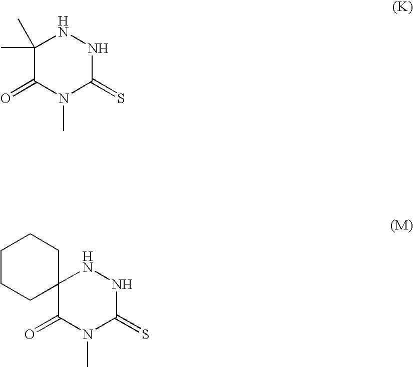

HIV infectivity assays were used to evaluate whether the compounds of formulas K and M effectively inhibit HIV replication. MT-4, C8166 or CEM cells (4 to 5×10⁵ cells per ml) were suspended in fresh culture medium (RPMI-1640 medium supplemented with 10% fetal bovine serum (FBS) (BioWittaker Europe, Verviers, Belgium), 2 mM L-glutamine and 0.075 M NaHCO₃). Subsequently, the cells were infected with HIV-1 (III_{B}) or HIV-2(ROD) at ˜100 CCID₅₀ (1 CCID₅₀ being the dose infective for 50% of the cell cultures) per ml of cell suspension. Then, 100 μl of the infected cell suspension were transferred to 96-well microplate wells, mixed with 100 μl of the appropriate (5-fold) dilutions of the test compounds and further incubated at 37° C. After 4 to 5 days, giant cell formation was recorded microscopically in the CEM and C8166 cell cultures. The MT-4 cell cultures were treated with trypan blue and the number of viable cells was determined. The 50% effective concentration (EC₅₀) corresponded to the compound concentrations required to prevent syncytium formation by 50% in the virus-infected CEM and C8166 cell cultures or the compound concentrations required to reduce cell death by 50% in the MT-4 cell cultures. The results of these experiments are reported in TABLE 19.

As shown in TABLE 19, the compounds of formulas K and M inhibit the replication of HIV at a level that is comparable to AlphaHGA. Thus, two new compounds that inhibit HIV replication, which can be used to treat and/or prevent HIV infection and/or improve the function of the immune system (e.g., raise T cell count) of an HIV infected subject or ameliorate a condition associated with HIV infection, have been discovered.

TABLE 19

| | EC₅₀ (MT-4) (μM) | | EC₅₀ (CEM) (μM) | | EC₅₀ (C8166) (μM) | |
|---|---|---|---|---|---|---|
| | | HIV-2 | HIV-1 | HIV-2 | | HIV-2 |
| Compound | HIV-1 (III_{B}) | (ROD) | (III_{B}) | (ROD) | HIV-1 (III_{B}) | (ROD) |
| α-HGA | ≧500 | ≧500 | 40 ± 8.2 | 52 ± 23 | 200 | 125 |
| G-NH₂ | >500 | >500 | 25 ± 7.1 | 20 ± 0.0 | 150 | 150 |
| GPG-NH₂ | >500 | >500 | 30 ± 0.0 | 20 | 150 | 150 |
| Oxamide | >500 | >500 | >500 | — | — | — |
| Glycolamide | >500 | >500 | >500 | — | — | — |
| Formula K | >580 | >580 | 115 ± 0.0 | 65 ± 18 | 346 | >580 |
| Formula M | >480 | >480 | 97 ± 42 | 48 ± 14 | >480 | 480 |

In a related set of experiments, the activities of the compounds of formulas K and M were compared to alpha HGA. The results are provided in TABLE 20. As shown, the anti-HIV activities of the compounds of formulas K and M are comparable to that of alpha-hydroxyglycinamide.

TABLE 20

| | EC₅₀ (MT-4) | | EC₅₀ (CEM) | | EC₅₀ (C8166) | | | | |
|---|---|---|---|---|---|---|---|---|---|
| | (μM) | | (μM) | | (μM) | | CC₅₀ (μM) | | |
| | HIV- | HIV- | HIV- | HIV- | HIV- | | MT- | | |
| Compound | 1(III_{B}) | 2 (ROD) | 1(III_{B}) | 2 (ROD) | 1 (III_{B}) | HIV-2 (ROD) | 4 | CEM | C8166 |
| G-NH₂ | >500 | >500 | 25 ± 7.1 | 20 ± 0.0 | 150 | 150 | >2000 | >2000 | >2000 |
| α-HGA | ≧500 | ≧500 | 43 ± 5.8 | 52 ± 23 | 200 | 125 | 372 ± 107 | 606 ± 23 | — |
| Formula K | >1000 | >1000 | 115 ± 0.0 | 65 ± 18 | 289 ± 81 | >230 | 906 ± 179 | ≧1150 | 896 ± 265 |
| (C₆H₁₁N₃OS) | | | | | | | | | |
| Formula M | >900 | >900 | 97 ± 42 | 48 ± 14 | >190 | >190 | 455 ± 4.6 | ≧930 | 488 ± 3.3 |
| (C₉H₁₅N₃OS) | | | | | | | | | |

### Example 17

In these experiments, the toxicity of α-hydroxy glycinamide (AlphaHGA) and the compounds of formulas K and M were compared by employing a cell proliferation and mitogenic assay. The cell proliferation assay was conducted as follows. Human lymphocytic CEM cells were seeded in 96-well microtiter plates at ˜5×10⁴ cells/200 μl-well in RPMI-1640 cell culture medium in the presence of various concentrations (200, 40, 8, 1.6, 0.32 and 0.062 μg/ml) of the test compounds (alpha-HGA, Formula K and Formula M). The cell cultures were cultivated for 96 hr at 37° C. in a CO₂-controlled atmosphere. At the end of the incubation period, the cells were counted in a Coulter counter (Analis, Gent, Belgium).

The mitogenic activity assay was conducted as follows. Human PBMC were isolated from buffy coats and brought in RPMI-1640 cell culture medium supplemented with 10% FCS and 2 mM L-glutamine. The cell cultures were exposed to several concentrations of the test compounds for 3 days in the absence or presence of PHA (phytohemagglutinin) (Murex Biotech Ltd., Dartford, UK) at 2 μg/ml. Subsequently, 0.25 μCi [CH₃⁻³H]dThd was added and, after 20 hr incubation at 37° C., the cells were precipitated in the presence of 10% TCA and the precipitate was washed twice with 10% TCA, twice with 5% TCA and once with 70% ethanol. The precipitate, containing [³H]DNA, was then examined for radioactivity in a liquid scintillation counter.

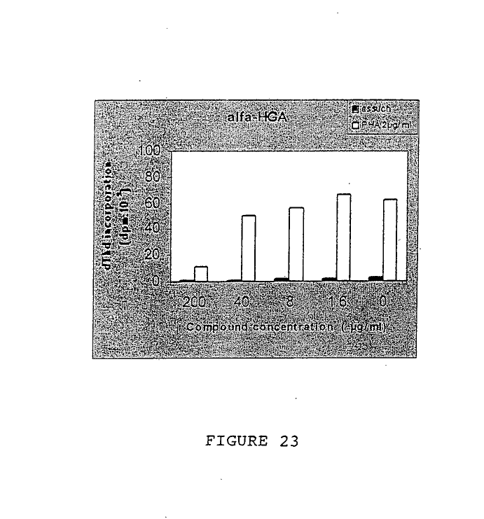

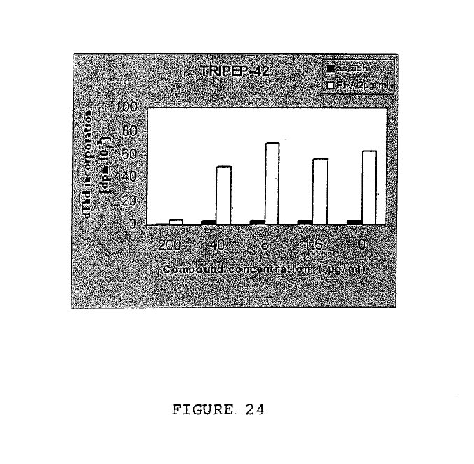

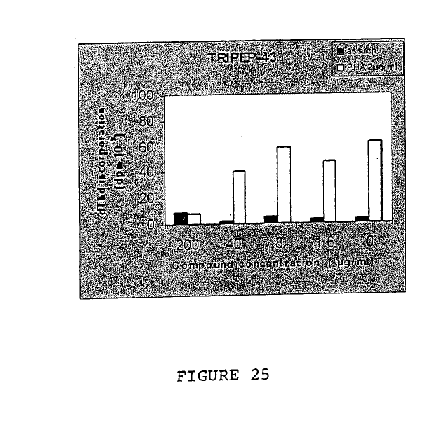

As shown in FIGS. 23-25 none of the tested compounds exhibited mitogenic activity against human PBMCs at concentrations up to 200 μg/ml. In contrast, 2 μg/ml PHA markedly stimulated dThd incorporation into PBMC DNA. When the compounds were added to PBMCs in the presence of PHA, no effect on PHA-induced stimulation of DNA synthesis (cell proliferation) was measured at concentrations up to 40 μg/ml. At 200 μg/ml, the PHA-induced stimulation was markedly inhibited by the test compounds, indicating toxicity at these high levels. The results in these experiments provided evidence that alphaHGA and the compounds of formulas K and M can be provided at concentrations that are non-mitogenic.

The antiretrovirus assay, the toxicity assay and the mitogenic activity assays described above can also be performed for any of the compounds described herein (e.g., the compounds of formulas A, B, C, D, E, F, G, H, I, J, K, L, M, N, O, P, Q, R, S, T, U, V, W, X, Y, Z, or AA) and similar results will be obtained.

### Example 18

The compound of formula Y is provided below.

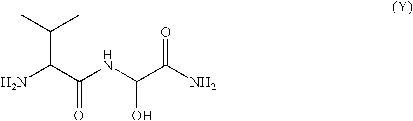

HUV infectivity assays were used to compare the effectivness of inhibition of HIV replication by the compound of formula Y and alpha-HGA. H9 cells in RPMI 1640 medium containing L-glutamine, 10% Fetal Calf Serum, and 0.1% Penicillin-Streptomycin were infected with SF2 laboratory strain HIV. All cells were infected at the same time and in the same tube in a batch infection. After infection, the cells were resuspended and 100,000H9 cells per aliquot were added to the wells of a 48-well plate.

The compound of formula Y or alpha-HGA was then added to corresponding wells and the plate was incubated at 37° C. in a 5% CO₂ enriched incubator. Alpha-HGA and the compound of formula Y was added either as powder or dissolved powder and incubated with the infected cells for 3 days or 7 days. Analysis was conducted at various concentrations of the compounds including 50, 25, 12.5, 6.25, 3.125, and 1.562 μM. The medium was changed at day 7 and fresh compound was added with the medium. The infection was stopped on day 11. Supernatant from the sample wells was collected on days 7 and 11 and analyzed. The supernatant was stored at −80° C. until analysis with an RT Quantification Assay (Lenti-RTTM Activity Assay, Cavidi Tech, Uppsala, Sweden) and/or a p24-Elisa. Infected, non-treated cells were used as a positive control. Uninfected, non-treated cells were used as a negative control.

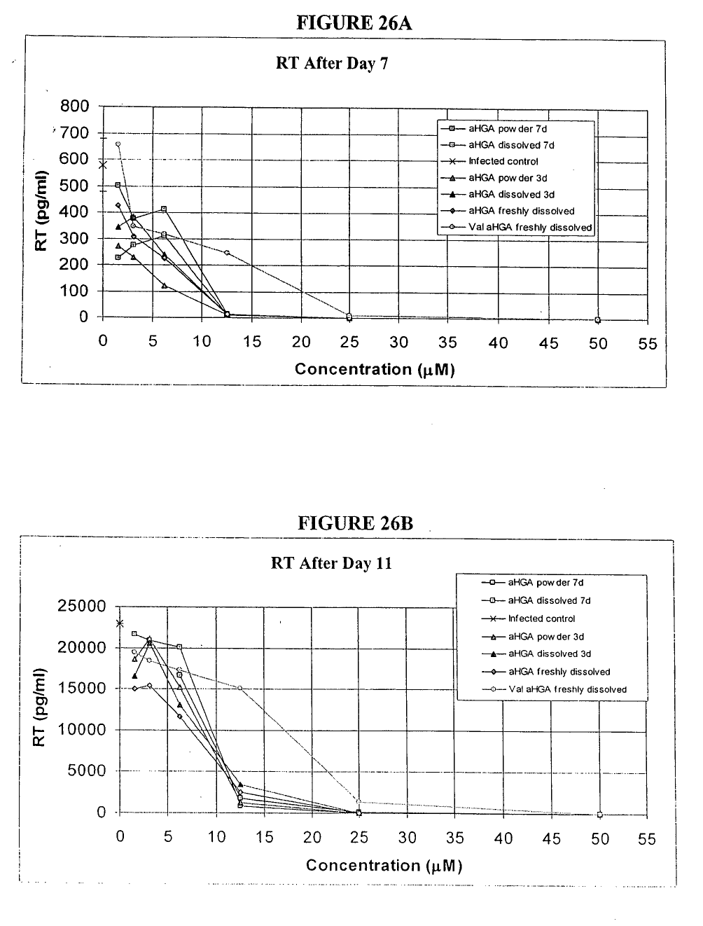

The results are shown on the graphs of RT quantification in FIGS. 26A (supernatant at day 7) and 26B (supernatant at day 11). The results show that the compound of formula Y effectively inhibits HIV replication having an IC₅₀ of about 16 μM at day 11 compared to an IC₅₀ of about 6-9 μM for alpha-HGA. Nevertheless, at 25 μM concentration both alphaHGA and the compound of formula Y reduced HIV replication to neglible levels.

The following examples describe some synthetic approaches in greater detail.

### Example 19

### 19-1

α-Hydroxy-N-tert-butoxycarbonylglycine methyl ester (4.11 g, 20 mmol) and imidazole are dissolved in DMF at room temperature and cooled to a temperature of 0° C. Then chlorinated tert-butyldimethylsilyl is added to the solution at this temperature and the components are stirred for 10 min. The solution is returned to room temperature and stirring is continued for 1 hour. Then, saturated brine is added and extraction is conducted with ethyl acetate. The organic layer is dried with anhydrous magnesium sulfate and the solvent is distilled off.

The oily substance obtained is then dissolved in ethanol (50 mL) and excess ammonia is blown into the solution at a temperature of 0° C. Next, the excess ammonia is removed under reduced pressure and ethanol is distilled off. The crude product thus obtained is purified by silica gel column chromatography and α-tert-butyldimethylsilyloxy-N-tert-butoxycarbonylglycinamide (6.10 g, quant.) is obtained. An expected profile includes: ¹HNMR δ (CDCl₃) 0.16 (s, 3H), 0.21 (s, 3H), 0.92 (s, 9H), 5.46 (d, 1H, J=9 Hz), 5.63 (d, 1H, J=9 Hz), 6.22-6.82 (br, 2H).

### 19-2

The α-hydroxy-N-tert-butoxycarbonylglycine methyl ester that is a starting substance in 19-1 above is prepared in the manner as follows: tert-Butyl carbamate (2.83 g, 23.6 mmol) and glyoxylic acid monohydrate (2.02 g, 21.5 mmol) are dissolved in acetone (50 mL) and refluxed overnight. The solution is then cooled to a temperature of 0° C. and treated with excess diazomethane-ether solution at this temperature. The solvent is then distilled off.

Saturated brine is then added, extraction is conducted with chloroform, the organic layer is dried with anhydrous magnesium sulfate and the solvent is distilled off. The crude product thus obtained is purified by silica gel column chromatography and α-hydroxy-N-tert-butoxycarbonylglycine methyl ester (2.56 g, 58%) is obtained. An expected profile includes: ¹HNMR δ (CDCl₃) 1.46 (s, 9H), 1.65 (br s, 1H), 3.84 (s, 3H), 5.27-5.52 (br, 1H), 5.59-5.90 (br, 1H). IR(NaCl) 1755 (s), 1690 (s), 1528 (s) cm⁻¹.

### 19-3

The α-hydroxy-N-tert-butoxycarbonylglycine methyl ester that is a starting substance in 19-1 above can be prepared by a method other than that of 19-2. Accordingly, tert-Butyl carbamate (11.35 g, 95.0 mmol) and 1-hydroxy-1-methoxyacetic acid methyl ester (14.35 g, 119.5 mmol) are dissolved in anhydrous THF (50 mL) and refluxed overnight. The temperature is then returned to room temperature, 1-hydroxy-1-methoxyacetic acid methyl ester (1.15 g, 9.6 mmol) is then added and the components are further refluxed for 8 h. The reaction liquid is allowed to sit until the temperature returns to room temperature and the solvent is then distilled off. The crude product thus obtained is recrystallized from a chloroform-hexane solution and pure α-hydroxy-N-tert-butoxycarbonylglycine methyl ester (16.42 g, 84%) is obtained.

### Example 20

The α-hydroxy-N-tert-butoxycarbonylglycine methyl ester (1.21 g, 5.9 mmol) obtained in 19-2 or 19-3 above is dissolved in DMF (10 mL), and then silver oxide (1.04 g, 4.5 mmol) and benzene iodide (1.99 g, 9.1 mmol) are added at room temperature. The components are stirred overnight at room temperature, the precipitate is filtered, water is added to the mother liquor, and extraction is conducted with ethyl acetate. The extracted solution is dried with anhydrous magnesium sulfate, then the solvent is distilled off and crude purification is conducted with silica gel column chromatography.

The oily substance thus obtained is dissolved in ethanol (50 mL) and excess ammonia is blown into the solution at a temperature of 0° C. The excess ammonia is then removed under reduced pressure and the solvent is distilled off. The crude product thus obtained is purified by silica gel column chromatography and α-benzyloxy-N-tert-butoxycarbonylglycinamide (0.397 g, 22%) is obtained. An expected profile includes: m.p. 115-120° C., ¹HNMR δ (CDCl₃) 1.44 (s, 9H), 4.61 (d, 1H, J=11.3 Hz), 4.79 (d, 1H, J=11.3 Hz), 5.4 (d, 1H, J=9.0 Hz), 5.75 (brd, 1H, J=9.0 Hz), 6.00 (br, 1H), 6.52 (br, 1H), 7.35 (s, 5H). IR(NaCl) 1698 (s), 1664 (s), 1502 (s), 732 (m), 695 (m) cm⁻¹. Analytical values for elements (C₁₄H₂₀O₄N₂): Calcd. C, 59.99; H, 7.19; N, 9.99 Obsd. C, 59.94; H, 7.33; N, 10.28 are expected.

### Example 21

The α-hydroxy-N-tert-buthoxycarbonylglycinemethyl ester (2.07 g, 10.1 mmol) prepared according to 19-2 or 19-3 above is dissolved in DMF (20 mL), and silver oxide (1.39 g, 6.0 mmol) and allyl iodide (1.2 mL, 12.9 mmol) are added at room temperature. After overnight stirring at room temperature, the precipitate is filtered out, water is added to the mother liquor, and extraction with ethyl acetate is conducted. The extracted solution is dried with anhydrous magnesium sulfate, then the solvent is distilled off, and an aqueous solution of sodium thiosulfate is added, followed by extraction with ethyl acetate and removal of iodine as a reaction byproduct.

The oily substance thus obtained is dissolved in ethanol, excess ammonia is blown into the solution at a temperature of 0° C., the excess ammonia is thereafter removed under reduced pressure, and the solvent is distilled off. The crude product obtained is purified with silica gel column chromatography to obtain α-allyloxy-N-tert-butoxycarbonylglycinamide (0.625 g, 27%). An expected profile includes: ¹HNMR δ (CDCl₃) 1.45 (s, 9H), 4.14 (dd, 2H, J=7.2, 1.8 Hz), 5.11-5.56 (m, 3H), 5.70-6.20 (m, 2H), 6.33-7.01 (m, 2H). IR(CDCl₃) 2975 (w), 1705 (s, br), 1498 (m), 990 (sh. w) cm⁻¹.

### Example 22

### 22-1

α-Hydroxy-N-benzyloxycarbonylglycine (4.44 g, 19.7 mmol) is dissolved in methanol (20 mL). Thionyl chloride (2.9 mL, 40.0 mmol) is dropwise added to the solution at a temperature of 0° C., and stirring is conducted for 30 minutes at this temperature and then for 2 hours at room temperature. The solvent is then distilled off and the crude product obtained is dissolved in methanol (50 mL). The solution is cooled to 0° C., and excess ammonia is blown therein.

Upon completion of the reaction, the excess ammonia is removed under reduced pressure, the solvent is distilled off, and the white crystals obtained are purified with silica gel column chromatography to obtain α-methoxy-N-benzyloxycarbonylglycinamide (3.42 g, 73%). An expected profile includes: m.p. 110-112° C., ¹HNMR δ (CDCl₃) 3.44 (s, 3H), 5.16 (s, 2H), 5.31 (d, 1H, J=8.8 Hz), 5.45-5.98 (br, 2H), 6.28-6.68 (br, 1H), 7.36 (s, 5H). IR(NaCl) 1680 (s. br), 1540 (s), 1520 (s), 860 (m), 700 (m) cm⁻¹. Analytical values of elements (C₁₁H₁₄O₄N₂); Calcd. C, 55.46; H, 5.92; N, 11.76 Obsd. C, 55.70; H, 5.94; N, 11.58 are expected.

### 22-2

The α-hydroxy-N-benzyloxycarbonylglycine that is the starting material in 16-4 above is prepared in the manner as follows. Benzyl carbamate (30.24 g, 0.2 mol) and glyoxylic acid monohydrate (20.26 g, 0.22 mol) are dissolved in diethyl ether (200 mL) and the solution is stirred overnight at room temperature. The crystals produced are filtered and then washed with ether to obtain pure α-hydroxy-N-benzyloxycarbonylglycine (33.78 g, 75%). An expected profile includes: m.p. 200-205° C., ¹HNMR δ (CD₃OD) 5.12 (s, 2H), 5.40 (s, 1H), 7.34 (s, 5H).

### Example 23

The α-hydroxy-N-benzyloxycarbonylglycine (2.26 g, 10.0 mmol) produced according to 22-2 above is dissolved in ethanol (20 mL). Thionyl chloride (2 mL, 27.4 mmol) is dropwise added to the solution at a temperature of −10° C., and stirring is conducted overnight at room temperature. The solvent is then distilled off and the crude product thus obtained is purified with silica gel column chromatography to obtain α-ethoxy-N-benzyloxycarbonylglycine ethyl ester (2.81 g, quant.). An expected profile includes: m.p. 66-68° C., ¹HNMR δ (CDCL₃) 1.22 (t, 3H, J=7.2 Hz), 1.30 (t, 3H, J=7.2 Hz), 3.70 (q, 2H, J=7.2 Hz), 4.24 (q, 2H, J=7.2 Hz), 5.15 (s, 2H), 5.33 (d, 1H, J=9.7 Hz), 5.93 (brd, 1H, J=9.7 Hz), 7.35 (s, 5H). IR(NaCl) 1740 (s), 1700 (s), 1540 (s), 760 (m), 700 (m) cm⁻¹. Analytical values of elements (C₁₄H₁₉O₅N); Calcd. C, 59.78; H, 6.81; N, 4.98; Obsd. C, 60.03; H, 6.88; N, 4.89 are expected.

### Example 24

The α-hydroxy-N-benzyloxycarbonylglycine (2.26 g, 10.0 mmol) produced according to 22-2 above is dissolved in isopropyl alcohol (20 mL). Thionyl chloride (2 mL, 27.4 mmol) is dropwise added to the solution at a temperature of −10° C., and stirring is conducted overnight at room temperature. The solvent is then distilled off and the crude product thus obtained is purified with silica gel column chromatography to obtain α-isopropoxy-N-benzyloxycarbonylglycine isopropyl ester (3.10 g, quant.). An expected profile includes: ¹HNMR δ (CDCL₃) 1.16-1.37 (m, 12H), 3.87-4.22 (m, 1H), 4.57-5.20 (m, 1H), 5.14 (s, 2H), 5.33 (d, 1H, J=9.7 Hz), 5.93 (brd, 1H, J=9.7 Hz), 7.35 (s, 5H). IR(Neat) 1728 (s, br), 1508 (m), 740 (m) cm⁻¹.

### Example 25

The α-ethoxy-N-benzyloxycarbonylglycine ethyl ester (2.29 g, 8.1 mmol) produced according to EXAMPLE 23 is dissolved in ethanol (80 mL) and cooled to 0° C. Excess ammonia is then blown into the solution at this temperature. Upon completion of the reaction, the excess ammonia is removed under reduced pressure, the solvent is distilled off, and the white crystals thus obtained are washed with a hexane-ethyl acetate mixed solution to obtain pure α-ethoxy-N-benzyloxycarbonylglycinamide (1.51 g, 77%). An expected profile includes: m.p 119-121° C., ¹HNMR δ (CDCL₃) 1.23 (t, 3H, J=7.1 Hz), 3.50-3.90 (m, 2H), 5.14 (s, 2H), 5.37 (d, 1H, J=9.0 Hz), 5.65-5.96 (br, 2H), 6.41-6.71 (br, 1H), 7.35 (s, 5H). IR(NaCl) 1680 (s), 1664 (s), 1542 (m), 1524 (m), 760 (w), 740 (w), 700 (m) cm⁻¹. Analytical values of elements (C₁₂H₁₆O₄N₂); Calcd. C, 57.13; H, 6.39; N, 11.10; Obsd. C, 57.09; H, 6.34; N, 11.37 are expected.

### Example 26

The α-isopropoxy-N-benzyloxycarbonylglycine isopropyl ester (2.48 g, 8.0 mmol) produced according to EXAMPLE 23 is dissolved in ethanol (40 mL) and cooled to 0° C. Then, excess ammonia is blown into the solution for 5 hours at this temperature and stirring is further conducted for 2 days in the ammonia saturated state. Upon completion of the reaction, the excess ammonia is removed under reduced pressure, the solvent is distilled off, and the white crystals thus obtained are washed with a hexane-ethyl acetate mixed solution to obtain pure α-isopropoxy-N-benzyloxycarbonylglycinamide (1.64 g, 77%). An expected profile includes: m.p 111-113° C., ¹HNMR δ (CDCL₃) 1.18 (d, 3H, J=4.4 Hz), 1.25 (d, 3H, J=4.4 Hz), 3.81-4.20 (m, 1H), 5.15 (s, 2H), 5.44 (d, 1H, J=9.0 Hz), 5.53-5.86 (br, 2H), 6.37-6.73 (br, 1H), 7.35 (s, 5H). IR(NaCl) 1668 (s), 1660 (s), 1538 (m), 1530 (m), 760 (w), 740 (w), 700 (m) cm⁻¹. Analytical values of elements (C₁₃H₁₈O₄N₂); Calcd. C, 58.63; H, 6.81; N, 10.52. Obsd. C, 58.60; H, 6.82; N, 10.54 are expected.

### Example 27

The α-tert-butyldimethylsilyloxy-N-tert-buthoxycarbonylglycinamide (5.08 g, 16.7 mmol) produced according to (19-1) of EXAMPLE 19 is dissolved in dioxane (10 mL) and cooled to 0° C. Then, a 4N hydrochloric acid-dioxane solution (17 mL) is added and stirring is conducted for 1 hour at this temperature.

In order to complete the reaction, a 4N hydrochloric acid-dioxane solution is further added, the temperature is raised to room temperature and stirring is conducted for 1 hour. Diethyl ether is then added to the solution, as large an amount of the product as possible is precipitated, filtered, and washed with ether. The precipitate is then dried under reduced pressure to obtain pure α-hydroxyglycinamide hydrochloride (1.86 g, 88%). An expected profile includes: ¹HNMR δ (DMSO-d₆) 4.99 (br sd, 1H), 7.62-8.03 (br, 2H), 8.32-8.85 (br, 3H). IR (KBr) 1686 (s), 1581 (m), 1546 (m), 1477 (s), 843 (m) cm⁻¹

### Example 28

The α-methoxy-N-benzyloxycarbonylglycinamide (0.24 g, 1.0 mmol) prepared according to EXAMPLE 21 (21-1) is dissolved in methanol, 12N hydrochloric acid (0.1 mL) and palladium-carbon (50 mg) are added to the solution at room temperature, and stirring is conducted for 30 minutes under hydrogen atmosphere. The palladium-carbon is then filtered out and the solvent of the mother liquor is distilled off to obtain α-methoxyglycinamide hydrochloride (0.14 g, quant). An expected profile includes: ¹HNMR δ (CD₃OD) 3.35 (s, 3H), 5.01 (s, 1H), ¹³CNMR δ (CD₃OD) 42.1, 84.3 (d, J=159.8 Hz), 170.3. The next example describes an approach that was used to synthesize α-hydroxy-glycinamide hydrochloride for formulation into a pharmaceutical, dietary supplement, or medicament.

### Example 29

### Preparation of α-Hydroxy-Glycinamide Hydrochloride

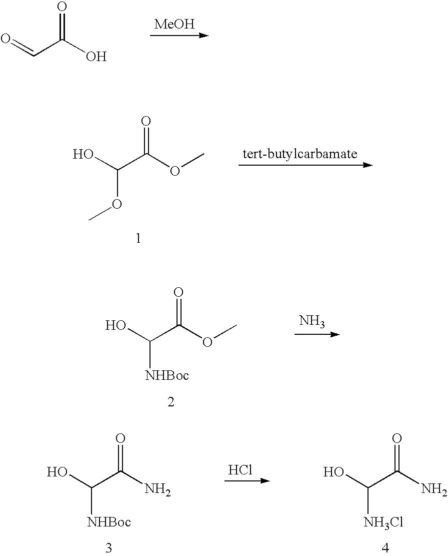

### 29-1 Methyl α-hydroxymethoxyacetate

A solution of glyoxylic acid monohydrate (7.0 g, 76 mmol) in methanol (35 mL) was refluxed overnight. The solution was then neutralized with saturated NaHCO₃ and evaporated. The residue was dissolved in CH₂Cl₂ and dried over Na₂SO₄. Evaporation afforded 3.23 g (40.0%) of crude oil that was used in the following reaction without further purification.

### 29-2 Methyl N-tert-butoxycarbonyl-α-hydroxyglycinate

A solution of methyl α-hydroxymethoxyacetate (2.0 g, 18.9 mmol) and tert-butyl carbamate (2.0 g, 17.18 mmol) in toluene (45 mL) was refluxed overnight. Evaporation afforded oil. This crude oil was purified by silica gel chromatography EtOAc/heptane 1/9 to 2/8 as eluent. The pure fractions gave 0.6 g oily product that was then crystallized with diethyl ether/heptane. The yield 0.39 g (10.1%). The NMR spectra observed were:

¹H NMR (300 MHz, CDCl₃) δ 5.74 (br s, 1H), 5.44 (br s, 1H), 3.84 (s, 3H), 1.46 (s, 9H).

¹³C NMR (300 MHz, DMSO-d₆) δ 170.3, 154.7, 78.6, 72.8, 51.9, 28.1.

29-3 N-tert-butoxycarbonyl-α-hydroxyglycinamide

Methyl N-tert-butoxycarboriyl-α-hydroxyglycinate (0.34 g, 1.66 mmol) was solved in 7N NH₃ in methanol (4 mL). The solution was stirred at room temperature overnight, evaporated and then co-evaporated twice with acetonitrile. The product was purified by silica gel chromatography EtOAc/heptane 3/7 to 5/5 as eluent. The yield 0.1 g (31.7%). The NMR spectra observed were:

¹H NMR (300 MHz, DMSO-d₆) δ 7.28 (br d, 2H), 6.20 (d, 1H), 5.09 (t, 1H), 1.39 (s, 9H).

¹³C NMR (300 MHz, DMSO-d₆) δ 171.7, 155.0, 78.3, 73.4, 28.2.

### 29-4 α-Hydroxy-glycinamide hydrochloride

N-tert-butoxycarbonyl-α-hydroxyglycinamide (40 mg, 0.2 mmol) was solved in dioxane (1.5 mL). 4N HCl in dioxane (0.5 mL) was added to the solution at 0° C. The cooling bath was removed and the solution was stirred for 40 min. at room temperature. Diethyl ether was added and the solution was stirred. Ether was decanted and the residue was evaporated. The yield was approximately 40 mg. The NMR spectra observed were:

¹H NMR (500 MHz, DMSO-d₆) δ 8.5-7.1 (m, 5H), 4.85 (s, 1H).

¹³C NMR (500 MHz, DMSO-d₆) δ 173.1, 87.4.

The following example describes an approach that was used to prepare α-methoxy-glycinamide, which can be incorporated into a pharmaceutical, medicament, or dietary supplement.

### Example 30

### Preparation of α-Methoxy-Glycinamide

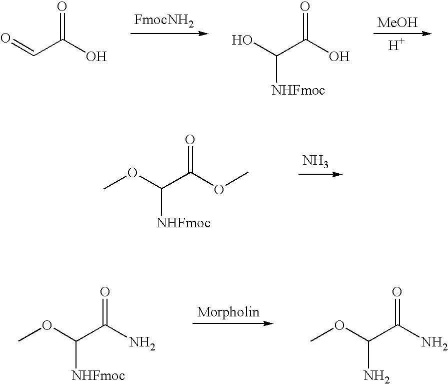

### 30-1 Methyl N-(9H-Fluoren-9-ylmethoxycarbonyl)-α-methoxyglycinate

Glyoxylic acid monohydrate (276 mg, 3 mmol) and 9H-fluoren-9-ylmethyl carbamate (320 mg, 1.33 mmol) were solved in dry diethylether (10 mL). The mixture was stirred at room temperature overnight. The solvent was evaporated and the residue was solved in methanol (20 mL) and 1 drop of sulfuric acid was added. The reaction mixture was stirred 3 days at room temperature. Sat. NaHCO₃ (100 mL) was added to the mixture and it was extracted with ethyl acetate, dried over Na₂SO₄ and evaporated. The residue was purified on silica gel column to give 250 mg (55%) of the titled compound. The NMR spectra observed were:

¹H NMR (300 MHz, CDCl₃) δ 7.76 (d, 2H), 7.59 (d, 2H), 7.40 (t, 2H), 7.31 (t, 2H), 5.90 (br d, 1H), 5.35 (d, 1H), 4.46 (m, 2H), 4.24 (t, 1H), 3.82 (s, 3H), 3.43 (s, 3H).

¹³C NMR (300 MHz, CDCl₃) δ 143.6, 143.5, 141.2, 127.7, 127.1, 124.9, 120.0, 80.5, 67.2, 56.2, 52.9.

### 30-2 α-Methoxyglycinamide

Methyl N-(9H-Fluoren-9-ylmethoxycarbonyl)-α-methoxyglycinate (240 mg, 0.7 mmol) was treated with 3N NH₃ in methanol (20 mL) at room temperature overnight. Methanol was removed by evaporation. The solid was solved in THF (30 mL) and morpholine (305 mg, 3.5 mmol) was added. The mixture was stirred at room temperature for 5 h. The solvent was evaporated and the product was purified on silica gel column to give 5 mg (6%) of the titled compound. The NMR spectrum observed was:

¹H NMR (300 MHz, CDCl₃) δ 4.40 (br s, 1H), 3.35 (s, 3H).

### Example 31

### Preparation of N-L-Valyl-α-Hydroxyglycinamide

31-1 Methyl N-Boc-L-Valyl α-hydroxyglycinate

Boc-L-Valine Amide (0.9 g; 4.2 mmol) and Methyl α-hydroxymethoxyacetate (see Example 29-1) (0.5 g; 4.7 mmol) were refluxed in acetonitrile (30 mL) for 22 h. The reaction mixture was evaporated and purified by flash chromatography EtOAc/Heptane 10/90 to 30/70 as eluent to give 0.28 g of product.

¹H NMR (300 MHz, DMSO-d₆) δ 8.66 (d, 1H), 6.61 (m, 2H), 5.46 (m, 1H), 3.78 (m, 1H), 3.64 (s, 3H), 1.91 (m, 1H), 1.38 (s, 9H), 0.83 (m, 6H).

### 31-2 N-Boc-L-Valyl α-hydroxyglycinamide

The product from Example 31-1 (0.5 g; 1.72 mmol) was dissolved in 7 N NH₃ in MeOH (2 mL) at −10° C. and the mixture was stirred for 5.5 h at −10° C.-0° C. The cooling bath was removed and stirring was continued for 2 h at room temperature and after that the mixture was evaporated. The crude material was purified by flash chromatography EtOAc/Heptane 10/90 to pure EtOAc as eluent to give 0.18 g of product.

¹H NMR (300 MHz, DMSO-d₆) δ 8.40 (m, 1H), 7.31 (m, 2H), 6.69 (m, 1H), 6.42 (m, 1H), 5.36 (m, 1H), 3.84 (m, 1H), 1.91 (m, 1H), 1.38 (s, 9H), 0.82 (m, 6H).

### 31-3 N-L-Valyl α-hydroxyglycinamide hydrochloride

Concentrated HCl (1 mL) was added to 75 mg of the product from Example 31-2 at room temperature. Acetone was added and the mixture was evaporated. Acetone was added to the residue which formed a precipitate. The solvent was decanted and the precipitate was dried in vacuum.

¹H NMR (300 MHz, DMSO-d₆) δ 9.05 (m, 1H), 8.32 (m), 7.45 (m, 2H), 5.38 (m), 2.07 (m, 1H), 0.92 (m, 6H).

The following example describes a microdosing study that was conducted in humans to determine the bioavailability of alpha-hydroxyglycinamide.

### Example 32

In this example, the pharmacokinetics (PK) and oral bioavailability of AlphaHGA in adult healthy volunteers following a single ‘microdose’ in a sequence-randomized, cross-over study design (two periods 7 days apart) was determined. Clinical experiments were conducted with low-level radiolabelled (¹⁴C) AlphaHGA administered intravenously (IV) orally (PO) with serial blood and urine samples were collected for concentration analysis utilizing accelerator mass spectrometry (AMS).

Accelerator Mass Spectrometry (AMS) is a highly sensitive and quantitative analytical tool that quantifies trace concentrations of a ¹⁴C-labeled drug or test compound in human tissues and fluids at attomolar (10⁻¹⁸) levels of sensitivity. This sensitivity has enabled the development of microdosing, a technique whereby sub-toxic ‘microdoses’ are tested in man early in the drug development process.

AMS is a type of isotope ratio mass spectrometry (IRMS) that uses ion acceleration to increase the sensitivity of IRMS to the level of counting individual nuclei of a very rare (usually radioactive) isotope in a sample comprised primarily of one or more stable isotopes of much greater abundance. The sample is introduced into the ion source as a solid filamentous graphite. The sample is ionized by the addition of an electron to the individual elemental or molecular ions that are removed from the sample material by collisional processes. Both common stable isotopes and rare isotopes are similarly emitted from the sample.

The individual rare isotope ions are counted in an identifying detector that discriminates the desired isotope counts from ions of other elements and isotopes by one or more physical measurements that are sensitive to the protonic charge and the nuclear mass of the impinging ions. A single determination of the rare isotope abundance is provided by the ratio of rare ion counts in the detector to the total stable isotope ion current, with both quantities summed over a common period. One or more such cycles are said to comprise a single measurement of the isotope ratio of the sample. The cycles can be continued until a preset number of counts of the rare isotope are received or until a preset time expires after the completion of one of the cycles.

AMS provides the lowest limit of accurate quantitation of any known bioanalytical detector: Measurements can be performed down to 2 attomole of ¹⁴C with <3% precision. Moreover, the analysis is largely free of matrix effects or contaminating backgrounds that lead to spurious signal. It is the optimal tool in present practice for human microdose studies using sub-toxic quantities of a drug candidate. It can also be coupled to modern chromatographic systems to provide information on metabolite profiles, biotransformations, and covalent binding, or can be used to quantify substances present in small biopsies.

Accordingly, eight subjects were enrolled into a single dose cross-over study. The subjects were randomly assigned to one of two administration sequences. Subjects that were assigned to sequence 1 received AlphaHGA as an oral preparation first (Period 1) followed by the intravenous (IV) preparation 1 week later (Period 2). Subjects assigned to sequence 2 received AlphaHGA as an intravenous preparation first followed by the oral preparation 1 week later. Four subjects were assigned to sequence 1 and four subjects were assigned to sequence 2.

For both oral and IV doses subjects were administered 100 nCi (3.7 Kbq) of alphaHGA, which was determined to contain 1.602888×10ˆ6 fmol of ¹⁴C. The specific activity of each dose was 46.619 MBq/mol (or 1.260 mCi/mol). A stock blend of ¹⁴C-labelled alphaHGA was produced prior to the first study period. The actual formulations used for dosing were prepared on the morning prior to each study period. The infusion was a single bolus injection with both the infusion and flush were completed in less than 30 seconds.

Blood (5 ml) was collected following the administration of AlphaHGA orally or as an IV bolus at the following times: pre-dose (T=0) and 15, 30, 45, 60 minutes, and at 2, 3, 4, 6, 8, 10, 12, 16, 24 hours post-dose. All of the 224 scheduled blood samples were obtained. Blood specimens were drawn by intravenous in-dwelling catheter into 2×10 mL EDTA containing Vacutainer tubes and RBC's separated by centrifugation.

Urine samples were collected at pre-dose and during post-dose time intervals: 0 to 4, 4 to 8, 8 to 12, 12 to 16, and 16 to 24 hours. All urine collections were obtained. Cumulative urine voidings for the specified time intervals were collected in standard collection containers without additional modifiers or preservatives

Samples were received and stored according to 1-SOP-INVT-1. In brief, received samples were checked against the electronic sample submission sheet provided by the client. Samples were assigned a unique internal tracking number (ITN) with barcode and a freezer-safe label (Brady) was printed and placed on the received specimens. Sample inventory records were verified prior to initiation of analysis. Subsequent sample manipulations were conducted using the ITN exclusively. Samples were kept on a bed of dry ice during inventory and will be kept at −60° C. for a maximum of 6 months after receipt.

The samples were then combusted and reduced to filamentous graphite using the procedures and conversion chemistries originally defined by Vogel, J. S Radiocarbon 34: 344-350 (1992), as modified by Ognibene, International Journal of Mass Spectrometry 218:255-264 (2002) and performed according to 1-BioAMS-2 SOP. In brief, neat plasma and diluted urine were dried under reduced pressure and combusted in evacuated quartz combustion break-point tubes to CO₂. The CO₂ was transferred to seal septa-vials containing Zinc powder and cobalt and reduced to filamentous graphite by heating at >500° C. for at least 4 hours. The graphite was pounded into a custom aluminum target vial that was presented to the AMS for determination of carbon isotopic ratios (¹⁴C/total carbon). Samples were generally processed in batches ranging from 20-60 samples. Each batch included a set of 4 standardized calibrants (ANUs) and depleted (sub-modern) lipid sample (tributyrin) that served as a ‘sentinel’ sample or for the detection of low level contamination.

Plasma specimens (30 μL) were analyzed directly via the combustive procedure without further chemical manipulation. For the purposes of calculating ¹⁴C concentration per volume of plasma, a general carbon content of 45% (dry mass) was applied. The urine was mixed with equal volumes (0.5 mL) of a sucrose carbon diluent solution (200 g/L) and mixed by inversion in a disposable polypropylene centrifuge tube. A 20-μL sample was graphitized for AMS analysis according to standard procedures. The mass fraction of the urine represented in the measured sample was inverted and multipled by the ¹⁴C contents above background derived from the AMS measurement to calculate the ¹⁴C content in the total urine void. A density value of 1 g/mL for urine was assumed to calculate its volume from the recorded masses.

Individual target vials containing the specimen after graphitization were transported in individual sealed polyethylene bags to LLNL via overnight courier. Each sample was tracked with a unique ‘V’ designator that was generated from the sample data base (FileMaker). LLNL received no information of the samples identity or source. AMS carbon ratio data was returned via an ASCII format containing the ‘V’ number, and its associated modern value, standard deviation, and instrument ion current.

Measurements were performed on a 1 MV compact bio-AMS instrument (National Electrostatics Corporation) at Lawrence Livermore National Lab (through a contractual partnership with Vitalea Science). System sensitivity was determined to be <1 amol ¹⁴C/mg carbon on milligram-sized samples with a dynamic range that extends over 4 orders magnitude (see Ognibene, International Journal of Mass Spectrometry 218:255-264 (2002)). The useful limit of quantification is a product of careful sample handling and processing procedures used by the organization. Vitalea used state-of-the-art procedures that are under continuous development with Vitalea and Lawrence Livermore. The quality of the results were tracked with calibrants of known and universally accepted ¹⁴C content that have a legacy of widespread use in the AMS community. Sample imprecision was predicted by Poisson statistics. Submitted biochemical samples were measured for a minimum of 4 measurements until the last 4 measurements fall within a set criteria (standard deviation <3% of each other and the last measurement is not the greatest or the least ratio of the set to a maximum of 8 measurements).

The ¹⁴C isotope content of a sample was calculated from the AMS-measured ratios of integrated ¹⁴C counts divided by ¹³C ion current integrated over the same period: R═¹⁴C/¹³C (counts per nanoCoulomb), as detailed in Brown & Southon, Nucl Inst. Meth. B123: 208-213 (1997). These ratios were determined for the sample, Rx, the standard, Rs, and the background material, Rb. If the sample, standard, and backgrounds were all determined on similar sized aliquot (˜1 mg carbon), then the sample concentration, Cx, was found with respect to the known standard concentration, Cs: Cx=Cs (Rx−Rb)/(Rs−Rb). The known concentrations of the commonly used standard material was ¹⁴C per milligram carbon IAEA C-6 sucrose (Scott, et al. 1998): 1.508 Modern=147.5 attomole ¹⁴C per milligram carbon.

Pharmacokinetic parameters were estimated by noncompartmental analysis employing the PK software WinNonlin version 4.1 (Pharsight Corporation, Palo Alto, Calif.). Statistical analyses were performed using either WinNonlin 4.1 or SPlus 2000 (Insightful Inc, Seattle, Wash.). All parameter calculations are reported from observed values and not from predicted values. However, observed and predicted values were very close and parameter estimates from both approaches were nearly equal.

Model 200 (extravascular input) of WinNonlin noncompartmental menu options was employed for oral doses and Model 201 (intravascular input) was employed for IV doses. Values for the following PK parameters were estimated from plasma alphaHGA concentrations:

Maximum observed concentration (Cmax) and the time to reach Cmax (Tmax), the last quantified concentration (Clast), the time to Clast (Tlast), and time to the first quantified concentration (Tfirst) came from the experimentally observed values.

Terminal elimination rate constant (λz), was estimated from log-linear regression analysis of the apparent terminal phase of the plasma concentration-time profile. Weighting for the regression was 1/Y². The associated plasma half-life (T½) was calculated as T½=ln 2/λz. The point employed for estimating λz, were selected by the WinNonlin default curve stripping algorithm.

Area-under-the-plasma-concentration-time curve from time zero to the last quantified concentration (AUClast), was calculated by the linear trapezoidal method from the observed concentrations. Area-under-the concentration-time curve from time zero to infinity (AUCinf) was obtained as the sum of AUClast and the extrapolation term Clast/λz, where Clast denotes the last observed quantifiable concentration. The percentage of AUCinf obtained by extrapolation (AUC % extrap) was calculated as [(AUCinf−AUClast)/AUCinf]*100. It should be noted that the area for the IV plasma data will result in an underestimation of the AUC in the first trapezoid because the concentration at time 0 was set to 0 and there is not reasonable way to impute a value at time 0. Area-under-the-concentration-time curve from time zero to the maximum concentration (AUCTmax) was calculated by the linear trapezoidal method. For both oral and IV doses, urine PK analysis of alphaHGA urine amount-time data was conducted using the plasma noncompartmental Model 201 (intravascular input). This approach was necessary given the nature of the parameters requested by the sponsor.

Values for the following PK parameters were estimated from urine levels of AlphaHGA during the various time intervals. Maximum fmol per collection (Dmax) and the time to reach Dmax (Tmax), the last quantified drug amount (Dlast), the time to Dlast (Tlast), and the time of the first quantified drug (Tfirst) came from the experimentally observed values. Area under the amount-time curve from time zero to the last quantified concentration (AUClast), was calculated by the linear trapezoidal method. Area under the amount-time curve from time zero to the maximum concentration (AUCTmax) was calculated by the linear trapezoidal method.

The PK parameter results from the plasma, after both oral and IV dosing are presented in TABLES 22 and 23. For all subjects the AUC % extrap was less than 20% indicating accurate estimation of the PK parameters. Drug was present at the time of the first sample in all cases. The “no points” in the table was the number of data points selected as the default by WinNonlin to estimate λz. There appears to be no period or route effect for plasma parameters. AlphaHGA appears to be completely bioavailable, based on AUCinf. The AUCinf for the oral and IV routes were 378.84 and 361.41 fmol*hr/mL, respectively. The lower AUC_{inf} for the IV route could be explained by the underestimation of the first trapezoid in the AUC calculations which occurred because the value at time 0 was set to 0. The approach to analysis and the absence of a concentration immediately after the bolus, did not allow back extrapolation to the value at time 0 or accurate estimation of the first trapezoid. T_{first} occurred at the first available sample for all subjects and for all routes of administration, indicating that absorption began soon after administration.

Urine pharmacokinetic parameters of all subjects stratified by route of administration are presented in TABLE 24. Greater urinary excretion was detected among all subjects in period 2, indicated by higher Dmax, Dlast and area under excretion-time curve. TABLE 25 presents the parameter estimates for urine data stratified by periods. Descriptive statistics of each parameter are included, and paired t-test were performed that confirm a period effect.

Accordingly, the administered doses, of 100 nCi of ¹⁴C provided ample signal to easily trace the fate of the dose in plasma and urine over the 24 hr duration of the study. Radiochemical doses of significantly less than this quantity can be enlisted for future in vivo experimentation when AMS is used. AlphaHGA was rapidly and completely absorbed after oral administration. The AUCinf value after po (oral) dosing was in fact slightly greater than for IV dosing, a value that suggests slightly greater than 100% bioavailability. While not explicable without additional analyses, the >100% availability could be partially or fully attributed to an underestimation of the first trapezoid in the IV analysis, differential carrier kinetics, or differential metabolism (e.g., gut wall metabolism for po dose) related to the routes of entry. The chemical form of the ¹⁴C label is assumed to be parent alphaHGA in this analysis; this assumption should be confirmed by additional analyses that join liquid chromatographic separations with AMS detection. The low levels pertinent to microdose studies preclude detection by traditional analytic methods. For alphaHGA administered during the second period there appeared to be an increase in the quantity of label appearing in urine. This effect was identified only in the urine (not the plasma) and confirmed by multiple analyses at Vitalea's facility using 2 analytical procedures (AMS and LSC).

In sum, pharmacokinetic parameters for alphaHGA were estimated by noncompartmental analysis. Plasma PK parameters included exposure (AUC), Cmax, Tmax, clearance and terminal elimination rate constant and half-life. Urine PK parameters included exposure, Dmax and fraction of the drug excreted in urine. The results of the experiments described in this example confirmed that AlphaHGA was completely (100%) orally bioavailable and that bioavailability was relatively consistent across all subjects. Furthermore, the compound was rapidly absorbed after oral administration and urine was the dominant excretory route. That is, AlphaHGA was rapidly and completely absorbed into the blood following oral administration and the terminal elimination half-life in the plasma was approximately 10 hours following both routes of administration. PK data were stratified by periods of administration (1 and 2) and by routes of administration (Oral and IV). There were no significant differences observed in PK parameters between the two routes of administration indicating that AlphaHGA was highly bioavailable. In terms of administered dose, cumulative recovery of the dose in the urine over 24 hr ranged from 34-100% after oral administration (mean 67:1), and 35-100% after IV administration (mean 63.7). The unrecovered dose may undergo excretion via bile or be sequested in tissue pools. Analyses of urine data stratified by period of dosing detected significant differences of PK parameters, suggesting a sequence effect. Statistically significant increase of urinary elimination of label was quantified during the second period in all subjects at each time of collection. A dosing period (time of administration) effect was observable in the urine excretion: accelerated excretion and increased cumulative elimination during the second period (after initial dose). This effect was not observed in plasma response.

TABLE 22
Individual Plasma PK Parameters for AlphaHGA

| | | | | | | | | | | | | | AUCₗₐₛₜ | AUC_{inf} | | AUC_{Tmax} |
|---|---|---|---|---|---|---|---|---|---|---|---|---|---|---|---|---|
| | | | | No. | λz | T_{1/2} | Tₘₐₓ | Cₘₐₓ | | Tₗₐₛₜ | Cₗₐₛₜ | T_{first} | (fmol * | (fmol * | | (fmol * |
| Subject | Route | Period | r² | Pts. | (hr⁻¹) | (hr) | (hr) | (fmol/mL) | C₀ | (hr) | (fmol/mL) | (hr) | hr/mL) | hr/mL) | AUC_{%extrap} | hr/mL) |
| 1 | po | 2 | 1.000 | 3 | 0.061 | 11.29 | 0.5 | 47.94 | 0 | 24 | 3.26 | 0.5 | 270.76 | 323.88 | 16.40 | 11.99 |
| 2 | po | 2 | 0.983 | 5 | 0.075 | 9.19 | 0.5 | 58.07 | 0 | 24 | 3.76 | 0.25 | 341.45 | 391.30 | 12.74 | 13.53 |
| 3 | po | 1 | 1.000 | 3 | 0.074 | 9.40 | 0.5 | 58.35 | 0 | 24 | 3.3 | 0.25 | 323.18 | 367.95 | 12.17 | 14.89 |
| 4 | po | 1 | 1.000 | 3 | 0.074 | 9.39 | 0.5 | 40.63 | 0 | 24 | 2.86 | 0.25 | 282.21 | 320.94 | 12.07 | 12.20 |
| 5 | po | 2 | 0.996 | 8 | 0.118 | 5.88 | 0.75 | 76 | 0 | 24 | 3.1 | 0.25 | 464.42 | 490.70 | 5.36 | 31.49 |
| 6 | po | 1 | 0.999 | 3 | 0.068 | 10.18 | 1 | 35.69 | 0 | 24 | 3.41 | 0.25 | 266.99 | 317.04 | 15.79 | 17.12 |
| 7 | po | 2 | 1.000 | 3 | 0.067 | 10.36 | 2 | 39.21 | 0 | 24 | 4.59 | 0.25 | 346.36 | 414.99 | 16.54 | 49.03 |
| 8 | po | 1 | 0.999 | 3 | 0.073 | 9.53 | 0.75 | 49.42 | 0 | 24 | 3.95 | 0.25 | 349.63 | 403.94 | 13.45 | 23.79 |
| 1 | iv | 1 | 1.000 | 3 | 0.069 | 10.07 | 0.25 | 33.72 | 0 | 24 | 2.67 | 0.25 | 240.83 | 279.62 | 13.87 | 4.22 |
| 2 | iv | 1 | 0.996 | 4 | 0.082 | 8.46 | 0.25 | 54.63 | 0 | 24 | 3.04 | 0.25 | 291.53 | 328.62 | 11.29 | 6.83 |
| 3 | iv | 2 | 0.994 | 3 | 0.075 | 9.31 | 0.25 | 72.17 | 0 | 24 | 2.92 | 0.25 | 327.82 | 367.03 | 10.68 | 9.02 |
| 4 | iv | 2 | 1.000 | 3 | 0.077 | 8.96 | 0.25 | 51.74 | 0 | 24 | 2.99 | 0.25 | 291.11 | 329.74 | 11.72 | 6.47 |
| 5 | iv | 1 | 1.000 | 3 | 0.084 | 8.26 | 0.5 | 66.79 | 0 | 24 | 3.34 | 0.25 | 391.84 | 431.66 | 9.23 | 23.96 |
| 6 | iv | 2 | 0.989 | 3 | 0.054 | 12.80 | 0.25 | 49.19 | 0 | 24 | 3.75 | 0.25 | 285.87 | 355.14 | 19.51 | 6.15 |
| 7 | iv | 1 | 0.999 | 3 | 0.067 | 10.32 | 0.25 | 42.23 | 0 | 24 | 4.62 | 0.25 | 325.85 | 394.63 | 17.43 | 5.28 |
| 8 | iv | 2 | 0.975 | 3 | 0.055 | 12.71 | 0.25 | 65.03 | 0 | 24 | 4.1 | 0.25 | 329.66 | 404.82 | 18.56 | 8.13 |

TABLE 23
The Mean Estimates of Plasma PK Parameters by Period of Adminstration and Route

| | | T ½ | Tmax | Cmax | Clast | AUClast | AUCinf | | AUCTmax |
|---|---|---|---|---|---|---|---|---|---|
| Statistic | Az (hr⁻¹) | (hr) | (hr) | (fmol/mL) | (fmol/mL) | (fmol * hr/mL) | (fmol * hr/mL) | AUC % Extrap | (fmol * hr/mL) |
| Period 1 | | | | | | | | | |
| Mean | 0.074 | 9.45 | 0.50 | 47.68 | 3.40 | 309.01 | 355.55 | 13.16 | 13.54 |
| SD | 0.006 | 0.76 | 0.27 | 11.64 | 0.63 | 48.45 | 52.08 | 2.59 | 7.84 |
| Min | 0.067 | 8.26 | 0.25 | 33.72 | 2.67 | 240.83 | 279.62 | 9.23 | 4.22 |
| Max | 0.084 | 10.32 | 1.00 | 66.79 | 4.62 | 391.84 | 431.66 | 17.43 | 23.96 |
| Period 2 | | | | | | | | | |
| Mean | 0.073 | 10.06 | 0.59 | 57.42 | 3.56 | 332.18 | 384.70 | 13.94 | 16.98 |
| SD | 0.020 | 2.27 | 0.60 | 12.78 | 0.59 | 60.24 | 54.13 | 4.72 | 15.32 |
| Min | 0.054 | 5.88 | 0.25 | 39.21 | 2.92 | 270.76 | 323.88 | 5.36 | 6.15 |
| Max | 0.118 | 12.80 | 2.00 | 76.00 | 4.59 | 464.42 | 490.70 | 19.51 | 49.03 |
| Route | | | | | | | | | |
| po | | | | | | | | | |
| Mean | 0.076 | 9.40 | 0.81 | 50.66 | 3.53 | 330.63 | 378.84 | 13.07 | 21.76 |
| SD | 0.018 | 1.58 | 0.51 | 13.21 | 0.55 | 63.99 | 59.66 | 3.63 | 12.90 |
| Min | 0.061 | 5.88 | 0.50 | 35.69 | 2.86 | 266.99 | 317.04 | 5.36 | 11.99 |
| Max | 0.118 | 11.29 | 2.00 | 76.00 | 4.59 | 464.42 | 490.70 | 16.54 | 49.03 |
| Route iv | | | | | | | | | |
| Mean | 0.070 | 10.11 | 0.28 | 54.44 | 3.43 | 310.56 | 361.41 | 14.04 | 8.76 |
| SD | 0.011 | 1.78 | 0.09 | 13.05 | 0.67 | 44.26 | 48.93 | 3.95 | 6.32 |
| Min | 0.054 | 8.26 | 0.25 | 33.72 | 2.67 | 240.83 | 279.62 | 9.23 | 4.22 |
| Max | 0.084 | 12.80 | 0.50 | 72.17 | 4.62 | 391.84 | 431.66 | 19.51 | 23.96 |

TABLE 24
Individual Urine PK Parameters for AlphaHGA

| | | | Tₘₐₓ | Dₘₐₓ | | Tₗₐₛₜ | T_{first} | Dₗₐₛₜ | AUCₗₐₛₜ | AUC_{Tmax} |
|---|---|---|---|---|---|---|---|---|---|---|
| Subject | Route | Period | (hr) | (fmol) | C₀ | (hr) | (hr) | (fmol) | (fmol * hr) | (fmol * hr) |
| 1 | po | 2 | 4 | 712956 | 0 | 24 | 4 | 109798 | 6093252 | 1425912 |
| 2 | po | 2 | 4 | 741313 | 0 | 24 | 4 | 103226 | 6026045 | 1482625 |
| 3 | po | 1 | 4 | 363559 | 0 | 24 | 4 | 19395 | 2618588 | 727119 |
| 4 | po | 1 | 4 | 374090 | 0 | 24 | 4 | 31737 | 2620500 | 748180 |
| 5 | po | 2 | 4 | 941085 | 0 | 24 | 4 | 80465 | 6993866 | 1882171 |
| 6 | po | 1 | 4 | 363920 | 0 | 24 | 4 | 22280 | 2696170 | 727839 |
| 7 | po | 2 | 4 | 663266 | 0 | 24 | 4 | 133040 | 6127092 | 1326533 |
| 8 | po | 1 | 4 | 355130 | 0 | 24 | 4 | 30936 | 2239453 | 710260 |
| 1 | iv | 1 | 4 | 342297 | 0 | 24 | 4 | 32699 | 2934335 | 684594 |
| 2 | iv | 1 | 4 | 271688 | 0 | 24 | 4 | 40072 | 2573446 | 543376 |
| 3 | iv | 2 | 4 | 603861 | 0 | 24 | 4 | 48888 | 5505102 | 1207722 |
| 4 | iv | 2 | 4 | 739597 | 0 | 16 | 4 | 88098 | 5554250 | 1479193 |
| 5 | iv | 1 | 4 | 319661 | 0 | 24 | 4 | 41355 | 2551259 | 639322 |
| 6 | iv | 2 | 4 | 744576 | 0 | 24 | 4 | 109317 | 6540408 | 1489152 |
| 7 | iv | 1 | 4 | 229590 | 0 | 24 | 4 | 19716 | 2368127 | 459180 |
| 8 | iv | 2 | 4 | 604909 | 0 | 24 | 4 | 114767 | 5220702 | 1209818 |

TABLE 25
Individual Urine PK Parameters for AlphaHGA*

| | | | | AUCTmax |
|---|---|---|---|---|
| | | | AUClast | (fmol * hr) |
| | Dmax (fmol) | Dlast (fmol) | (fmol * hr) | Mean |
| Period 1 | | | | |
| Mean | 327492 | 29774 | 2575235 | 654984 |
| SD | 51468 | 8610 | 208302 | 102935 |
| Min | 229590 | 19395 | 2239453 | 459180 |
| Max | 374090 | 41355 | 2934335 | 748180 |
| Period 2 | | | | |
| Mean | 718945 | 98450 | 6007590 | 1437891 |
| SD | 107114 | 25689 | 580123 | 214229 |
| Min | 603861 | 48888 | 5220702 | 1207722 |
| Max | 941085 | 133040 | 6993866 | 1882171 |
| Route po | | | | |
| Mean | 564415 | 66360 | 4426871 | 1128830 |
| SD | 228528 | 45503 | 2039866 | 457056 |
| Min | 355130 | 19395 | 2239453 | 710260 |
| Max | 941085 | 133040 | 6993866 | 1882171 |
| Route iv | | | | |
| Mean | 482022 | 61864 | 4155954 | 964045 |
| SD | 213499 | 36703 | 1705571 | 426997 |
| Min | 229590 | 19716 | 2368127 | 459180 |
| Max | 744576 | 114767 | 6540408 | 1489152 |

*The table presents mean estimates of selected urine PK parameters stratified based on period and route of administration. The parameters (Dmax, Dlast, AUClast, and AUCTmax) all differed significantly according to period at the p < 0.001 level using a paired-t-test. Such an effect was not observed in data stratified by route of administration. As the data analysis is exploratory no adjustment for multiple comparison was implemented.

### Abbreviations Used

AlphaHGA Alpha-hydroxy-glycinamide
AUCinf Area under the plasma concentration versus time curve from time 0 and extrapolated to infinity
AUC % extrap Percent of AUCinf that is extrapolated beyond the last quantified concentration
AUClast Area under the plasma concentration versus time curve from time 0 to the time of the last measured concentration
AUCTmax The area under the curve from time 0 to the time at which the maximum concentration (Cmax) occurred
Clast The last quantified concentration
Cmax Maximum concentration
% CV Percent coefficient of variation
Dlast The last quantified drug in urine
Dmax The highest quantified drug in urine
fmol Femtomoles
IV or iv Intravenous route of administration
LLOQ Lower limit of quantitation
LLNL Lawrence Livermore National Lab
λ_{z} Terminal elimination rate constant
NS No sample
PO or po Oral route of administration
PK Pharmacokinetic(s)
T_{1/2} Terminal-phase half-life
Tlast The time of Clast
Tmax Time of maximum concentration
Tfirst The time of the first quantifiable drug

The following example describes a human clinical trial that is conducted to determine the efficacy and safety of alphaHGA.

### Example 33

A phase H randomized, double-blind, placebo controlled, parallel group, safety and dose finding study of oral alpha-HGA administration to anti-retroviral treatment naïve HIV-1 patients is conducted. The study demonstrates the safety and tolerability of oral administration of alpha-HGA over a range of doses and demonstrates antiviral activity by reduction of plasma HIV RNA.

Three different dosages of alpha-HGA are compared. Treatment is for four weeks followed by a four week treatment free (study drug) follow-up period. Twelve patients on active investigational medicinal product and 4 patients on a corresponding amount of placebo are included in each dose group. Thus, a total of 48 HIV-1 positive patients are included. The dosing strategy is presented in Table 26.

TABLE 26
Dosing Strategy

| | | Dose alpha-HGA | | |
|---|---|---|---|---|
| | | (for patients on active treatment) | | |
| | | | | Total daily |
| | N of patients | | Total daily | dose |
| Group | Active (Placebo) | Dosing | dose (mg) | (mmoles/day*) |
| 1 | 12 (4) | 400 mg × 3 | 1200 | 9.5 |
| 2 | 12 (4) | 800 mg × 3 | 2400 | 19.0 |
| 3 | 12 (4) | 1200 mg × 2 | 2400 | 19.0 |

*Molecular weight is 126.54 g/mole

The tests that are conducted at each patient visit are outlined in Table 27. A pVL test and CD4+ cell count is performed 30 to 10 days prior to first dose of study drug to screen patients. At inclusion (and screening) the HIV-1 RNA and the CD4+ cell count is not less than 5000 copies/ml and 350 cells/μl respectively. An average of the screening and the baseline values for HIV-1 RNA and CD4+ cell counts is used in the evaluation while the screening values are used for inclusion.

TABLE 27

| | Study week | | | | | | |
|---|---|---|---|---|---|---|---|
| Tests Conducted | | 0² | | | | | 8 |
| During StudyEvent | Screen¹ | Baseline | 1 | 2 | 3 | 4 | Follow-up |
| Study information | x | | | | | | |
| Informed Consent | x | | | | | | |
| Demography and | | x | | | | | |
| Medical history | | | | | | | |
| Adverse events | | | x | x | x | x | x |
| Concomitant | | x | x | x | x | x | x |
| medication | | | | | | | |
| Pregnancy test³ | x | | | | | | x |
| Eligibility Criteria | | x | | | | | |
| Vital signs | | x | x | x | x | x | x |
| Physical exam. | | x | | | x | | x |
| Clinical Chemistry | x | x | x | x | x | x | x |
| and Haematology | | | | | | | |
| Plasma samples | | | x | x | | x | |
| Urine | | x | x | x | x | x | x |
| CD4/CD8⁴ | x | x | x | x | x | x | x |
| HIV-1 RNA^{4,5} | x | x | x | x | x | x | x |
| HBV and HCV⁶ | x | | | | | | |
| ECG | | x | | | | | x |
| Randomisation | | x | | | | | |
| Dispense of study | | x | x | x | x | | |
| drug | | | | | | | |
| Compliance | | | x | x | x | x | |

¹Screening is performed day −30 to −10.
²The sampling at the baseline visit is done prior to first dose.
³For females only.
⁴The screening value is used for fulfilling the inclusion criteria. All samples from day 0 and onwards are frozen at −70° C. and stored at a central laboratory. The efficacy analysis is performed in batches including all six samples (week 0, 1, 2, 3, 4, 8) from one patient. After analysis of HIV-1 RNA, the sample is kept at −70° C. for future genotype analysis if required.
⁵Samples are frozen and analyzed in case of emergence of virus potentially resistant to alpha-HGA. For patients who withdraw prematurely, a sample is obtained at the time point of withdrawal.
⁶If the patient is known to be HBV or HCV seropositive, testing is not required.

The scheduled days of hospital visits are adhered to ±3 days. If the patient misses a scheduled visit by more than three days, the reason is recorded.

Patients are included who are male or female of non-childbearing potential. Any included females are documented to be surgically sterile or post-menopausal (amenorrhea>1 year and FSH>30 mU/ml) with a negative pregnancy test. Patients are between 18 and 55 years of age; have a HIV-1 infection documented by licensed HIV antibody ELISA and confirmed either by positive HIV blood culture, positive HIV serum antigen or plasma viremia; have a CDT4+T-cell count: ≧200 350 cells/mm³ within 30 days of the first dose of the study drug; have a plasma HIV-1−RNA level of ≧5,000 copies/ml within 30 days of the first dose of study drug; and have no significant diseases in their medical history. Patients are excluded from the study who have a history of or current opportunistic infections characteristic of AIDS (Category C according to the CDC Classification System for HIV-1 Infection, 1993 Revised version); have malabsorption syndromes affecting drug absorption (e.g. Crohn's disease, chronic pancreatitis); have acute hepatitis or chronic active hepatitis; have the following laboratory parameters within 30 days prior to first dose of study drug: Hemoglobin <10.0 g/dl for men and <9.0 g/dl for women, Neutrophil count <1000/mm³, Platelet count <75,000/mm³, or AST or ALT >2.5 times the upper limit of normal; have received radiation therapy or cytotoxic chemotherapeutic agents within 4 weeks prior to the first dose of study drug; have received treatment with immunomodulating agents such as systemic corticosteroids, IL-2, α-IFN, β-IFN, or γ-IFN within 4 weeks prior to the first dose of study drug or patients who have received an HIV immunotherapeutic vaccine; have taken other prescription drugs within seven days of the first dose of study drug; have a positive hepatitis B surface antigen (HbsAg); have a positive antibody to hepatitis C(HCV); have had prior treatment with anti-retroviral therapy; are breast feeding; have been immunized within 30 days of the first dose of the study drug; have a new AIDS defining condition within 30 days of study entry; have unstable or severe intercurrent medical conditions; have serum creatinine of more than 2 times the upper normal level at the time of screening; or have liver enzyme (P-ALT) levels above 2 times the upper normal level at the time of screening.

Twelve patients in each dosage group receive active treatment and four patients in corresponding placebo control groups receive placebo. Thus, a total of 48 patients are involved in the study. Each patient is assigned a patient number. Patients withdrawn and/or that drop-out (having been randomized and initiated treatment) are not replaced. Patients are withdrawn from the study for drug-related toxicity; development of any health-related problem that interferes with study continuation; if the CDT4+T-cell count is reduced to ≦200 cells/μL; if they require prohibited concomitant medications; if they require concomitant medication that interferes with study continuation; if they request withdrawal; or if they become pregnant.

Patients are assigned to treatment groups by computer generating a randomization list that allocates patient numbers to patients in the treatment groups. At randomization each patient is assigned a consecutive patient number, which are then allocated to a treatment arm. The start of the second dose period is not initiated until all patients in dose period one have completed treatment.

The alpha-HGA is provided in vials containing 400, 800, or 1200 mg of alpha-HGA as a water solution. These solutions have a salty taste. The placebo dosages are also provided in vials containing an identical volume of a solution of sodium chloride and citric acid. All treatments are administered orally and the duration of the treatment is 4 weeks. Doses of alpha-HGA are administered per os (p.o.) two (2) or three (3) times daily, with approx. 12 or 8 hour intervals respectively by adding the content of one vial (400, 800, or 1200 mg, depending on dose) into half a glass (approximately 100 ml) of standardized fruit juice with no food intake 30 min prior and post dosing. It is recommended that alpha-HGA is taken in a fasting state. If a patient misses one or several doses, the exact time-point for the missing dose(s) and reason(s) is noted. The patient is instructed to not compensate for the lost dose, but to continue dosing correctly in the future. Patient compliance is evaluated by unused vial counts.

Safety during the administration periods is assessed by recording all adverse events; monitoring of vital signs (systolic and diastolic blood pressure, heart rate and weight); obtaining full blood count with differential and platelet count, including MCV and MCH, clinical chemistries (electrolytes, calcium, blood urea nitrogen, creatinine, uric acid, bilirubin, LDH, ALT, AST, alkaline phosphates, glucose, protein, amylase and albumin); obtaining urinalysis including protein, pH, glucose, red blood cells, white blood cells, and nitrite; performing physical examination including dermatological, throat, respiratory, cardiovascular, gastrointestinal (including hepatomegaly and splenomegaly), lymphatic, urogenital, neurological (including reflexes), and musculoskeletal (including extremities); and performing routine electrocardiograms. Adverse events are any symptom, sign, illness or experience that develops or worsens in severity during the course of the study. Inter-current illnesses or injuries are regarded as adverse events. Abnormal results of diagnostic procedures are considered adverse events if the abnormality results in study withdrawal, is associated with a serious adverse event, is associated with clinical signs or symptoms, leads to additional treatment or to further diagnostic tests, or is considered to be of clinical significance. A serious adverse event is any adverse event that is fatal, life-threatening, requires or prolongs hospital stay, results in persistent or significant disability or incapacity, results in a congenital anomaly or birth defect, or results in an important medical event (i.e., of major clinical significance).

Efficacy is assessed by obtaining total lymphocyte (absolute and percentage), CD4+, and CD8+ lymphocyte counts by flow cytometry. Samples for HIV-1 RNA copy number in plasma is taken for analysis. Unused amount of plasma is stored for future genotyping if required. A sample for determining the genotype is taken according to the schedule presented in Table 27. If a patient withdraws from treatment, a plasma sample is obtained for viral load and possible genotyping at the time of the withdrawal. The log 10 HIV-1 RNA is summarized per dose level across visits as well as the change from baseline. The number of patients with a decrease from baseline of HIV-1 RNA ≧0.5 log 10 is tabulated for each dose level. Data is analyzed with an ANOVA model using a contrast to test for linear trend among treatment groups. If there is a significant finding, pair-wise comparisons of all treatment groups is performed. If the log transformed data shows non-normality, all tests are performed using nonparametric alternatives. The CD4+ T cell count is summarized across visits per dose level.

While the present invention has been described in some detail for purposes of clarity and understanding, one skilled in the art will appreciate that various changes in form and detail can be made without departing from the true scope of the invention. All figures and tables, as well as patents, applications, and publications referred to above are hereby expressly incorporated by reference in their entireties.

1.-62. (canceled)

63. A method of inhibiting in-vitro replication of human immunodeficiency virus (HIV) comprising providing an HIV inhibiting amount of a composition comprising as an active ingredient, with or without other active ingredients, a compound of formula N: 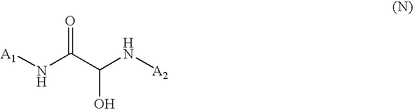
or a pharmaceutically acceptable salt, amide, or ester thereof; wherein A₁ is Valine when A2 is hydrogen and A2 is Valine when A1 is hydrogen.

64. A method of inhibiting in-vitro replication of human immunodeficiency virus (HIV) comprising providing an HIV inhibiting amount of a composition that comprises a compound of formula N: 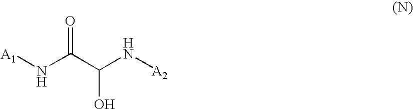
or a pharmaceutically acceptable salt, amide, or ester thereof, wherein A1 and A2 are separately selected from the group consisting of a chain of 1 to 5 amino acids and hydrogen, to a cell culture infected with HIV.

65. The method of claim 64, wherein A1 and A2 are separately selected from the group consisting of a chain of 1 to 3 amino acids and hydrogen.

66. The method of claim 64, wherein A1 and A2 are separately selected from the group consisting of a chain of 1 to 2 amino acids and hydrogen.

67. The method of claim 64, wherein A1 and A2 are separately selected from the group consisting of an amino acid and hydrogen.

68. The method of claim 64, wherein A1 and A2 are separately selected from the group consisting of Valine and hydrogen.

69. The method of claim 64, wherein the compound is Valine-alphahydroxyglycinamide.

70. The method of claim 64, further comprising measuring the presence or absence of HIV or a marker thereof in said cell culture.

71. The method of claim 70, wherein the presence or absence of HIV is measured by a p24 detection assay.

72. The method of claim 70, wherein the presence or absence of HIV is measured by reverse transcriptase activity.

73. The method of claim 70, wherein the presence or absence of HIV is measured by syncytia formation.

74. The method of claim 70, wherein said composition is provided in a septum sealed vial.

75. The method of claim 70, wherein said composition is provided in a container comprising a certification that said composition is a good manufacturing practice (OMP) formulation.

76. The method of claim 70, wherein said composition is provided in a container comprising indicia of approval from a government agency.

77. A method of inhibiting replication of human immunodeficiency virus (HIV) comprising:
identifying a subject in need of a compound that inhibits replication of HIV; and providing to said subject an HIV inhibiting amount of a composition comprising as an active ingredient, with or without other active ingredients, a compound of formula N: 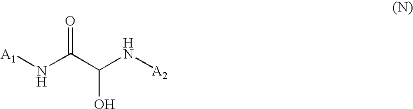
or a pharmaceutically acceptable salt, amide, or ester thereof; wherein A₁ is Valine when A2 is hydrogen and A2 is Valine when A1 is hydrogen.

78. The method of claim 77, further comprising measuring the inhibition of replication of HIV.

79. The method of claim 77, wherein said composition is formulated for oral administration.

80. A method of inhibiting replication of human immunodeficiency virus (HIV) comprising:
identifying a subject in need of a compound that inhibits replication of HIV; and providing to said subject an HIV inhibiting amount of a composition comprising as an active ingredient, with or without other active ingredients, a compound of formula N: 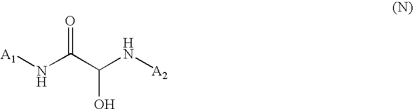
or a pharmaceutically acceptable salt, amide, or ester thereof, wherein A1 and A2 are separately selected from the group consisting of a chain of 1 to 5 amino acids and hydrogen, to a cell culture infected with HIV.

81. The method of claim 80, wherein A₁ and A₂ are separately selected from the group consisting of a chain of 1 to 3 amino acids and hydrogen.

82. The method of claim 80, wherein A₁ and A₂ are separately selected from the group consisting of a chain of 1 to 2 amino acids and hydrogen.

83. The method of claim 80, wherein A₁ and A₂ are separately selected from the group consisting of an amino acid and hydrogen.

84. The method of claim 80, wherein A₁ and A₂ are separately selected from the group consisting of Valine and hydrogen.

85. The method of claim 80, wherein the compound is Valine-alphahydroxyglycinamide.

86. The method of claim 80, further comprising measuring the inhibition of replication of HIV.

87. The method of claim 80, further comprising identifying said subject as one in need of a compound that inhibits replication of HIV.
